(19) United States
(12) Patent Application Publication
(10) Pub. No.: US 2023/0113609 A1
(43) Pub. Date: Apr. 13, 2023

(54) SSTR5 ANTAGONISTS

(71) Applicant: Kallyope, Inc., New York, NY (US)

(72) Inventors: Iyassu SEBHAT, New York, NY (US); Shuwen HE, New York, NY (US); Christopher MOYES, New York, NY (US); Simon MATHIEU, New York, NY (US); Michael LUZUNG, New York, NY (US)

(21) Appl. No.: 17/782,438

(22) PCT Filed: Dec. 2, 2020
(86) PCT No.: PCT/US2020/062898

§ 371 Date: Jun. 3, 2022

Related U.S. Application Data
(60) Provisional application No. 62/943,099, filed on Dec. 3, 2019.

(51) Int. Cl. A61K 31/438; A61K 45/06; C07D 498/10; C07D 471/10; C07D 519/00; C07F 9/6561; C07D 513/10; A61K 31/675; A61K 31/4995

(52) U.S. Cl. CPC A61K 31/438; A61K 45/06; C07D 498/10; C07D 471/10; C07D 519/00; C07F 9/6561; C07D 513/10; A61K 31/675; A61K 31/4995

(57) ABSTRACT

This disclosure is directed, at least in part, to SSTR5 antagonists useful for the treatment of conditions or disorders involving the gut-brain axis. In some embodiments, the SSTR5 antagonists are gut-restricted compounds. In some embodiments, the condition or disorder is a metabolic disorder, such as diabetes, obesity, nonalcoholic steatohepatitis (NASH), or a nutritional disorder such as short bowel syndrome.

### RELATED APPLICATIONS

This application claims the benefit of U.S. Provisional Patent Application No. 62/943,099 filed on Dec. 3, 2019, which is incorporated herein by reference in its entirety.

### BRIEF SUMMARY OF THE INVENTION

Disclosed herein, in certain embodiments, are somatostatin receptor 5 (SSTR5) antagonists useful for the treatment of conditions or disorders involving the gut-brain axis. In some embodiments, the SSTR5 antagonists are gut-restricted or selectively modulate SSTR5 located in the gut. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

Disclosed herein, in certain embodiments, is a compound of Formula (I):

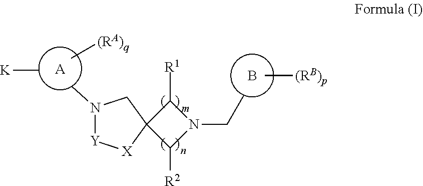

or a pharmaceutically acceptable salt, solvate, stereoisomer, or prodrug thereof, wherein:
X is —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —Z—NR⁶R⁷;
   Z is *—(CH₂)ᵣ—C(═O)—, or *—(CH₂)ᵣ—S(═O)₂—, where * represents attachment to Ring A;
   R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
   R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
      each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—;
   or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
   r is 0-4;
   each s is independently 1-6;
   each t is independently 1-6;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;
each R^{C} is independently —OH, —NH₂, —NH(R^{D}), —N(R^{D})₂, —N(R^{D})₃⁺, ═O, ═S, —C(═O)OH,

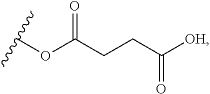 [SMILES: *OC(=O)CCC(=O)O]

### G, or G¹;

each G is independently —S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

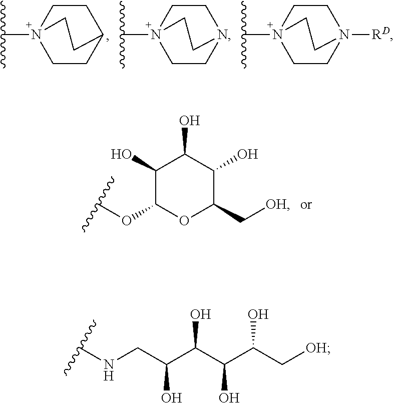

each G¹ is independently a 4- to 6-membered heterocycle which is unsubstituted or substituted with 1, 2, 3, or 4 substituents selected from C₁-C₆ alkyl, —O—(C₁-C₆ alkyl), —OH, ═O and ═S;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)2, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

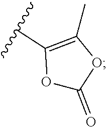 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

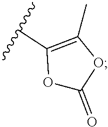 [SMILES: *c1oc(=O)oc1C]

m is 1 or 2;
n is 1 or 2;
p is 0-4; and
q is 0-4.

Also disclosed herein, in certain embodiments, is a compound of Formula (II):

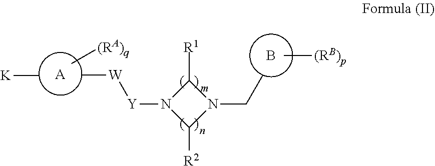

or a pharmaceutically acceptable salt, solvate, stereoisomer, or prodrug thereof, wherein:
W is a bond, —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —Z—NR⁶R⁷;
   Z is *—(CH₂)ᵣ—C(═O)—, or *—(CH₂)ᵣ—S(═O)₂—, where * represents attachment to Ring A;
   R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
   R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
      each V is independently —CH₂O—, —CH₂NR^{D}—, —CH₂N⁺(R^{D})₂—, —NH—C(═O)— NH—, —C(═O)NH—, —CH₂S(═O)₂—, or —CH₂S(═O)—;
   or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
   r is 0-4;
   each s is independently 1-6;
   each t is independently 1-6;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;
each R^{C} is independently —OH, —NH₂, —NH(R^{D}), —N(R^{D})₂, —N(R^{D})₃⁺, ═O, ═S, —C(═O)OH,

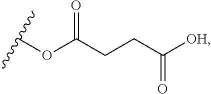 [SMILES: *OC(=O)CCC(=O)O]

### G, or G¹;

each G is independently —S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

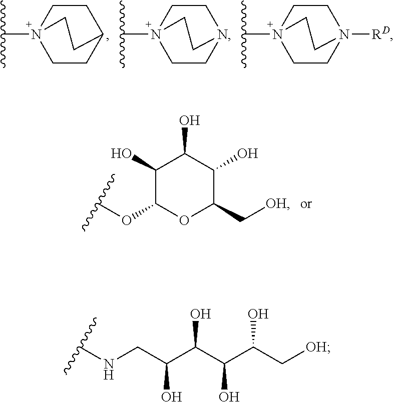

each G¹ is independently a 4- to 6-membered heterocycle which is unsubstituted or substituted with 1, 2, 3, or 4 substituents selected from C₁-C₆ alkyl, -O-(C₁-C₆ alkyl), —OH, ═O and ═S;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

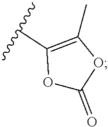 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

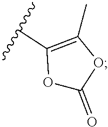 [SMILES: *c1oc(=O)oc1C]

m is 1 or 2;
n is 1 or 2;
p is 1-4; and
q is 0-4.

Disclosed herein, in certain embodiments, are pharmaceutical compositions comprising a compound disclosed herein, or a pharmaceutically acceptable salt, solvate, stereoisomer, or prodrug thereof, and at least one pharmaceutically acceptable excipient.

Disclosed herein, in certain embodiments, are methods of treating a condition or disorder involving the gut-brain axis in a subject in need thereof, the method comprising administering to the subject a therapeutically effective amount of a compound disclosed herein, or a pharmaceutically acceptable salt, solvate, stereoisomer, or prodrug thereof. In some embodiments, the condition or disorder is associated with SSTR5 activity. In some embodiments, the condition or disorder is a metabolic disorder. In some embodiments, the condition or disorder is type 2 diabetes, hyperglycemia, metabolic syndrome, obesity, hypercholesterolemia, nonalcoholic steatohepatitis, or hypertension. In some embodiments, the condition or disorder is a nutritional disorder. In some embodiments, the condition or disorder is short bowel syndrome, intestinal failure, or intestinal insufficiency.

In some embodiments, the condition or disorder is gastrointestinal injury resulting from toxic insults such as radiation or chemotherapy.

In some embodiments, disclosed herein are methods of augmenting weight loss or preventing weight gain or weight regain, the method comprising administering to the subject a therapeutically effective amount of a compound disclosed herein, or a pharmaceutically acceptable salt, solvate, stereoisomer, or prodrug thereof. In some embodiments, the subject has had bariatric surgery.

In some embodiments, the compound disclosed herein is gut-restricted. In some embodiments, the compound disclosed herein has low systemic exposure.

In some embodiments, the methods disclosed herein further comprise administering one or more additional therapeutic agents to the subject. In some embodiments, the one or more additional therapeutic agents are selected from a TGR5 agonist, a GPR40 agonist, a GPR119 agonist, a CCK1 agonist, a PDE4 inhibitor, a DPP-4 inhibitor, a GLP-1 receptor agonist, metformin, or a combination thereof. In some embodiments, the TGR5 agonist, GPR40 agonist, GPR119 agonist, or CCK1 agonist is gut-restricted.

Also disclosed herein, in certain embodiments, is the use of a compound disclosed herein, or a pharmaceutically acceptable salt, solvate, stereoisomer, or prodrug thereof, for the preparation of a medicament for the treatment of a condition or disorder involving the gut-brain axis in a subject in need thereof.

Also disclosed herein, in certain embodiments, are methods of treating a condition or disorder involving the gut-brain axis in a subject in need thereof, the method comprising administering to the subject a therapeutically effective amount of a gut-restricted SSTR5 modulator.

Also disclosed herein, in certain embodiments, is the use of a gut-restricted SSTR5 modulator for the preparation of a medicament for the treatment of a condition or disorder involving the gut-brain axis in a subject in need thereof.

### DETAILED DESCRIPTION OF THE INVENTION

This disclosure is directed, at least in part, to SSTR5 antagonists useful for the treatment of conditions or disorders involving the gut-brain axis. In some embodiments, the SSTR5 antagonists are gut-restricted compounds.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulas, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

“Alkyl” refers to an optionally substituted straight-chain, or optionally substituted branched-chain saturated hydrocarbon monoradical having from one to about ten carbon atoms, or more preferably, from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3 -dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁C₈ alkyl, a C₁C₇ alkyl, a C₁-C₆ alkyl, a C₁-C₅ alkyl, a C₁C₄ alkyl, a C₁-C₃ alkyl, a C₁-C₂ alkyl, or a C₁ alkyl. Unless stated otherwise specifically in the specification, an alkyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{a}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon or an sp³-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl (—C(CH₃)═CH₂), butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. Unless stated otherwise specifically in the specification, an alkenyl group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms, wherein an sp-hybridized carbon or an sp³-hybridized carbon of the alkynyl residue is attached to the rest of the molecule by a single bond. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂C₁₀ alkynyl, a C₂C₉ alkynyl, a C₂C₈ alkynyl, a C₂C₇ alkynyl, a C₂C₆ alkynyl, a C₂C₅ alkynyl, a C₂C₄ alkynyl, a C₂C₃ alkynyl, or a C₂ alkynyl. Unless stated otherwise specifically in the specification, an alkynyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. Unless stated otherwise specifically in the specification, an alkylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), -S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. Unless stated otherwise specifically in the specification, an alkenylene group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, -C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. Unless stated otherwise specifically in the specification, an alkynylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkoxy” or “alkoxyl” refers to a radical bonded through an oxygen atom of the formula-O-alkyl, where alkyl is an alkyl chain as defined above.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from 6 to 18 carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some embodiments, the aryl is a C₆-C₁₀ aryl. In some embodiments, the aryl is a phenyl. Unless stated otherwise specifically in the specification, the term “aryl” or the prefix “ar-” (such as in “aralkyl”) is meant to include aryl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, R^{b}—OR^{a}, —R^{b}—Sr^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

An “arylene” refers to a divalent radical derived from an “aryl” group as described above linking the rest of the molecule to a radical group. The arylene is attached to the rest of the molecule through a single bond and to the radical group through a single bond. In some embodiments, the arylene is a phenylene. Unless stated otherwise specifically in the specification, an arylene group is optionally substituted as described above for an aryl group.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃C₈ cycloalkyl), from three to six carbon atoms (C₃-C₆ cycloalkyl), from three to five carbon atoms (C₃C₅ cycloalkyl), or three to four carbon atoms (C₃C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, 7,7-dimethyl-bicyclo[2.2.1]heptanyl, and the like. Unless otherwise stated specifically in the specification, the term “cycloalkyl” is meant to include cycloalkyl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R_{b}—N⁺(R^{a})₃, —R^{b}—C(O)R^{a}, —R^{b}C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “cycloalkylene” refers to a divalent radical derived from a “cycloalkyl” group as described above linking the rest of the molecule to a radical group. The cycloalkylene is attached to the rest of the molecule through a single bond and to the radical group through a single bond. Unless stated otherwise specifically in the specification, a cycloalkylene group is optionally substituted as described above for a cycloalkyl group.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more hydroxy radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like.

“Haloalkoxy” or “haloalkoxyl” refers to an alkoxyl radical, as defined above, that is substituted by one or more halo radicals, as defined above.

“Fluoroalkoxy” or “fluoroalkoxyl” refers to an alkoxy radical, as defined above, that is substituted by one or more fluoro radicals, as defined above, for example, trifluoromethoxy, difluoromethoxy, fluoromethoxy, and the like.

“Hydroxyalkyl” refers to an alkyl radical, as defined above, that is substituted by one or more halo radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 8-membered heterocycloalkyl. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxo1-4-yl, and 2-oxo-1,3-dioxo1-4-yl. The term heterocycloalkyl also includes all ring forms of the carbohydrates, including but not limited to the monosaccharides, the disaccharides and the oligosaccharides. More preferably, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals.

“C-heterocycloalkyl ” refers to a heterocycloalkyl radical as defined above and where the point of attachment of the heterocycloalkyl radical to the rest of the molecule is through a carbon atom in the heterocycloalkyl radical. A C-heterocycloalkyl radical is optionally substituted as described above for heterocycloalkyl radicals.

A “heterocycloalkylene” refers to a divalent radical derived from a “heterocycloalkyl” group as described above linking the rest of the molecule to a radical group. The heterocycloalkylene is attached to the rest of the molecule through a single bond and to the radical group through a single bond. Unless stated otherwise specifically in the specification, a heterocycloalkylene group is optionally substituted as described above for a heterocycloalkyl group.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In some embodiments, the heteroaryl is a 5- to 10-membered heteroaryl. In some embodiments, the heteroaryl is a monocyclic heteroaryl, or a monocyclic 5- or 6-membered heteroaryl. In some embodiments, the heteroaryl is a 6,5-fused bicyclic heteroaryl. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “heteroarylene” refers to a divalent radical derived from a “heteroaryl” group as described above linking the rest of the molecule to a radical group. The heteroarylene is attached to the rest of the molecule through a single bond and to the radical group through a single bond. Unless stated otherwise specifically in the specification, a heteroarylene group is optionally substituted as described above for a heteroaryl group.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF_{2,} etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “modulate” or “modulating” or “modulation” refers to an increase or decrease in the amount, quality, or effect of a particular activity, function or molecule. By way of illustration and not limitation, agonists, partial agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or target enzyme and activates the receptor or enzyme to produce a biological response. By way of example, “GPR119 agonist” can be used to refer to a compound that exhibits an EC₅₀ with respect to GPR119 activity of no more than about 100 μM, as measured in the as measured in the inositol phosphate accumulation assay. In some embodiments, the term “agonist” includes full agonists or partial agonists.

The term “full agonist” refers to a modulator that binds to and activates a receptor or target enzyme with the maximum response that an agonist can elicit at the receptor or enzyme.

The term “partial agonist” refers to a modulator that binds to and activates a receptor or target enzyme, but has partial efficacy, that is, less than the maximal response, at the receptor or enzyme relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or target enzyme by a modulator, or antagonist. Antagonism of a receptor, for example, is when a molecule binds to the receptor or target enzyme and does not allow activity to occur.

The term “antagonist” or “neutral antagonist” as used herein refers to a modulator that binds to a receptor or target enzyme and blocks a biological response. By way of example, “SSTR5 antagonist” can be used to refer to a compound that exhibits an IC₅₀ with respect to SSTR5 activity of no more than about 100 μM, as measured in the as measured in the inositol phosphate accumulation assay. An antagonist has no activity in the absence of an agonist or inverse agonist but can block the activity of either, causing no change in the biological response.

The term “inverse agonist” refers to a modulator that binds to the same receptor or target enzyme as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of an agonist (e.g., a GPR119 agonist) that is required for 50% activation of a receptor or target enzyme (e.g., GPR119).

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an SSTR5 antagonist) that is required for 50% inhibition of a receptor or a target enzyme (e.g., SSTR5).

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., an SSTR5 antagonist, that is predominantly active in the gastrointestinal system. In some embodiments, the biological activity of the gut-restricted compound, e.g., a gut-restricted SSTR5 antagonist, is restricted to the gastrointestinal system. In some embodiments, gastrointestinal concentration of a gut-restricted modulator, e.g., a gut-restricted SSTR5 antagonist, is higher than the IC₅₀ value or the EC₅₀ value of the gut-restricted modulator against its receptor or target enzyme, e.g., SSTR5, while the plasma levels of said gut-restricted modulator, e.g., gut-restricted SSTR5 antagonist, are lower than the IC₅₀ value or the EC₅₀ value of the gut-restricted modulator against its receptor or target enzyme, e.g., SSTR5. In some embodiments, the gut-restricted compound, e.g., a gut-restricted SSTR5 antagonist, is non-systemic. In some embodiments, the gut-restricted compound, e.g., a gut-restricted SSTR5 antagonist, is a non-absorbed compound. In other embodiments, the gut-restricted compound, e.g., a gut-restricted SSTR5 antagonist, is absorbed, but is rapidly metabolized to metabolites that are significantly less active than the modulator itself toward the target receptor or enzyme, i.e., a “soft drug.” In other embodiments, the gut-restricted compound, e.g., a gut-restricted SSTR5 antagonist, is minimally absorbed and rapidly metabolized to metabolites that are significantly less active than the modulator itself toward the target receptor or enzyme.

In some embodiments, the gut-restricted modulator, e.g., a gut-restricted SSTR5 antagonist, is non-systemic but is instead localized to the gastrointestinal system. For example, the modulator, e.g., a gut-restricted SSTR5 antagonist, may be present in high levels in the gut, but low levels in serum. In some embodiments, the systemic exposure of a gut-restricted modulator, e.g., a gut-restricted SSTR5 antagonist, is, for example, less than 100, less than 50, less than 20, less than 10, or less than 5 nM, bound or unbound, in blood serum. In some embodiments, the intestinal exposure of a gut-restricted modulator, e.g., a gut-restricted SSTR5 antagonist, is, for example, greater than 1000, 5000, 10000, 50000, 100000, or 500000 nM. In some embodiments, a modulator, e.g., a SSTR5 antagonist, is gut-restricted due to poor absorption of the modulator itself, or because of absorption of the modulator which is rapidly metabolized in serum resulting in low systemic circulation, or due to both poor absorption and rapid metabolism in the serum. In some embodiments, a modulator, e.g., a SSTR5 antagonist, is covalently bonded to a kinetophore, optionally through a linker, which changes the pharmacokinetic profile of the modulator.

In particular embodiments, the gut-restricted SSTR5 antagonist is a soft drug. The term “soft drug” as used herein refers to a compound that is biologically active but is rapidly metabolized to metabolites that are significantly less active than the compound itself toward the target receptor. In some embodiments, the gut-restricted SSTR5 antagonist is a soft drug that is rapidly metabolized in the blood to significantly less active metabolites. In some embodiments, the gut-restricted SSTR5 antagonist is a soft drug that is rapidly metabolized in the liver to significantly less active metabolites. In some embodiments, the gut-restricted SSTR5 antagonist is a soft drug that is rapidly metabolized in the blood and the liver to significantly less active metabolites. In some embodiments, the gut-restricted SSTR5 antagonist is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug gut-restricted SSTR5 antagonist.

The term “kinetophore” as used herein refers to a structural unit tethered to a small molecule modulator, e.g., an SSTR5 antagonist, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator, e.g., an SSTR5 antagonist, and has minimal changes to the binding to or association with a receptor or target enzyme. The defining feature of a kinetophore is not its interaction with the target, for example a receptor, but rather its effect on specific physiochemical characteristics of the modulator to which it is attached, e.g., an SSTR5 antagonist. In some instances, kinetophores are used to restrict a modulator, e.g., an SSTR5 antagonist, to the gut.

The term “linked” as used herein refers to a covalent linkage between a modulator, e.g., an SSTR5 antagonist, and a kinetophore. The linkage can be through a covalent bond, or through a “linker.” As used herein, “linker” refers to one or more bifunctional molecules which can be used to covalently bond to the modulator, e.g., an SSTR5 antagonist, and kinetophore. In some embodiments, the linker is attached to any part of the modulator, e.g., an SSTR5 antagonist, so long as the point of attachment does not interfere with the binding of the modulator to its receptor or target enzyme. In some embodiments, the linker is non-cleavable. In some embodiments, the linker is cleavable. In some embodiments, the linker is cleavable in the gut. In some embodiments, cleaving the linker releases the biologically active modulator, e.g., an SSTR5 antagonist, in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

### Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need. Diseases and conditions affected by the gut-brain axis, include central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

### SSTR5 in the Gut-Brain Axis

Somatostatin acts at many sites to inhibit the release of many hormones and other secretory proteins. Somatostatin is predominantly expressed in two forms, SST-14 in gastric and pancreatic delta cells and neurons and SST-28 in intestinal muscosal cells. In some instances, the biological effects of somatostatin are mediated by a family of G protein-coupled receptors that are expressed in a tissue-specific manner. SSTR5 is a member of the superfamily of receptors and is expressed on β cells of pancreatic islets, GI epithelium and enteroendocrine cells, and cardiac tissue. In some instances, somatostatin binding to SSTR5 inhibits the release of GLP-1, GLP-2, GIP, PYY, or other hormones in enteroendocrine cells. SSTR5 antagonists may be useful in the treatment of metabolic disorders such as diabetes and obesity, and other diseases involving the gut-brain axis.

In some instances, inhibiting SSTR5 activity results in an elevated level of GLP-1, GLP-2, GIP, PYY, and other hormones in enteroendocrine cells. In some instances, modulators of SSTR5, for example, SSTR5 antagonists, facilitate the release of GLP-1, GLP-2, GIP, PYY, and other hormones in enteroendocrine cells by blocking the activity of somatostatin. In some instances, modulators of SSTR5, for example, SSTR5 antagonists, lead to increased cAMP levels by blocking the activity of somatostatin. In some instances, SSTR5 activity, upon binding of somatostatin, inhibits intracellular cAMP production and GLP-1, GLP-2, GIP, PYY, and other hormone secretion. In some instances, inhibiting SSTR5 activity results in elevated intracellular cAMP levels and elevated GLP-1, GIP, PYY, or other hormone secretion. In some instances, inhibiting SSTR5 activity results in elevated intracellular cAMP levels and elevated GLP-1 secretion.

Described herein is a method of treating a condition or disorder involving the gut-brain axis in an individual in need thereof, the method comprising administering to the individual a SSTR5 receptor antagonist. In other embodiments, the method comprises administering to the individual a SSTR5 inverse agonist.

In some embodiments, the condition or disorder involving the gut-brain axis is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is type 2 diabetes, hyperglycemia, metabolic syndrome, obesity, hypercholesterolemia, nonalcoholic steatohepatitis, or hypertension. In some embodiments, the metabolic disorder is diabetes. In other embodiments, the metabolic disorder is obesity. In other embodiments, the metabolic disorder is nonalcoholic steatohepatitis. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome. In some embodiments, the condition involving the gut-brain axis is gastrointestinal injury. In some embodiments, the condition involving the gut-brain axis is gastrointestinal injury resulting from toxic insults such as radiation or chemotherapy. In some embodiments, the condition involving the gut-brain axis is weight loss or preventing weight gain or weight regain. In some embodiments, the condition involving the gut-brain axis is weight loss or preventing weight gain or weight regain post-bariatric surgery. In some embodiments, the condition involving the gut-brain axis is weight loss or preventing weight gain or weight regain, wherein the subject has had bariatric surgery.

### Gut-Restricted Antagonists

In some instances, differentiation of systemic effects of an SSTR5 antagonist from beneficial, gut-driven effects would be critical for the development of an SSTR5 antagonist for the treatment of disease.

In some embodiments, the SSTR5 antagonist is gut-restricted. In some embodiments, the SSTR5 antagonist is designed to be substantially non-permeable or substantially non-bioavailable in the blood stream. In some embodiments, the SSTR5 antagonist is designed to inhibit SSTR5 activity in the gut and is substantially non-systemic. In some embodiments, the SSTR5 antagonist has low systemic exposure.

In some embodiments, a gut-restricted SSTR5 antagonist has low oral bioavailability. In some embodiments, a gut-restricted SSTR5 antagonist has <10% oral bioavailability, <8% oral bioavailability, <5% oral bioavailability, <3% oral bioavailability, or <2% oral bioavailability.

In some embodiments, the unbound plasma levels of a gut-restricted SSTR5 antagonist are lower than the IC₅₀ value of the SSTR5 antagonist against SSTR5. In some embodiments, the unbound plasma levels of a gut-restricted SSTR5 antagonist are significantly lower than the IC₅₀ value of the gut-restricted SSTR5 antagonist against SSTR5. In some embodiments, the unbound plasma levels of the SSTR5 antagonist are 2-fold, 10-fold, 20-fold, 30-fold, 40-fold, 50-fold, or 100-fold lower than the IC₅₀ value of the gut-restricted SSTR5 antagonist against SSTR5.

In some embodiments, a gut-restricted SSTR5 antagonist has low systemic exposure. In some embodiments, the systemic exposure of a gut-restricted SSTR5 antagonist is, for example, less than 500, less than 200, less than 100, less than 50, less than 20, less than 10, or less than 5 nM, bound or unbound, in blood serum. In some embodiments, the systemic exposure of a gut-restricted SSTR5 antagonist is, for example, less than 500, less than 200, less than 100, less than 50, less than 20, less than 10, or less than 5 ng/mL, bound or unbound, in blood serum.

In some embodiments, a gut-restricted SSTR5 antagonist has low permeability. In some embodiments, a gut-restricted SSTR5 antagonist has low intestinal permeability. In some embodiments, the permeability of a gut-restricted SSTR5 antagonist is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted SSTR5 antagonist has low absorption. In some embodiments, the absorption of a gut-restricted SSTR5 antagonist is less than less than 20%, or less than 10%, less than 5%, or less than 1%.

In some embodiments, a gut-restricted SSTR5 antagonist has high plasma clearance. In some embodiments, a gut-restricted SSTR5 antagonist is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments of the methods described herein, the SSTR5 antagonist is gut-restricted. In some embodiments, the SSTR5 antagonist is covalently bonded to a kinetophore. In some embodiments, the SSTR5 antagonist is covalently bonded to a kinetophore through a linker. In some embodiments, the SSTR5 antagonist is a soft drug.

In other embodiments, the methods described herein comprise administering an SSTR5 inverse agonist. In some emboidments, the SSTR5 inverse agonist is gut-restricted. In some embodiments, the SSTR5 inverse agonist is covalently bonded to a kinetophore. In some embodiments, the SSTR5 inverse agonist is covalently bonded to a kinetophore through a linker. In some embodiments, the SSTR5 inverse agonist is a soft drug.

### Compounds

Disclosed herein, in certain embodiments, is a compound of Formula (I):

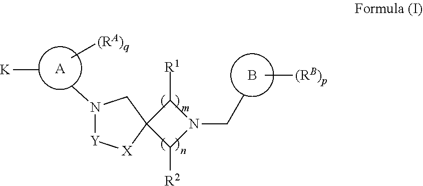

or a pharmaceutically acceptable salt, solvate, stereoisomer, or prodrug thereof, wherein:

X is —O—, —NR³—, or —C(R⁴)₂—;

Y is —C(═O)—, or —S(═O)₂—;

Ring A is aryl, heteroaryl, cycloalkyl, or heterocycloalkyl;

Ring B is aryl or heteroaryl;

K is —Z—NR⁶R⁷;
Z is *—(CH₂)ᵣ—C(═O)—, or *—(CH₂)ᵣ—S(═O)₂—, where * represents attachment to Ring A;
R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
   each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—;
or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
r is 0-4;
each s is independently 1-6;
each t is independently 1-6;

each R¹ and R² is independently hydrogen, C₁₋₆ alkyl, or C₁₋₆ fluoroalkyl;

or one R¹ and one R² are taken together to form a ring;

R³ is hydrogen, C₁₋₆ alkyl, C₁₋₆ fluoroalkyl, or C₃₋₆ cycloalkyl;

each R⁴ is independently hydrogen, C₁₋₆ alkyl, C₁₋₆ fluoroalkyl, or C₃₋₆ cycloalkyl;

R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;

each R^{C} is independently —OH, —NH₂, —NH(R^{D}), —N(R^{D})₂, —N(R^{D})₃⁺, ═O, ═S, —C(═O)OH,

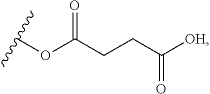 [SMILES: *OC(=O)CCC(=O)O]

### G, or G¹;

each G is independently —S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

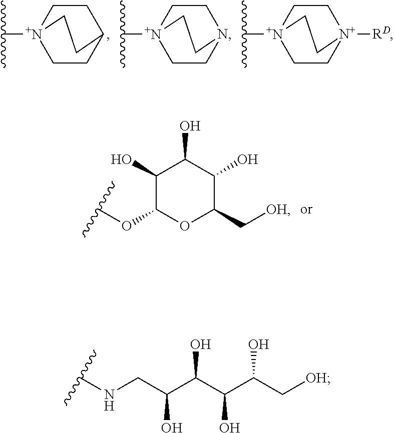

each G¹ is independently a 4- to 6-membered heterocycle which is unsubstituted or substituted with 1, 2, 3, or 4 substituents selected from C₁-C₆ alkyl, —O—(C₁-C₆ alkyl), —OH, ═O and ═S;

each R^{D} is independently C₁₋₆ alkyl or C₃₋₆ cycloalkyl; wherein the alkyl and cycloalkyl are unsubstituted or substituted by 1-3 halogen or —OH groups;

each R^{A} is independently halogen, —OH, —O-(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O-(C₁-C₆ alkyl), C₁-C₆ alkyl, C₁-C₆ fluoroalkyl, C₁-C₆ hydroxyalkyl, —O-(C₁-C₆ fluoroalkyl), C₃-C₆ cycloalkyl, and 3- to 6-membered heterocycloalkyl;

each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;

each R⁹ is independently selected from hydrogen, C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

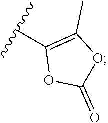 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;

each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

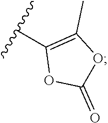 [SMILES: *c1oc(=O)oc1C]

m is 1 or 2;

n is 1 or 2;

p is 0-4; and

q is 0-4.

In some embodiments, each R¹ and R² is independently hydrogen, C₁₋₆ alkyl, or C₁₋₆ fluoroalkyl. In some embodiments, each R¹ and R² is independently hydrogen or C₁₋₆ alkyl. In some embodiments, each R¹ and R² is independently —H, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂F, —CHF₂, —CF₃, —CH₂CH₂F, —CH₂CHF₂, or —CH₂CF₃. In some embodiments, each R¹ and R² is independently —H, —CH₃, —CH₂CH₃, or —CH₂CH₂CH₃. In some embodiments, each R¹ and R² is —H.

In some embodiments, one R¹ and one R² are taken together to form a ring. In some embodiments, one R¹ and one R² are taken together to form a 3- to 6-membered heterocycloalkyl ring.

In some embodiments, m is 1. In some embodiments, m is 2. In some embodiments, n is 1. In some embodiments, n is 2. In some embodiments, m is 1 and n is 1. In some embodiments, m is 1 and n is 2. In some embodiments, m is 2 and n is 1. In some embodiements, m is 2 and n is 2.

In some embodiments, Ring B is phenyl, naphthyl, monocyclic 6-membered heteroaryl, monocyclic 5-membered heteroaryl, or bicyclic heteroaryl.

In some embodiments, Ring B is phenyl or monocyclic heteroaryl. In some embodiments, Ring B is phenyl, monocyclic 6-membered heteroaryl, or monocyclic 5-membered heteroaryl. In some embodiments, Ring B is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring B is phenyl or 6-membered heteroaryl. In some embodiments, Ring B is phenyl, pyridinyl, pyrimidinyl, pyrazinyl, or pyridazinyl.

In some embodiments, Ring B is phenyl, or pyridinyl.

In some embodiments, Ring B is

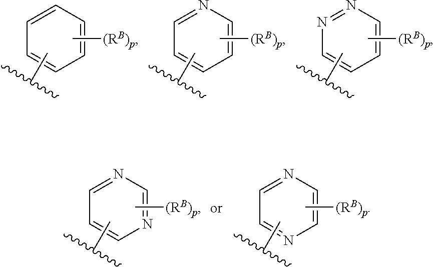

In some embodiments, Ring B is

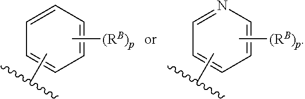

In some embodiments, Ring B is

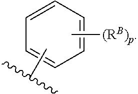

In some embodiments, Ring B is

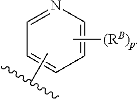

In some embodiments, Ring B is

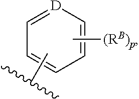

where D is CH or N.

In some embodiments, Ring B is phenyl or 6-membered heteroaryl; each R¹ and R² is independently hydrogen or C₁₋₆ alkyl; m is 2; and n is 2.

In some embodiments, the compound of Formula (I) has the structure of Formula (Ia), or a pharmaceutically acceptable salt, solvate, stereoisomer, or prodrug thereof:

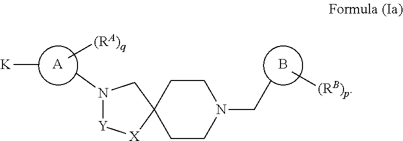

In some embodiments, the compound of Formula (I) has the structure of Formula (Ia-1), or a pharmaceutically acceptable salt, solvate, stereoisomer, or prodrug thereof:

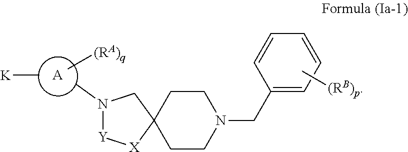

In some embodiments, the compound of Formula (I) has the structure of Formula (Ia-2), or a pharmaceutically acceptable salt, solvate, stereoisomer, or prodrug thereof:

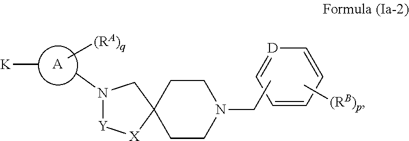

wherein D is CH or N.

In some embodiments, the compound of Formula (I) has the structure of Formula (Ia-3), or a pharmaceutically acceptable salt, solvate, stereoisomer, or prodrug thereof:

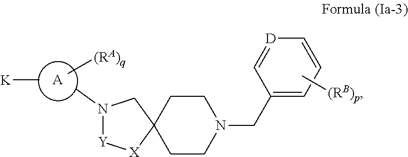

wherein D is CH or N.

In some embodiments, X is —O—. In some embodiments, X is —NR³—. In some embodiments, X is —C(R⁴)₂—.

In some embodiments, Y is —C(═O)—. In some embodiments, Y is —S(═O)₂—.

In some embodiments, X is —O—, and Y is —C(═O)—. In some embodiments, X is —NR³—, and Y is —C(═O)—. In some embodiments, X is —C(R⁴)₂—; and Y is —C(═O)—. In some embodiments, X is —O—, and Y is —S(═O)₂—. In some embodiments, X is —NR³—, and Y is —S(═O)₂—. In some embodiments, X is —C(R⁴)₂—; and Y is —S(═O)_{2—.}

In some embodiments, X is —O—, and Y is —C(═O)—; or X is —NR³—, and Y is —C(═O)—; or X is —C(R⁴)₂—; and Y is —C(═O)—; or X is —O—, and Y is —S(═O)₂—; or X is —NR³—, and Y is —S(═O)₂—; or X is —C(R⁴)₂—; and Y is —S(═O)₂—. In some embodiments, X is —O—, and Y is —C(═O)—; or X is —NR³—, and Y is —C(═O)—; or X is —C(R⁴)₂—; and Y is —C(═O)—; or X is —NR³—, and Y is —S(═O)₂—.

In some embodiments, X is —NR³—, and Y is —C(═O)—; or X is —C(R⁴)₂—; and Y is —C(═O)—; or X is —O—, and Y is —S(═O)₂—; or X is —NR³—, and Y is —S(═O)₂—; or X is —C(R⁴)₂—; and Y is —S(═O)₂—.

In some embodiments, the compound of Formula (I) has the structure of Formula (Ib), Formula (Ic), Formula (Id), or Formula (Ie), or a pharmaceutically acceptable salt, solvate, stereoisomer, or prodrug thereof:

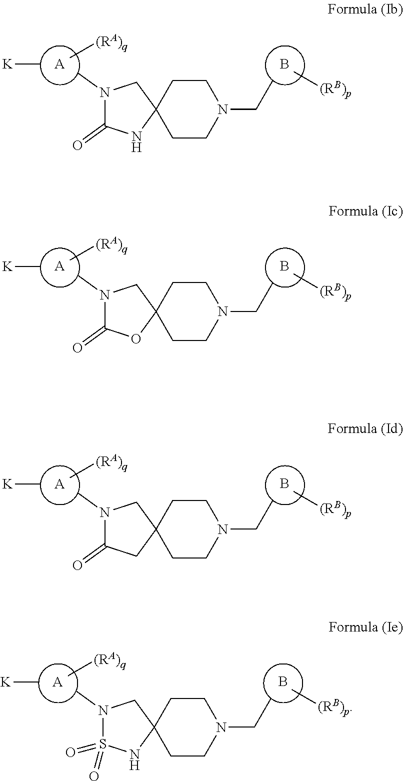

In some embodiments, the compound of Formula (I) has the structure of Formula (Ib), Formula (Ic), or Formula (Id), or a pharmaceutically acceptable salt, solvate, stereoisomer, or prodrug thereof. In some embodiments, the compound of Formula (I) has the structure of Formula (Ib) or Formula (Ic). In some embodiments, the compound of Formula (I) has the structure of Formula (Ib). In some embodiments, the compound of Formula (I) has the structure of Formula (Ic). In some embodiments, the compound of Formula (I) has the structure of Formula (Id). In some embodiments, the compound of Formula (I) has the structure of Formula (Ie).

In some embodiments, the compound of Formula (I) has the structure of Formula (Ib), or a pharmaceutically acceptable salt, solvate, stereoisomer, or prodrug thereof:

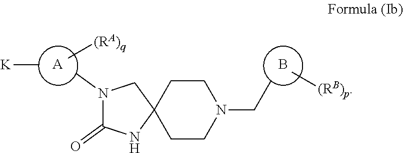

In some embodiments, the compound of Formula (I) has the structure of Formula (Ib-1), (Ib-2), or (Ib-3), or a pharmaceutically acceptable salt, solvate, stereoisomer, or prodrug thereof:

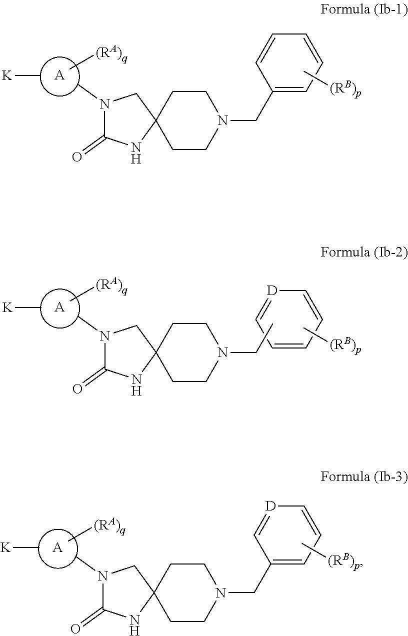

wherein D is CH or N.

In some embodiments, the compound of Formula (I) has the structure of Formula (Ic), or a pharmaceutically acceptable salt, solvate, stereoisomer, or prodrug thereof:

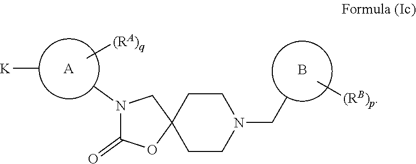

In some embodiments, the compound of Formula (I) has the structure of Formula (Ic-1), (Ic-2), or (Ic-3), or a pharmaceutically acceptable salt, solvate, stereoisomer, or prodrug thereof:

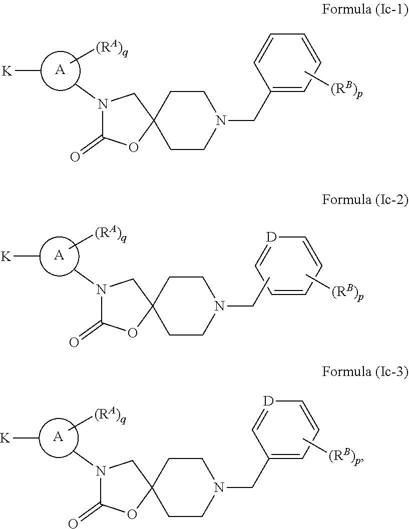

wherein D is CH or N.

In some embodiments, the compound of Formula (I) has the structure of Formula (Id), or a pharmaceutically acceptable salt, solvate, stereoisomer, or prodrug thereof:

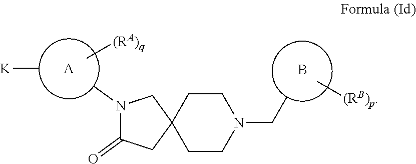

In some embodiments, the compound of Formula (I) has the structure of Formula (Id-1), (Id-2), or (Id-3), or a pharmaceutically acceptable salt, solvate, stereoisomer, or prodrug thereof:

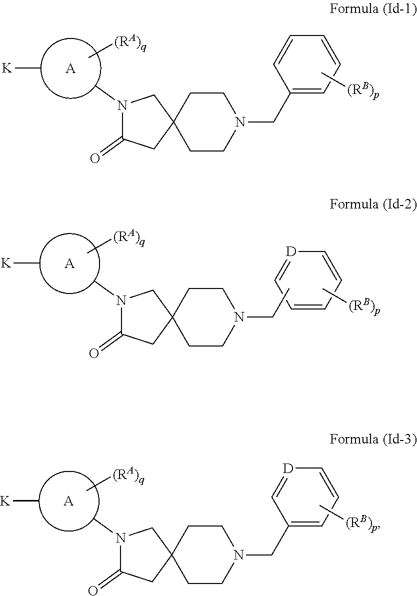

wherein D is CH or N.

In some embodiments, the compound of Formula (I) has the structure of Formula (Ie), or a pharmaceutically acceptable salt, solvate, stereoisomer, or prodrug thereof:

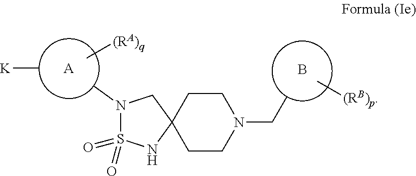

In some embodiments, the compound of Formula (I) has the structure of Formula (Ie-1), (Ie-2), or (Ie-3), or a pharmaceutically acceptable salt, solvate, stereoisomer, or prodrug thereof:

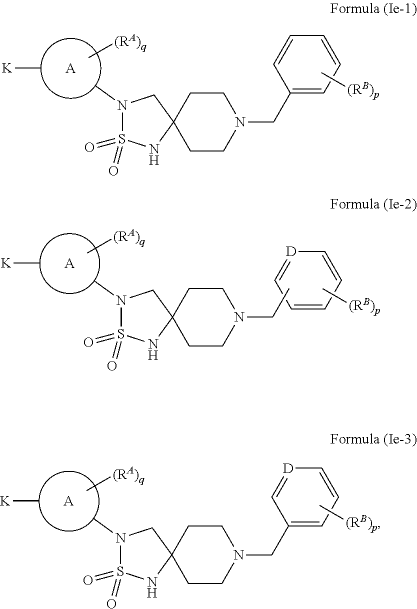

wherein D is CH or N.

In some embodiments, each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O— (C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl. In some embodiments, each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH2OH, —O—(C₁-C₆ alkyl), C₁-C₆ alkyl, C₁-C₆ fluoroalkyl. In some embodiments, each R^{B} is independently phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, and C₁-C₆ fluoroalkyl.

In some embodiments, p is 0. In some embodiments, p is 1. In some embodiments, p is 2. In some embodiments, p is 3. In some embodiments, p is 4. In some embodiments, p is 1-4. In some embodiments, p is 2 or 3.

In some embodiments, each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(0R⁹)2, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and p is 1-4.

In some embodiments, the compound of Formula (I) has the structure of Formula (If), or a pharmaceutically acceptable salt, solvate, stereoisomer, or prodrug thereof:

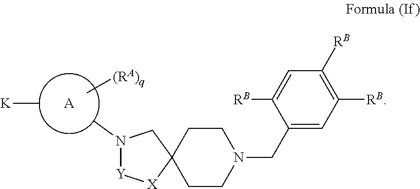

In some embodiments, the compound of Formula (I) has the structure of Formula (Ig), or a pharmaceutically acceptable salt, solvate, stereoisomer, or prodrug thereof:

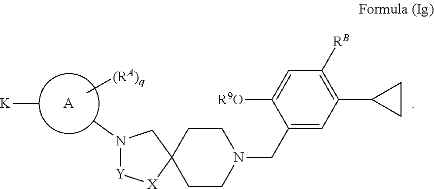

In some embodiments, R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

In some embodiments, Ring A is phenyl, naphthyl, monocyclic 6-membered heteroaryl, monocyclic 5-membered heteroaryl, bicyclic heteroaryl, monocyclic C₃-C₈ cycloalkyl, bridged C₅-C₁₀ cycloalkyl, spiro C₅-C₁₀ cycloalkyl, monocyclic C₂C₈ heterocycloalkyl, bridged C₅-C₁₀ heterocycloalkyl, or spiro C₅-C₁₀ heterocycloalkyl.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is phenyl, monocyclic 6-membered heteroaryl, monocyclic 5-membered heteroaryl, monocyclic C₃-C₈ cycloalkyl, bridged C₅-C₁₀ cycloalkyl, spiro C₅-C₁₀ cycloalkyl, monocyclic C₂C₈ heterocycloalkyl, bridged C₅-C₁₀ heterocycloalkyl, or spiro C₅-C₁₀ heterocycloalkyl.

In some embodiments, Ring A is phenyl or heteroaryl. In some embodiments, Ring A is phenyl or monocyclic heteroaryl. In some embodiments, Ring A is phenyl, monocyclic 6-membered heteroaryl, or monocyclic 5-membered heteroaryl. In some embodiments, Ring A is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring A is phenyl or 6-membered heteroaryl. In some embodiments, Ring A is phenyl, pyridinyl, pyrimidinyl, pyrazinyl, or pyridazinyl.

In some embodiments, Ring A is phenyl, monocyclic C₃-C₆ cycloalkyl, or bridged cycloalkyl. In some embodiments, Ring A is phenyl, monocyclic C₃-C₈ cycloalkyl, or bridged C₅-C₁₀ cycloalkyl. In some embodiments, Ring A is phenyl, cyclopropyl, cyclobutyl, cyclopentyl, cyclohexyl, or bridged C₅-C₁₀ cycloalkyl. In some embodiments, Ring A is phenyl, cyclohexyl, or

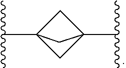 [SMILES: *C12CC(*)(C1)C2]

In some embodiments, Ring A is phenyl. In some embodiments, Ring A is cyclohexyl. In some embodiments, Ring A is

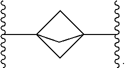 [SMILES: *C12CC(*)(C1)C2]

In some embodiments, Ring A is phenyl, naphthyl, indanyl, indenyl, tetrahyodronaphthyl, cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl.

In some embodiments, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl. In some embodiments, Ring A is monocyclic C₃-C₆ cycloalkyl, or bridged cycloalkyl. In some embodiments, Ring A is cyclopropyl, cyclobutyl, cyclopentyl, cyclohexyl, or bridged C₅-C₁₀ cycloalkyl. In some embodiments, Ring A is cyclohexyl or

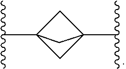 [SMILES: *C12CC(*)(C1)C2]

In some embodiments, Ring A is furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, cinnolinyl, phthalazinyl, quinazolinyl, quinoxalinyl, naphthyridinyl, pteridinyl, indolizinyl, azaindolizinyl, indolyl, azaindolyl, indazolyl, azaindazolyl, benzimidazolyl, azabenzimidazolyl, benzotriazolyl, azabenzotriazolyl, benzoxazolyl, azabenzoxazolyl, benzisoxazolyl, azabenzisoxazolyl, benzofuranyl, azabenzofuranyl, benzothienyl, azabenzothienyl, benzothiazolyl, azabenzothiazolyl, or purinyl.

In some embodiments, Ring A is aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, piperazinyl, homopiperidinyl, oxepanyl, thiepanyl, oxazepinyl, diazepinyl, thiazepinyl, azaspiro[3.3]heptanyl, azaspiro[3.4]octanyl, azaspiro[3.4]octanyl, or azaspiro[4.4]nonyl.

In some embodiments, Ring A is phenyl, pyridinyl, pyrimidinyl, pyrazinyl, or pyridazinyl.

In some embodiments, Ring A is an aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, or piperazinyl.

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl. In some embodiments, each R^{A} is independently halogen, —OH, —O—(C₁-C₆ alkyl), or C₁-C₆ alkyl. In some embodiments, each R^{A} is independently —F, —Cl, —Br, —OH, —OCH₃, —OCH₂CH₃, —OCH₂CH₂CH₃, —OCH(CH₃)₂, —CH3, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{A} is independently C₁-C₆ alkyl. In some embodiments, each R^{A} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, q is 0. In some embodiments, q is 1-4. In some embodiments, q is 0-2. In some embodiments, q is 0-1. In some embodiments, q is 1. In some embodiments, q is 2. In some embodiments, q is 3. In some embodiments, q is 4.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl; each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2.

In some embodiments, Ring A is phenyl, monocyclic C₃-C₆ cycloalkyl, or bridged cycloalkyl; each R^{A} is independently halogen, —OH, —O—(C₁-C₆ alkyl), or C₁-C₆ alkyl; and q is 0-2.

In some embodiments, Ring A is phenyl, cyclohexyl, or

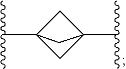 [SMILES: *C12CC(*)(C1)C2]

each R^{A} is independently halogen, —OH, —O—(C₁-C₆ alkyl), or C₁-C₆ alkyl; and q is 0-2.

In some embodiments, Ring A is phenyl; and q is 0. In some embodiments, Ring A is cyclohexyl; and q is 0. In some embodiments, Ring A is cyclohexyl; R^{A} is C₁-C₆ alkyl, and q is 1. In some embodiments, Ring A is

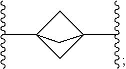 [SMILES: *C12CC(*)(C1)C2]

and q is 0.

In some embodiments, when X is —O—, and Y is —C(═O)—, Ring A is phenyl or heteroaryl. In some embodiments, Ring A is phenyl.

In some embodiments, when X is —O—, and Y is —C(═O)—, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is monocyclic C₃-C₆ cycloalkyl, or bridged cycloalkyl. In some embodiments, Ring A is cyclohexyl or

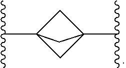 [SMILES: *C12CC(*)(C1)C2]

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2. In some embodiments, each R^{A} is independently halogen, —OH, —O—(C₁-C₆ alkyl), or C₁-C₆ alkyl; and q is 0-2. In some embodiments, each R^{A} is independently C₁-C₆ alkyl; and q is 0-2. In some embodiments, q is 0.

In some embodiments, the compound of Formula (I) has the structure of Formula (Ih), or a pharmaceutically acceptable salt, solvate, stereoisomer, or prodrug thereof:

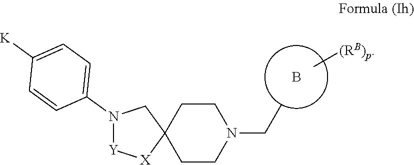

In some embodiments, the compound of Formula (I) has the structure of Formula (Ih-1), (Ih-2), or (Ih-3), or a pharmaceutically acceptable salt, solvate, stereoisomer, or prodrug thereof:

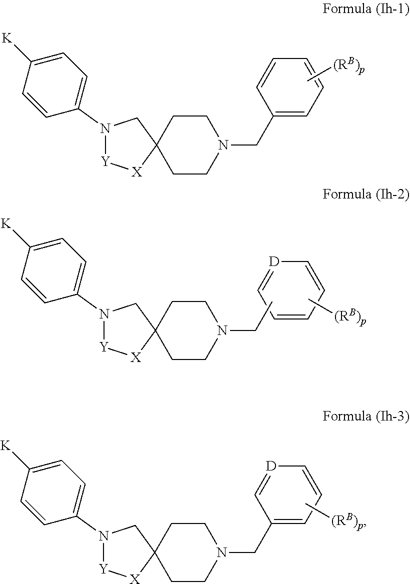

wherein D is CH or N.

In some embodiments, the compound of Formula (I) has the structure of Formula (Ii), Formula (Ij), Formula (Ik), or Formula (Il), or a pharmaceutically acceptable salt, solvate, stereoisomer, or prodrug thereof:

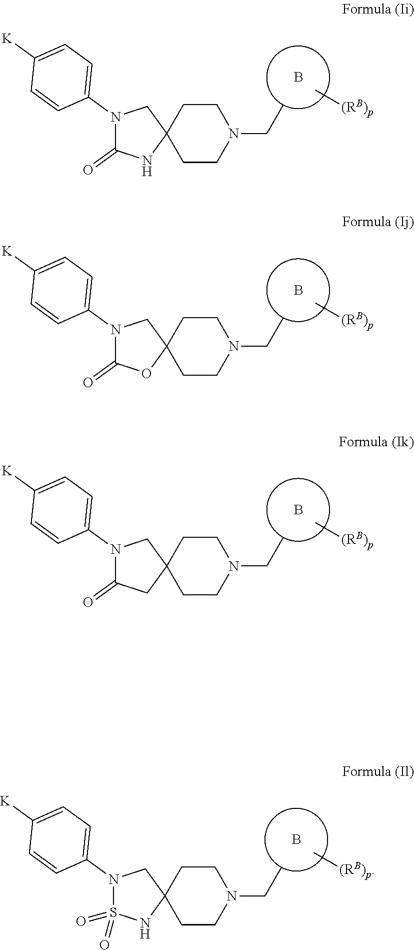

In some embodiments, the compound of Formula (I) has the structure of Formula (Ii), or a pharmaceutically acceptable salt, solvate, stereoisomer, or prodrug thereof:

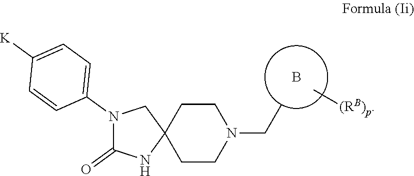

In some embodiments, the compound of Formula (I) has the structure of Formula (Ii-1), (Ii-2), or (Ii-3), or a pharmaceutically acceptable salt, solvate, stereoisomer, or prodrug thereof:

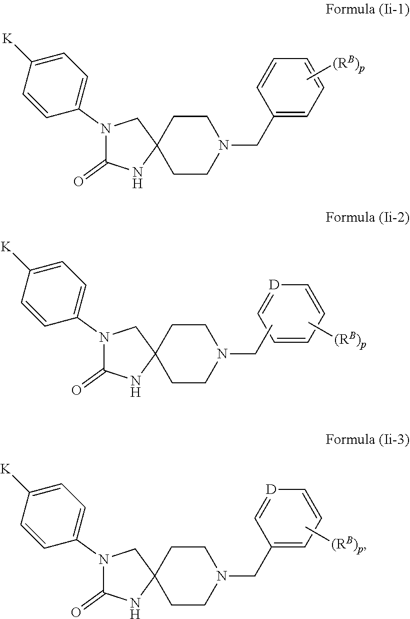

wherein D is CH or N.

In some embodiments, the compound of Formula (I) has the structure of Formula (Ij), or a pharmaceutically acceptable salt, solvate, stereoisomer, or prodrug thereof:

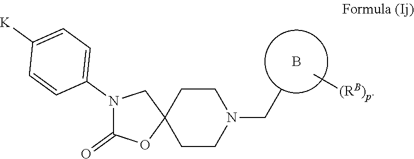

In some embodiments, the compound of Formula (I) has the structure of Formula (Ij-1), (Ij-2), or (Ij-3), or a pharmaceutically acceptable salt, solvate, stereoisomer, or prodrug thereof:

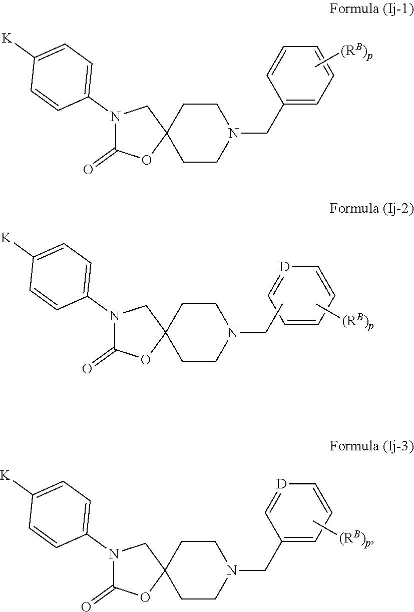

wherein D is CH or N.

In some embodiments, the compound of Formula (I) has the structure of Formula (Ik), or a pharmaceutically acceptable salt, solvate, stereoisomer, or prodrug thereof:

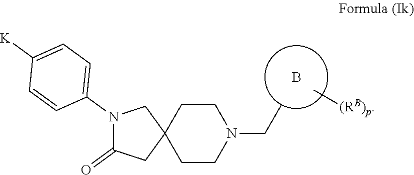

In some embodiments, the compound of Formula (I) has the structure of Formula (Ik-1), (Ik-2), or (Ik-3), or a pharmaceutically acceptable salt, solvate, stereoisomer, or prodrug thereof:

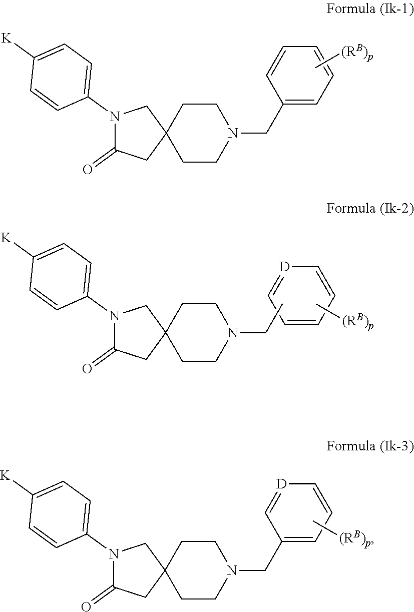

wherein D is CH or N.

In some embodiments, the compound of Formula (I) has the structure of Formula (Il), or a pharmaceutically acceptable salt, solvate, stereoisomer, or prodrug thereof:

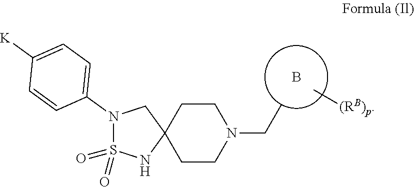

In some embodiments, the compound of Formula (I) has the structure of Formula (Il-1), (I1-2), or (I1-3), or a pharmaceutically acceptable salt, solvate, stereoisomer, or prodrug thereof:

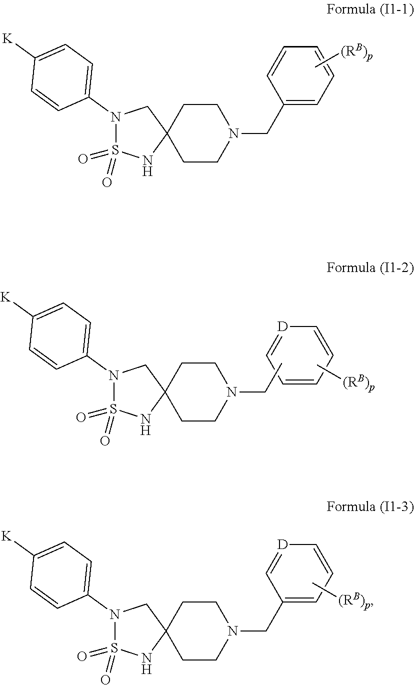

wherein D is CH or N.

In some embodiments, K is —Z—NR⁶R⁷.

In some embodiments, Z is *—(CH₂)ᵣ—C(═O)—, or *—(CH₂)ᵣ—S(═O)₂—, where * represents attachment to Ring A; and r is 0 or 1. In some embodiments, r is 0. In some embodiments, r is 1. In some embodiments, r is 2. In some embodiments, r is 3. In some embodiments, r is 4.

In some embodiments, Z is —C(═O)— or —S(═O)2—. In some embodiments, Z is —C(═O)—. In some embodiments, Z is —S(═O)₂—.

In some embodiments, R⁶ is hydrogen or C₁₋₆ alkyl which is unsubstituted or substituted by 1-6 R^{C} groups. In some embodiments, R⁶ is hydrogen or C₁₋₆ alkyl which is unsubstituted or substituted by 1-6 —OH groups. In some embodiments, R⁶ is hydrogen.

In some embodiments, R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]t—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—.

In some embodiments, R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-6; and R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups.

In some embodiments, R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; and t is 1-3; and R⁸ is hydrogen or C₁₋₈ alkyl, wherein the alkyl is substituted by 1-6 R^{C} groups.

In some embodiments, R⁷ is C₁₋₈ alkyl which is substituted by 1-6 R^{C} groups.

In some embodiments, R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups. In some embodiments, R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups. In some embodiments, R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups.

In some embodiments, each R^{C} is independently —OH, —NH₂, —N(R^{D})₂, —N(R^{D})₃⁺, —C(═O)OH, G, or G¹.

In some embodiments, each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

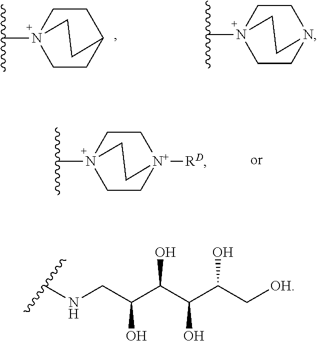

In some embodiments, each G is independently —S(═O)₂OH, —S(═O)₂NH₂, —P(═O)(OH)₂, —P(═O)(OH)(R^{D}),

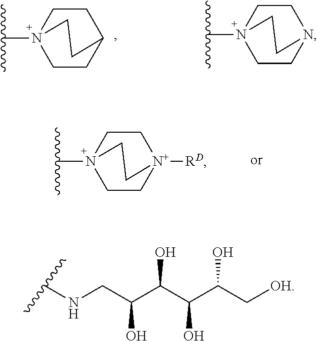

In some embodiments, each G is independently —S(═O)₂OH, —S(═O)₂NH₂, —P(═O)(OH)₂, or —P(═O)(OH)(R^{D}).

In some embodiments, G¹ is

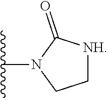 [SMILES: *N1CCNC1=O]

In some embodiments, each R^{D} is independently C₁₋₆ alkyl.

In some embodiments, each R^{C} is independently —OH, —NH₂, —N(R^{D})₂, —N(R^{D})₃⁺, —C(═O)OH, or G; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

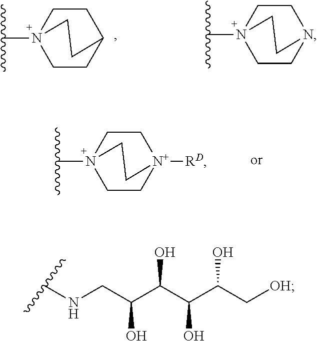

and each R^{D} is independently C₁₋₆ alkyl.

In some embodiments, each R^{C} is independently —OH, —NH₂, —N(R^{D})_{2, —N(R}^{D})₃⁺, —C(═O)OH, or G; each G is independently —S(═O)₂OH, —S(═O)₂NH₂, —P(═O)(OH)₂, —P(═O)(OH)(R^{D}),

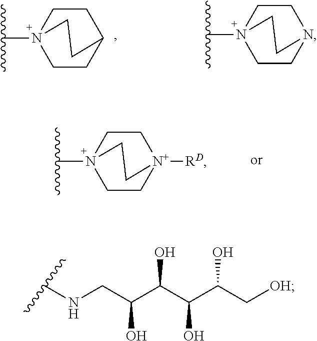

and each R^{D} is independently C₁₋₆ alkyl.

In some embodiments, each R^{C} is independently —OH, —NH₂, —N(R^{D})₂, —N(R^{D})₃⁺, —C(═O)OH, or G; each G is independently —S(═O)₂OH, —S(═O)₂NH₂, —P(═O)(OH)₂, —P(═O)(OH)(R^{D}),

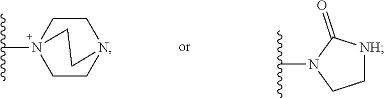

and each R^{D} is independently C₁₋₆ alkyl.

In some embodiments, each R^{C} is independently —OH, —NH_{2, —N(R}^{D})₂, —N(R^{D})₃⁺, —C(═O)OH, or G; each G is independently —S(═O)₂OH, —S(═O)₂NH₂, —P(═O)(OH)₂, or —P(═O)(OH)(R^{D}); and each R^{D} is independently C₁₋₆ alkyl.

In some embodiments, R⁶ is hydrogen or C₁₋₆ alkyl which is unsubstituted or substituted by 1-6 R^{C} groups; R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—.

In some embodiments, R⁶ is hydrogen or C₁₋₆ alkyl which is unsubstituted or substituted by 1-6 —OH groups; R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-6; R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

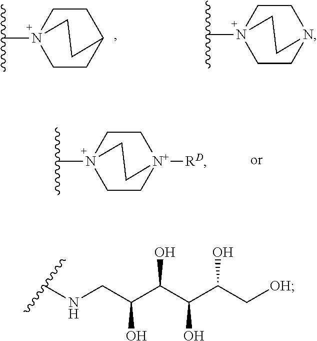

### G¹ is

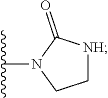 [SMILES: *N1CCNC1=O]

and each R^{D} is independently C₁₋₆ alkyl.

In some embodiments, R⁶ is hydrogen; R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-3; R⁸ is hydrogen or C₁₋₈ alkyl, wherein the alkyl is substituted by 1-6 R^{C} groups; each R^{C} is independently —OH, —NH₂, —N(R^{D})₂, —N(R^{D})₃⁺, —C(═O)OH, or G; each G is independently —S(═O)₂OH, —S(═O)₂NH₂, —P(═O)(OH)₂, —P(═O)(OH)(R^{D}),

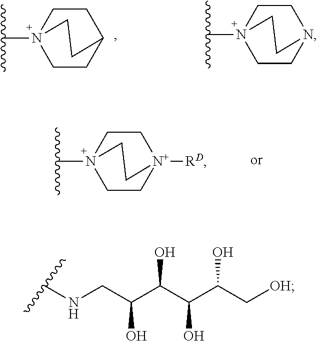

### G¹ is

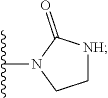 [SMILES: *N1CCNC1=O]

and each R^{D} is independently C₁₋₆ alkyl.

In some embodiments, R⁶ is hydrogen; R⁷ is C₁₋₈ alkyl which is substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}), or

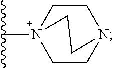

### G¹ is

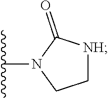 [SMILES: *N1CCNC1=O]

and each R^{D} is independently C₁₋₆ alkyl.

In some embodiments, R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

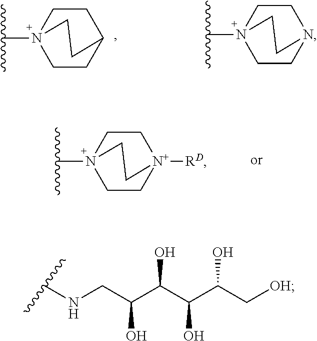

### G¹ is

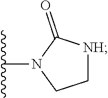 [SMILES: *N1CCNC1=O]

and each R^{D} is independently C₁₋₆ alkyl.

In some embodiments, R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen or C₁₋₈ alkyl which is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}),

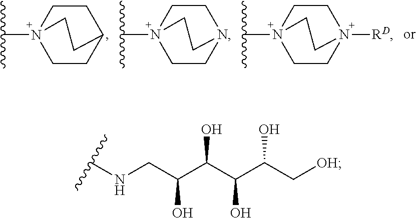

### G¹ is

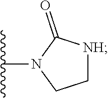 [SMILES: *N1CCNC1=O]

and each R^{D} is independently C₁₋₆ alkyl.

In some embodiments, R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen or C₁₋₈ alkyl which is unsubstituted or substituted by 1-6 R^{C} groups; each R^{C} is independently —OH, —NH₂, —N(R^{D})₂, —N(R^{D})₃⁺, —C(═O)OH, or G; each G is independently —S(═O)₂OH, —S(═O)₂NH₂, —P(═O)(OH)₂, or —P(═O)(OH)(R^{D}); and each R^{D} is independently C₁₋₆ alkyl.

In some embodiments, the compound of Formula (I) has the structure of Formula (B), or a pharmaceutically acceptable salt, solvate, stereoisomer, or prodrug thereof:

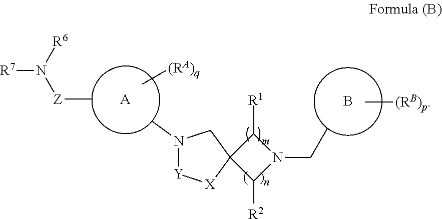

In some embodiments, the compound of Formula (I) has the structure of Formula (B1), (B2), or (B3), or a pharmaceutically acceptable salt, solvate, stereoisomer, or prodrug thereof:

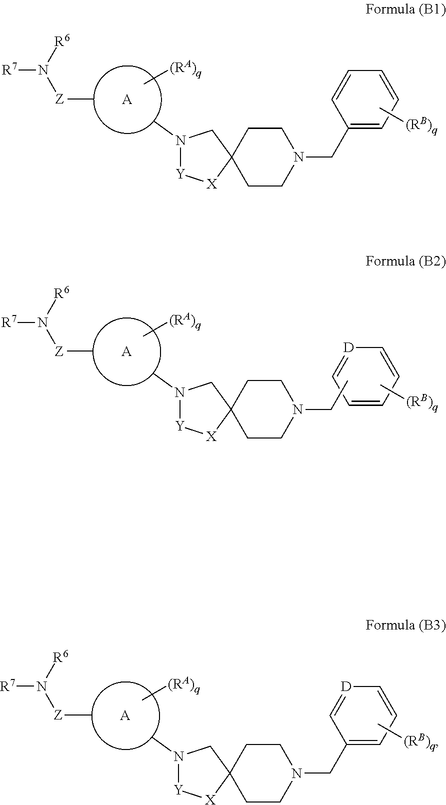

wherein D is CH or N.

In some embodiments, the compound of Formula (I) has the structure of Formula (B1), or a pharmaceutically acceptable salt, solvate, stereoisomer, or prodrug thereof. In some embodiments, the compound of Formula (I) has the structure of Formula (B2), or a pharmaceutically acceptable salt, solvate, stereoisomer, or prodrug thereof. In some embodiments, the compound of Formula (I) has the structure of Formula (B3), or a pharmaceutically acceptable salt, solvate, stereoisomer, or prodrug thereof.

In some embodiments, the compound of Formula (I) has the structure of Formula (B4), (B5), or (B6), or a pharmaceutically acceptable salt, solvate, stereoisomer, or prodrug thereof:

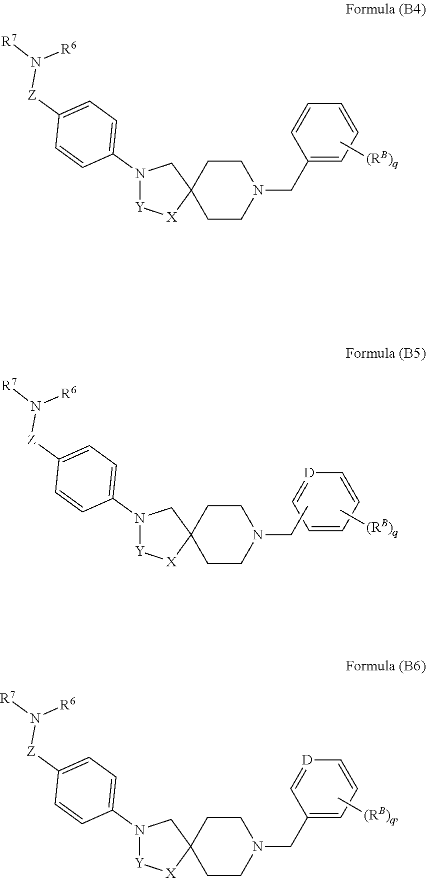

wherein D is CH or N.

In some embodiments, the compound of Formula (I) has the structure of Formula (B4), or a pharmaceutically acceptable salt, solvate, stereoisomer, or prodrug thereof. In some embodiments, the compound of Formula (I) has the structure of Formula (B5), or a pharmaceutically acceptable salt, solvate, stereoisomer, or prodrug thereof. In some embodiments, the compound of Formula (I) has the structure of Formula (B6), or a pharmaceutically acceptable salt, solvate, stereoisomer, or prodrug thereof.

In some embodiments, the compound of Formula (I) has the structure of Formula (B7), (B8), (B9), or (B10), or a pharmaceutically acceptable salt, solvate, stereoisomer, or prodrug thereof:

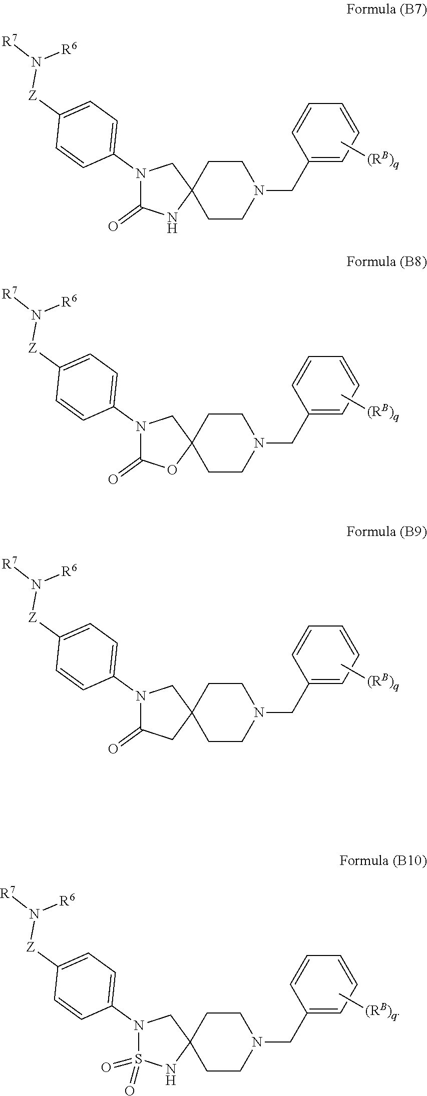

In some embodiments, the compound of Formula (I) has the structure of Formula (B7). In some embodiments, the compound of Formula (I) has the structure of Formula (B8). In some embodiments, the compound of Formula (I) has the structure of Formula (B9). In some embodiments, the compound of Formula (I) has the structure of Formula (B10). In some embodiments, Z is *—(CH₂)ᵣ—C(═O)—, or *—(CH₂)ᵣ—S(═O)₂—, where * represents attachment to Ring A. In some embodiments, r is 0 and Z is *—C(═O)—, or *—S(═O)₂—, where * represents attachment to Ring A.

In some embodiments, the compound of Formula (I) has the structure of Formula (B11) or (B12), or a pharmaceutically acceptable salt, solvate, stereoisomer, or prodrug thereof:

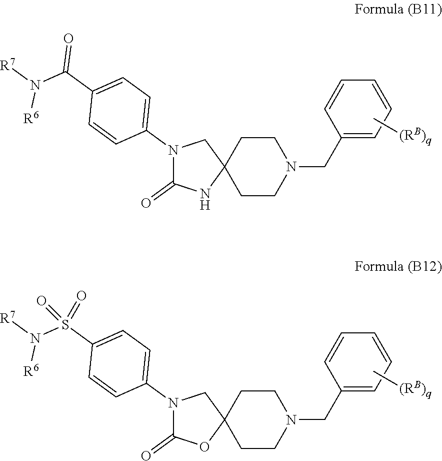

In some embodiments, the compound of Formula (I) has the structure of Formula (B7), or a pharmaceutically acceptable salt, solvate, stereoisomer, or prodrug thereof. In some embodiments, the compound of Formula (I) has the structure of Formula (B8), or a pharmaceutically acceptable salt, solvate, stereoisomer, or prodrug thereof. In some embodiments, the compound of Formula (I) has the structure of Formula (B9), or a pharmaceutically acceptable salt, solvate, stereoisomer, or prodrug thereof. In some embodiments, the compound of Formula (I) has the structure of Formula (B10), or a pharmaceutically acceptable salt, solvate, stereoisomer, or prodrug thereof.

Also disclosed herein, in certain embodiments, is a compound of Formula (II):

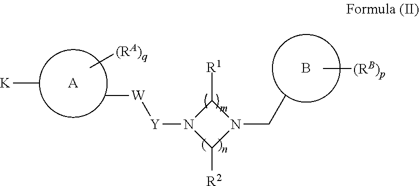

or a pharmaceutically acceptable salt, solvate, stereoisomer, or prodrug thereof, wherein:

W is a bond, —O—, —NR³—, or —C(R⁴)₂—;

Y is —C(═O)—, or —S(═O)₂—;

Ring A is aryl, heteroaryl, cycloalkyl, or heterocycloalkyl;

Ring B is aryl or heteroaryl;

K is —Z—NR⁶R⁷;
Z is *—(CH₂)ᵣ—C(═O)—, or *—(CH₂),—S(═O)₂—, where * represents attachment to Ring A;
R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
   each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—;
or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
r is 0-4;
each s is independently 1-6;
each t is independently 1-6;

each R¹ and R² is independently hydrogen, C₁₋₆ alkyl, or C₁₋₆ fluoroalkyl;

or one R¹ and one R² are taken together to form a ring;

R³ is hydrogen, C₁₋₆ alkyl, C₁₋₆ fluoroalkyl, or C₃₋₆ cycloalkyl;

each R⁴ is independently hydrogen, C₁₋₆ alkyl, C₁₋₆ fluoroalkyl, or C₃₋₆ cycloalkyl;

R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;

each R^{C} is independently —OH, —NH₂, —NH(R^{D}), —N(R^{D})₂, —N(R^{D})₃⁺, ═O, ═S, —C(═O)OH,

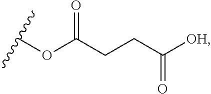 [SMILES: *OC(=O)CCC(=O)O]

### G, or G¹;

each G is independently —S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

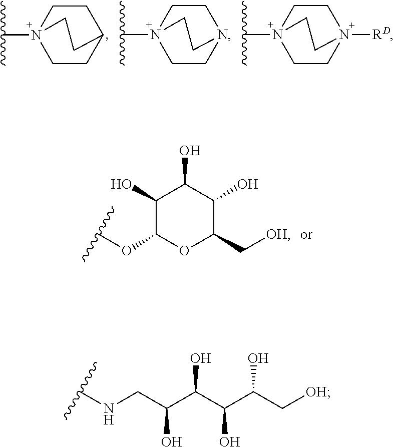

each G¹ is independently a 4- to 6-membered heterocycle which is unsubstituted or substituted with 1, 2, 3, or 4 substituents selected from C₁-C₆ alkyl, —O—(C₁-C₆ alkyl), —OH, ═O and ═S;

each R^{D} is independently C₁₋₆ alkyl or C₃₋₆ cycloalkyl; wherein the alkyl and cycloalkyl are unsubstituted or substituted by 1-3 halogen or —OH groups;

each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;

each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;

each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

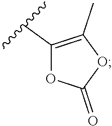 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;

each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

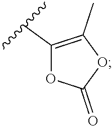 [SMILES: *c1oc(=O)oc1C]

m is 1 or 2;

n is 1 or 2;

p is 1-4; and

q is 0-4.

In some embodiments, each R¹ and R² is independently hydrogen, C₁₋₆ alkyl, or C₁₋₆ fluoroalkyl. In some embodiments, each R¹ and R² is independently hydrogen or C₁₋₆ alkyl. In some embodiments, each R¹ and R² is independently —H, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂F, —CHF₂, —CF₃, —CH₂CH₂F, —CH₂CHF₂, or —CH₂CF₃. In some embodiments, each R¹ and R² is independently —H, —CH₃, —CH₂CH₃, or —CH₂CH₂CH₃. In some embodiments, each R¹ and R² is —H.

In some embodiments, one R¹ and one R² are taken together to form a ring. In some embodiments, one R¹ and one R² are taken together to form a 3- to 6-membered heterocycloalkyl ring.

In some embodiments, m is 1. In some embodiments, m is 2. In some embodiments, n is 1. In some embodiments, n is 2. In some embodiments, m is 1 and n is 1. In some embodiments, m is 1 and n is 2. In some embodiments, m is 2 and n is 1. In some embodiements, m is 2 and n is 2.

In some embodiments, Ring B is phenyl, naphthyl, monocyclic 6-membered heteroaryl, monocyclic 5-membered heteroaryl, or bicyclic heteroaryl.

In some embodiments, Ring B is phenyl or monocyclic heteroaryl. In some embodiments, Ring B is phenyl, monocyclic 6-membered heteroaryl, or monocyclic 5-membered heteroaryl. In some embodiments, Ring B is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring B is phenyl or 6-membered heteroaryl. In some embodiments, Ring B is phenyl, pyridinyl, pyrimidinyl, pyrazinyl, or pyridazinyl.

In some embodiments, Ring B is phenyl, or pyridinyl.

In some embodiments, Ring B is

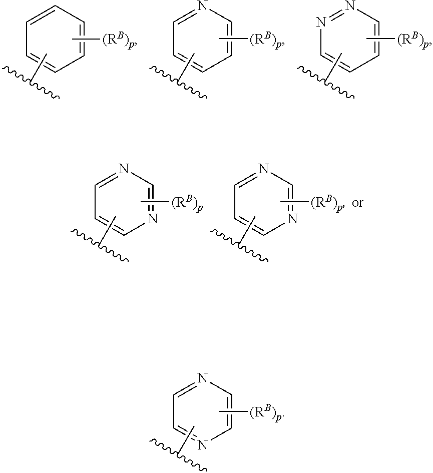

In some embodiments, Ring B is

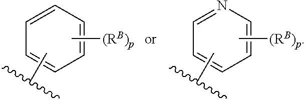

In some embodiments, Ring B is

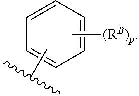

In some embodiments, Ring B is

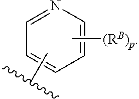

In some embodiments, Ring B is

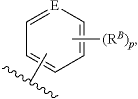

where E is CH or N.

In some embodiments, Ring B is phenyl or 6-membered heteroaryl; each R¹ and R² is independently hydrogen or C₁₋₆ alkyl; m is 2; and n is 2.

In some embodiments, the compound of Formula (II) has the structure of Formula (IIa), or a pharmaceutically acceptable salt, solvate, stereoisomer, or prodrug thereof:

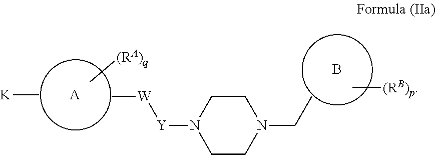

In some embodiments, the compound of Formula (II) has the structure of Formula (IIa-1), or a pharmaceutically acceptable salt, solvate, stereoisomer, or prodrug thereof:

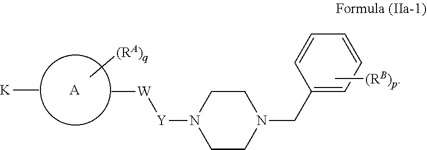

In some embodiments, the compound of Formula (II) has the structure of Formula (IIa-2), or a pharmaceutically acceptable salt, solvate, stereoisomer, or prodrug thereof:

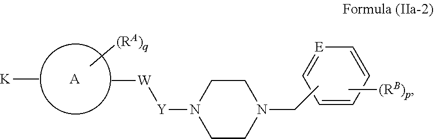

wherein E is CH or N.

In some embodiments, the compound of Formula (II) has the structure of Formula (IIa-3), or a pharmaceutically acceptable salt, solvate, stereoisomer, or prodrug thereof:

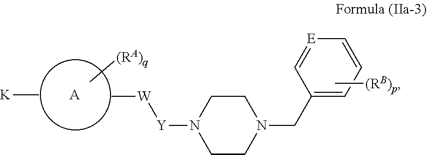

wherein E is CH or N.

In some embodiments, W is —O—. In some embodiments, W is —NR³—. In some embodiments, W is —C(R⁴)₂—. In some embodiments, W is a bond.

In some embodiments, Y is —C(═O)—. In some embodiments, Y is —S(═O)₂—.

In some embodiments, W is a bond, and Y is —C(═O)—. In some embodiments, W is —O—, and Y is —C(═O)—. In some embodiments, W is —NR³—, and Y is —C(═O)—. In some embodiments, W is —C(R⁴)₂—; and Y is —C(═O)—. In some embodiments, W is a bond, and Y is —S(═O)₂—. In some embodiments, W is —O—, and Y is —S(═O)₂—. In some embodiments, W is —NR³—, and Y is —S(═O)₂—. In some embodiments, W is —C(R⁴)₂—; and Y is —S(═O)₂—.

In some embodiments, W is a bond, and Y is —C(═O)—; or W is —O—, and Y is —C(═O)—; or W is —NR³—, and Y is —C(═O)—; or W is —C(R⁴)₂—; and Y is —C(═O)—; or W is a bond, and Y is —S(═O)₂—; or W is —O—, and Y is —S(═O)₂—; or W is —NR³—, and Y is —S(═O)₂—; or W is —C(R⁴)₂—; and Y is —S(═O)₂—. In some embodiments, W is —O—, and Y is —C(═O)—; or W is —NR³—, and Y is —C(═O)—; or W is —C(R⁴)₂—; and Y is —C(═O)—; or W is —O—, and Y is —S(═O)₂—; or W is —NR³—, and Y is —S(═O)₂—; or W is —C(R⁴)₂—; and Y is —S(═O)₂—. In some embodiments, W is —O—, and Y is —C(═O)—; or W is —NR³—, and Y is —C(═O)—; or W is —C(R⁴)₂—; and Y is —C(═O)—; or W is a bond, and Y is —C(═O)—.

In some embodiments, the compound of Formula (II) has the structure of Formula (IIb), or a pharmaceutically acceptable salt, solvate, stereoisomer, or prodrug thereof:

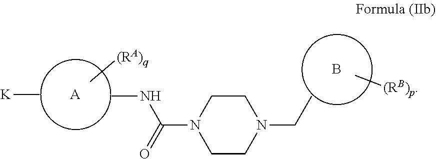

In some embodiments, the compound of Formula (II) has the structure of Formula (IIb-1), (IIb-2), or (IIb-3), or a pharmaceutically acceptable salt, solvate, stereoisomer, or prodrug thereof:

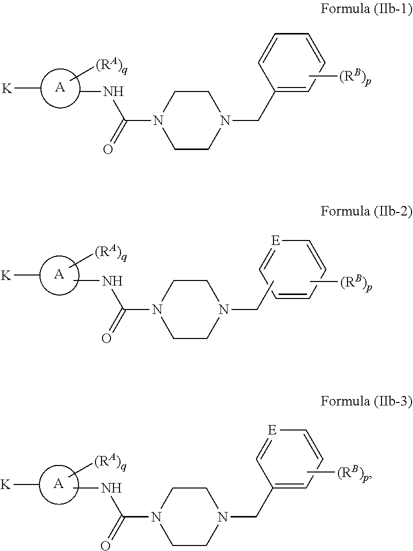

wherein E is CH or N.

In some embodiments, the compound of Formula (II) has the structure of Formula (IIc), or a pharmaceutically acceptable salt, solvate, stereoisomer, or prodrug thereof:

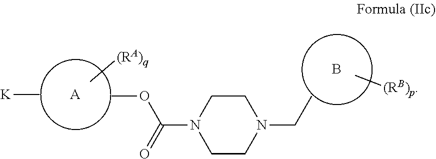

In some embodiments, the compound of Formula (II) has the structure of Formula (IIc-1), (IIc-2), or (IIc-3), or a pharmaceutically acceptable salt, solvate, stereoisomer, or prodrug thereof:

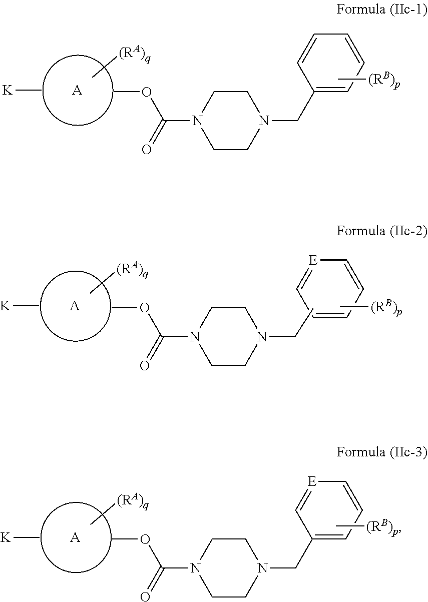

wherein E is CH or N.

In some embodiments, the compound of Formula (I) has the structure of Formula (Id), or a pharmaceutically acceptable salt, solvate, stereoisomer, or prodrug thereof:

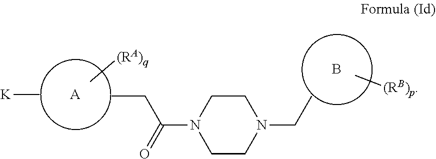

In some embodiments, the compound of Formula (II) has the structure of Formula (IId-1), (IId-2), or (IId-3), or a pharmaceutically acceptable salt, solvate, stereoisomer, or prodrug thereof:

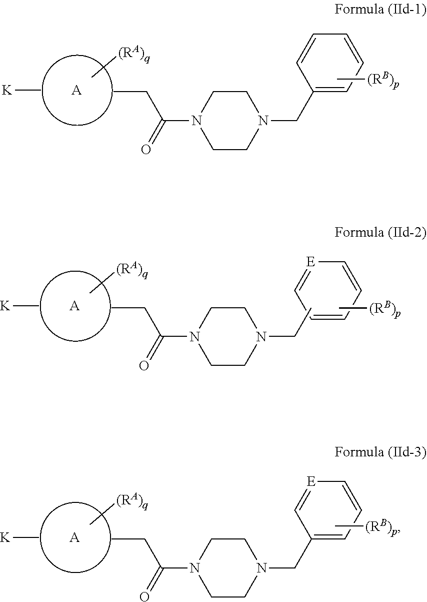

wherein E is CH or N.

In some embodiments, the compound of Formula (II) has the structure of Formula (IIe), or a pharmaceutically acceptable salt, solvate, stereoisomer, or prodrug thereof:

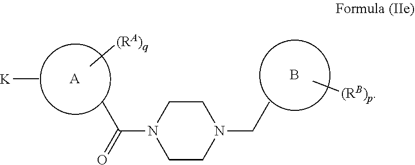

In some embodiments, the compound of Formula (II) has the structure of Formula (IIe-2), or (IIe-3), or a pharmaceutically acceptable salt, solvate, stereoisomer, or prodrug thereof:

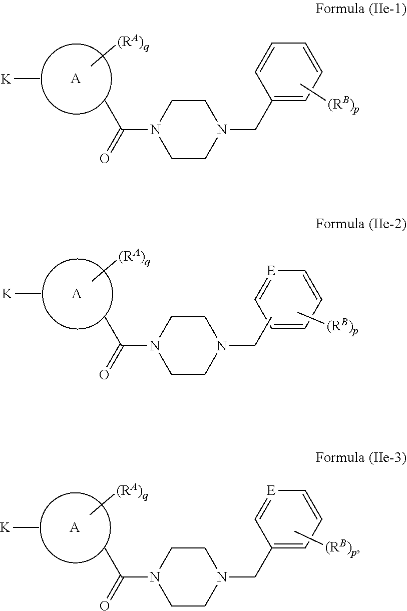

wherein E is CH or N.

In some embodiments, the compound of Formula (II) has the structure of Formula (IIb), Formula (IIc), Formula (IId), or Formula (IIe), or a pharmaceutically acceptable salt, solvate, stereoisomer, or prodrug thereof.

In some embodiments, each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl. In some embodiments, each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl. In some embodiments, each R^{B} is independently phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH2OH, —O—(C₁-C₆ alkyl), C₁-C₆ alkyl, C₁-C₆ fluoroalkyl.

In some embodiments, p is 1. In some embodiments, p is 2. In some embodiments, p is 3. In some embodiments, p is 4. In some embodiments, p is 1-3. In some embodiments, p is 2 or 3.

In some embodiments, the compound of Formula (II) has the structure of Formula (IIf), or a pharmaceutically acceptable salt, solvate, stereoisomer, or prodrug thereof:

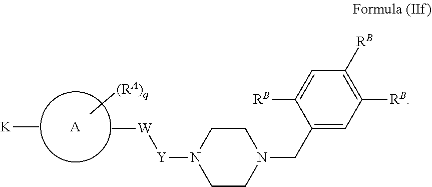

In some embodiments, the compound of Formula (II) has the structure of Formula (IIg), or a pharmaceutically acceptable salt, solvate, stereoisomer, or prodrug thereof:

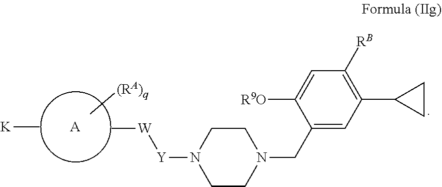

In some embodiments, R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

In some embodiments, Ring A is phenyl, naphthyl, monocyclic 6-membered heteroaryl, monocyclic 5-membered heteroaryl, bicyclic heteroaryl, monocyclic C₃-C₈ cycloalkyl, bridged C₅-C₁₀ cycloalkyl, spiro C₅-C₁₀ cycloalkyl, monocyclic C₂-C₈ heterocycloalkyl, bridged C₅-C₁₀ heterocycloalkyl, or spiro C₅-C₁₀ heterocycloalkyl.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is phenyl, monocyclic 6-membered heteroaryl, monocyclic 5-membered heteroaryl, monocyclic C₃-C₈ cycloalkyl, bridged C₅-C₁₀ cycloalkyl, spiro C₅-C₁₀ cycloalkyl, monocyclic C₂-C₈ heterocycloalkyl, bridged C₅-C₁₀ heterocycloalkyl, or spiro C₅-C₁₀ heterocycloalkyl.

In some embodiments, Ring A is phenyl or heteroaryl. In some embodiments, Ring A is phenyl or monocyclic heteroaryl. In some embodiments, Ring A is phenyl, monocyclic 6-membered heteroaryl, or monocyclic 5-membered heteroaryl. In some embodiments, Ring A is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring A is phenyl or 6-membered heteroaryl. In some embodiments, Ring A is phenyl, pyridinyl, pyrimidinyl, pyrazinyl, or pyridazinyl.

In some embodiments, Ring A is phenyl, monocyclic C₃-C₆ cycloalkyl, or bridged cycloalkyl. In some embodiments, Ring A is phenyl, monocyclic C₃-C₈ cycloalkyl, or bridged C₅-C₁₀ cycloalkyl. In some embodiments, Ring A is phenyl, cyclopropyl, cyclobutyl, cyclopentyl, cyclohexyl, or bridged C₅-C₁₀ cycloalkyl. In some embodiments, Ring A is phenyl, cyclohexyl, or

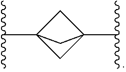 [SMILES: *C12CC(*)(C1)C2]

In some embodiments, King A is phenyl. In some embodiments, Ring A is cyclohexyl. In some embodiments, Ring A is

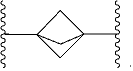 [SMILES: *C12CC(*)(C1)C2]

In some embodiments, Ring A is phenyl, naphthyl, indanyl, indenyl, tetrahyodronaphthyl, cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl.

In some embodiments, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl. In some embodiments, Ring A is monocyclic C₃-C₆ cycloalkyl, or bridged cycloalkyl. In some embodiments, Ring A is cyclopropyl, cyclobutyl, cyclopentyl, cyclohexyl, or bridged C₅-C₁₀ cycloalkyl. In some embodiments, Ring A is cyclohexyl or

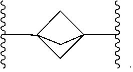 [SMILES: *C12CC(*)(C1)C2]

In some embodiments, Ring A is furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, cinnolinyl, phthalazinyl, quinazolinyl, quinoxalinyl, naphthyridinyl, pteridinyl, indolizinyl, azaindolizinyl, indolyl, azaindolyl, indazolyl, azaindazolyl, benzimidazolyl, azabenzimidazolyl, benzotriazolyl, azabenzotriazolyl, benzoxazolyl, azabenzoxazolyl, benzisoxazolyl, azabenzisoxazolyl, benzofuranyl, azabenzofuranyl, benzothienyl, azabenzothienyl, benzothiazolyl, azabenzothiazolyl, or purinyl.

In some embodiments, Ring A is aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, piperazinyl, homopiperidinyl, oxepanyl, thiepanyl, oxazepinyl, diazepinyl, thiazepinyl, azaspiro[3.3]heptanyl, azaspiro[3.4]octanyl, azaspiro[3.4]octanyl, or azaspiro[4.4]nonyl.

In some embodiments, Ring A is phenyl, pyridinyl, pyrimidinyl, pyrazinyl, or pyridazinyl.

In some embodiments, Ring A is an aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, or piperazinyl.

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl. In some embodiments, each R^{A} is independently halogen, —OH, —O—(C₁-C₆ alkyl), or C₁-C₆ alkyl. In some embodiments, each R^{A} is independently —F, —Cl, —Br, —OH, —OCH₃, —OCH₂CH₃, —OCH₂CH₂CH₃, —OCH(CH₃)₂, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{A} is independently C₁-C₆ alkyl. In some embodiments, each R^{A} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, q is 0. In some embodiments, q is 1-4. In some embodiments, q is 0-2. In some embodiments, q is 0-1. In some embodiments, q is 1. In some embodiments, q is 2. In some embodiments, q is 3. In some embodiments, q is 4.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl; each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2.

In some embodiments, Ring A is phenyl, monocyclic C₃-C₆ cycloalkyl, or bridged cycloalkyl; each R^{A} is independently halogen, —OH, —O—(C₁-C₆ alkyl), or C₁-C₆ alkyl; and q is 0-2.

In some embodiments, Ring A is phenyl, cyclohexyl, or

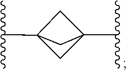 [SMILES: *C12CC(*)(C1)C2]

each R^{A} is independently halogen, —OH, —O—(C₁-C₆ alkyl), or C₁-C₆ alkyl; and q is 0-2.

In some embodiments, Ring A is phenyl; and q is 0.

In some embodiments, the compound of Formula (II) has the structure of Formula (IIh), or a pharmaceutically acceptable salt, solvate, stereoisomer, or prodrug thereof:

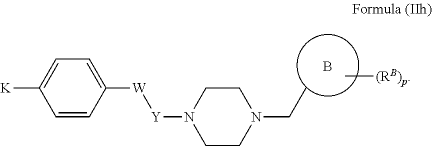

In some embodiments, the compound of Formula (II) has the structure of Formula (IIh-1), (IIh-2), or (IIh-3), or a pharmaceutically acceptable salt, solvate, stereoisomer, or prodrug thereof:

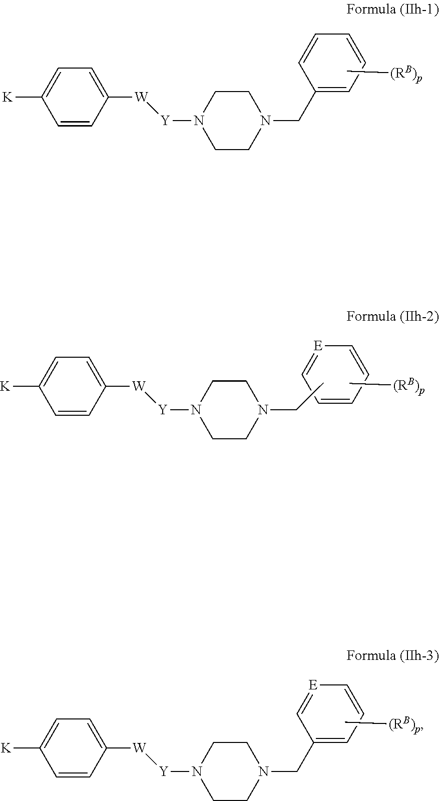

wherein E is CH or N.

In some embodiments, the compound of Formula (IIi) has the structure of Formula (IIi), or a pharmaceutically acceptable salt, solvate, stereoisomer, or prodrug thereof:

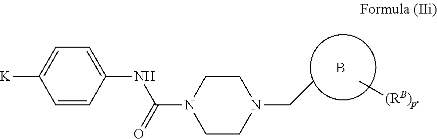

In some embodiments, the compound of Formula (II) has the structure of Formula (IIi-2), or (IIi-3), or a pharmaceutically acceptable salt, solvate, stereoisomer, or prodrug thereof:

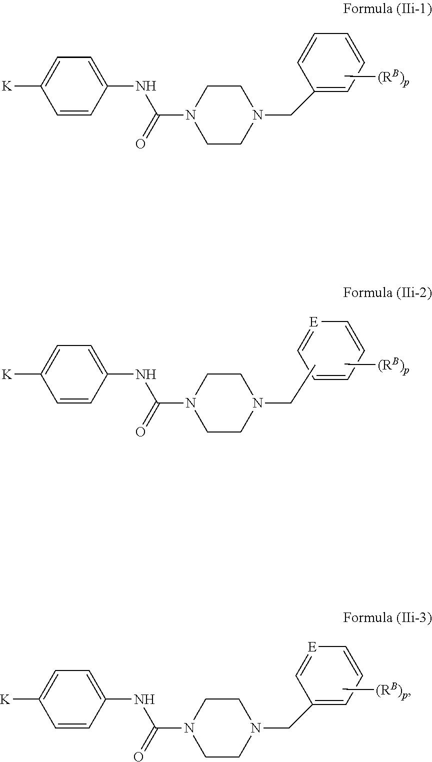

wherein E is CH or N.

In some embodiments, the compound of Formula (II) has the structure of Formula (IIj), or a pharmaceutically acceptable salt, solvate, stereoisomer, or prodrug thereof:

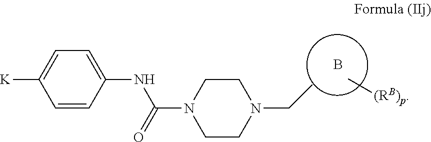

In some embodiments, the compound of Formula (II) has the structure of Formula (IIj-1), (IIj-2), or (IIj-3), or a pharmaceutically acceptable salt, solvate, stereoisomer, or prodrug thereof:

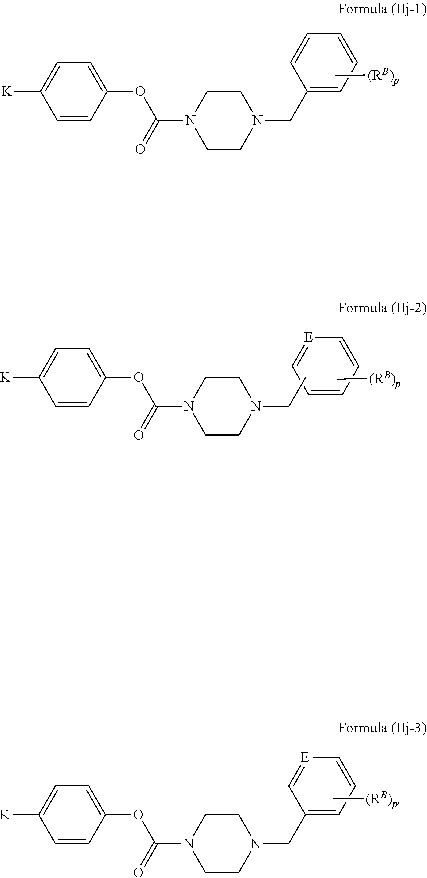

wherein E is CH or N.

In some embodiments, the compound of Formula (I) has the structure of Formula (Ik), or a pharmaceutically acceptable salt, solvate, stereoisomer, or prodrug thereof:

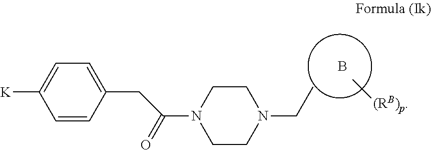

In some embodiments, the compound of Formula (II) has the structure of Formula (IIk-1), (IIk-2), or (IIk-3), or a pharmaceutically acceptable salt, solvate, stereoisomer, or prodrug thereof:

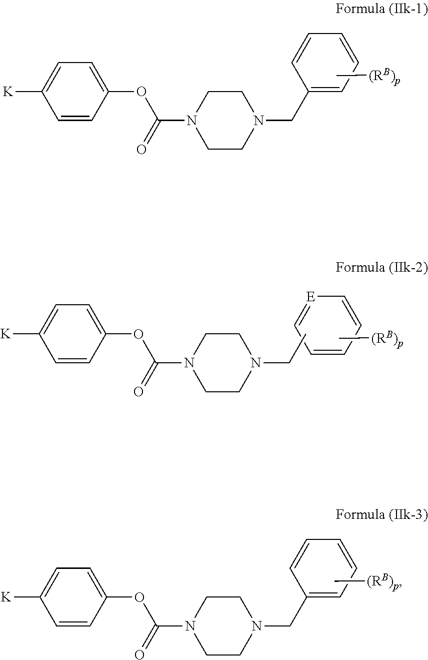

wherein E is CH or N.

In some embodiments, the compound of Formula (II) has the structure of Formula (IIl), or a pharmaceutically acceptable salt, solvate, stereoisomer, or prodrug thereof:

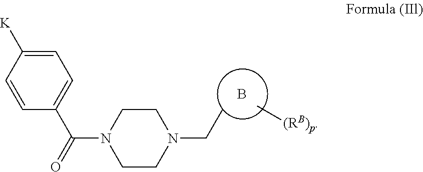

In some embodiments, the compound of Formula (II) has the structure of Formula (IIl-1), (II1-2), or (II1-3), or a pharmaceutically acceptable salt, solvate, stereoisomer, or prodrug thereof:

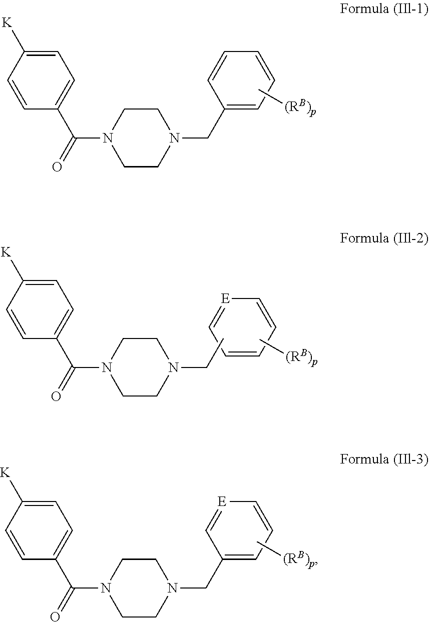

wherein E is CH or N.

In some embodiments, K is —Z—NR⁶R⁷.

In some embodiments, Z is *—(CH₂)ᵣ—C(═O)—, or *—(CH₂)ᵣ—S(═O)₂—, where * represents attachment to Ring A; and r is 0 or 1. In some embodiments, r is 0. In some embodiments, r is 1. In some embodiments, r is 2. In some embodiments, r is 3. In some embodiments, r is 4.

In some embodiments, Z is —C(═O)— or —S(═O)₂—. In some embodiments, Z is —C(═O)—. In some embodiments, Z is —S(═O)₂—.

In some embodiments, R⁶ is hydrogen or C₁₋₆ alkyl which is unsubstituted or substituted by 1-6 R^{C} groups. In some embodiments, R⁶ is hydrogen or C₁₋₆ alkyl which is unsubstituted or substituted by 1-6 —OH groups. In some embodiments, R⁶ is hydrogen.

In some embodiments, R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and each V is independently —CH₂O—, —CH₂NR^{D}—, —CH₂N⁺(R^{D})₂—, —NH—C(═O)—NH—, —C(═O)NH—, —CH₂ S(═O)₂—, or —CH₂S(═O)—.

In some embodiments, R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-6; and R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups.

In some embodiments, R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; and t is 1-3; and R⁸ is hydrogen or C₁₋₈ alkyl, wherein the alkyl is substituted by 1-6 R^{C} groups.

In some embodiments, R⁷ is C₁₋₈ alkyl which is substituted by 1-6 R^{C} groups.

In some embodiments, R⁶ and R⁷ are taken together with the nitrogen to which they are attached to form a 3- to 8-membered heterocycloalkyl, which is substituted by 1-6 groups selected from R^{C}, —C(═O)NRH⁸, —CH₂NHR⁸, or C₁₋₈ alkyl which is unsubstituted or substituted by 1-6 R^{C} groups. In some embodiments, R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups. In some embodiments, R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups.

In some embodiments, each R^{C} is independently —OH, —NH₂, —N(R^{D})₂, —N(R^{D})₃⁺, —C(═O)OH, G, or G¹.

In some embodiments, each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

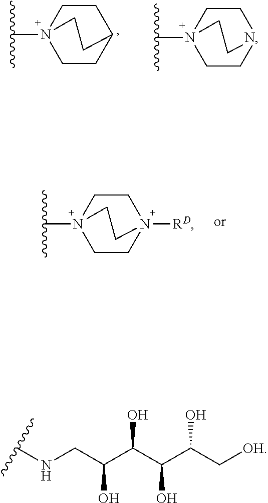

In some embodiments, each G is independently —S(═O)₂OH, —S(═O)₂NH₂, —P(═O)(OH)₂, —P(═O)(OH)(R^{D}),

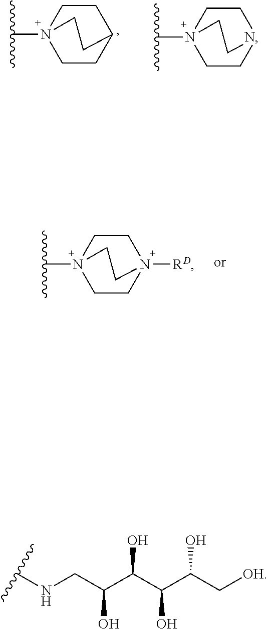

In some embodiments, each G is independently —S(═O)₂OH, —S(═O)₂NH₂, —P(═O)(OH)₂, or —P(═O)(OH)(R^{D}).

In some embodiments, G¹ is

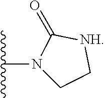 [SMILES: *N1CCNC1=O]

In some embodiments, each R^{D} is independently C₁₋₆ alkyl.

In some embodiments, each R^{C} is independently —OH, —NH₂, —N(R^{D})₂, —N(R^{D})₃⁺, —C(═O)OH, G, or G¹; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

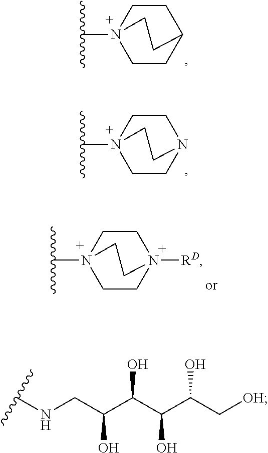

### G¹ is

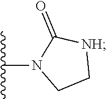 [SMILES: *N1CCNC1=O]

and each R^{D} is independently C₁₋₆ alkyl.

In some embodiments, each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}),

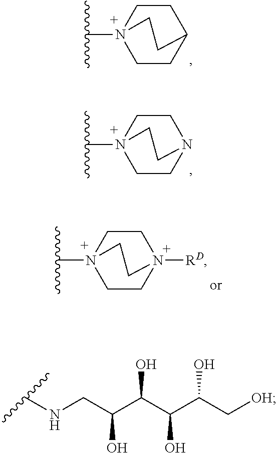

### G¹ is

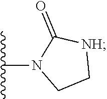 [SMILES: *N1CCNC1=O]

and each R^{D} is independently C₁₋₆ alkyl.

In some embodiments, each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}), or

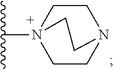

### G¹ is

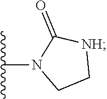 [SMILES: *N1CCNC1=O]

and each R^{D} is independently C₁₋₆ alkyl.

In some embodiments, each R^{C} is independently —OH, —NH₂, —N(R^{D})₂, —N(R^{D})₃⁺, —C(═O)OH, or G; each G is independently —S(═O)₂OH, —S(═O)₂NH₂, —P(═O)(OH)₂, or —P(═O)(OH)(R^{D}); and each R^{D} is independently C₁₋₆ alkyl.

In some embodiments, R⁶ is hydrogen or C₁₋₆ alkyl which is unsubstituted or substituted by 1-6 R^{C} groups; R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—.

In some embodiments, R⁶ is hydrogen or C₁₋₆ alkyl which is unsubstituted or substituted by 1-6 —OH groups; R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-6; R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

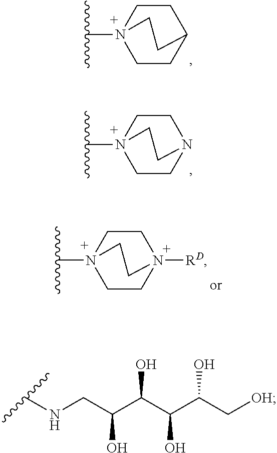

### G¹ is

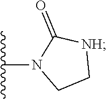 [SMILES: *N1CCNC1=O]

and each R^{D} is independently C₁₋₆ alkyl.

In some embodiments, R⁶ is hydrogen; R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl is substituted by 1-6 R^{C} groups; each V is independently —CH₂O—, —CH₂NR^{D}—, —CH₂N⁺(R^{D})₂—, —NH—C(═O)—NH—, or —C(═O)NH—; s is 1-4; t is 1-3; R⁸ is hydrogen or C₁₋₈ alkyl, wherein the alkyl is substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}),

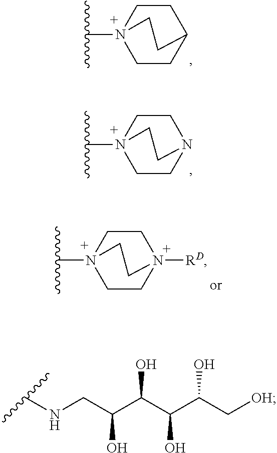

### G¹ is

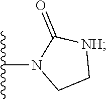 [SMILES: *N1CCNC1=O]

and each R^{D} is independently C₁₋₆ alkyl.

In some embodiments, R⁶ is hydrogen; R⁷ is C₁₋₈ alkyl which is substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}), or

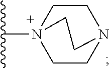

### G¹ is

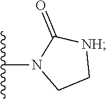 [SMILES: *N1CCNC1=O]

and each R^{D} is independently C₁₋₆ alkyl.

In some embodiments, R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂,

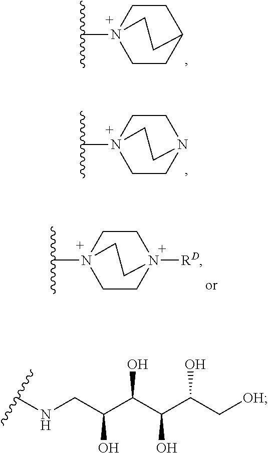

### G¹ is

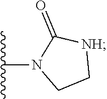 [SMILES: *N1CCNC1=O]

and each R^{D} is independently C₁₋₆ alkyl.

In some embodiments, R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen or C₁₋₈ alkyl which is unsubstituted or substituted by 1-6 R^{C} groups; each R^{C} is independently —OH, —NH₂, —N(R^{D})₂, —N(R^{D})₃⁺, —C(═O)OH, G, or G¹; each G is independently —S(═O)₂OH, —S(═O)₂NH₂, —P(═O)(OH)₂, —P(═O)(OH)(R^{D}),

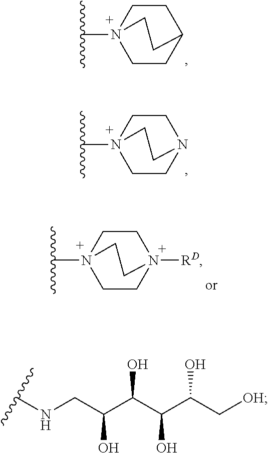

### G¹ is

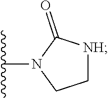 [SMILES: *N1CCNC1=O]

and each R^{D} is independently C₁₋₆ alkyl.

In some embodiments, R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups; R⁸ is hydrogen or C₁₋₈ alkyl which is unsubstituted or substituted by 1-6 R^{C} groups; each R^{C} is independently —OH, —NH₂, —N(R^{D})₂, —N(R^{D})₃⁺, —C(═O)OH, or G; each G is independently —S(═O)₂OH, —S(═O)₂NH₂, —P(═O)(OH)₂, or —P(═O)(OH)(R^{D}); and each R^{D} is independently C₁₋₆ alkyl.

In some embodiments, the compound of Formula (II) has the structure of Formula (D), or a pharmaceutically acceptable salt, solvate, stereoisomer, or prodrug thereof:

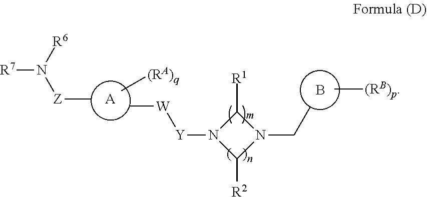

In some embodiments, the compound of Formula (II) has the structure of Formula (D1), (D2), or (D3), or a pharmaceutically acceptable salt, solvate, stereoisomer, or prodrug thereof:

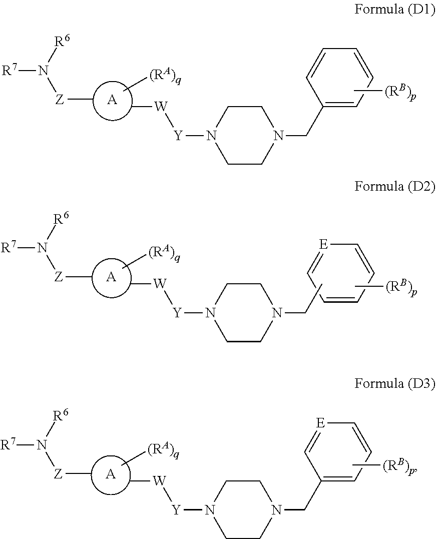

wherein E is CH or N.

In some embodiments, the compound of Formula (II) has the structure of Formula (D1), or a pharmaceutically acceptable salt, solvate, stereoisomer, or prodrug thereof. In some embodiments, the compound of Formula (II) has the structure of Formula (D2), or a pharmaceutically acceptable salt, solvate, stereoisomer, or prodrug thereof. In some embodiments, the compound of Formula (II) has the structure of Formula (D3), or a pharmaceutically acceptable salt, solvate, stereoisomer, or prodrug thereof.

In some embodiments, the compound of Formula (II) has the structure of Formula (D4), (D5), or (D6), or a pharmaceutically acceptable salt, solvate, stereoisomer, or prodrug thereof:

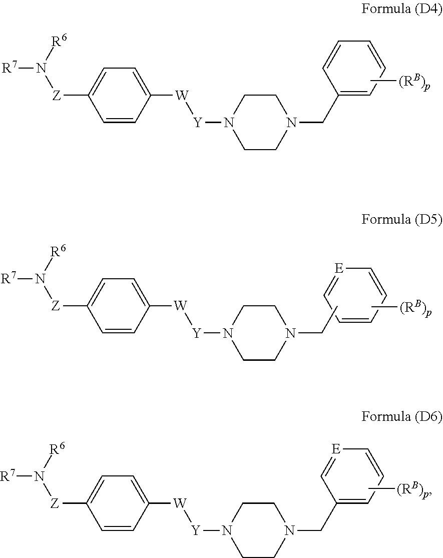

wherein E is CH or N.

In some embodiments, the compound of Formula (II) has the structure of Formula (D4), or a pharmaceutically acceptable salt, solvate, stereoisomer, or prodrug thereof. In some embodiments, the compound of Formula (II) has the structure of Formula (D5), or a pharmaceutically acceptable salt, solvate, stereoisomer, or prodrug thereof. In some embodiments, the compound of Formula (II) has the structure of Formula (D6), or a pharmaceutically acceptable salt, solvate, stereoisomer, or prodrug thereof.

In some embodiments, the compound of Formula (II) has the structure of Formula (D7), (D8), (D9), or (D10), or a pharmaceutically acceptable salt, solvate, stereoisomer, or prodrug thereof:

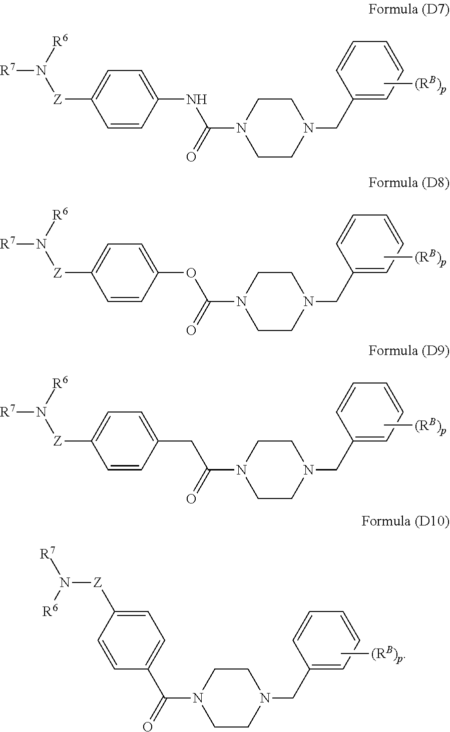

In some embodiments, the compound of Formula (II) has the structure of Formula (D7), or a pharmaceutically acceptable salt, solvate, stereoisomer, or prodrug thereof. In some embodiments, the compound of Formula (II) has the structure of Formula (D8), or a pharmaceutically acceptable salt, solvate, stereoisomer, or prodrug thereof. In some embodiments, the compound of Formula (II) has the structure of Formula (D9), or a pharmaceutically acceptable salt, solvate, stereoisomer, or prodrug thereof. In some embodiments, the compound of Formula (II) has the structure of Formula (D10), or a pharmaceutically acceptable salt, solvate, stereoisomer, or prodrug thereof.

Any combination of the groups described above for the various variables is contemplated herein. Throughout the specification, groups and substituents thereof are chosen by one skilled in the field to provide stable moieties and compounds.

Exemplary compounds of Formulas (I) or Formula (II) include the compounds described in the following tables.

TABLE 1

| Ex. # | Structure | Name |
|---|---|---|
| 1 | 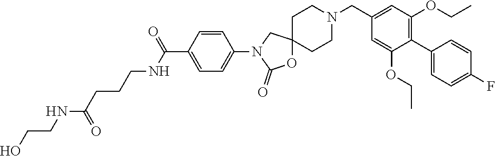 [SMILES: CCOc1cc(CN2CCC3(CC2)CN(c2ccc(C(=O)NCCCC(=O)NCCO)cc2)C(=O)O3)cc(OCC)c1-c1ccc(F)cc1] | 4-(8-((2,6-diethoxy-4′-fluoro-[1,1′-biphenyl]-4- yl)methyl)-2-oxo-1-oxa-3,8-diazaspiro[4.5] decan-3- yl)-N-(4-((2-hydroxyethyl)amino)-4- oxobutyl)benzamide |
| 2 | 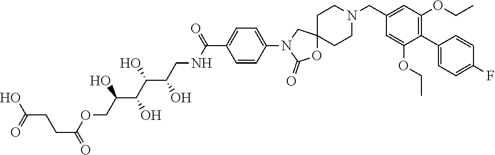 [SMILES: CCOc1cc(CN2CCC3(CC2)CN(c2ccc(C(=O)NC[C@H](O)[C@@H](O)[C@H](O)[C@H](O)COC(=O)CCC(=O)O)cc2)C(=O)O3)cc(OCC)c1-c1ccc(F)cc1] | 4-(((2R,3R,4R,5S)-6-(4-(8-((2,6-diethoxy-4′-fluoro- [1,1′-biphenyl]-4-yl)methyl)-2-oxo-1-oxa-3,8- diazaspiro[4.5]decan-3-yl)benzamido)-2,3,4,5- tetrahydroxyhexyl)oxy)-4-oxobutanoic acid |
| 3 | 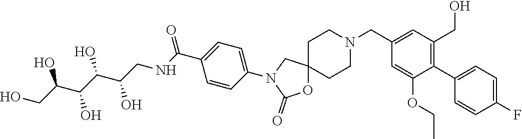 [SMILES: CCOc1cc(CN2CCC3(CC2)CN(c2ccc(C(=O)NC[C@H](O)[C@@H](O)[C@H](O)[C@H](O)CO)cc2)C(=O)O3)cc(CO)c1-c1ccc(F)cc1] | 4-(8-((2-ethoxy-4′-fluoro-6-(hydroxymethyl)-[1,1′- biphenyl]-4-yl)methyl)-2-oxo-1-oxa-3,8- diazaspiro[4.5]decan-3-yl)-N-((2S,3R,4R,5R)- 2,3,4,5,6-pentahydroxyhexyl)benzamide |
| 4 | 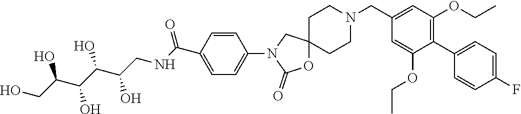 [SMILES: CCOc1cc(CN2CCC3(CC2)CN(c2ccc(C(=O)NC[C@H](O)[C@@H](O)[C@H](O)[C@H](O)CO)cc2)C(=O)O3)cc(OCC)c1-c1ccc(F)cc1] | 4-(8-((2,6-diethoxy-4′-fluoro-[1,1′-biphenyl]-4- yl)methyl)-2-oxo-1-oxa-3,8-diazaspiro[4.5]decan-3- yl)-N-((2S,3R,4R,5R)-2,3,4,5,6- pentahydroxyhexyl)benzamide |
| 5 | 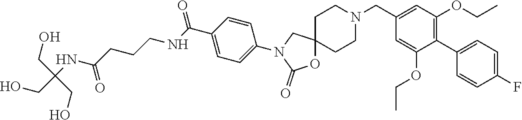 [SMILES: CCOc1cc(CN2CCC3(CC2)CN(c2ccc(C(=O)NCCCC(=O)NC(CO)(CO)CO)cc2)C(=O)O3)cc(OCC)c1-c1ccc(F)cc1] | 4-(8-((2,6-diethoxy-4′-fluoro-[1,1′-biphenyl]-4- methyl)-2-oxo-1-oxa-3,8-diazaspiro[4.5]decan-3- yl)-N-(4-((1,3-dihydroxy-2- (hydroxymethyl)propan-2-yl)amino)-4- oxobutyl)benzamide |
| 6 | 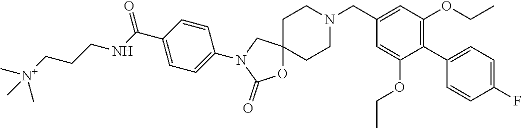 [SMILES: CCOc1cc(CN2CCC3(CC2)CN(c2ccc(C(=O)NCCC[N+](C)(C)C)cc2)C(=O)O3)cc(OCC)c1-c1ccc(F)cc1] | 3-(4-(8-((2,6-diethoxy-4′-fluoro-[1,1′-biphenyl]-4- yl)methyl)-2-oxo-1-oxa-3,8-diazaspiro[4.5]decan-3- yl)benzamido)-N,N,N-trimethylpropan-1-aminium |
| 7 | 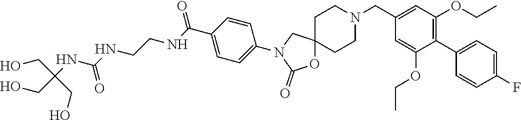 [SMILES: CCOc1cc(CN2CCC3(CC2)CN(c2ccc(C(=O)NCCNC(=O)NC(CO)(CO)CO)cc2)C(=O)O3)cc(OCC)c1-c1ccc(F)cc1] | 4-(8-((2,6-diethoxy-4′-fluoro-[1,1′-biphenyl]-4- yl)methyl)-2-oxo-1-oxa-3,8-diazaspiro[4.5]decan-3- yl)-N-(2-(3-(1,3-dihydroxy-2- (hydroxymethyl)propan-2- yl)ureido)ethyl)benzamide |
| 8 | 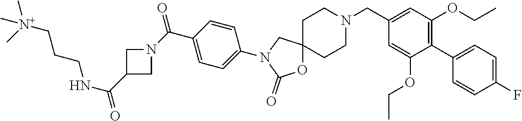 [SMILES: CCOc1cc(CN2CCC3(CC2)CN(c2ccc(C(=O)N4CC(C(=O)NCCC[N+](C)(C)C)C4)cc2)C(=O)O3)cc(OCC)c1-c1ccc(F)cc1] | 3-(1-(4-(8-((2,6-diethoxy-4′-fluoro-[1,1′-biphenyl]- 4-yl)methyl)-2-oxo-1-oxa-3,8-diazaspiro[4.5]decan- 3-yl)benzoyl)azetidine-3-carboxamido)-N,N,N- trimethylpropan-1-aminium |
| 9 | 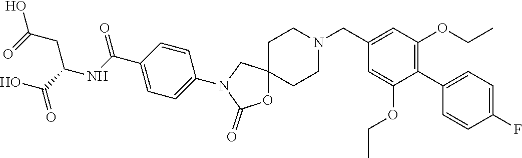 [SMILES: CCOc1cc(CN2CCC3(CC2)CN(c2ccc(C(=O)N[C@@H](CC(=O)O)C(=O)O)cc2)C(=O)O3)cc(OCC)c1-c1ccc(F)cc1] | (4-(8-((2,6-diethoxy-4′-fluoro-[1,1′-biphenyl]-4- yl)methyl)-2-oxo-1-oxa-3,8-diazaspiro[4.5]decan-3- yl)benzoyl)-L-aspartic acid |
| 10 | 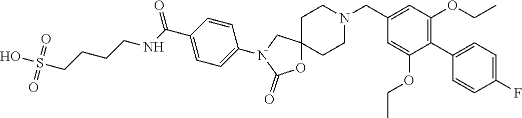 [SMILES: CCOc1cc(CN2CCC3(CC2)CN(c2ccc(C(=O)NCCCCS(=O)(=O)O)cc2)C(=O)O3)cc(OCC)c1-c1ccc(F)cc1] | 4-(4-(8-((2,6-diethoxy-4′-fluoro-[1,1′-biphenyl]-4- yl)methyl)-2-oxo-1-oxa-3,8-diazaspiro[4.5]decan-3- yl)benzamido)butane-1-sulfonic acid |
| 11 | 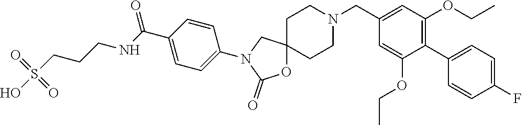 [SMILES: CCOc1cc(CN2CCC3(CC2)CN(c2ccc(C(=O)NCCCS(=O)(=O)O)cc2)C(=O)O3)cc(OCC)c1-c1ccc(F)cc1] | 3-(4-(8-((2,6-diethoxy-4′-fluoro-[1,1′-biphenyl]-4- yl)methyl)-2-oxo-1-oxa-3,8-diazaspiro[4.5]decan-3- yl)benzamido)propane-1-sulfonic acid |
| 12 | 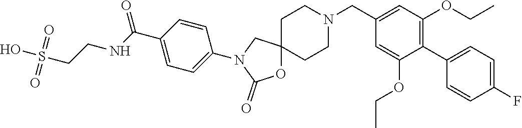 [SMILES: CCOc1cc(CN2CCC3(CC2)CN(c2ccc(C(=O)NCCS(=O)(=O)O)cc2)C(=O)O3)cc(OCC)c1-c1ccc(F)cc1] | 2-(4-(8-((2,6-diethoxy-4′-fluoro-[1,1′-biphenyl]-4- yl)methyl)-2-oxo-1-oxa-3,8-diazaspiro[4.5]decan-3- yl)benzamido)ethane-1-sulfonic acid |
| 13 | 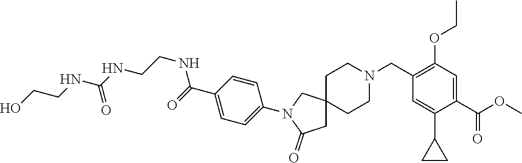 [SMILES: CCOc1cc(C(=O)OC)c(C2CC2)cc1CN1CCC2(CC1)CC(=O)N(c1ccc(C(=O)NCCNC(=O)NCCO)cc1)C2] | methyl 2-cyclopropyl-5-ethoxy-4-((2-(4-((2-(3-(2- hydroxyethyl)ureido)ethyl)carbamoyl)phenyl)-3- oxo-2,8-diazaspiro[4.5]decan-8-yl)methyl)benzoate |
| 14 | 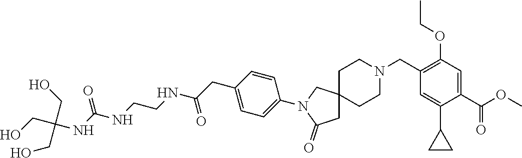 [SMILES: CCOc1cc(C(=O)OC)c(C2CC2)cc1CN1CCC2(CC1)CC(=O)N(c1ccc(CC(=O)NCCNC(=O)NC(CO)(CO)CO)cc1)C2] | methyl 2-cyclopropyl-4-((2-(4-(2-((2-(3-(1,3- dihydroxy-2-(hydroxymethyl)propan-2- yl)ureido)ethyl)amino)-2-oxoethyl)phenyl)-3-oxo- 2,8-diazaspiro[4.5]decan-8-yl)methyl)-5- ethoxy benzoate |
| 15 | 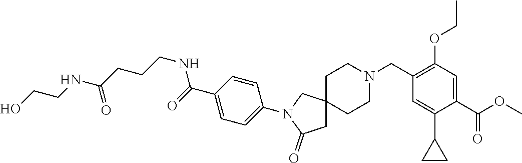 [SMILES: CCOc1cc(C(=O)OC)c(C2CC2)cc1CN1CCC2(CC1)CC(=O)N(c1ccc(C(=O)NCCCC(=O)NCCO)cc1)C2] | methyl 2-cyclopropyl-5-ethoxy-4-((2-(4-((4-((2- hydroxyethyl)amino)-4- oxobutyl)carbamoyl)phenyl)-3-oxo-2,8- diazaspiro[4.5]decan-8-yl)methyl)benzoate |
| 16 | 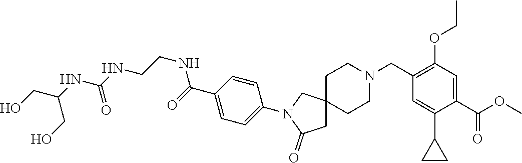 [SMILES: CCOc1cc(C(=O)OC)c(C2CC2)cc1CN1CCC2(CC1)CC(=O)N(c1ccc(C(=O)NCCNC(=O)NC(CO)CO)cc1)C2] | methyl 2-cyclopropyl-4-((2-(4-((2-(3-(1,3- dihydroxypropan-2- yl)ureido)ethyl)carbamoyl)phenyl)-3-oxo-2,8- diazaspiro[4.5]decan-8-yl)methyl)-5- ethoxybenzoate |
| 17 | 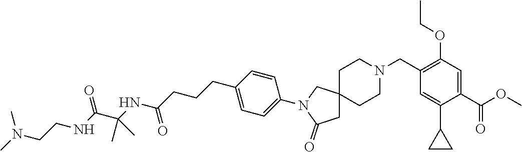 | methyl 2-cyclopropyl-4-((2-(4-(4-((1-((2- (dimethylamino)ethyl)amino)-2-methyl-1- oxopropan-2-yl)amino)-4-oxobutyl)phenyl)-3-oxo- 2.8-diazaspiro[4.5]decan-8-yl)methyl)-5- ethoxybenzoate |
| 18 | 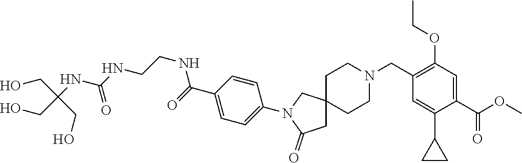 [SMILES: CCOc1cc(C(=O)OC)c(C2CC2)cc1CN1CCC2(CC1)CC(=O)N(c1ccc(C(=O)NCCNC(=O)NC(CO)(CO)CO)cc1)C2] | methyl 2-cyclopropyl-4-((2-(4-((2-(3-(1,3- dihydroxy-2-(hydroxymethyl)propan-2- yl)ureido)ethyl)carbamoyl)phenyl)-3-oxo-2,8- diazaspiro[4.5]decan-8-yl)methyl)-5- ethoxybenzoate |
| 19 | 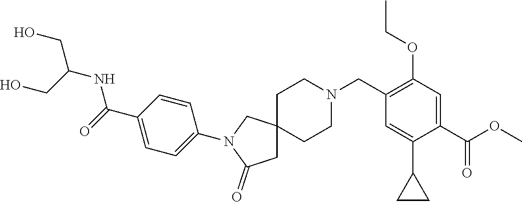 [SMILES: CCOc1cc(C(=O)OC)c(C2CC2)cc1CN1CCC2(CC1)CC(=O)N(c1ccc(C(=O)NC(CO)CO)cc1)C2] | methyl 2-cyclopropyl-4-((2-(4-((1,3- dihydroxypropan-2-yl)carbamoyl)phenyl)-3-oxo- 2.8-diazaspiro[4.5]decan-8-yl)methyl)-5- ethoxybenzoate |
| 20 | 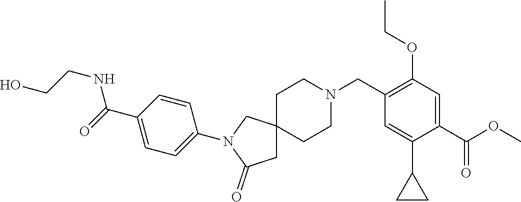 [SMILES: CCOc1cc(C(=O)OC)c(C2CC2)cc1CN1CCC2(CC1)CC(=O)N(c1ccc(C(=O)NCCO)cc1)C2] | methyl 2-cyclopropyl-5-ethoxy-4-((2-(4-((2- hydroxyethyl)carbamoyl)phenyl)-3-oxo-2,8- diazaspiro[4.5]decan-8-yl)methyl)benzoate |
| 21 | 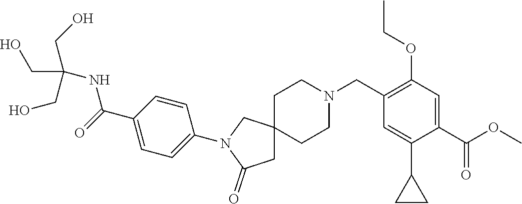 [SMILES: CCOc1cc(C(=O)OC)c(C2CC2)cc1CN1CCC2(CC1)CC(=O)N(c1ccc(C(=O)NC(CO)(CO)CO)cc1)C2] | methyl 2-cyclopropyl-4-((2-(4-((1,3-dihydroxy-2- (hydroxymethyl)propan-2-yl)carbamoyl)phenyl)-3- oxo-2,8-diazaspiro[4.5]decan-8-yl)methyl)-5- ethoxybenzoate |
| 22 | 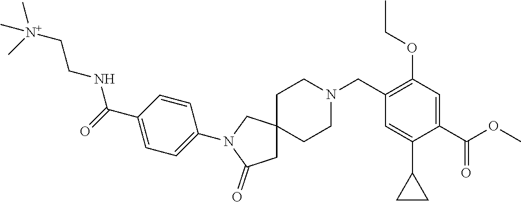 | 2-(4-(8-(5-cyclopropyl-2-ethoxy-4- (methoxycarbonyl)benzyl)-3-oxo-2,8- diazaspiro[4.5]decan-2-yl)benzamido)-N,N,N- trimethylethan-1-aminium |
| 23 | 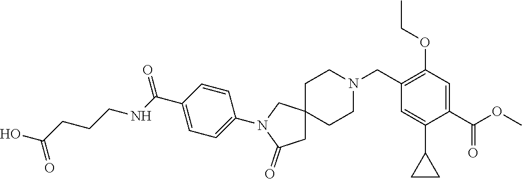 [SMILES: CCOc1cc(C(=O)OC)c(C2CC2)cc1CN1CCC2(CC1)CC(=O)N(c1ccc(C(=O)NCCCC(=O)O)cc1)C2] | 4-(4-(8-(5-cyclopropyl-2-ethoxy-4- (methoxycarbonyl)benzyl)-3-oxo-2,8- diazaspiro[4.5]decan-2-yl)benzamido) butanoic acid |
| 24 | 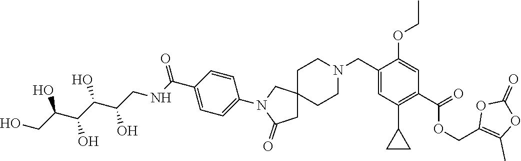 [SMILES: CCOc1cc(C(=O)OCc2oc(=O)oc2C)c(C2CC2)cc1CN1CCC2(CC1)CC(=O)N(c1ccc(C(=O)NC[C@H](O)[C@@H](O)[C@H](O)[C@H](O)CO)cc1)C2] | (5-methyl-2-oxo-1,3-dioxol-4-yl)methyl 2- cyclopropyl-5-ethoxy-4-((3-oxo-2-(4- (((2S,3R,4R,5R)-2,3,4,5,6- pentahydroxyhexyl)carbamoyl)phenyl)-2,8- diazaspiro[4.5]decan-8-yl)methyl)benzoate |
| 25 | 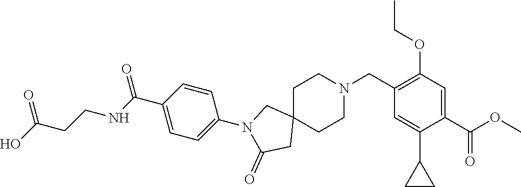 [SMILES: CCOc1cc(C(=O)OC)c(C2CC2)cc1CN1CCC2(CC1)CC(=O)N(c1ccc(C(=O)NCCC(=O)O)cc1)C2] | 3-(4-(8-(5-cyclopropyl-2-ethoxy-4- (methoxycarbonyl)benzyl)-3-oxo-2,8- diazaspiro[4.5]decan-2-yl)benzamido)propanoic acid |
| 26 | 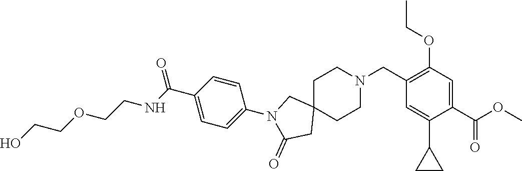 [SMILES: CCOc1cc(C(=O)OC)c(C2CC2)cc1CN1CCC2(CC1)CC(=O)N(c1ccc(C(=O)NCCOCCO)cc1)C2] | methyl 2-cyclopropyl-5-ethoxy-4-((2-(4-((2-(2- hydroxyethoxy)ethyl)carbamoyl)phenyl)-3-oxo-2,8- diazaspiro[4.5]decan-8-yl)methyl)benzoate |
| 27 | 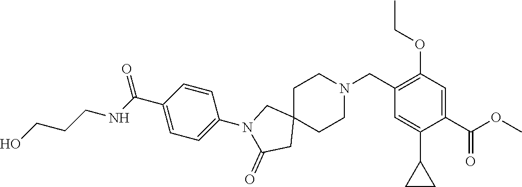 [SMILES: CCOc1cc(C(=O)OC)c(C2CC2)cc1CN1CCC2(CC1)CC(=O)N(c1ccc(C(=O)NCCCO)cc1)C2] | methyl 2-cyclopropyl-5-ethoxy-4-((2-(4-((3- hydroxypropyl)carbamoyl)phenyl)-3-oxo-2,8- diazaspiro[4.5]decan-8-yl)methyl)benzoate |
| 28 | 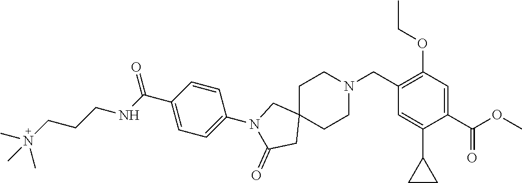 | 3-(4-(8-(5-cyclopropyl-2-ethoxy-4- (methoxycarbonyl)benzyl)-3-oxo-2,8- diazaspiro[4.5]decan-2-yl)benzamido)-N,N,N- trimethylpropan-1-aminium |
| 29 | 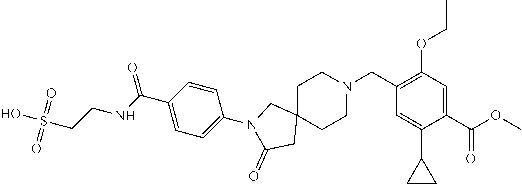 [SMILES: CCOc1cc(C(=O)OC)c(C2CC2)cc1CN1CCC2(CC1)CC(=O)N(c1ccc(C(=O)NCCS(=O)(=O)O)cc1)C2] | 2-(4-(8-(5-cyclopropyl-2-ethoxy-4- (methoxycarbonyl)benzyl)-3-oxo-2,8- diazaspiro[4.5]decan-2-yl)benzamido)ethane-1- sulfonic acid |
| 30 | 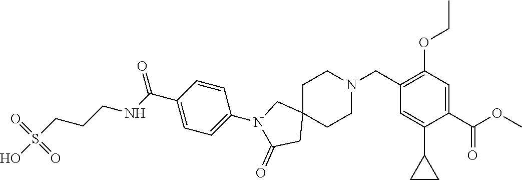 [SMILES: CCOc1cc(C(=O)OC)c(C2CC2)cc1CN1CCC2(CC1)CC(=O)N(c1ccc(C(=O)NCCCS(=O)(=O)O)cc1)C2] | 3-(4-(8-(5-cyclopropyl-2-ethoxy-4- (methoxycarbonyl)benzyl)-3-oxo-2,8- diazaspiro[4.5]decan-2-yl)benzamido)propane-1- sulfonic acid |
| 31 | 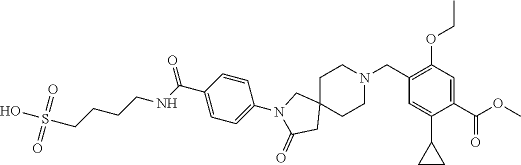 [SMILES: CCOc1cc(C(=O)OC)c(C2CC2)cc1CN1CCC2(CC1)CC(=O)N(c1ccc(C(=O)NCCCCS(=O)(=O)O)cc1)C2] | 4-(4-(8-(5-cyclopropyl-2-ethoxy-4- (methoxycarbonyl)benzyl)-3-oxo-2,8- diazaspiro[4.5] decan-2-yl)benzamido)butane-1- sulfonic acid |
| 32 | 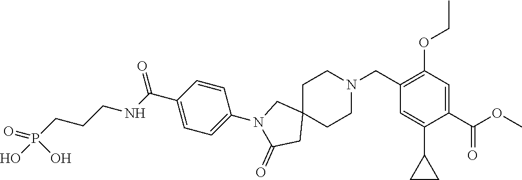 [SMILES: CCOc1cc(C(=O)OC)c(C2CC2)cc1CN1CCC2(CC1)CC(=O)N(c1ccc(C(=O)NCCCP(=O)(O)O)cc1)C2] | (3-(4-(8-(5-cyclopropyl-2-ethoxy-4- (methoxycarbonyl)benzyl)-3-oxo-2,8- diazaspiro[4.5]decan-2- yl)benzamido)propyl)phosphonic acid |
| 33 | 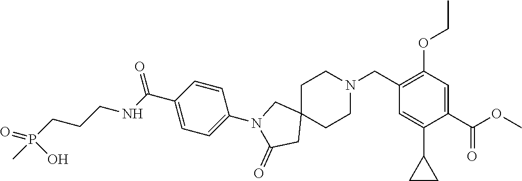 [SMILES: CCOc1cc(C(=O)OC)c(C2CC2)cc1CN1CCC2(CC1)CC(=O)N(c1ccc(C(=O)NCCCP(C)(=O)O)cc1)C2] | (3-(4-(8-(5-cyclopropyl-2-ethoxy-4- (methoxycarbonyl)benzyl)-3-oxo-2,8- diazaspiro[4.5]decan-2- yl)benzamido)propyl)(methyl)phosphinic acid |
| 34 | 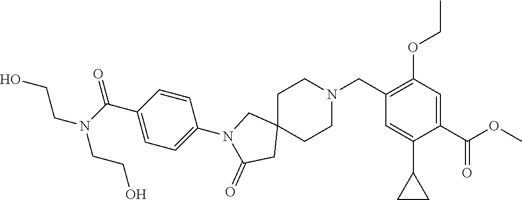 [SMILES: CCOc1cc(C(=O)OC)c(C2CC2)cc1CN1CCC2(CC1)CC(=O)N(c1ccc(C(=O)N(CCO)CCO)cc1)C2] | methyl 4-((2-(4-(bis(2- hydroxyethyl)carbamoyl)phenyl)-3-oxo-2,8- diazaspiro[4.5]decan-8-yl)methyl)-2-cyclopropyl-5- ethoxybenzoate |
| 35 | 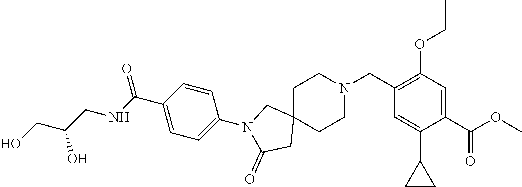 [SMILES: CCOc1cc(C(=O)OC)c(C2CC2)cc1CN1CCC2(CC1)CC(=O)N(c1ccc(C(=O)NC[C@H](O)CO)cc1)C2] | methyl (S)-2-cyclopropyl-4-((2-(4-((2,3- dihydroxypropyl)carbamoyl)phenyl)-3-oxo-2,8- diazaspiro[4.5]decan-8-yl)methyl)-5- ethoxybenzoate |
| 36 | 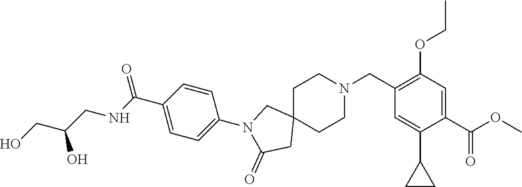 [SMILES: CCOc1cc(C(=O)OC)c(C2CC2)cc1CN1CCC2(CC1)CC(=O)N(c1ccc(C(=O)NC[C@@H](O)CO)cc1)C2] | methyl (R)-2-cyclopropyl-4-((2-(4-((2,3- dihydroxypropyl)carbamoyl)phenyl)-3-oxo-2,8- diazaspiro[4.5]decan-8-yl)methyl)-5- ethoxybenzoate |
| 37 | 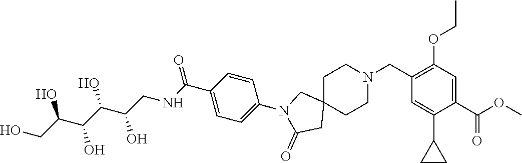 [SMILES: CCOc1cc(C(=O)OC)c(C2CC2)cc1CN1CCC2(CC1)CC(=O)N(c1ccc(C(=O)NC[C@H](O)[C@@H](O)[C@H](O)[C@H](O)CO)cc1)C2] | methyl 2-cyclopropyl-5-ethoxy-4-((3-oxo-2-(4- (((2S,3R,4R,5R)-2,3,4,5,6- pentahydroxyhexyl)carbamoyl)phenyl)-2,8- diazaspiro[4.5]decan-8-yl)methyl)benzoate |
| 38 | 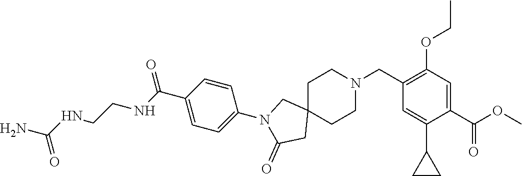 [SMILES: CCOc1cc(C(=O)OC)c(C2CC2)cc1CN1CCC2(CC1)CC(=O)N(c1ccc(C(=O)NCCNC(N)=O)cc1)C2] | methyl 2-cyclopropyl-5-ethoxy-4-((3-oxo-2-(4-((2- ureidoethyl)carbamoyl)phenyl)-2,8- diazaspiro[4.5]decan-8-yl)methyl)benzoate |
| 39 | 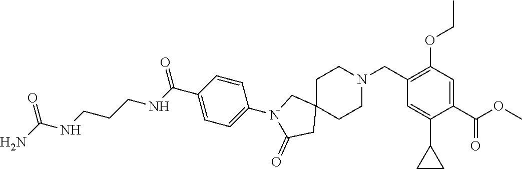 [SMILES: CCOc1cc(C(=O)OC)c(C2CC2)cc1CN1CCC2(CC1)CC(=O)N(c1ccc(C(=O)NCCCNC(N)=O)cc1)C2] | methyl 2-cyclopropyl-5-ethoxy-4-((3-oxo-2-(4-((3- ureidopropyl)carbamoyl)phenyl)-2,8- diazaspiro[4.5]decan-8-yl)methyl)benzoate |
| 40 | 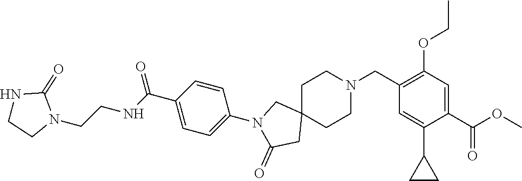 [SMILES: CCOc1cc(C(=O)OC)c(C2CC2)cc1CN1CCC2(CC1)CC(=O)N(c1ccc(C(=O)NCCN3CCNC3=O)cc1)C2] | methyl 2-cyclopropyl-5-ethoxy-4-((3-oxo-2-(4-((2- (2-oxoimidazolidin-1-yl)ethyl)carbamoyl)phenyl)- 2,8-diazaspiro[4.5]decan-8-yl)methyl)benzoate |
| 41 | 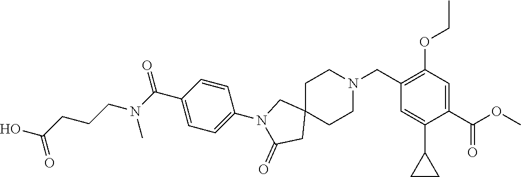 [SMILES: CCOc1cc(C(=O)OC)c(C2CC2)cc1CN1CCC2(CC1)CC(=O)N(c1ccc(C(=O)N(C)CCCC(=O)O)cc1)C2] | 4-(4-(8-(5-cyclopropyl-2-ethoxy-4- (methoxycarbonyl)benzyl)-3-oxo-2,8- diazaspiro[4.5]decan-2-yl)-N- methylbenzamido)butanoic acid |
| 42 | 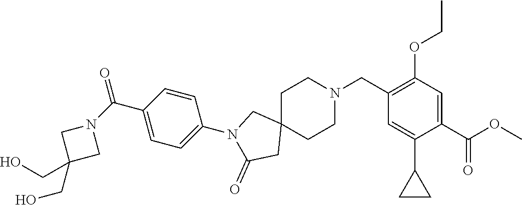 [SMILES: CCOc1cc(C(=O)OC)c(C2CC2)cc1CN1CCC2(CC1)CC(=O)N(c1ccc(C(=O)N3CC(CO)(CO)C3)cc1)C2] | methyl 4-((2-(4-(3,3-bis(hydroxymethyl)azetidine- 1-carbonyl)phenyl)-3-oxo-2,8-diazaspiro[4.5]decan- 8-yl)methyl)-2-cyclopropyl-5-ethoxybenzoate |
| 43 | 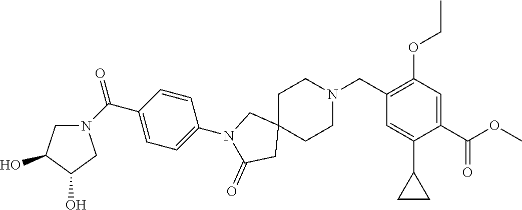 [SMILES: CCOc1cc(C(=O)OC)c(C2CC2)cc1CN1CCC2(CC1)CC(=O)N(c1ccc(C(=O)N3C[C@H](O)[C@@H](O)C3)cc1)C2] | methyl 2-cyclopropyl-4-((2-(4-((3S,4S)-3,4- dihydroxypyrrolidine-1-caibonyl)phenyl)-3-oxo- 2,8-diazaspiro[4.5]decan-8-yl)methyl)-5- ethoxy benzoate |
| 44 | 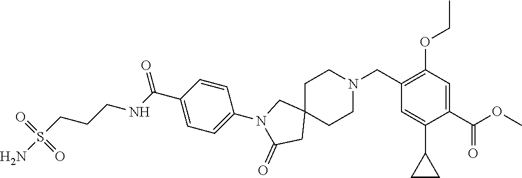 [SMILES: CCOc1cc(C(=O)OC)c(C2CC2)cc1CN1CCC2(CC1)CC(=O)N(c1ccc(C(=O)NCCCS(N)(=O)=O)cc1)C2] | methyl 2-cyclopropyl-5-ethoxy-4-((3-oxo-2-(4-((3- sulfamoylpropyl)carbamoyl)phenyl)-2,8- diazaspiro[4.5]decan-8-yl)methyl)benzoate |
| 45 | 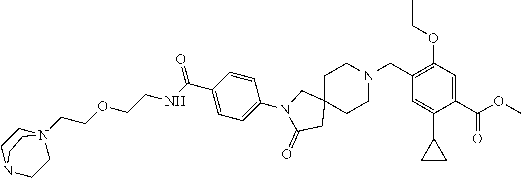 | 1-(2-(2-(4-(8-(5-cyclopropyl-2-ethoxy-4- (methoxycarbonyl)benzyl)-3-oxo-2,8- diazaspiro[4.5 ]decan-2- yl)benzamido)ethoxy)ethyl)-1,4- diazabicyclo[2.2.2]octan-1-ium |
| 46 | 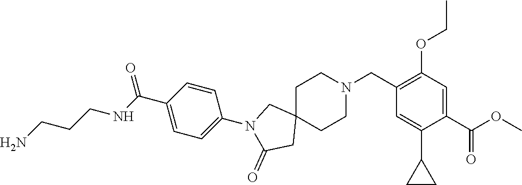 [SMILES: CCOc1cc(C(=O)OC)c(C2CC2)cc1CN1CCC2(CC1)CC(=O)N(c1ccc(C(=O)NCCCN)cc1)C2] | methyl 4-((2-(4-((3- aminopropyl)carbamoyl)phenyl)-3-oxo-2,8- diazaspiro[4.5]decan-8-yl)methyl)-2-cyclopropyl-5- ethoxybenzoate |
| 47 | 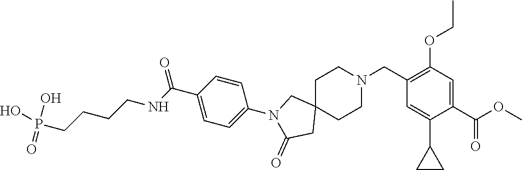 [SMILES: CCOc1cc(C(=O)OC)c(C2CC2)cc1CN1CCC2(CC1)CC(=O)N(c1ccc(C(=O)NCCCCP(=O)(O)O)cc1)C2] | (4-(4-(8-(5-cyclopropyl-2-ethoxy-4- (methoxycarbonyl)benzyl)-3-oxo-2,8- diazaspiro[4.5]decan-2- yl)benzamido)butyl)phosphonic acid |
| 48 | 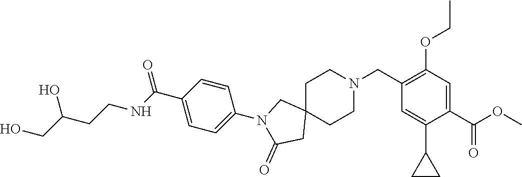 [SMILES: CCOc1cc(C(=O)OC)c(C2CC2)cc1CN1CCC2(CC1)CC(=O)N(c1ccc(C(=O)NCCC(O)CO)cc1)C2] | methyl 2-cyclopropyl-4-((2-(4-((3,4- dihydroxybutyl)carbamoyl)phenyl)-3-oxo-2,8- diazaspiro[4.5]decan-8-yl)methyl)-5- ethoxybenzoate |
| 49 | 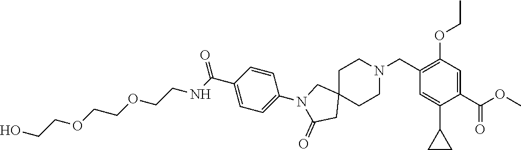 [SMILES: CCOc1cc(C(=O)OC)c(C2CC2)cc1CN1CCC2(CC1)CC(=O)N(c1ccc(C(=O)NCCOCCOCCO)cc1)C2] | methyl 2-cyclopropyl-5-ethoxy-4-((2-(4-((2-(2-(2- hydroxyethoxy)ethoxy)ethyl)carbamoyl)phenyl)-3- oxo-2,8-diazaspiro[4.5]decan-8-yl)methyl)benzoate |
| 50 | 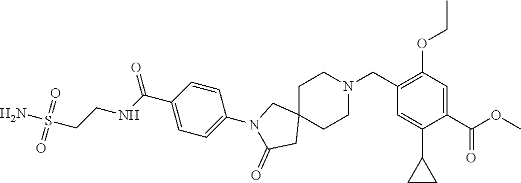 [SMILES: CCOc1cc(C(=O)OC)c(C2CC2)cc1CN1CCC2(CC1)CC(=O)N(c1ccc(C(=O)NCCS(N)(=O)=O)cc1)C2] | methyl 2-cyclopropyl-5-ethoxy-4-((3-oxo-2-(4-((2- sulfamoylethyl)carbamoyl)phenyl)-2,8- diazaspiro[4.5]decan-8-yl)methyl)benzoate |
| 51 | 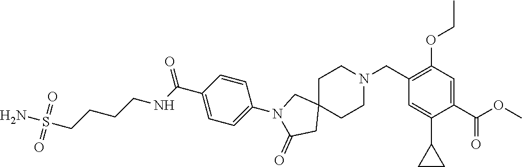 [SMILES: CCOc1cc(C(=O)OC)c(C2CC2)cc1CN1CCC2(CC1)CC(=O)N(c1ccc(C(=O)NCCCCS(N)(=O)=O)cc1)C2] | methyl 2-cyclopropyl-5-ethoxy-4-((3-oxo-2-(4-((4- sulfamoylbutyl)carbamoyl)phenyl)-2,8- diazaspiro[4.5]decan-8-yl)methyl)benzoate |
| 52 | 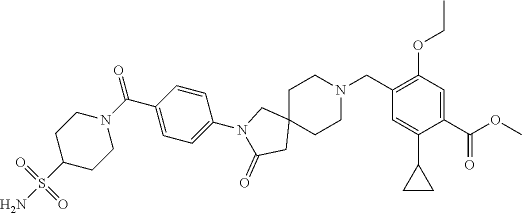 [SMILES: CCOc1cc(C(=O)OC)c(C2CC2)cc1CN1CCC2(CC1)CC(=O)N(c1ccc(C(=O)N3CCC(S(N)(=O)=O)CC3)cc1)C2] | methyl 2-cyclopropyl-5-ethoxy-4-((3-oxo-2-(4-(4- sulfamoylpiperidine-1-carbonyl)phenyl)-2,8- diazaspiro[4.5]decan-8-yl)methyl)benzoate |
| 53 | 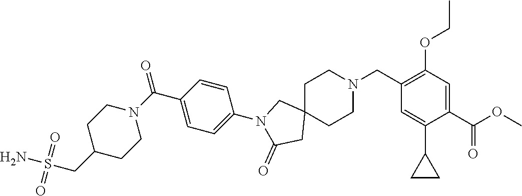 [SMILES: CCOc1cc(C(=O)OC)c(C2CC2)cc1CN1CCC2(CC1)CC(=O)N(c1ccc(C(=O)N3CCC(CS(N)(=O)=O)CC3)cc1)C2] | methyl 2-cyclopropyl-5-ethoxy-4-((3-oxo-2-(4-(4- (sulfamoylmethyl)piperidine-1-carbonyl)phenyl)- 2,8-diazaspiro[4.5]decan-8-yl)methyl)benzoate |
| 54 | 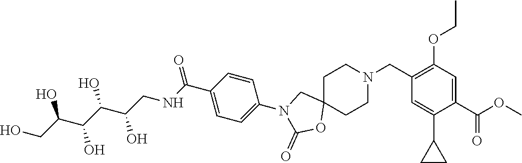 [SMILES: CCOc1cc(C(=O)OC)c(C2CC2)cc1CN1CCC2(CC1)CN(c1ccc(C(=O)NC[C@H](O)[C@@H](O)[C@H](O)[C@H](O)CO)cc1)C(=O)O2] | methyl 2-cyclopropyl-5-ethoxy-4-((2-oxo-3-(4- (((2S,3R,4R,5R)-2,3,4,5,6- pentahydroxyhexyl)carbamoyl)phenyl)-1-oxa-3,8- diazaspiro[4.5]decan-8-yl)methyl)benzoate |
| 55 | 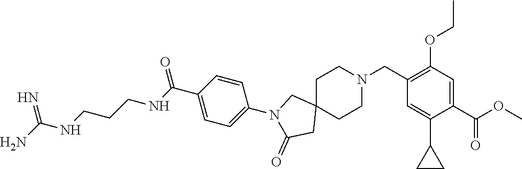 [SMILES: CCOc1cc(C(=O)OC)c(C2CC2)cc1CN1CCC2(CC1)CC(=O)N(c1ccc(C(=O)NCCCNC(=N)N)cc1)C2] | methyl 2-cyclopropyl-5-ethoxy-4-((2-(4-((3- guanidinopropyl)carbamoyl)phenyl)-3-oxo-2,8- diazaspiro [4.5]decan-8-yl)methy l)benzoate |
| 56 | 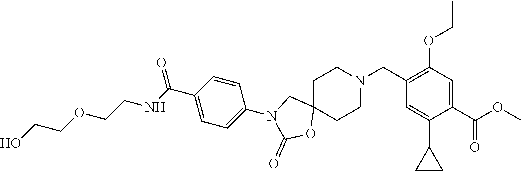 [SMILES: CCOc1cc(C(=O)OC)c(C2CC2)cc1CN1CCC2(CC1)CN(c1ccc(C(=O)NCCOCCO)cc1)C(=O)O2] | methyl 2-cyclopropyl-5-ethoxy-4-((3-(4-((2-(2- hydroxyethoxy)ethyl)carbamoyl)phenyl)-2-oxo-1- oxa-3,8-diazaspiro[4.5]decan-8-yl)methyl)benzoate |
| 57 | 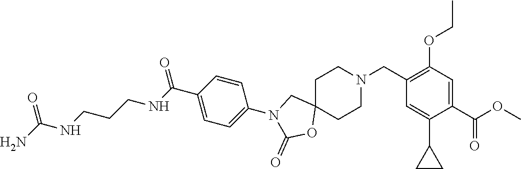 [SMILES: CCOc1cc(C(=O)OC)c(C2CC2)cc1CN1CCC2(CC1)CN(c1ccc(C(=O)NCCCNC(N)=O)cc1)C(=O)O2] | methyl 2-cyclopropyl-5-ethoxy-4-((2-oxo-3-(4-((3- ureidopropyl)carbamoyl)phenyl)-1-oxa-3,8- diazaspiro [4.5]decan-8-yl)methyl)benzoate |
| 58 | 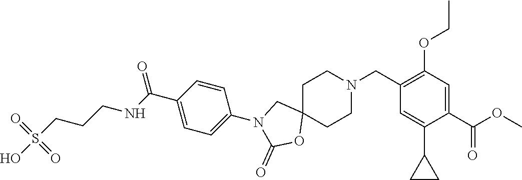 [SMILES: CCOc1cc(C(=O)OC)c(C2CC2)cc1CN1CCC2(CC1)CN(c1ccc(C(=O)NCCCS(=O)(=O)O)cc1)C(=O)O2] | 3-(4-(8-(5-cyclopropyl-2-ethoxy-4- (methoxycarbonyl)benzyl)-2-oxo-1-oxa-3,8- diazaspiro[4.5]decan-3-yl)benzamido)propane-1- sulfonic acid |
| 59 | 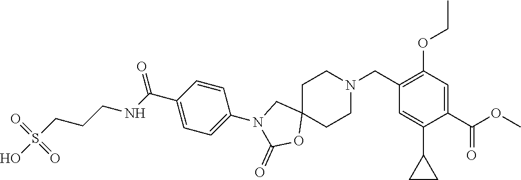 [SMILES: CCOc1cc(C(=O)OC)c(C2CC2)cc1CN1CCC2(CC1)CN(c1ccc(C(=O)NCCCS(=O)(=O)O)cc1)C(=O)O2] | methyl 2-cyclopropyl-5-ethoxy-4-((2-oxo-3-(4-((3- sulfamoylpropyl)carbamoyl)phenyl)-1-oxa-3,8- diazaspiro[4.5]decan-8-yl)methyl)benzoate |
| 60 | 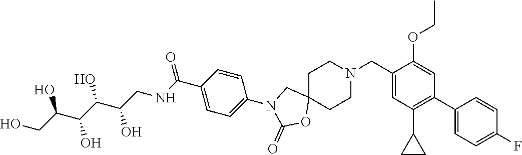 [SMILES: CCOc1cc(-c2ccc(F)cc2)c(C2CC2)cc1CN1CCC2(CC1)CN(c1ccc(C(=O)NC[C@H](O)[C@@H](O)[C@H](O)[C@H](O)CO)cc1)C(=O)O2] | 4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)-N-((2S,3R,4R,5R)- 2,3,4,5,6-pentahydroxyhexyl)benzamide |
| 61 | 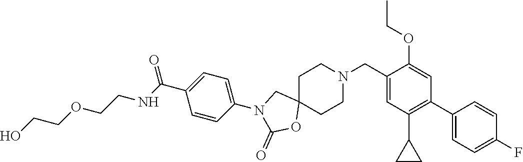 [SMILES: CCOc1cc(-c2ccc(F)cc2)c(C2CC2)cc1CN1CCC2(CC1)CN(c1ccc(C(=O)NCCOCCO)cc1)C(=O)O2] | 4-(8-((2-cyclopropyl-5-ethoxy-4′-fluoro-[1,1′- biphenyl]-4-yl)methyl)-2-oxo-1-oxa-3,8- diazaspiro [4.5]decan-3-yl)-N-(2-(2- hydroxyethoxy)ethyl)benzamide |
| 62 | 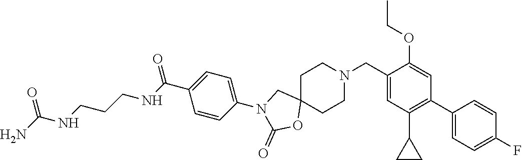 [SMILES: CCOc1cc(-c2ccc(F)cc2)c(C2CC2)cc1CN1CCC2(CC1)CN(c1ccc(C(=O)NCCCNC(N)=O)cc1)C(=O)O2] | 4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)-N-(3- ureidopropyl)benzamide |
| 63 | 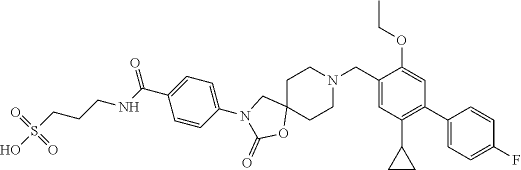 [SMILES: CCOc1cc(-c2ccc(F)cc2)c(C2CC2)cc1CN1CCC2(CC1)CN(c1ccc(C(=O)NCCCS(=O)(=O)O)cc1)C(=O)O2] | 3-(4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)benzamido)propane-1- sulfonic acid |
| 64 | 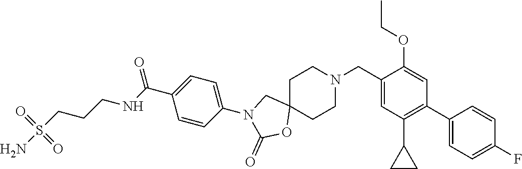 [SMILES: CCOc1cc(-c2ccc(F)cc2)c(C2CC2)cc1CN1CCC2(CC1)CN(c1ccc(C(=O)NCCCS(N)(=O)=O)cc1)C(=O)O2] | 4-(8-((2-cyclopropyl-5-ethoxy-4′-fluoro-[1,1′- biphenyl]-4-yl)methyl)-2-o xo-1-oxa-3,8- diazaspiro[4.5]decan-3-yl)-N-(3- sulfamoylpropyl)benzamide |
| 65 | 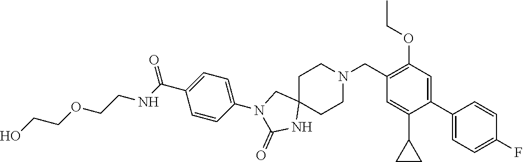 [SMILES: CCOc1cc(-c2ccc(F)cc2)c(C2CC2)cc1CN1CCC2(CC1)CN(c1ccc(C(=O)NCCOCCO)cc1)C(=O)N2] | 4-(8-((2-cyclopropyl-5-ethoxy-4′-fluoro-[1,1′- biphenyl]-4-yl)methyl)-2-oxo-1,3,8- triazaspiro[4.5] decan-3-yl) -N-(2-(2- hydroxyethoxy)ethyl)benzamide |
| 66 | 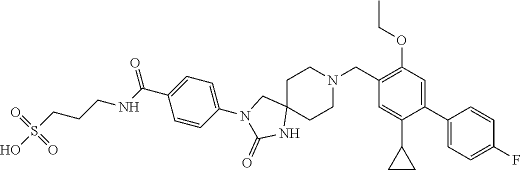 [SMILES: CCOc1cc(-c2ccc(F)cc2)c(C2CC2)cc1CN1CCC2(CC1)CN(c1ccc(C(=O)NCCCS(=O)(=O)O)cc1)C(=O)N2] | 3-(4-(8-((2-cyclopropyl-5-ethoxy-4′-fluoro-[1,1′- biphenyl]-4-yl)methyl)-2-oxo-1,3,8- triazaspiro[4.5]decan-3-yl)benzamido)propane-1- sulfonic acid |
| 67 | 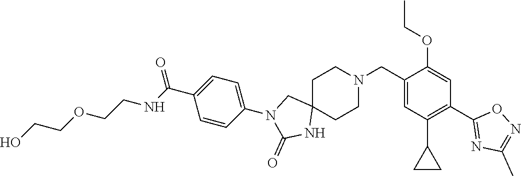 [SMILES: CCOc1cc(-c2nc(C)no2)c(C2CC2)cc1CN1CCC2(CC1)CN(c1ccc(C(=O)NCCOCCO)cc1)C(=O)N2] | 4-(8-(5-cyclopropyl-2-ethoxy-4-(3-methyl-1,2,4- oxadiazol-5-yl)benzyl)-2-oxo-1,3,8- triazaspiro[4.5]decan-3-yl)-N-(2-(2- hydroxyethoxy)ethyl)benzamide |
| 68 | 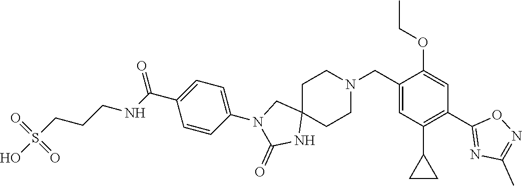 [SMILES: CCOc1cc(-c2nc(C)no2)c(C2CC2)cc1CN1CCC2(CC1)CN(c1ccc(C(=O)NCCCS(=O)(=O)O)cc1)C(=O)N2] | 3-(4-(8-(5-cyclopropyl-2-ethoxy-4-(3-methyl-1,2,4- oxadiazol-5-yl)benzyl)-2-oxo-1,3,8- triazaspiro[4.5]decan-3-yl)benzamido)propane-1- sulfonic acid |
| 69 | 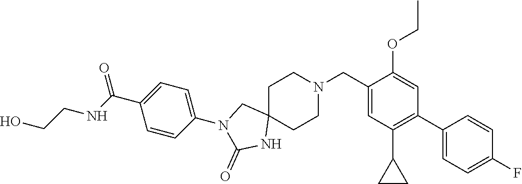 [SMILES: CCOc1cc(-c2ccc(F)cc2)c(C2CC2)cc1CN1CCC2(CC1)CN(c1ccc(C(=O)NCCO)cc1)C(=O)N2] | 4-(8-((2-cyclopropyl-5-ethoxy-4′-fluoro-[1,1′- biphenyl]-4-yl)methyl)-2-oxo-1,3,8- triazaspiro[4.5]decan-3-yl)-N-(2- hydroxyethyl)benzamide |
| 70 | 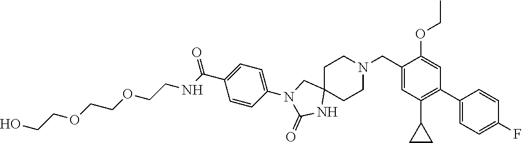 [SMILES: CCOc1cc(-c2ccc(F)cc2)c(C2CC2)cc1CN1CCC2(CC1)CN(c1ccc(C(=O)NCCOCCOCCO)cc1)C(=O)N2] | 4-(8-((2-cyclopropyl-5-ethoxy-4′-fluoro-[1,1′- biphenyl]-4-yl)methyl)-2-oxo-1,3,8- triazaspiro[4.5]decan-3-yl)-N-(2-(2-(2- hydroxyethoxy)ethoxy)ethyl)benzamide |
| 71 | 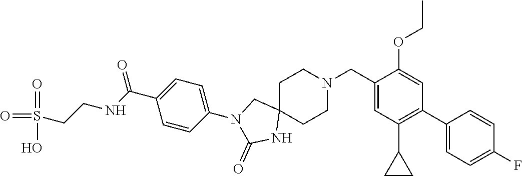 [SMILES: CCOc1cc(-c2ccc(F)cc2)c(C2CC2)cc1CN1CCC2(CC1)CN(c1ccc(C(=O)NCCS(=O)(=O)O)cc1)C(=O)N2] | 2-(4-(8-((2-cyclopropyl-5-ethoxy-4′-fluoro-[1,1′- biphenyl]-4-yl)methyl)-2-oxo-1,3,8- triazaspiro[4.5]decan-3-yl)benzamido)ethane-1- sulfonic acid |
| 72 | 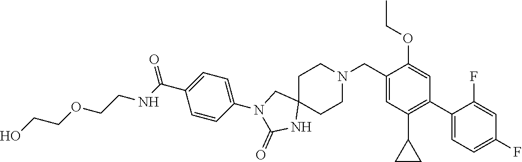 [SMILES: CCOc1cc(-c2ccc(F)cc2F)c(C2CC2)cc1CN1CCC2(CC1)CN(c1ccc(C(=O)NCCOCCO)cc1)C(=O)N2] | 4-(8-((2-cyclopropyl-5-ethoxy-2′,4′-difluoro-[1,1′- biphenyl]-4-yl)methyl)-2-oxo-1,3,8- triazaspiro[4.5]decan-3-yl)-N-(2-(2- hydroxyethoxy)ethyl)benzamide |
| 73 | 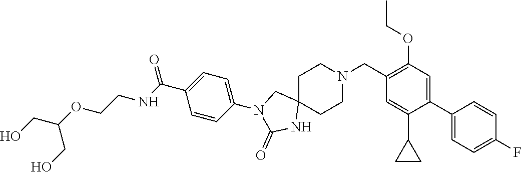 [SMILES: CCOc1cc(-c2ccc(F)cc2)c(C2CC2)cc1CN1CCC2(CC1)CN(c1ccc(C(=O)NCCOC(CO)CO)cc1)C(=O)N2] | 4-(8-((2-cyclopropyl-5-ethoxy-4′-fluoro-[1,1′- biphenyl]-4-yl)methyl)-2-oxo-1,3,8- triazaspiro[4.5]decan-3-yl)-N-(2-((1,3- dihydroxypropan-2-yl)oxy)ethyl)benzamide |
| 74 | 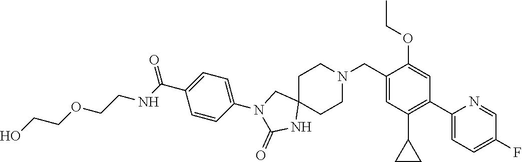 [SMILES: CCOc1cc(-c2ccc(F)cn2)c(C2CC2)cc1CN1CCC2(CC1)CN(c1ccc(C(=O)NCCOCCO)cc1)C(=O)N2] | 4-(8-(5-cyclopropyl-2-ethoxy-4-(5-fluoropyridin-2- yl)benzyl)-2-oxo-1,3,8-triazaspiro[4.5]decan-3-yl)- N-(2-(2-hydroxyethoxy)ethyl)benzamide |
| 75 | 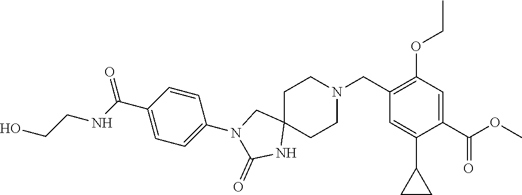 [SMILES: CCOc1cc(C(=O)OC)c(C2CC2)cc1CN1CCC2(CC1)CN(c1ccc(C(=O)NCCO)cc1)C(=O)N2] | methyl 2-cyclopropyl-5-ethoxy-4-((3-(4-((2- hydroxyethyl)carbamoyl)phenyl)-2-oxo-1,3,8- triazaspiro[4.5]decan-8-yl)methyl)benzoate |
| 76 | 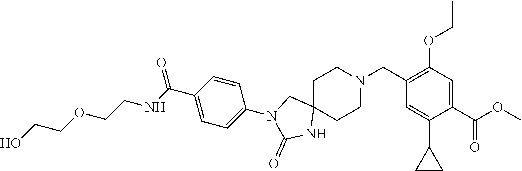 [SMILES: CCOc1cc(C(=O)OC)c(C2CC2)cc1CN1CCC2(CC1)CN(c1ccc(C(=O)NCCOCCO)cc1)C(=O)N2] | methyl 2-cyclopropyl-5-ethoxy-4-((3-(4-((2-(2- hydroxyethoxy)ethyl)carbamoyl)phenyl)-2-oxo- 1,3,8-triazaspiro[4.5]decan-8-yl)methyl)benzoate |
| 77 | 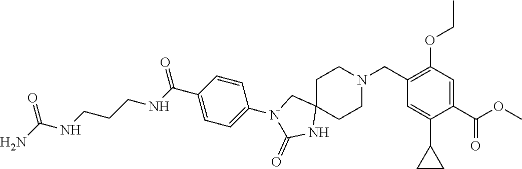 [SMILES: CCOc1cc(C(=O)OC)c(C2CC2)cc1CN1CCC2(CC1)CN(c1ccc(C(=O)NCCCNC(N)=O)cc1)C(=O)N2] | methyl 2-cyclopropyl-5-ethoxy-4-((2-oxo-3-(4-((3- ureidopropyl)carbamoyl)phenyl)-1,3,8- triazaspiro[4.5 ]decan-8-yl)methyl)benzoate |
| 78 | 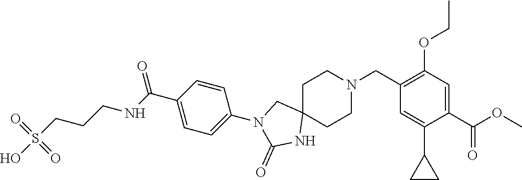 [SMILES: CCOc1cc(C(=O)OC)c(C2CC2)cc1CN1CCC2(CC1)CN(c1ccc(C(=O)NCCCS(=O)(=O)O)cc1)C(=O)N2] | 3-(4-(8-(5-cyclopropyl-2-ethoxy-4- (methoxycarbonyl)benzyl)-2-oxo-1,3,8- triazaspiro[4.5]decan-3-yl)benzamido)propane-1- sulfonic acid |
| 79 | 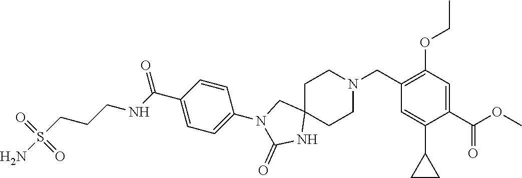 [SMILES: CCOc1cc(C(=O)OC)c(C2CC2)cc1CN1CCC2(CC1)CN(c1ccc(C(=O)NCCCS(N)(=O)=O)cc1)C(=O)N2] | methyl 2-cyclopropyl-5-ethoxy-4-((2-oxo-3-(4-((3- sulfamoylpropyl)carbamoyl)phenyl)-1,3,8- triazaspiro[4.5]decan-8-yl)methyl)benzoate |
| 80 | 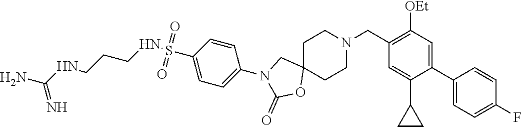 [SMILES: CCOc1cc(-c2ccc(F)cc2)c(C2CC2)cc1CN1CCC2(CC1)CN(c1ccc(S(=O)(=O)NCCCNC(=N)N)cc1)C(=O)O2] | 4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)-N-(3- guanidinopropyl)benzenesulfonamide |
| 81 | 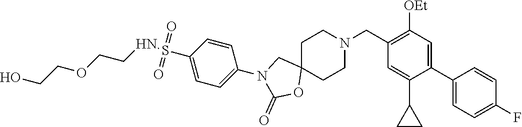 [SMILES: CCOc1cc(-c2ccc(F)cc2)c(C2CC2)cc1CN1CCC2(CC1)CN(c1ccc(S(=O)(=O)NCCOCCO)cc1)C(=O)O2] | 4-(8-((2-cyclopropyl-5-ethoxy-4′-fluoro-[1,1- biphenyl]-4-yl)methyl)-2-oxo-1-oxa-3,8- diazaspiro[4.5]decan-3-yl)-N-(2-(2- hydroxyethoxy)ethyl)benzenesulfonamide |
| 82 | 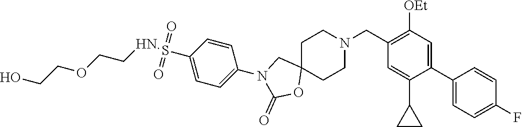 [SMILES: CCOc1cc(-c2ccc(F)cc2)c(C2CC2)cc1CN1CCC2(CC1)CN(c1ccc(S(=O)(=O)NCCOCCO)cc1)C(=O)O2] | 4-(8-(5-cyclopropyl-2-ethoxy-4-(5-fluoropyridin-2- yl)benzyl)-2-oxo-1-oxa-3,8-diazaspiro[4.5]decan-3- yl)-N-(2-(2- hydroxyethoxy)ethyl)benzenesulfonamide |
| 83 | 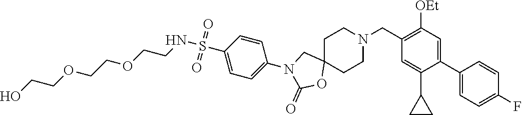 [SMILES: CCOc1cc(-c2ccc(F)cc2)c(C2CC2)cc1CN1CCC2(CC1)CN(c1ccc(S(=O)(=O)NCCOCCOCCO)cc1)C(=O)O2] | 4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)-N-(2-(2-(2- hydroxyethoxy)ethoxy)ethyl)benzenesulfonamide |
| 84 | 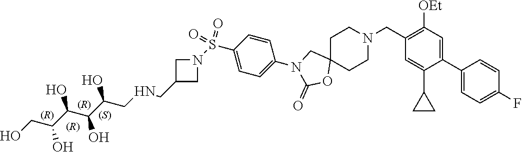 | 8-[[5-cyclopropyl-2-ethoxy-4-(4- fluorophenyl)phenyl]methyl]-3-[4-[3- [[[(2S,3R,4R,5R)-2,3,4,5,6- pentahydroxyhexyl]amino]methyl]azetidin-1- yl]sulfonylphenyl]-1-oxa-3,8-diazaspiro[4.5]decan- 2-one |
| 85 | 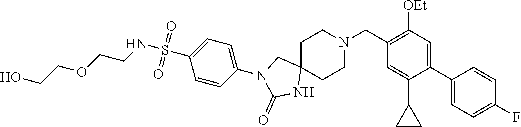 [SMILES: CCOc1cc(-c2ccc(F)cc2)c(C2CC2)cc1CN1CCC2(CC1)CN(c1ccc(S(=O)(=O)NCCOCCO)cc1)C(=O)N2] | 4-(8-((2-cyclopropyl-5-ethoxy-4′-fluoro-[1,1′- biphenyl]-4-yl)methyl)-2-oxo-1,3,8- triazaspiro[4.5]decan-3-yl)-N-(2-(2- hydroxyethoxy)ethyl)benzenesulfonamide |
| 86 | 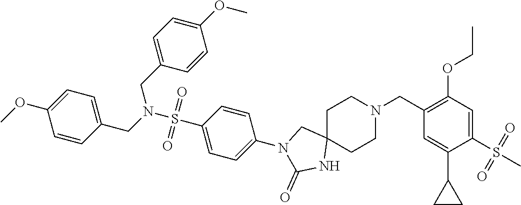 | 4-(8-(5-cyclopropyl-2-ethoxy-4- (methylsulfonyl)benzyl)-2-oxo-1,3,8- triazaspiro[4.5]decan-3-yl)-N,N-bis(4- methoxybenzyl)benzenesulfonamide |
| 87 | 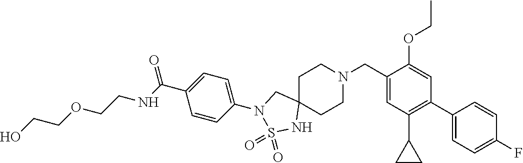 [SMILES: CCOc1cc(-c2ccc(F)cc2)c(C2CC2)cc1CN1CCC2(CC1)CN(c1ccc(C(=O)NCCOCCO)cc1)S(=O)(=O)N2] | 4-(8-((2-cyclopropyl-5-ethoxy-4′-fluoro-[1,1′- biphenyl]-4-yl)methyl)-2,2-dioxido-2-thia-1,3,8- triazaspiro[4.5]decan-3-yl)-N-(2-(2- hydroxyethoxy)ethyl)benzamide |
| 88 | 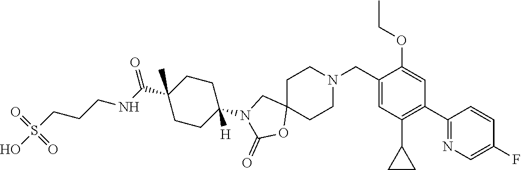 [SMILES: CCOc1cc(-c2ccc(F)cn2)c(C2CC2)cc1CN1CCC2(CC1)CN([C@H]1CC[C@@](C)(C(=O)NCCCS(=O)(=O)O)CC1)C(=O)O2] | 3-((1s,4s)-4-(8-(5-cyclopropyl-2-ethoxy-4-(5- fluoropyridin-2-yl)benzyl)-2-oxo-1-oxa-3,8- diazaspiro[4.5]decan-3-yl)-1-methylcyclohexane-1- carboxamido)propane-1-sulfonic acid |
| 89 | 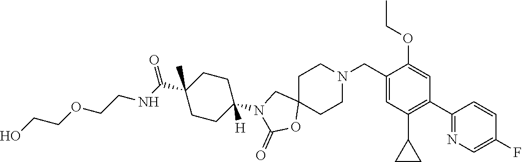 [SMILES: CCOc1cc(-c2ccc(F)cn2)c(C2CC2)cc1CN1CCC2(CC1)CN([C@H]1CC[C@@](C)(C(=O)NCCOCCO)CC1)C(=O)O2] | (1s,4s)-4-(8-(5-cyclopropyl-2-ethoxy-4-(5- fluoropyridin-2-yl)benzyl)-2-oxo-1-oxa-3,8- diazaspiro[4.5]decan-3-yl)-N-(2-(2- hydroxyethoxy)ethyl)-1-methylcyclohexane-1- carboxamide |
| 90 | 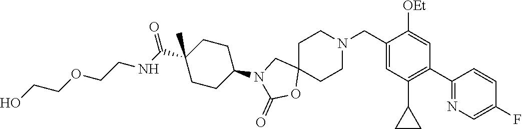 [SMILES: CCOc1cc(-c2ccc(F)cn2)c(C2CC2)cc1CN1CCC2(CC1)CN([C@H]1CC[C@](C)(C(=O)NCCOCCO)CC1)C(=O)O2] | (1r,4r)-4-(8-(5-cyclopropyl-2-ethoxy-4-(5- fluoropyridin-2-yl)benzyl)-2-oxo-1-oxa-3,8- diazaspiro[4.5]decan-3-yl)-N-(2-(2- hydroxyethoxy)ethyl)-1-methylcyclohexane-1- carboxamide |
| 91 | 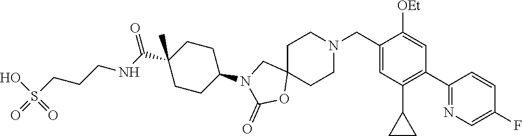 [SMILES: CCOc1cc(-c2ccc(F)cn2)c(C2CC2)cc1CN1CCC2(CC1)CN([C@H]1CC[C@](C)(C(=O)NCCCS(=O)(=O)O)CC1)C(=O)O2] | 3-((1r,4r)-4-(8-(5-cyclopropyl-2-ethoxy-4-(5- fluoropyridin-2-yl)benzyl)-2-oxo-1-oxa-3,8- diazaspiro[4.5] decan-3-yl)-1-methylcyclohexane-1- carboxamido)propane-1-sulfonic acid |
| 92 | 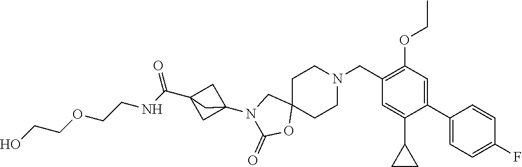 [SMILES: CCOc1cc(-c2ccc(F)cc2)c(C2CC2)cc1CN1CCC2(CC1)CN(C13CC(C(=O)NCCOCCO)(C1)C3)C(=O)O2] | 3-(8-((2-cyclopropyl-5-ethoxy-4′-fluoro-[1,1′- biphenyl]-4-yl)methyl)-2-oxo-1-oxa-3,8- diazaspiro[4.5]decan-3-yl)-N-(2-(2- hydroxyethoxy)ethyl)bicyclo[1.1.1]pentane-1- carboxamide |

In some embodiments, the compounds of Table 1 are provided as pharmaceutically acceptable salts.

TABLE 2

| Ex. # | Structure | Name |
|---|---|---|
| 93 | 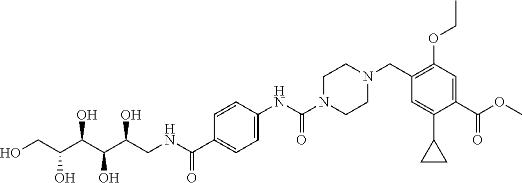 [SMILES: CCOc1cc(C(=O)OC)c(C2CC2)cc1CN1CCN(C(=O)Nc2ccc(C(=O)NC[C@H](O)[C@@H](O)[C@H](O)[C@H](O)CO)cc2)CC1] | methyl 2-cyclopropyl-5-ethoxy-4-((4-((4- (((2S,3R,4R,5R)-2,3,4,5,6- pentahydroxyhexyl)carbamoyl)phenyl)carbamoyl) piperazin-1-yl)methyl)benzoate |
| 94 | 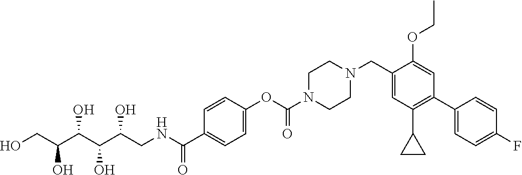 [SMILES: CCOc1cc(-c2ccc(F)cc2)c(C2CC2)cc1CN1CCN(C(=O)Oc2ccc(C(=O)NC[C@@H](O)[C@H](O)[C@@H](O)[C@@H](O)CO)cc2)CC1] | 4-(((2R,3S,4S,5S)-2,3,4,5,6- pentahydroxyhexyl)carbamoyl)phenyl 4-((2- cyclopropyl-5-ethoxy-4′-fluoro-[1,1′-biphenyl]-4- yl)methyl)piperazine-1-carboxylate |
| 95 | 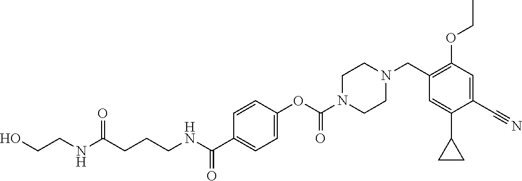 [SMILES: CCOc1cc(C#N)c(C2CC2)cc1CN1CCN(C(=O)Oc2ccc(C(=O)NCCCC(=O)NCCO)cc2)CC1] | 4-((4-((2-hydroxyethyl)amino)-4- oxobutyl)carbamoyl)phenyl 4-(4-cyano-5- cyclopropyl-2-ethoxybenzyl)piperazine-1- carboxy late |
| 96 | 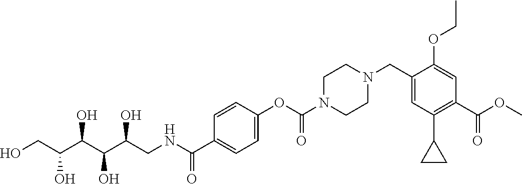 [SMILES: CCOc1cc(C(=O)OC)c(C2CC2)cc1CN1CCN(C(=O)Oc2ccc(C(=O)NC[C@H](O)[C@@H](O)[C@H](O)[C@H](O)CO)cc2)CC1] | 4-(((2S,3R,4R,5R)-2,3,4,5,6- pentahydroxyhexyl)carbamoyl)phenyl 4-(5- cyclopropyl-2-ethoxy-4- methoxycarbonyl)benzyl)piperazine-1-carboxylate |
| 97 | 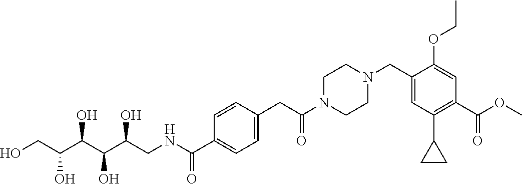 [SMILES: CCOc1cc(C(=O)OC)c(C2CC2)cc1CN1CCN(C(=O)Cc2ccc(C(=O)NC[C@H](O)[C@@H](O)[C@H](O)[C@H](O)CO)cc2)CC1] | methyl 2-cyclopropyl-5-ethoxy-4-((4-(2-(4- (((2S,3R,4R,5R)-2,3,4,5,6- pentahydroxyhexyl)carbamoyl)phenyl)acetyl) piperazin-1-yl)methyl)benzoate |
| 98 | 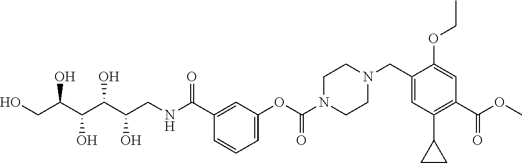 [SMILES: CCOc1cc(C(=O)OC)c(C2CC2)cc1CN1CCN(C(=O)Oc2cccc(C(=O)NC[C@H](O)[C@@H](O)[C@H](O)[C@H](O)CO)c2)CC1] | 3-(((2S,3R,4R,5R)-2,3,4,5,6- pentahydroxyhexyl)carbamoyl)phenyl 4-(5- cyclopropyl-2-ethoxy-4- methoxycarbonyl)benzyl)piperazine-1-carboxylate |
| 99 | 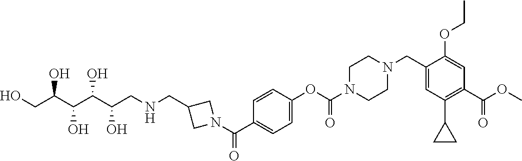 [SMILES: CCOc1cc(C(=O)OC)c(C2CC2)cc1CN1CCN(C(=O)Oc2ccc(C(=O)N3CC(CNC[C@H](O)[C@@H](O)[C@H](O)[C@H](O)CO)C3)cc2)CC1] | 4-(3-((((2S,3R,4R,5R)-2,3,4,5,6- pentahydroxyhexyl)amino)methyl)azetidine-1- carbonyl)phenyl 4-(5-cyclopropyl-2-ethoxy-4- (methoxycarbonyl)benzyl)piperazine-1-carboxylate |

In some embodiments, the compounds of Table 2 are provided as pharmaceutically acceptable salts.

### Further Forms of Compounds

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

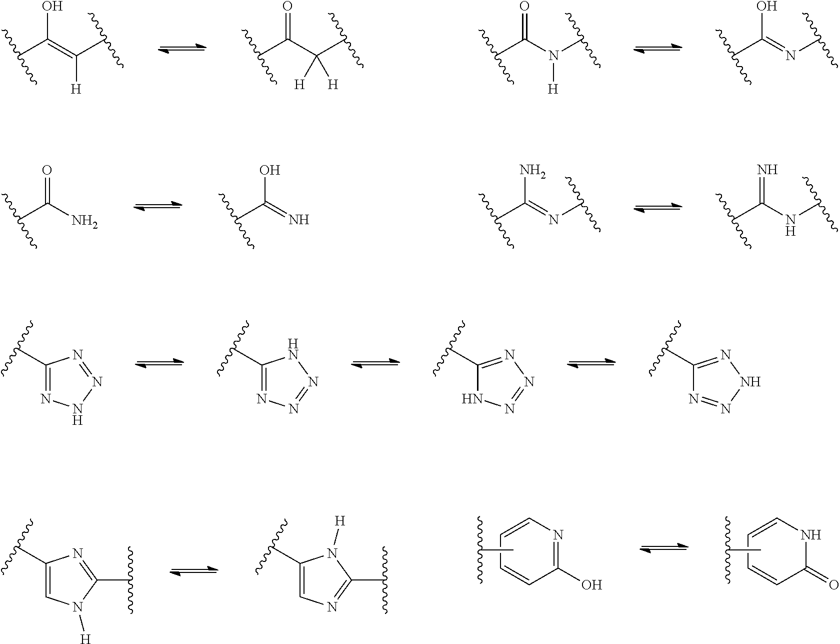

In some situations, the compounds described herein possess one or more chiral centers and each center exists in the (R)-configuration or (S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

The term “positional isomer” refers to structural isomers around a central ring, such as ortho-, meta-, and para-isomers around a benzene ring.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that is, in some embodiments, converted under physiological conditions or by solvolysis to an active compound described herein. Thus, the term prodrug refers to a precursor of an active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, carboxy, or mercapto group is bonded to any group that, when the prodrug of the active compound is administered to a mammalian subject, cleaves to form a free hydroxy, free amino, free carboxy, or free mercapto group, respectively. Examples of prodrugs include, but are not limited to, acetate, formate and benzoate derivatives of alcohol or amine functional groups in the active compounds and the like.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. “Hydrates” are formed when the solvent is water, or “alcoholates” are formed when the solvent is alcohol. Solvates of compounds described herein are conveniently prepared or formed during the processes described herein. The compounds provided herein optionally exist in either unsolvated as well as solvated forms.

The compounds disclosed herein, in some embodiments, are used in different enriched isotopic forms, e.g., enriched in the content of ²H, ³H, ¹¹C ¹³C and/or ¹⁴C. In some embodiments, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ³H, ¹¹C, ¹³C, ¹⁴C, ¹⁵C, ¹²N, ¹³N, ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art. In some embodiments deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments, the compounds described herein are labeled by other means, including, but not limited to, the use of chromophores or fluorescent moieties, bioluminescent labels, or chemiluminescent labels.

In certain embodiments, the compounds described herein, or a pharmaceutically acceptable salt, solvate, stereoisomer, or prodrug thereof, as described herein are substantially pure, in that it contains less than about 5%, or less than about 1%, or less than about 0.1%, of other organic small molecules, such as contaminating intermediates or by-products that are created, for example, in one or more of the steps of a synthesis method.

### Preparation of the Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described as outlined in the Examples.

### Pharmaceutical Compositions

In some embodiments, disclosed herein is a pharmaceutical composition comprising an SSTR5 antagonist described herein, or a pharmaceutically acceptable salt, solvate, stereoisomer, or prodrug thereof, and a pharmaceutically acceptable excipient. In some embodiments, the SSTR5 antagonist is combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt, solvate, stereoisomer, or prodrug thereof, together with a pharmaceutically acceptable excipient.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

### Combination Therapies

In certain embodiments, it is appropriate to administer at least one compound described herein, or a pharmaceutically acceptable salt, solvate, stereoisomer, or prodrug thereof, in combination with one or more other therapeutic agents. In some embodiments, a compound described herein, or a pharmaceutically acceptable salt, solvate, stereoisomer, or prodrug thereof, is administered in combination with a TGR5 agonist, a GPR40 agonist, a GPR119 agonist, a CCK1 agonist, a PDE4 inhibitor, a DPP-4 inhibitor, a GLP-1 receptor agonist, metformin, or combinations thereof. In certain embodiments, the pharmaceutical composition further comprises one or more anti-diabetic agents. In certain embodiments, the pharmaceutical composition further comprises one or more anti-obesity agents. In certain embodiments, the pharmaceutical composition further comprises one or more agents to treat nutritional disorders.

Examples of a TGR5 agonist to be used in combination with a compound described herein, or a pharmaceutically acceptable salt, solvate, stereoisomer, or prodrug thereof, include: INT-777, XL-475, SRX-1374, RDX-8940, RDX-98940, SB-756050, and those disclosed in WO-2008091540, WO-2010059853, WO-2011071565, WO-2018005801, WO-2010014739, WO-2018005794, WO-2016054208, WO-2015160772, WO-2013096771, WO-2008067222, WO-2008067219, WO-2009026241, WO-2010016846, WO-2012082947, WO-2012149236, WO-2008097976, WO-2016205475, WO-2015183794, WO-2013054338, WO-2010059859, WO-2010014836, WO-2016086115, WO-2017147159, WO-2017147174, WO-2017106818, WO-2016161003, WO-2014100025, WO-2014100021, WO-2016073767, WO-2016130809, WO-2018226724, WO-2018237350, WO-2010093845, WO-2017147137, WO-2015181275, WO-2017027396, WO-2018222701, WO-2018064441, WO-2017053826, WO-2014066819, WO-2017079062, WO-2014200349, WO-2017180577, WO-2014085474.

Examples of a GPR40 agonist to be used in combination with a compound described herein, or a pharmaceutically acceptable salt, solvate, stereoisomer, or prodrug thereof, include: fasiglifam, MR-1704, SCO-267, SHR-0534, HXP-0057-SS, LY-2922470, P-11187, JTT-851, ASP-4178, AMG-837, ID-11014A, HD-C715, CNX-011-67, JNJ-076, TU-5113, HD-6277, MK-8666, LY-2881835, CPL-207-280, ZYDG-2, and those described in US-07750048, WO-2005051890, WO-2005095338, WO-2006011615, WO-2006083612, WO-2006083781, WO-2007088857, WO-2007123225, WO-2007136572, WO-2008054674, WO-2008054675, WO-2008063768, WO-2009039942, WO-2009039943, WO-2009054390, WO-2009054423, WO-2009054468, WO-2009054479, WO-2009058237, WO-2010085522, WO-2010085525, WO-2010085528, WO-2010091176, WO-2010123016, WO-2010123017, WO-2010143733, WO-2011046851, WO-2011052756, WO-2011066183, WO-2011078371, WO-2011161030, WO-2012004269, WO-2012004270, WO-2012010413, WO-2012011125, WO-2012046869, WO-2012072691, WO-2012111849, WO-2012147518, WO-2013025424, WO-2013057743, WO-2013104257, WO-2013122028, WO-2013122029, WO-2013128378, WO-2013144097, WO-2013154163, WO-2013164292, WO-2013178575, WO-2014019186, WO-2014073904, WO-2014082918, WO-2014086712, WO-2014122067, WO-2014130608, WO-2014146604,WO-2014169817,WO-2014170842,WO-2014187343, WO-2015000412, WO-2015010655, WO-2015020184, WO-2015024448, WO-2015024526, WO-2015028960, WO-2015032328, WO-2015044073, WO-2015051496, WO-2015062486, WO-2015073342, WO-2015078802, WO-2015084692, WO-2015088868, WO-2015089809, WO-2015097713, WO-2015105779, WO-2015105786, WO-2015119899, WO-2015176267, WO-201600771, WO-2016019587, WO-2016022446, WO-2016022448, WO-2016022742, WO-2016032120, WO-2016057731, WO-2017025368, WO-2017027309, WO-2017027310, WO-2017027312, WO-2017042121, WO-2017172505, WO-2017180571, WO-2018077699, WO-2018081047, WO-2018095877, WO-2018106518, WO-2018111012, WO-2018118670, WO-2018138026, WO-2018138027, WO-2018138028, WO-2018138029, WO-2018138030, WO-2018146008, WO-2018172727, WO-2018181847, WO-2018182050, WO-2018219204, WO-2019099315, and WO-2019134984.

Examples of a GPR119 agonist to be used in combination with a compound described herein, or a pharmaceutically acceptable salt, solvate, stereoisomer, or prodrug thereof, include: DS-8500a, HD-2355, LC34AD3, PSN-491, HM-47000, PSN-821, MBX-2982, GSK-1292263, APD597, DA-1241, and those described in WO-2009141238, WO-2010008739, WO-2011008663, WO-2010013849, WO-2012046792, WO-2012117996, WO-2010128414, WO-2011025006, WO-2012046249, WO-2009106565, WO-2011147951, WO-2011127106, WO-2012025811, WO-2011138427, WO-2011140161, WO-2011061679, WO-2017175066, WO-2017175068, WO-2015080446, WO-2013173198, US-20120053180, WO-2011044001, WO-2010009183, WO-2012037393, WO-2009105715, WO-2013074388, WO-2013066869, WO-2009117421, WO-201008851, WO-2012077655, WO-2009106561, WO-2008109702, WO-2011140160, WO-2009126535, WO-2009105717, WO-2013122821, WO-2010006191, WO-2009012275, WO-2010048149, WO-2009105722, WO-2012103806, WO-2008025798, WO-2008097428, WO-2011146335, WO-2012080476, WO-2017106112, WO-2012145361, WO-2012098217, WO-2008137435, WO-2008137436, WO-2009143049, WO-2014074668, WO-2014052619, WO-2013055910, WO-2012170702, WO-2012145604, WO-2012145603, WO-2011030139, WO-2018153849, WO-2017222713, WO-2015150565, WO-2015150563, WO-2015150564, WO-2014056938, WO-2007120689, WO-2016068453, WO-2007120702, WO-2013167514, WO-2011113947, WO-2007003962, WO-2011153435, WO-2018026890, WO-2011163090, WO-2011041154, WO-2008083238, WO-2008070692, WO-2011150067, and WO-2009123992.

Examples of a CCK1 agonist to be used in combination with a compound described herein, or a pharmaceutically acceptable salt, solvate, stereoisomer, or prodrug thereof, include: A-70874, A-71378, A-71623, A-74498, CE-326597, GI-248573, GSKI-181771X, NN-9056, PD-149164, PD-134308, PD-135158, PD-170292, PF-04756956, SR-146131, SSR-125180, and those described in EP-00697403, US-20060177438, WO-2000068209, WO-2000177108, WO-2000234743, WO-2000244150, WO-2009119733, WO-2009314066, WO-2009316982, WO-2009424151, WO-2009528391, WO-2009528399, WO-2009528419, WO-2009611691, WO-2009611940, WO-2009851686, WO-2009915525, WO-2005035793, WO-2005116034, WO-2007120655, WO-2007120688, WO-2008091631, WO-2010067233, WO-2012070554, and WO-2017005765.

Examples of a PDE4 inhibitor to be used in combination with a compound described herein, or a pharmaceutically acceptable salt, solvate, stereoisomer, or prodrug thereof, include: apremilast, cilomilast, crisaborole, diazepam, luteolin, piclamilast, and roflumilast.

Examples of a DPP-4 inhibitor to be used in combination with a compound described herein, or a pharmaceutically acceptable salt, solvate, stereoisomer, or prodrug thereof, include: sitagliptin, vildagliptin, saxagliptin, linagliptin, gemigliptin, teneligliptin, alogliptin, trelagliptin, omarigliptin, evogliptin, gosogliptin, and dutogliptin.

Examples of a GLP-1 receptor agonist to be used in combination with a compound described herein, or a pharmaceutically acceptable salt, solvate, stereoisomer, or prodrug thereof, include: albiglutide, dulaglutide, exenatide, extended-release exenatide, liraglutide, lixisenatide, and semaglutide.

Examples of anti-diabetic agents to be used in combination with a compound described herein, or a pharmaceutically acceptable salt, solvate, stereoisomer, or prodrug thereof, include: GLP-1 receptor agonists such as exenatide, liraglutide, taspoglutide, lixisenatide, albiglutide, dulaglutide, semaglutide, OWL833 and ORMD 0901; SGLT2 inhibitors such as dapagliflozin, canagliflozin, empagliflozin, ertugliflozin, ipragliflozin, luseogliflozin, remogliflozin, sergliflozin, sotagliflozin, and tofogliflozin; biguinides such as metformin; insulin and insulin analogs.

Examples of anti-obesity agents to be used in combination with a compound described herein, or a pharmaceutically acceptable salt, solvate, stereoisomer, or prodrug thereof, include: GLP-1 receptor agonists such as liraglutide, semaglutide; SGLT1/2 inhibitors such as LIK066, pramlintide and other amylin analogs such as AM-833, AC2307, and BI 473494; PYY analogs such as NN-9747, NN-9748, AC-162352, AC-163954, GT-001, GT-002, GT-003, and RHS-08; GIP receptor agonists such as APD-668 and APD-597; GLP-1/GIP co-agonists such as tirzepatide (LY329176), BHM-089, LBT-6030, CT-868, SCO-094, NNC-0090-2746, RG-7685, NN-9709, and SAR-438335; GLP-1/glucagon co-agonist such as cotadutide (MEDI0382), BI 456906, TT-401, G-49, H&D-001A, ZP-2929, and HM-12525A; GLP-1/GIP/glucagon triple agonist such as SAR-441255, HM-15211, and NN-9423; GLP-1/secretin co-agonists such as GUB06-046; leptin analogs such as metreleptin; GDF15 modulators such as those described in WO2012138919, WO2015017710, WO2015198199, WO-2017147742 and WO-2018071493; FGF21 receptor modulators such as NN9499, NGM386, NGM313, BFKB8488A (RG7992), AKR-001, LLF-580, CVX-343, LY-2405319, BI089-100, and BMS-986036; MC4 agonists such as setmelanotide; MetAP2 inhibitors such as ZGN-1061; ghrelin receptor modulators such as HM04 and AZP-531; ghrelin O-acyltransferase inhibitors such as T-3525770 (RM-852) and GLWL-01; and oxytocin analogs such as carbetocin.

Examples of agents for nutritional disorders to be used in combination with a compound described herein, or a pharmaceutically acceptable salt, solvate, stereoisomer, or prodrug thereof, include: GLP-2 receptor agonists such as tedaglutide, glepaglutide (ZP1848), elsiglutide (ZP1846), apraglutide (FE 203799), HM-15912, NB-1002, GX-G8, PE-0503, SAN-134, and those described in WO-2011050174, WO-2012028602, WO-2013164484, WO-2019040399, WO-2018142363, WO-2019090209, WO-2006117565, WO-2019086559, WO-2017002786, WO-2010042145, WO-2008056155, WO-2007067828, WO-2018229252, WO-2013040093, WO-2002066511, WO-2005067368, WO-2009739031, WO-2009632414, and WO2008028117; and GLP-1/GLP-2 receptor co-agonists such as ZP-GG-72 and those described in WO-2018104561, WO-2018104558, WO-2018103868, WO-2018104560, WO-2018104559, WO-2018009778, WO-2016066818, and WO-2014096440.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt, solvate, stereoisomer, or prodrug thereof, is co-administered with one or more additional therapeutic agents, wherein the compound described herein, or a pharmaceutically acceptable salt, solvate, stereoisomer, or prodrug thereof, and the additional therapeutic agent(s) modulate different aspects of the disease, disorder or condition being treated, thereby providing a greater overall benefit than administration of either therapeutic agent alone. In some embodiments, the additional therapeutic agent(s) is a TGR5 agonist, a GPR40 agonist, a GPR119 agonist, a CCK1 agonist, a PDE4 inhibitor, a DPP-4 inhibitor, a GLP-1 receptor agonist, metformin, or combinations thereof. In some embodiments, the additional therapeutic agent is an anti-diabetic agent. In some embodiments, the additional therapeutic agent is an anti-obesity agent. In some embodiments, the additional therapeutic agent is an agent to treat nutritional disorders.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or pharmaceutically acceptable salts, solvates, stereoisomers, or prodrugs thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease.

In some embodiments, a compound described herein, or a pharmaceutically acceptable salt thereof, is administered in combination with anti-inflammatory agent, anti-cancer agent, immunosuppressive agent, steroid, non-steroidal anti-inflammatory agent, antihistamine, analgesic, hormone blocking therapy, radiation therapy, monoclonal antibodies, or combinations thereof.

### EXAMPLES

### LIST OF ABBREVIATIONS

As used above, and throughout the description of the invention, the following abbreviations, unless otherwise indicated, shall be understood to have the following meanings:

| | |
|---|---|
| ACN or MeCN | acetonitrile |
| Boc or BOC | tert-butyloxycarbonyl |
| Bn | benzyl |
| BnBr | benzyl bromide |
| Cbz | carboxybenzyl |
| CbzCl | benzyl chloroformate |
| CDI | 1,1′-Carbonyldiimidazole |
| Cy | cyclohexyl |
| DCC | N,N′-dicyclohexylcarbodiimide |
| DCM | dichloromethane (CH₂Cl₂) |
| DIBAL-H | diisobutylaluminium hydride |
| DIPEA or DIEA | diisopropylethylamine |
| DMA | dimethylacetamide |
| DMAP | 4-dimethylaminopyridine |
| DMEDA | 1,2-dimethylethylenediamine |
| DMEM | Dulbecco's Modified Eagle Medium |
| DMF | dimethylformamide |
| DMFDMA | dimethylformamide dimethylacetal |
| DMSO | dimethylsulfoxide |
| DPPF | 1,1′-Bis(diphenylphosphino)ferrocene |
| EDCI | 1-ethyl-3-(3-dimethylaminopropyl)carbodiimide |
| eq | equivalent(s) |
| Et | ethyl |
| EtI | ethyl iodide |
| EtOH | ethanol |
| EtOAc or EA | ethyl acetate |
| FA | formic acid |
| FBS | fetal bovine serum |
| Fmoc-Cl | fluorenylmethyloxycarbonyl chloride |
| h, hr(s) | hour(s) |
| HATU | 1-[bis(dimethylamino)methylene]-1H-1,2,3- |
| | triazolo[4,5-b]pyridinium 3-oxid hexafluorophosphate |
| HPLC | high performance liquid chromatography |
| HTRF | homogeneous time resolved fluorescence |
| i-pr or ipr | isopropyl |
| iPrMgCl | isopropylmagnesium chloride |
| i-PrOH or IPA | isopropanol |
| LCMS | liquid chromatography-mass spectrometry |
| Me | methyl |
| MeOH | methanol |
| MS | mass spectroscopy |
| Ms | methanesulfonyl (mesyl) |
| MsCl | methanesulfonyl chloride (mesyl chloride) |
| MTBE | methyl tert-butyl ether |
| NBS | N-bromosuccinimide |
| NMR | nuclear magnetic resonance |
| PCy₃ | tricyclohexylphosphine |
| Pd(dba)₂ | bis(dibenzylideneacetone)palladium(0) |
| Pd(dppf)Cl₂ | [1,1′-Bis(diphenylphosphino)ferrocene]dichloro- |
| | palladium(II) |
| PE | petroleum ether |
| PMB | p-methoxybenzyl |
| psi | pounds per square inch |
| Py | pyridine |
| Rt or RT | room temperature |
| SPhos | 2-dicyclohexylphosphino-2′,6′-dimethoxybiphenyl |
| SPhos-Pd-G2 | chloro(2-dicyclohexylphosphino-2′,6′-dimethoxy-1,1′- |
| | biphenyl)[2-(2′-amino-1,1′-biphenyl)]palladium(II) |
| t-Bu | tert-butyl |
| t-Bu₃P-Pd-G2 | chloro[(tri-tert-butylphosphine)-2-(2-aminobiphenyl)] |
| palladium(II) | |
| TEA | triethylamine |
| Tf | trifluoromethylsulfonyl (triflyl) |
| TFA | trifluoroacetic acid |
| THF | tetrahydrofuran |
| TLC | thin layer chromatography |
| Tol or tol | toluene |
| TR-FRET | time-resolved Forster resonance energy transfer |
| Ts | toluenesulfonyl (tosyl) |
| TsOH | p-toluenesulfonic acid |
| XPhos | 2-dicyclohexylphosphino-2′,4′,6′-triisopropylbiphenyl |
| XPhos-Pd-G2 | chloro(2-dicyclohexylphosphino-2′,4′,6′-triisopropyl- |
| | 1,1′-biphenyl) [2-(2′-amino-1,1′-biphen- |
| | yl)]palladium(II) |

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: 4-(8-((2,6-diethoxy-4′-fluoro-[1,1′-biphenyl]-4-yl)methyl)-2-oxo-1-oxa-3,8-diazaspiro [4.5] decan-3-yl)-N-(4-((2-hydroxyethyl)amino)-4-oxobutyl)benzamide (Compound 1)

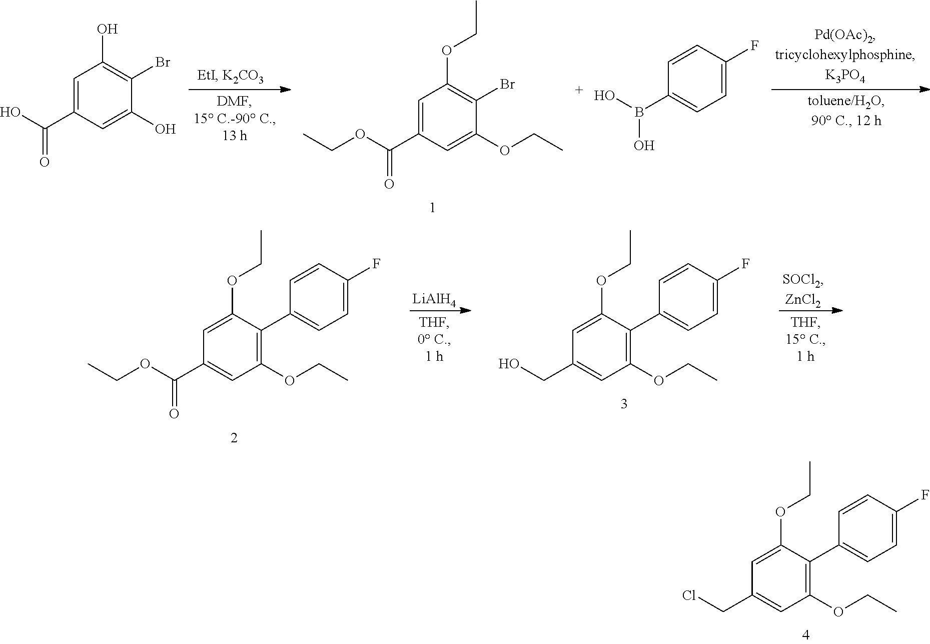

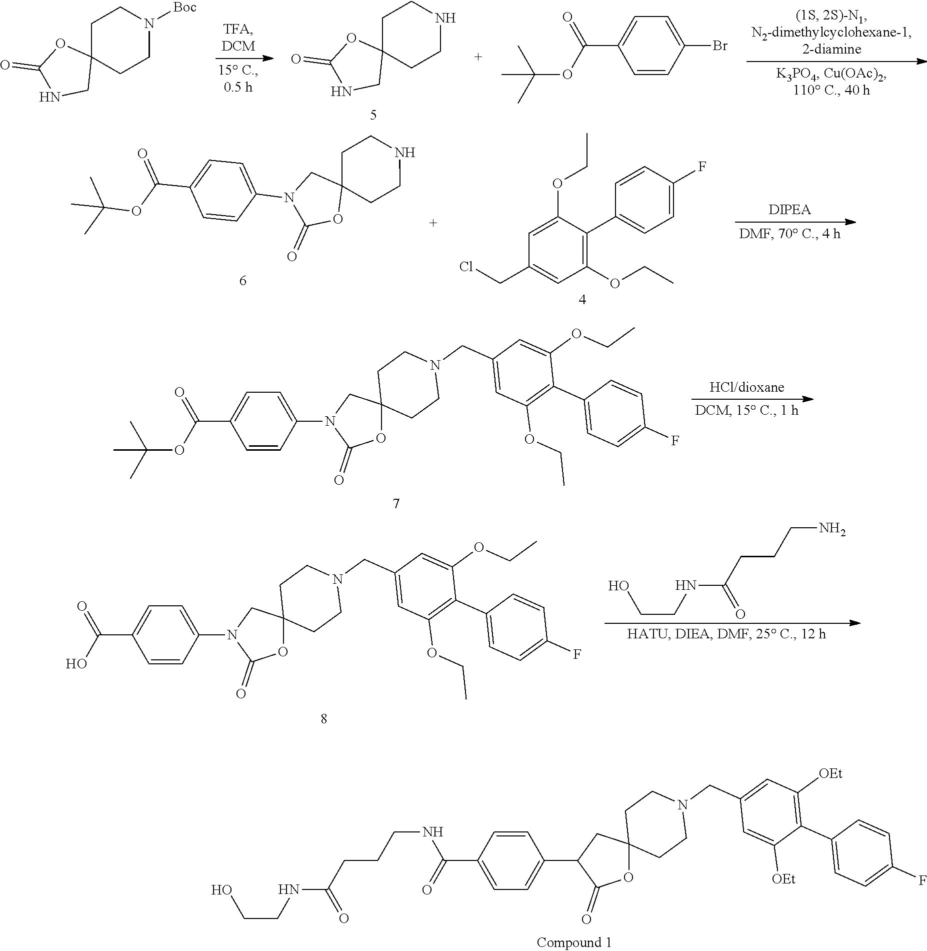

Step 1: ethyl 4-bromo-3,5-diethoxybenzoate (1): To a mixture of 4-bromo-3,5-dihydroxy-benzoic acid (25 g, 107 mmol, 1 eq) and K₂CO₃ (44.5 g, 322 mmol, 3 eq) in DMF (250 mL) was added iodoethane (67 g, 429 mmol, 34 mL, 4 eq) at 15° C. Then the mixture was stirred at 15° C. for 12 hours and at 90° C. for 1 hour. On completion, the solution was poured into water (300 mL) and extracted with ethyl acetate (100×2 mL). The organic layer was washed with water (50 mL), brine (30 mL×2), dried with Na₂SO₄, filrtated and concentrated in vacuo to give 1 (34 g, 99% yield) as a yellow solid. LCMS: (ES+) m/z (M+H)⁺=317.0.

Step 2: ethyl 2,6-diethoxy-4′-fluoro-[1,1′-biphenyl]-4-carboxylate (2): To a mixture of 1 (34 g, 107 mmol, 1 eq), (4-fluorophenyl)boronic acid (26 g, 188 mmol, 1.75 eq), K₃PO₄ (68 g, 322 mmol, 3 eq), and tricyclohexylphosphine (3.0 g, 11 mmol, 0.1 eq) in toluene (340 mL) and H₂O (170 mL) was added Pd(OAc)₂ (1.2 g, 5.4 mmol, 0.05 eq) under N₂. Then the mixture was stirred at 90° C. for 12 hours. On completion, the mixture was poured into H₂O (300 mL) then extracted with EA (3×100 mL). The organic phase was concentrated in vacuo to give the residue. The residue was purified by column chromatography (SiO₂, Petroleum ether : Ethyl acetate=1:0 to 50:1) to give 2 (47 g, crude) as an off-white solid. ¹H NMR (400 MHz, CDCl₃) δ 7.33-7.29 (m, 4H), 7.04 (t, J=8.8 Hz, 2H), 4.36 (q, J=7.2 Hz , 2H), 4.00 (q, J=6.8 Hz , 4H), 1.38 (t, J=6.8 Hz , 3H), 1.24 (t, J=7.2 Hz , 6H).

Step 3: (2,6-diethoxy-4′-fluoro-[1,1′-biphenyl]-4-yl)methanol (3): To a solution of 2 (35 g, 105 mmol, 1 eq) in THF (200 mL) was added LiAlH₄ (4.0 g, 105 mmol, 1 eq) at 0° C. under N₂ protection. Then the mixture was stirred at 0° C. for 1 hour. On completion, the solution was quenched with water (4 mL) at 10° C. and stirred for 0.5 hour. Then 50 mL water was added to the mixture, adjust pH=2 with 2 M HCl. Then the mixture was extracted with EA (2×150 mL). The combined organic layer was washed with sat.NaHCO₃ (80 mL) and brine (50 mL), dried over Na₂SO₄, filtered and concentrated in vacuo to give 3 (34.5 g, crude) as a off-white solid.

Step 4: 4-(chloromethyl)-2,6-diethoxy-4′-fluoro-1,1′-biphenyl (4): To a solution of 3 (29.4 g, 101 mmol, 1 eq) and SOCl₂ (18 g, 152 mmol, 11 mL, 1.5 eq) in THF (300 mL) was added ZnCl₂ (1.38 g, 10 mmol, 0.1 eq). The mixture was stirred at 15° C. for 1 hour. On completion, the solution quenched with slow addition of saturated aqueous NaHCO₃ (40 mL) and extracted with ethyl acetate (3×200 ml). The organic layer was washed with saturated brine (100 mL), dried over Na₂SO₄, filtered and concentrated in vacuo to give the residue. The residue was purified by column chromatography (SiO₂, Petroleum ether: Ethyl acetate=100:1 to 50:1) to give 4 (22.6 g, 72% yield) as a white solid.

Step 5: 1-oxa-3,8-diazaspiro[4.5]decan-2-one (5): To a solution of tert-butyl 2-oxo-1-oxa-3,8-diazaspiro[4.5]decane-8-carboxylate (11 g, 43 mmol, 1 eq) in DCM (75 mL) was added TFA (37 mL, 495 mmol, 12 eq). The mixture was stirred at 15° C. for 0.5 hour. On completion, the mixture was concentrated in vacuo to give 5 (11.6 g, crude, TFA salt) as a white solid. ¹H NMR (400 MHz, CD₃OD) δ 3.26-3.15 (m, 6H), 2.07-2.04 (m, 2 H), 1.94-1.88 (m, 2H).

Step 6: tert-butyl 4-(2-oxo-1-oxa-3,8-diazaspiro[4.5]decan-3-yl)benzoate (6): To a solution of 5 (11.3 g, 42 mmol, 1 eq, TFA), tert-butyl 4-bromobenzoate (11 g, 42 mmol 1 eq), K₃PO₄ (27 g, 125 mmol, 3 eq), and (1S,2S)-N1,N2-dimethylcyclohexane-1,2-diamine (1.8 g, 13 mmol, 0.3 eq) in dioxane (42 mL) was added Cu(OAc)₂ (1.9 g, 10 mmol, 0.25 eq) under N₂. Then the mixture was stirred at 110° C. for 16 hours. Then Cu(OAc)₂ (1.90 g, 10 mmol, 0.25 eq) and K₂CO₃ (17 g, 125 mmol, 3 eq) was added to the reaction mixture. The mixture was stirred at 110° C. for another 24 hours. On completion, the mixture was added MeOH (200 mL), then filtered and concentrated under reduced pressure to give a residue. Water (80 mL) was added and the mixture was extracted with ethyl acetate (3×100 mL). The combined organic layers were washed with saturated brine 40 mL, dried over Na₂SO₄, filtered and concentrated under reduced pressure to give a crude residue. The crude product was purified by reversed phase MPLC (column: Phenomenex luna C18 250×50 mm×10 μm, mobile phase: [A: water (0.1% FA, v/v), B: ACN]; B%: 5%-35% gradient over 60 min). The resultant solution was concentrated in vacuo to give a pure 6 (10 g, 59% yield) as a yellow solid. LCMS: (ES+) m/z (M+H)⁺=333.1.

Step 7: tert-butyl 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzoate (7): To a solution of 6 (12.5 g, 33 mmol, 1 eq, FA) in DMF (130 mL) was added 4 (10.2 g, 33 mmol, 1 eq) and DIPEA (17.3 mL, 99 mmol, 3 eq). Then the mixture was stirred at 70° C. for 4 hours. On completion, the reaction mixture was diluted with EA (100 mL) and washed with water (2×50 mL). The aqueous layer was extracted with EA (2×40 mL). The combined organic layer was washed with water (40 mL) and brine (2×40 mL), dried over Na₂SO₄, filtered and concentrated in vacuo to give a pure 7 (19 g, 95% yield) as a white solid. LCMS: (ES+) m/z (M+H)⁺=605.3.

Step 8: 4-(8-((2,6-diethoxy-4′-fluoro-[1,1′-biphenyl]-4-yl)methyl)-2-oxo-1-oxa-3,8-diazaspiro[4.5]decan-3-yl)benzoic acid hydrochloride (8): To a solution of 7 (25 g, 41 mmol, 1 eq) in DCM (250 mL) was added HCl/dioxane (4 M, 0.3 mol, 75 mL) . The mixture was stirred at 15° C. for 1 hour. On completion, the mixture was concentrated in vacuo to give crude residue. The residue was triturated with THF (200 mL) overnight and filtered to give pure 8 (18 g, 77% yield, 98% purity, HCl salt) as a white solid. LCMS: (ES⁺) m/z (M+H)⁺=549.2. ¹H NMR (400 MHz, CD₃OD) δ 8.05 (d, J=8.8 Hz, 2H), 7.70 (d, J=8.8, 2H), 7.30-7.26 (m, 2H), 7.07 (t, J=8.8 Hz, 2H), 6.87 (s, 1H), 4.40 (s, 2H), 4.05-3.97 (m, 6H), 3.58-3.48 (m, 2H), 3.47-3.40 (m, 2H), 2.39-2.27 (m, 4H), 1.25 (t, J=6.8 Hz, 6H).

Step 9: 4-[8-[[3,5-diethoxy-4-(4-fluorophenyl)phenyl]methyl]-2-oxo-1-oxa-3,8-diazaspiro[4.5]decan-3-yl]-N-[4-(2-hydroxyethylamino)-4-oxo-butyl]benzamide (Compound 1): To a solution of 8 (500 mg, 911 μmol, 1 eq) and 4-amino-N-(2-hydroxyethyl)butanamide (160 mg, 1.1 mmol, 1.2 eq) in DMF (5 mL) was added HATU (520 mg, 1.4 mmol, 1.5 eq) and DIEA (236 mg, 1.8 mmol, 318 μL, 2 eq). The mixture was stirred at 25° C. for 12 hours. The reaction was concentrated under reduced pressure, and the residue was purified by prep-HPLC (column: Phenomenex Synergi Max-RP 250×50 mm×10 μm; mobile phase: [A: water (0.2% FA, v/v), B: ACN]; B%: 18%-56%, gradient over 11 min) to give crude Compound 1 (420 mg, 63% yield, 93% purity) as a white solid. 100 mg of crude Compound 1 was purified by prep-HPLC (column: Boston Green ODS 150×30mm×5 μm; mobile phase: [A: water (0.225%FA. v/v), B: ACN]; B%:15%-45% gradient over 10 min) to give pure Compound 1 (42.47 mg, 41.62% yield, 98% purity) as a pink solid. LCMS: (ES⁺) m/z (M+H)⁺=677.5. ¹H NMR (400 MHz, CD₃OD) δ 7.84 (d, J=8.4 Hz, 2H), 7.61 (d, J=8.8 Hz, 2H), 7.34 (m, 2H), 7.13-7.02 (m, 3H), 6.69-6.61 (m, 3H), 3.98 (q, J=7.2 Hz, 4H), 3.82 (s, 2H), 3.72 (t, J=4.4 Hz, 2H), 3.65 (br s, 2H), 3.52 (d, J=6.0 Hz, 2H), 3.42 (d, J=4.8 Hz, 2H), 2.87-2.67 (m, 4H), 2.37-2.31 (m, 2H), 2.14-1.93 (m, 6H), 1.26 (t, J=6.8 Hz, 6H).

The following compounds were prepared according to the procedures described in Example 1 using the appropriate intermediates.

| Cpd | Characterization Data |
|---|---|
| 2 | (ES⁻) m/z (M − H)⁻ = 810.5. 1H NMR (400 MHz, CD₃OD) δ 7.89 (d, J = 8.8Hz, 2H), |
| | 7.73-7.65 (m, 2H), 7.28 (dd, J = 5.6, 8.8 Hz, 2H), 7.08-7.01 (m, 2H), 6.73 (s, 2H), |
| | 4.42-4.15 (m, 1H), 4.01-3.94 (m, 7H), 3.92-3.74 (m, 2H), 3.70 (s, 4H), 3.48 (t, J = |
| | 6.8 Hz, 2H), 2.73 (s, 4H), 2.63-2.51 (m, 4H), 2.14-1.96 (m, 4H), 1.22 (t, J = 6.8 Hz, 6H). |
| 3 | (ES⁺) m/z (M + H)⁺ = 698.2. 1H NMR (400 MHz, CD₃OD) δ 7.89 (d, J = 8.8 Hz, 2H), |
| | 7.70 (d, J = 8.8 Hz, 2H), 7.25-7.19 (m, 3H), 7.12 (t, J = 8.8 Hz, 2H), 7.06 (s, 1H), |
| | 4.33 (s, 2H), 4.01-3.95 (m, 7H), 3.82-3.77 (m, 2H), 3.70-3.61 (m, 4H), 3.49-3.46 (m, |
| | 1H), 3.13-2.95 (m, 4H), 2.16 (s, 2H), 2.09 (s, 2H), 1.20 (t, J = 6.8 Hz, 3H). |
| 4 | (ES⁺) m/z (M + H)⁺ = 712.5 |
| 5 | (ES⁺) m/z (M + H)⁺ = 737.6 |
| 6 | (ES⁺) m/z (M)⁺ = 647.5 |
| 7 | (ES⁺) m/z (M + H)⁺ = 738.5 |
| 8 | (ES⁺) m/z (M)⁺ = 730.6 |
| 9 | (ES⁺) m/z (M + H)⁺ = 664.4 |
| 10 | (ES⁺) m/z (M + H)⁺ = 684.5 |
| 11 | (ES⁺) m/z (M + H)⁺ = 670.4 |
| 12 | (ES⁺) m/z (M + H)⁺ = 656.4 |

### Example 2: Methyl 2-cyclopropyl-5-ethoxy-4-[[2-[4-[2-(2-hydroxyethylcarbamoylamino)ethylcarbamoyl]phenyl]-3-oxo-2,8-diazaspiro[4.5]decan-8-yl]methyl]benzoate (Compound 13)

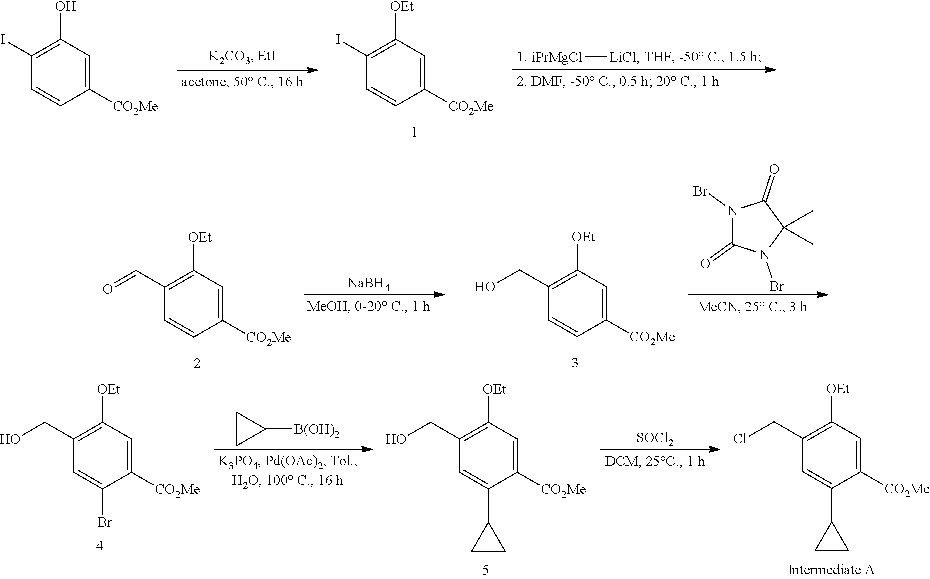

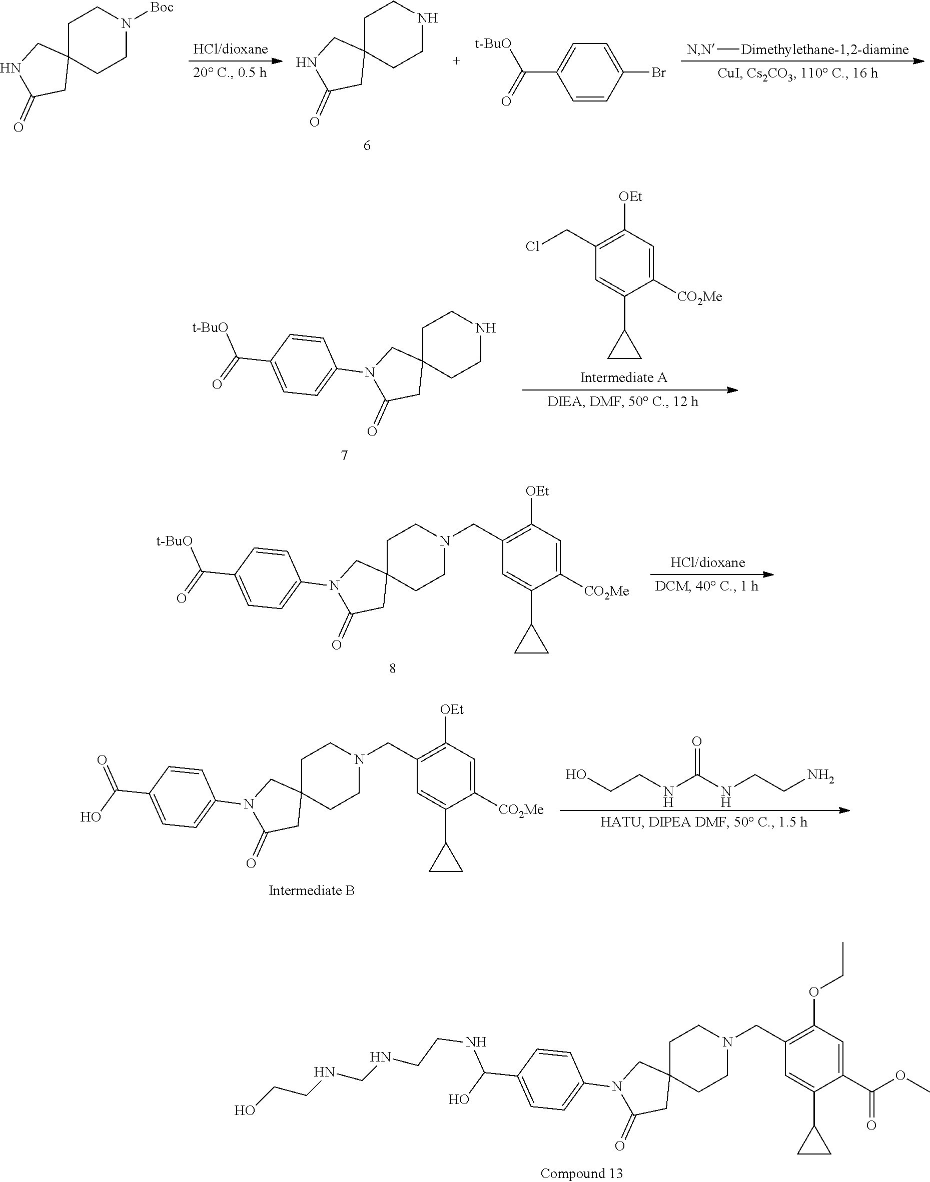

Step 1: methyl 3-ethoxy-4-iodobenzoate (1): To the suspension of methyl 3-hydroxy-4-iodobenzoate (25 g, 90 mmol, 1 eq) and K₂CO₃ (18.6 g, 135 mmol, 1.5 eq) in acetone (250 mL) was added EtI (18.2 g, 117 mmol, 9.35 mL, 1.3 eq), and the mixture was stirred at 50° C. for 16 hours. The reaction suspension was filtered, the filtrate was concentrated at vacuum (50° C.). The residue was dissolved in EA (400 mL), washed with brine (100 mL×3), dried over Na₂SO₄ and concentrated at vacuum (50° C.) to give 1 (26.4 g, 96% yield) as a light yellow solid. ¹H NMR (400 MHz, CDCl₃) δ 7.85 (d, J=8.0 Hz, 1 H), 7.42 (d, J=1.6 Hz, 1 H), 7.36 (dd, J₁=8.0 Hz, J₂=1.6 Hz, 1 H), 4.16 (q, J=7.2 Hz, 2 H), 3.92 (s, 3 H), 1.51 (t, J=7.2 Hz, 3 H).

Step 2: methyl 3-ethoxy-4-formylbenzoate (2): To the solution of 1 (20 g, 65 mmol, 1 eq) in THF (80 mL) was added i-PrMgCl-LiCl (1.3 M, 101 mL, 2 eq) dropwise at −50° C. under N₂ atmosphere ,and the mixture was stirred for 1.5 hours. Then DMF (95.5 g, 1.31 mol, 101 mL, 20 eq) was added dropwise to above reaction mixture, and the mixture was stirred for 0.5 hour at −50° C. The reaction suspension was cooled to 20° C. and stirred for 1 hour. The reaction suspension was quenched with citric acid (300 mL), extracted with EA (100 mL×3), washed with brine (100 mL×3), dried over Na₂SO₄ and concentrated under vacuum (50° C.) to give 2 (14.6 g, crude) as a white solid. ¹H NMR (400 MHz, CDCl₃) δ 10.53 (s, 1 H), 7.83-7.90 (m, 1 H), 7.59-7.75 (m, 2 H), 4.22 (q, J=7.2 Hz, 2 H), 3.93 (s, 3 H), 1.50 (t, J=7.2 Hz, 3 H).

Step 3: methyl 3-ethoxy-4-(hydroxymethyl)benzoate (3): To the solution of 2 (14.6 g, 70 mmol, 1 eq) in MeOH (150 mL) was added NaBH₄ (3.2 g, 84 mmol, 1.2 eq) portion-wise at 0° C., and the reaction mixture was stirred for 1 hour at 20° C. under N₂ atmosphere. The solvent was removed under vacuum (50° C.), and the residue was quenched with citric acid (200 mL), extracted with EA (100 mL×2), dried over Na₂SO₄ and concentrated under vacuum (50° C.). The residue was purified by flash silica gel chromatography (80 g Silica Flash Column, eluent of 0 to 30% ethyl acetate/petroleum ether gradient) to give 3 (9.5 g, 59% yield, 92% purity) as a white solid. LCMS: (ES⁺) m/z (M-17)⁺=193.7.

Step 4: methyl 2-bromo-5-ethoxy-4-(hydroxymethyl)benzoate (4): To the solution of 3 (9 g, 43 mmol, 1 eq) in MeCN (100 mL) was added 1,3-dibromo-5,5-dimethylimidazolidine-2,4-dione (6.7 g, 24 mmol, 0.55 eq), and the mixture was stirred at 25° C. for 3 hours. The solvent was removed under vacuum (40° C.). The residue was dissolved in EA (300 mL), washed with brine (100 mL×3), dried over Na₂SO₄ and concentrated at vacuum (50° C.) to give 4 (12.7 g, crude) as a yellow solid. ¹H NMR (400 MHz, CDCl₃) δ 7.54 (s, 1 H), 7.21 (s, 1 H), 4.62 (s, 2 H), 3.99-4.05 (m, 2 H), 3.86 (s, 3 H), 2.10 (s, 1 H), 1.37 (t, J=7.2 Hz, 3 H).

Step 5: methyl 2-cyclopropyl-5-ethoxy-4-(hydroxymethyl)benzoate (5): The reaction mixture of 4 (11.5 g, 40 mmol, 1 eq), cyclopropylboronic acid (10 g, 119 mmol, 3 eq), K₃PO₄ (25 g, 119 mmol, 3 eq), PCy₃ (558 mg, 1.99 mmol, 0.05 eq) and Pd(OAc)₂ (447 mg, 2.0 mmol, 0.05 eq) in toluene (120 mL) and H₂O (12 mL) was stirred at 100° C. for 16 hours under N₂ atmosphere. The reaction suspension was filtered, and the filtrate was concentrated under vacuum (50° C.). The residue was dissolved in EA (200 mL) and concentrated under vacuum (50° C.). The residue was purified by flash silica gel chromatography (80 g Silica Flash Column, Eluent of 0 to 30% ethyl acetate/petroleum ether gradient) to give 5 (6.2 g, 56% yield, 90% purity) as a light yellow oil. ¹H NMR (400 MHz, CDCl₃) δ 7.31 (s, 1 H), 6.98 (s, 1 H), 4.68 (d, J=6.4 Hz, 2 H), 4.12 (q, J=6.4 Hz, 2 H), 3.92 (s, 3 H), 2.49-2.60 (m, 1 H), 1.44 (t, J=7.2 Hz, 3 H), 0.91-0.97 (m, 2 H), 0.64 (m, 2 H).

Step 6: methyl 4-(chloromethyl)-2-cyclopropyl-5-ethoxybenzoate (Intermediate A): To a solution of 5 (5.9 g, 21 mmol, 1 eq) in DCM (60 mL) was added SOCl₂ (5.1 g, 42 mmol, 3.1 mL, 2 eq), and the mixture was stirred for 1 hour at 25° C. under N₂ atmosphere. The solvent was removed in vacuo (50° C.) to give the crude product Intermediate A (6.5 g, crude) as a brown oil. LCMS: (ES+) m/z (M+H)⁺=269.5. ¹H NMR (400 MHz, CDCl₃) δ 7.31 (s, 1 H), 7.07 (s, 1 H), 4.62 (s, 2 H), 4.12 (q, J=6.8 Hz, 2 H), 3.92 (s, 3 H), 2.51 (tt, J₁=8.4 Hz, J₂=5.6 Hz, 1 H), 1.45 (t, J=7.2 Hz, 3 H), 0.92-0.98 (m, 2 H), 0.61-0.66 (m, 2 H).

Step 7: 2,8-diazaspiro[4.5]decan-3-one (6): A solution of tert-butyl 3-oxo-2,8-diazaspiro[4.5]decane-8-carboxylate (4 g, 16 mmol) in HCl/dioxane (4 M, 10 mL) was stirred at 20° C. for 0.5 hour. The reaction mixture was concentrated under reduced pressure to give crude 7 (4 g, HCl) as a white solid.

Step 8: tert-butyl 4-(3-oxo-2,8-diazaspiro[4.5]decan-2-yl)benzoate (7): A mixture of 6 (2.5 g, 13 mmol, HCl), tert-butyl 4-bromobenzoate (4.1 g, 16 mmol), N,N′-dimethylethane-1,2-diamine (1.2 g, 13 mmol), CuI (2.5 g, 13 mmol) and Cs₂CO₃ (17 g, 53 mmol) was degassed and purged with N₂ 3 times, and then the mixture was stirred at 110° C. for 16 hours under N₂ atmosphere. The reaction mixture was diluted with methyl alcohol (100 mL), filtered and concentrated under reduced pressure to give a residue. The residue was purified by reversed-phase MPLC (column: Phenomenex luna C18 250×50 mm×10 μm, 100 Å; flow rate: 200 mL/min; mobile phase: A: [water (0.1%TFA, v/v), B: ACN]; B%: 5%-59% gradient over 30 min) to give 7 (2 g) as a white solid.

Step 9: methyl 4((2-(4-(tert-butoxycarbonyl)phenyl)-3-oxo-2,8-diazaspiro[4.5]decan-8-yl)methyl)-2-cyclopropyl-5-ethoxybenzoate (8): To a solution of 7 (2 g, 6.1 mmol, 1 eq) and Intermediate A (1.5 g, 5.5 mmol, 0.9 eq) in DMF (10 mL) was added DIEA (1.6 g, 12 mmol, 2.1 mL, 2 eq). The mixture was stirred at 50° C. for 12 hours. The reaction mixture was poured into H₂O (30 mL) and extracted with EA (50 mL×2). The combined organic layer was washed with water (30 mL×2) and brine (30 mL×2), dried over Na₂SO₄ and concentrated in vacuo to give 8 (3 g, 4.75 mmol, 78.43% yield, 89% purity) as a yellow solid. LCMS: (ES⁺) m/z (M+H)⁺=563.3.

Step 10: 4-(8-(5-cyclopropyl-2-ethoxy-4-(methoxycarbonyl)benzyl)-3-oxo-2,8-diazaspiro[4.5]decan-2-yl)benzoic acid (Intermediate B): To a solution of 8 (3 g, 5.3 mmol, 1 eq) in DCM (10.5 mL) in HCl/dioxane (4 M, 60 mL, 45.02 eq) was stirred at 40° C. for 1 hour. The mixture was concentrated in vacuo. The crude product was purified by reversed-phase HPLC (column: Phenomenex luna C18 250×50 mm×10 μm; mobile phase: [A: water (0.225% FA, v/v), B:ACN]; B%: 20%-30%, gradient over 10 min) to give Intermediate B (1.35 g, 49% yield, 99% purity) as a white solid. LCMS: (ES⁺) m/z (M+H)⁺=507.5. ¹H NMR (CD₃OD, 400 MHz): δ 8.01 (br d, J=8.8 Hz, 2 H), 7.73 (br d, J=8.8 Hz, 2 H), 7.39 (s, 1 H), 7.26 (s, 1 H), 4.32 (br s, 2 H), 4.17 (q, J=6.8 Hz, 2 H), 3.92 (s, 3 H), 3.86 (s, 2 H), 3.35 (br s, 3 H), 2.66 (s, 2 H), 2.49-2.43 (m, 1 H), 2.12-1.91 (m, 5 H), 1.47 (t, J=6.8 Hz, 3 H), 0.97-0.90 (m, 2 H), 0.71-0.64 (m, 2 H).

Step 11: methyl 2-cyclopropyl-5-ethoxy-4-((2-(4-((2-(3-(2-hydroxyethyl)ureido)ethyl)carbamoyl)phenyl)-3-oxo-2,8-diazaspiro[4.5]decan-8-yl)methyl)benzoate (Compound 13): To a solution of Intermediate B (60 mg, 0.12 mmol) in DMF (1 mL) was added HATU (54 mg, 0.14 mmol) and DIPEA (46 mg, 0.36 mmol, 62 μL), and the mixture was stirred at 50° C. for 0.5 hour, then 1-(2-aminoethyl)-3-(2-hydroxyethyl)urea (17 mg, 0.12 mmol) was added. The mixture was stirred at 50° C. for 1 hour. The solution was purified by pre-HPLC (column: Phenomenex Synergi C18 150×25×10 μm; mobile phase: [A: water (0.225% FA,v/v), B: ACN]; B%: 6%-36%, gradient over 10 min) to give Compound 13 (30.7 mg , 38% yield, 99% purity, FA salt) as a white solid. LCMS: (ES⁺) m/z (M+H)⁺=636.6. ¹H NMR (400 MHz, CD₃OD) δ 7.89-7.84 (m, 2H), 7.75 (d, J=8.8 Hz, 2H), 7.37 (s, 1H), 7.16 (d, J=2.4 Hz, 1H), 4.14 (q, J=6.8 Hz, 2H), 4.02 (br s, 2H), 3.91 (s, 3H), 3.82 (s, 2H), 3.55 (t, J=5.6 Hz, 2H), 3.50-3.42 (m, 2H), 3.38-3.33 (m, 2H), 3.23 (t, J=5.6 Hz, 2H), 3.15-2.88 (m, 4H), 2.62 (s, 2H), 2.53-2.44 (m, 1H), 1.91 (br s, 4H), 1.45 (t, J=7.2 Hz, 3H), 0.99-0.91 (m, 2H), 0.68-0.61 (m, 2H).

The following compounds were prepared according to the procedures described in Example 2 using the appropriate intermediates.

| Cpd | Characterization Data |
|---|---|
| 14 | LCMS: (ES⁺) m/z (M + H)⁺ = 710.2. ¹H NMR (400 MHz , DMSO-d₆) δ 8.10-7.96 (m, |
| | 1H), 7.56 (d, J = 8.8 Hz, 2H), 7.23 (t, J = 4.0 Hz, 3H), 7.03 (s, 1H), 6.60-6.50 (m, 1H), |
| | 5.78 (s, 1H), 5.02 (s, 3H), 4.02 (q, J = 7.2 Hz, 2H), 3.84 (s, 3H), 3.64 (s, 2H), 3.49-3.41 |
| | (m, 8H), 3.37 (s, 2H), 3.12-2.95 (m, 4H), 2.49-2.40 (m, 5H), 1.77-1.54 (m, 4H), 1.33 (t, |
| | J = 6.8 Hz, 3H), 1.01-0.83 (m, 2H), 0.62-0.46 (m, 2H). |
| 15 | LCMS: (ES⁺) m/z (M + H)⁺ = 635.6. ¹H NMR (400 MHz, CD₃OD) δ 7.90-7.83 (m, 2 |
| | H), 7.75 (d, J = 8.8 Hz, 2 H), 7.38 (s, 1 H), 7.18 (d, J = 2.00 Hz, 1 H), 4.20-4.06 (m, 4 |
| | H), 3.91 (s, 3 H), 3.84 (s, 2 H), 3.59 (t, J = 5.6 Hz, 2 H), 3.41 (t, J = 6.8 Hz, 2 H), 3.30- |
| | 3.25 (m, 2 H), 3.21-2.99 (m, 4 H), 2.63 (s, 2 H), 2.52-2.43 (m, 1 H), 2.30 (t, J = 7.2 Hz, |
| | 2 H), 2.02-1.86 (m, 6 H), 1.46 (t, J = 6.8 Hz, 3 H), 1.00-0.91 (m, 2 H), 0.70-0.61 (m, 2H). |
| 16 | LCMS: (ES⁺) m/z (M + H)⁺ = 666.6. ¹H NMR (400 MHz, CD₃OD) δ 7.87 (d, J = 8.8 |
| | Hz, 2H), 7.75 (d, J = 8.8 Hz, 2H), 7.39 (s, 1H), 7.20 (s, 1H), 4.22-4.12 (m, 4H), 3.92 (s, |
| | 3H), 3.85 (s, 2H), 3.72 (q, J = 5.2 Hz, 1H), 3.63-3.53 (m, 4H), 3.49-3.43 (m, 2H), 3.38- |
| | 3.33 (m, 2H), 3.27-3.11 (m, 4H), 2.64 (s, 2H), 2.47 (tt, J = 8.4, 5.2 Hz, 1H), 2.03- |
| | 1.90 (m, 4H), 1.46 (t, ./ = 7, 2 Hz, 3H), 0.99-0.91 (m, 2H), 0.69-0.63 (m, 2H). |
| 17 | LCMS: (ES⁺) m/z (M + H)⁺ = 704.3. ¹H-NMR (400 MHz, CD3OD) δ 7.48 (d, J = 8.4 Hz, |
| | 2H), 7.37 (s, 1H), 7.24-7.20 (m, 3H), 4.18-4.09 (m, 4H), 3.91 (s, 3H), 3.77 (s, 2H), 3.57 |
| | (t, J = 5.2 Hz, 2H), 3.21 (t, J = 5.6 Hz, 2H), 3.11-3.03 (m, 4H), 2.91 (s, 6H), 2.64 (t, J = |
| | 7.6 Hz, 2H), 2.58 (s, 2H), 2.49-2.44 (m, 1H), 2.25 (t, J = 7.6 Hz, 2H), 1.99-1.86 (m, 6H), |
| | 1.48-1.42 (m, 9H), 0.97-0.92 (m, 2H), 0.67-0.63 (m, 2H). |
| 18 | LCMS: (ES⁺) m/z (M + H)⁺ = 696.3. ¹H NMR (400 MHz, CD₃OD) δ 7.89 (d, J = 8.8 Hz, |
| | 2H), 7.77 (d, J = 8.8 Hz, 2H), 7.40 (s, 1H), 7.20 (s, 1H), 4.21-4.12 (m, 4H), 3.94 (s, 3H), |
| | 3.86 (s, 2H), 3.66 (s, 6H), 3.50-3.45 (m, 2H), 3.37-3.34 (m, 2H), 3.23-3.04 (m, 4H), |
| | 2.65 (s, 2H), 2.49 (tt, J = 8.4, 5.4 Hz, 1H), 2.05-1.89 (m, 4H), 1.48 (t, J = 6.8 Hz, 3H), |
| | 1.01-0.94 (m, 2H), 0.70-0.63 (m, 2H) |
| 19 | LCMS: (ES⁺) m/z (M + H)⁺ = 580.2. ¹H NMR (400 MHz, CD3OD) δ 8.44 (s, 1H), 7.89 |
| | (d, J = 8.8 Hz, 2H), 7.74 (d, J = 8.8 Hz, 2H), 7.38 (s, 1H), 7.18 (s, 1H), 4.22-4.08 (m, |
| | 5H), 3.91 (s, 3H), 3.83 (s, 2H), 3.74 (d, J = 5.6 Hz, 4H), 3.19-2.99 (m, 4H), 2.63 (s, |
| | 2H), 2.50-2.45 (m, 1H), 1.94-1.93 (m, 4H), 1.45 (t, J = 7.2 Hz, 3H), 0.96-0.94 (m, |
| | 2H), 0.66-0.64 (m, 2H). |
| 20 | LCMS: (ES⁺) m/z (M + H)⁺ = 550.2. ¹H NMR (400 MHz, CD3OD) δ 8.34 (s, 1H), 7.89- |
| | 7.87 (m, 2H), 7.76-7.74 (m, 2H), 7.39 (s, 1H), 7.20 (s, 1H), 4.21-4.13 (m, 4H), 3.92 |
| | (s, 3H), 3.85 (s, 2H), 3.73-3.69 (m, 2H), 3.53-3.48 (m, 2H), 3.27-3.10 (m, 4H), 2.65 |
| | (s, 2H), 2.52-2.43 (m, 1H), 2.03-1.90 (m, 4H), 1.46 (t, J = 7.2 Hz, 3H), 0.99-0.93 |
| | (m, 2H), 0.66-0.64 (m, 2H). |
| 21 | LCMS: (ES⁺) m/z (M + H)⁺ = 610.2. ¹H NMR (400 MHz, CD3OD) δ 8.44 (s, 1H), 7.87- |
| | 7.82 (m, 2H), 7.76-7.75 (m, 2H), 7.37 (s, 1H), 7.17 (s, 1H), 4.14 (q, J = 7.2 Hz, 2H), |
| | 4.05 (s, 2H), 3.91 (s, 3H), 3.85 (s, 6H), 3.82 (s, 2H), 3.19-2.94 (m, 4H), 2.62 (s, 2H), |
| | 2.50-2.46 (m, 1H), 1.98-1.85 (m, 4H), 1.45 (t, J = 7.2 Hz, 3H), 0.98-0.91 (m, 2H), |
| | 0.67-0.62 (m, 2H) |
| 22 | LCMS: (ES⁺) m/z (M)⁺ = 591.3. 1H NMR (400 MHz, D₂O) δ 8.43 (s, 2H), 7.82 (d, J = |
| | 8.8 Hz, 2H), 7.61 (d, J = 8.8 Hz, 2H), 7.35 (s, 1H), 7.14 (s, 1H), 4.33 (s, 2H), 4.16 (q, J = |
| | 6.8 Hz, 2H), 3.95 (s, 3H), 3.89 (t, J = 6.8 Hz, 4H), 3.60 (t, J = 6.8 Hz, 2H), 3.54- |
| | 3.28 (m, 4H), 3.22 (s, 9H), 2.81-2.62 (m, 2H), 2.30-2.23 (m, 1H), 2.14-1.93 (m, |
| | 4H), 1.39 (t, J = 6.8 Hz, 3H), 1.00-0.90 (m, 2H), 0.65-0.57 (m, 2H). |
| 23 | LCMS: (ES⁺) m/z (M + H)⁺ = 592.6. ¹H NMR (400 MHz, CD₃OD) δ 7.89-7.82 (m, 2H), |
| | 7.74 (d, J = 8.8 Hz, 2H), 7.38 (s, 1H), 7.17 (s, 1H), 4.15 (q, J = 6.8 Hz, 2H), 4.08 (s, 2H), |
| | 3.92 (s, 3H), 3.86-3.80 (m, 2H), 3.42 (t, J = 6.8 Hz, 2H), 3.18-2.98 (m, 4H), 2.63 (s, 2H), |
| | 2.48 (tt, J = 8.4, 5.2 Hz, 1H), 2.37 (t, J = 7.2 Hz, 2H), 1.92 (q, J = 7.2 Hz, 6H), 1.46 (t, |
| | J = 6.8 Hz, 3H), 1.00-0.91 (m, 2H), 0.69-0.61 (m, 2H). |

### Example 3: (5-methyl-2-oxo-1,3-dioxo1-4-yl)methyl 2-cyclopropyl-5-ethoxy-4-((3-oxo-2-(4-(((2S,3R,4R,5R)-2,3,4,5,6-pentahydroxyhexyl)carbamoyl)phenyl)-2,8-diazaspiro [4.5] decan-8-yl)methyl)benzoate (Compound 24)

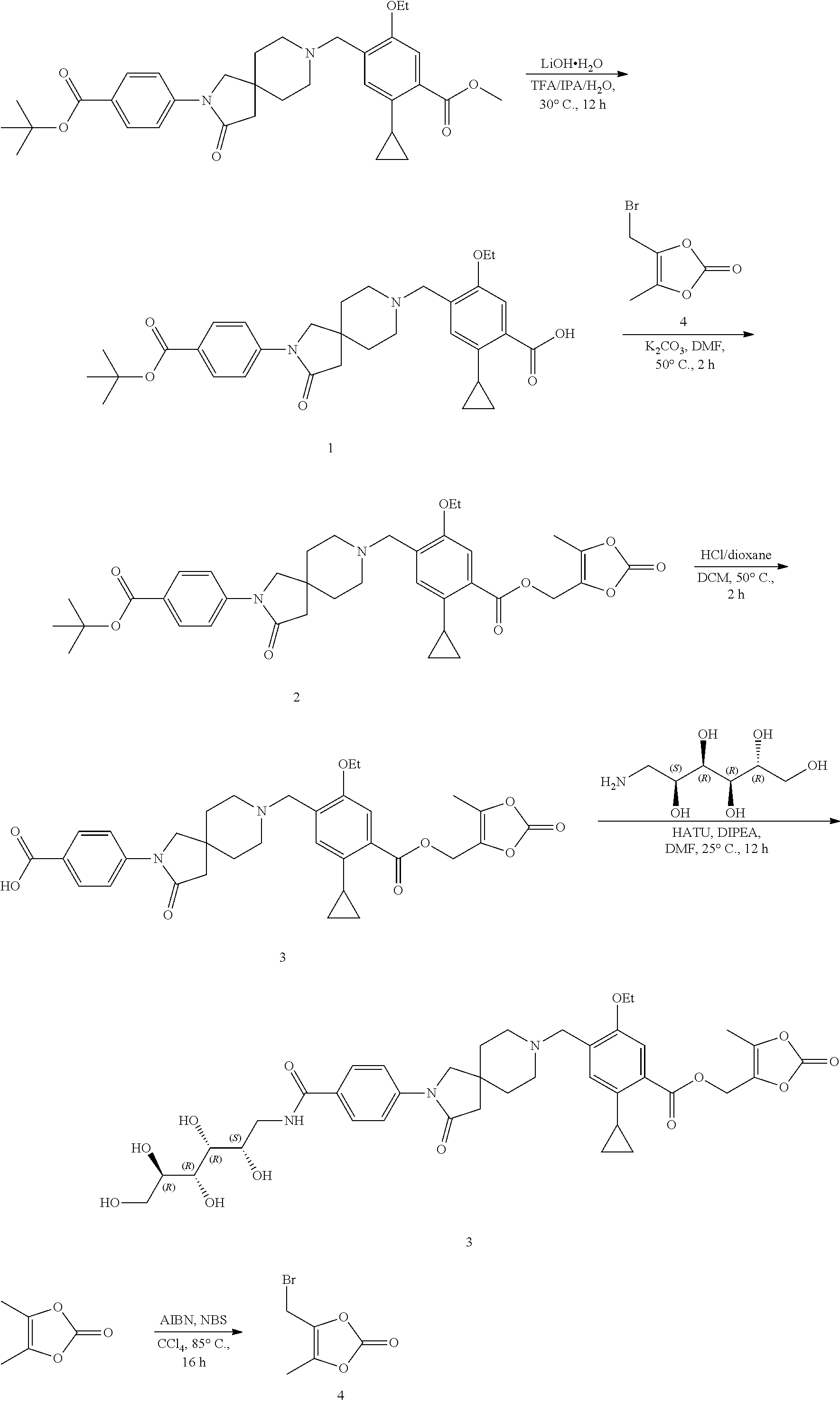

Step 1: 4-(bromomethyl)-5-methyl-1,3-dioxol-2-one (4): To a solution of 4,5-dimethyl-1,3-dioxol-2-one (15 g, 131 mmol) in CCl₄ (350 mL) was added NBS (23 g, 131 mmol) and AIBN (1.1 g, 6.6 mmol, 0.05) under N₂. Then the mixture was stirred at 85° C. for 16 hours. The mixture was concentrated to one-half the initial volume, cooled in an ice bath, and the white solid was filtered off. The filtrate was concentrated in vacuo and purified by column chromatography (SiO₂, petroleum ether/ethyl acetate, 6/1) to give 4 (24 g, 95% yield) as a pale brown liquid. ¹H NMR (400 MHz, CDCl₃) δ 4.19 (s, 2H), 2.14 (s, 3H)

Step 2: 4-((2-(4-(tert-butoxycarbonyl)phenyl)-3-oxo-2,8-diazaspiro[4.5]decan-8-yl) methyl)-2-cyclopropyl-5-ethoxybenzoic acid (1): To a solution of methyl 4-((2-(4-(tert-butoxycarbonyl)phenyl)-3-oxo-2,8-diazaspiro[4.5]decan-8-yl)methyl)-2-cyclopropyl-5-ethoxybenzoate (200 mg, 0.36 mmol) in THF (1.5 mL), IPA (1.5 mL) and H₂O (1.5 mL) was added LiOH·H₂O (45 mg, 1.1 mmol, 3 eq). The mixture was stirred at 30° C. for 12 hours. The mixture was diluted with water (20 mL), adjusted to pH 5 with 1 N aqueous HCl, and extracted with EtOAc (20 mL×2). The combined organic layer was washed with brine (20 mL×2), dried over Na₂SO₄, filtered and concentrated in vacuo to give 1 (210 mg, 90% purity) as a white solid. LCMS: (ES⁺) m/z (M+H)⁺=549.3.

Step 3: (5-methyl-2-oxo-1,3-dioxo1-4-yl)methyl 4-((2-(4-(tert-butoxycarbonyl)phenyl)-3-oxo-2,8-diazaspiro[4.5]decan-8-yl)methyl)-2-cyclopropyl ethoxybenzoate (2): To a solution of 1 (150 mg, 0.27 mmol, 1 eq) in DMF (3 mL) was added K₂CO₃ (151 mg, 1.09 mmol, 4 eq) and 4 (158 mg, 0.82 mmol, 3 eq). The mixture was stirred at 50° C. for 2 hours. The mixture was diluted with EtOAc (30 mL) and washed with water (10 mL) and brine (20 mL×2), dried over Na₂SO₄, and concentrated in vacuo. The residue was purified by column chromatography (SiO₂, petroleum ether/ethyl acetate, 1:2) to give 2 (85 mg, 45% yield, 96% purity) as a yellow oil. LCMS: (ES⁺) m/z (M+H)⁺=661.3.

Step 4: 4-(8-(5-cyclopropyl-2-ethoxy-4-(((5-methyl-2-oxo-1,3-dioxo1-4-yl)methoxy) carbonyl)benzyl)-3-oxo-2,8-diazaspiro[4.5]decan-2-yl)benzoic acid (3): To a solution of 2 (65 mg, 98 μmol, 1 eq) in DCM (1 mL) was added HCl/dioxane (4 M, 3 mL), and the mixture was stirred at 50° C. for 2 hours. The mixture was concentrated in vacuo to give 3 (82 mg, crude) as a yellow solid. LCMS: (ES⁺) m/z (M+H)⁺=605.3.

Step 5: (5-methyl-2-oxo-1,3-dioxo1-4-yl)methyl 2-cyclopropyl-5-ethoxy-4-((3-oxo-2-(4 (((2S,3R,4R,5R)-2,3,4,5,6-pentahydroxyhexyl)carbamoyl)phenyl)-2,8-diazaspiro[4.5]decan-8-yl)methyl) benzoate (Compound 24): To a solution of 3 (82 mg, crude) and HATU (58 mg, 153 μmol, 1.2 eq) in DMF (2 mL) was added DIPEA (50 mg, 384 μmol, 67 μL, 3 eq) and (2R,3R,4R,5S)-6-aminohexane-1,2,3,4,5-pentol (35 mg, 192 μmol, 1.5 eq). The mixture was stirred at 25° C. for 12 hours. The mixture was concentrated in vacuo. The residue was purified by prep-HPLC (column: Phenomenex Synergi C18 150×25 mm×10 μm; mobile phase: A: water (0.225%FA,v/v); B: ACN; B%: 7%-37%, gradient over 10 min) to give Compound 24 (25 mg, 97% purity, FA salt) as off-white solid. LCMS: (ES⁺) m/z (M+H)⁺=768.7. ¹H NMR (400 MHz, CD₃OD) δ 8.42 (br s, 1H), 7.87 (d, J=8.8 Hz, 1H), 7.75 (J=8.8 Hz, 1H), 7.39 (s, 1H), 7.20 (s, 1H), 5.19 (s, 2H), 4.14 (q, J=6.8 Hz, 2H), 3.83 (s, 2H), 3.82-3.80 (m, 1H), 3.71-3.65 (m, 8H), 3.50-3.44 (m, 1H), 3.07-2.95 (m, 4H), 2.62 (s, 2H), 2.48-2.44 (m, 1H), 2.25 (s, 3H), 1.95-1.91 (m, 4H), 1.45 (t, J=6.8 Hz, 3H), 0.95-0.92 (m, 2H), 0.66-0.64 (m, 2H).

The following compounds were prepared according to the procedures described above using the appropriate intermediates.

| Cpd | Characterization Data |
|---|---|
| 25 | (ES⁺) m/z (M + H)⁺ = 578.2. ¹H NMR (400 MHz, CDCl3) δ 7.79-7.77 (m, 2H), 7.52- |
| | 7.50 (m, 2H), 7.31 (s, 1H), 7.15 (s, 1H), 4.09 (q, J = 7.2 Hz, 2H), 3.93 (s, 5H), 3.77- |
| | 3.70 (m, 2H), 3.36 (s, 2H), 3.14-3.04 (m, 2H), 2.65-2.63 (m, 2H), 2.59-2.47 (m, |
| | 3H), 2.44 (s, 2H), 1.94-1.84 (m, 2H), 1.68-1.64 (m, 2H), 1.43 (t, J = 7.2 Hz, 3H), |
| | 0.99-0.92 (m, 2H), 0.66-0.65 (m, 2H). |
| 26 | (ES⁺) m/z (M + H)⁺ = 594.2. 1H NMR (400 MHz, CD₃OD) δ 7.87 (d, J = 8.8 Hz, 2H), |
| | 7.74 (d, J = 8.8 Hz, 2H), 7.37 (s, 1H), 7.18 (s, 1H), 4.14 (q, J = 7.2 Hz, 2H), 4.06 (s, 2H), |
| | 3.91 (s, 3H), 3.82 (s, 2H), 3.70-3.64 (m, 4H), 3.65-3.57 (m, 4H), 3.14-2.97 (m, 4H), |
| | 2.62 (s, 2H), 2.50-2.45 (m, 1H), 1.98-1.85 (m, 4H), 1.45 (t, J = 7.2 Hz, 3H), 0.98-0.90 |
| | (m, 2H), 0.68-0.61 (m, 2H). |
| 27 | (ES⁺) m/z (M + H)⁺ = 564.2. 1H NMR (400 MHz, CDCl3) δ 8.41 (s, 1H), 7.79-7.77 (m, |
| | 2H), 7.67-7.65 (m, 2H), 7.30 (s, 1H), 7.13 (s, 1H), 6.86 (t, J = 5.6 Hz, 1H), 4.59-4.31 |
| | (m, 2H), 4.09 (q, J = 7.2 Hz, 2H), 3.93 (s, 3H), 3.88 (s, 2H), 3.73 (t, J = 5.2 Hz, 2H), |
| | 3.66 (s, 2H), 3.65-3.59 (m, 2H), 2.90 (s, 2H), 2.74-2.63 (m, 2H), 2.51-2.47 (m, 1H), |
| | 2.01-1.91 (m, 2H), 1.87-1.77 (m, 4H), 1.43 (t, J = 6.8 Hz, 3H), 0.98-0.91 (m, 2H), |
| | 0.66-0.63 (m, 2H). |
| 28 | (ES⁺) m/z (M)⁺ = 605.3. 1H NMR (400 MHz, CD3OD) δ 8.45 (s, 1H), 7.90-7.85 (m, |
| | 2H), 7.78-7.75 (m, 2H), 7.35 (s, 1H), 7.14 (s, 1H), 4.11 (q, J = 6.8 Hz, 2H), 3.91 (s, |
| | 3H), 3.89 (s, 2H), 3.80 (s, 2H), 3.50-3.48 (m, 2H), 3.45-3.40 (m, 2H), 3.15 (s, 9H), |
| | 2.94-2.92 (m, 2H), 2.86-2.77 (m, 2H), 2.60 (s, 2H), 2.51-2.46 (m, 1H), 2.16-2.08 |
| | (m, 2H), 1.90-1.83 (m, 4H), 1.44 (t, J = 7.2 Hz, 3H), 0.97-0.91 (m, 2H), 0.65-0.60 |
| | (m, 2H). |
| 29 | (ES⁺) m/z (M + H)⁺ = 614.5 |
| 30 | (ES⁺) m/z (M + H)⁺ = 628.5 |
| 31 | (ES⁺) m/z (M + H)⁺ = 642.5 |
| 32 | (ES⁺) m/z (M + H)⁺ = 628.5 |
| 33 | (ES⁺) m/z (M + H)⁺ = 626.5 |
| 34 | (ES⁺) m/z (M + H)⁺ = 594.6 |
| 35 | (ES⁺) m/z (M + H)⁺ = 580.5 |
| 36 | (ES⁺) m/z (M + H)⁺ = 580.6 |
| 37 | (ES⁺) m/z (M + H)⁺ = 670.4 |
| 38 | (ES⁺) m/z (M + H)⁺ = 592.5 |
| 39 | (ES⁺) m/z (M + H)⁺ = 606.5 |
| 40 | (ES⁺) m/z (M + H)⁺ = 618.5 |
| 41 | (ES⁺) m/z (M + H)⁺ = 606.5 |
| 42 | (ES⁺) m/z (M + H)⁺ = 606.5 |
| 43 | (ES⁺) m/z (M + H)⁺ = 592.5 |
| 44 | (ES⁺) m/z (M + H)⁺ = 627.5 |
| 45 | (ES⁺) m/z (M)⁺ = 688.4 |
| 46 | (ES⁺) m/z (M + H)⁺ = 563.4 |
| 47 | (ES⁺) m/z (M + H)⁺ = 642.6 |
| 48 | (ES⁺) m/z (M + H)⁺ = 594.5 |
| 49 | (ES⁺) m/z (M + H)⁺ = 638.6 |
| 50 | (ES⁺) m/z (M + H)⁺ = 613.5 |
| 51 | (ES⁺) m/z (M + H)⁺ = 641.5 |
| 52 | (ES⁺) m/z (M + H)⁺ = 653.5 |
| 53 | (ES⁺) m/z (M + H)⁺ = 667.5 |
| 54 | (ES⁺) m/z (M + H)⁺ = 672.2 |
| 55 | (ES⁺) m/z (M + H)⁺ = 605.4 |
| 56 | (ES⁺) m/z (M + H)⁺ = 596.5 |
| 57 | (ES⁺) m/z (M + H)⁺ = 608.5 |
| 58 | (ES⁺) m/z (M + H)⁺ = 630.4 |
| 59 | (ES⁺) m/z (M + H)⁺ = 629.4 |

### Example 4: 4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzamide (Compound 65)

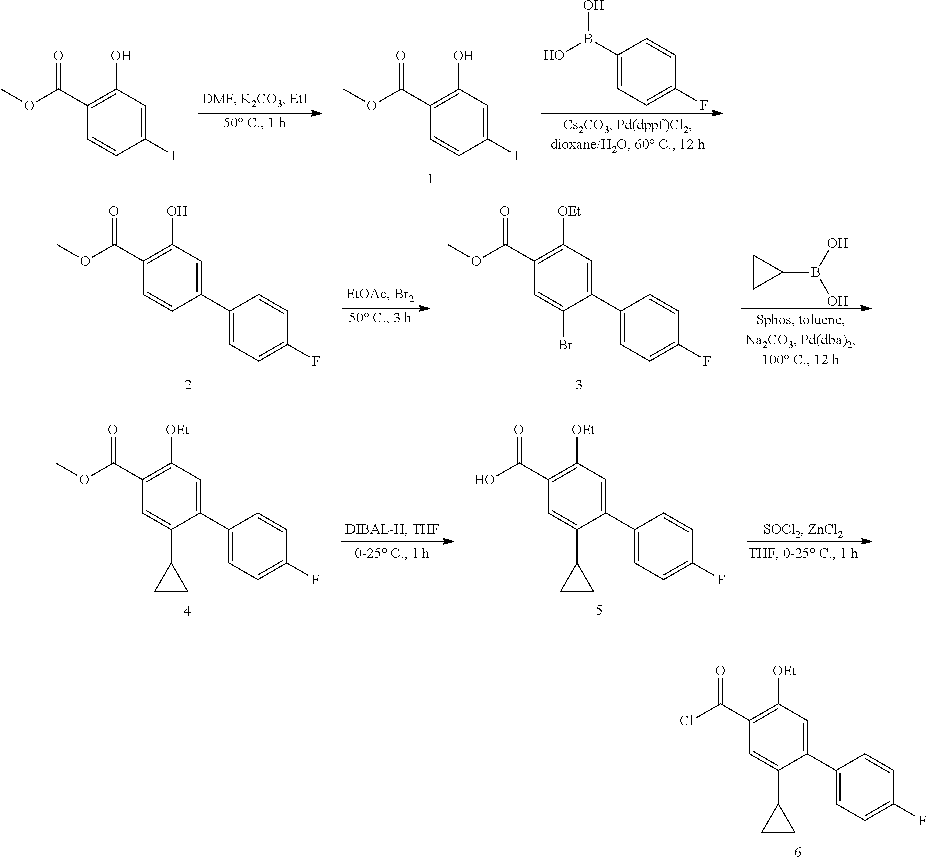

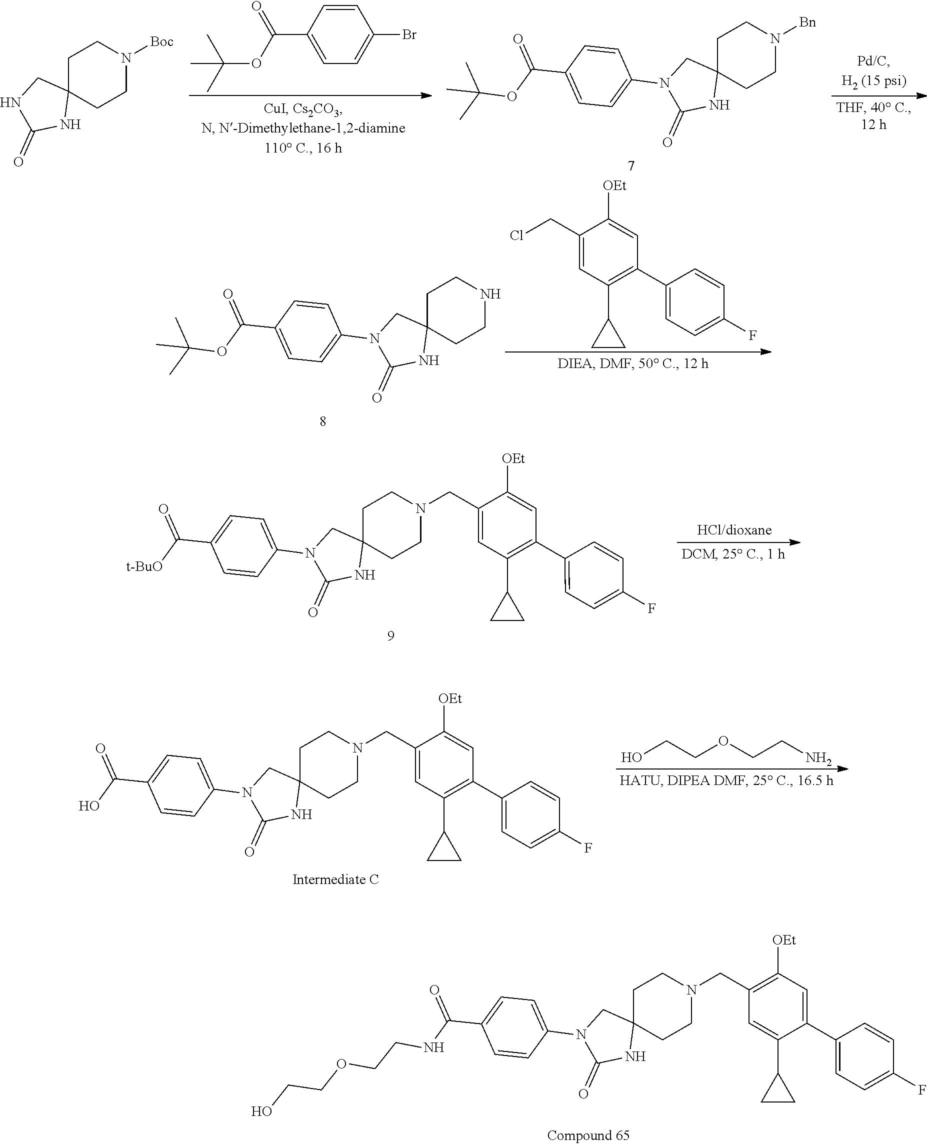

Step 1: methyl 2-ethoxy-4-iodo-benzoate (1): To a solution of methyl 2-hydroxy-4-iodo-benzoate (13 g, 47 mmol, 1 eq) in DMF (130 mL) was added K₂CO₃ (13 g, 94 mmol, 2 eq) and EtI (14.6 g, 94 mmol, 7.5 mL, 2 eq). The mixture was stirred at 50° C. for 1 hour. The reaction mixture was poured into H₂O (50 mL) and extracted with EA (50 mL×3). The combined organic layer was washed with water (50 mL×2) and brine (50 mL×2), dried over Na₂SO₄ and concentrated in vacuo to give 1 (14.2 g, crude) as a yellow oil.

Step 2: methyl 2-ethoxy-4-(4-fluorophenyl)benzoate (2): To a solution of 1 (7.5 g, 25 mmol, 1 eq), (4-fluorophenyl)boronic acid (3.8 g, 27 mmol, 1.1 eq), Cs₂CO₃ (16 g, 49 mmol, 2 eq) and Pd(dppf)Cl₂ (896 mg, 1.2 mmol, 0.05 eq) was added H₂O (20 mL) and dioxane (60 mL). Then the mixture was stirred at 60° C. for 12 hours. The reaction mixture was diluted with H₂O (100 mL) and extracted with EA (90 mL×2). The combined organic layers were washed with saturated brine (50 mL×2), dried over Na₂SO₄, filtered and concentrated under reduced pressure to give a residue. The residue was purified by column chromatography (SiO₂, petroleum ether/ethyl acetate=100/1 to 10/1) to give 2 (6.7 g, 99% yield) as a yellow solid.

Step 3: methyl 5-bromo-2-ethoxy-4-(4-fluorophenyl)benzoate (3): To a solution of 2 (7.2 g, 26 mmol, 1 eq) in EtOAc (72 mL) was added Br2 (5.0 g, 32 mmol, 1.6 mL, 1.2 eq). Then the mixture was stirred at 50° C. for 3 hours. The reaction mixture was diluted with H₂O (120 mL) and extracted with EA (75 mL×2). The combined organic layers were washed with saturated brine (50 mL×2), dried over Na₂SO₄, filtered and concentrated under reduced pressure to give a residue. The residue was purified by column chromatography (SiO₂, petroleum ether/ethyl acetate, 50/1 to 5/1) to give 3 (5.5 g, 59% yield) as a white solid. ¹H NMR (400 MHz, CD₃OD) δ 8.07 (s, 1 H), 7.35-7.42 (m, 2 H), 7.10-7.17 (m, 2 H), 6.90 (s, 1 H), 4.11 (q, J=7.2 Hz, 2 H), 3.91 (s, 3 H), 1.47 (t, J=6.8 Hz, 3 H).

Step 4: methyl 5-cyclopropyl-2-ethoxy-4-(4-fluorophenyl)benzoate (4): To a solution of 3 (5.5 g, 16 mmol, 1 eq), cyclopropylboronic acid (3.3 g, 39 mmol, 2.5 eq) and Na₂CO₃ (4.1 g, 39 mmol, 2.5 eq) in toluene (16 mL) was added SPhos (959 mg, 2.3 mmol, 0.15 eq) and Pd(dba)₂ (269 mg, 467 μmol, 0.03 eq) at N₂. Then the mixture was stirred at 100° C. for 12 hours. The reaction mixture was diluted with H₂O 120 mL and extracted with EA (75 mL×2). The combined organic layers were washed with saturated brine (50 mL×2), dried over Na₂SO₄, filtered and concentrated under reduced pressure and purified by column chromatography (SiO₂, petroleum ether/ethyl acetate, 50/1 to 5/1) to give 4 (4.8 g, 98% yield) as a yellow solid.

Step 5: [5-cyclopropyl-2-ethoxy-4-(4-fluorophenyl)phenyl]methanol (5): To a solution of 4 (4.8 g, 15 mmol, 1 eq) in THF (50 mL) was added DIBAL-H (1 M, 46 mL, 3 eq) at 0° C. Then the mixture was stirred at 25° C. for 1 hour. The reaction mixture was quenched by addition H₂O 70 mL at 0° C., and then added 1N HCl 60 mL and extracted with EA (50 mL×2). The combined organic layers were washed with saturated brine (40 mL×2), dried over Na₂SO₄, filtered and concentrated under reduced pressure to give 5 (5 g, crude) as a white solid.

Step 6: 1-(chloromethyl)-5-cyclopropyl-2-ethoxy-4-(4-fluorophenyl)benzene (6): To a solution of 5 (5 g, 17 mmol, 1 eq) in THF (50 mL) was added SOCl₂ (3.1 g, 26 mmol, 1.9 mL, 1.5 eq) and ZnCl₂ (238 mg, 1.8 mmol, 82 μL, 0.1 eq) at 0° C. Then the mixture was stirred at 25° C. for 1 hour. The reaction mixture was diluted with H₂O (100 mL) and extracted with EA (90 mL×2). The combined organic layers were washed with saturated brine (50 mL×2), dried over Na₂SO₄, filtered and concentrated under reduced pressure to give 6 (4.8 g, 90% yield) as a yellow oil.

Step 7: tert-butyl 4-(8-benzyl-2-oxo-1,3,8-triazaspiro[4.5]decan-3-yl)benzoate (7): To a solution of 8-benzyl-1,3,8-triazaspiro[4.5]decan-2-one (3 g, 12 mmol, 1 eq), CuI (2.3 g, 12 mmol, 1 eq), and Cs₂CO₃ (16 g, 49 mmol, 4 eq) in dioxane (13 mL) was added tert-butyl 4-bromobenzoate (3.1 g, 12 mmol, 1 eq) and N,N′-dimethylethane-1,2-diamine (1.1 g, 12 mmol, 1.3 mL, 1 eq). The mixture was stirred at 110° C. for 16 hours. The mixture was diluted with EA (40 mL) and then filtered. The residue was dissolved in water (40 mL) and NH₃.H₂O (60 mL) and extracted with EA (60 mL×3). The combined organic layers were washed with saturated brine (50 mL), dried over Na₂SO₄, filtered and concentrated under reduced pressure to give a residue. The crude product was triturated with 30 mL (EA:PE, 1:1) at 25° C. for 2 hours to give 7 (4.6 g, 89% yield) as a white solid. LCMS: (ES⁺) m/z (M+H)⁺=422.3.

Step 8: tert-butyl 4-(2-oxo-1,3,8-triazaspiro[4.5]decan-3-yl)benzoate (8): A mixture of 7 (4.6 g, 11 mmol, 1 eq) in THF (130 mL) was added 5% Pd/C (4.6 g, 2.2 mmol, 0.2 eq) under N₂ atmosphere. The suspension was degassed and purged with H₂ 3 times. The mixture was stirred under H₂ (15 psi) at 40° C. for 12 hours. The mixture was filtered, and the filtrate was concentrated in vacuo. The crude product was washed with EA (100 mL×3), and the filter cake was concentrated under reduced pressure to give 8 (2.2 g, 59% yield) as a white solid. LCMS: (ES⁺) m/z (M+H)⁺=332.4.

Step 9: tert-butyl 4-(84(2-cyclopropyl-5-ethoxy-4′-fluoro-[1,1′-biphenyl]-4-yl)methyl)-2-oxo-1,3,8-triazaspiro[4.5]decan-3-yl)benzoate (9): To a solution of 8 (1.5 g, 4.4 mmol, 1 eq) and 1-(chloromethyl)-5-cyclopropyl-2-ethoxy-4-(4-fluorophenyl)benzene 6 (1.2 g, 3.9 mmol, 0.9 eq) in DMF (20 mL) was added DIEA (1.7 g, 13 mmol, 2.3 mL, 3 eq). The mixture was stirred at 50° C. for 12 hours. The reaction mixture was poured into H₂O (30 mL) and extracted with EA (50 mL×3). The combined organic layer was washed with water (30 mL×2) and brine (30 mL×2), dried over Na₂SO₄ and concentrated in vacuo. The residue was purified by flash silica gel chromatography (20 g SepaFlash® Silica Flash Column, eluent of 0-100% ethyl acetate/petroleum ether gradient, 40 mL/min) to give 9 (1.4 g, 59% yield) as a white solid. LCMS: (ES⁺) m/z (M+H)⁺=600.3.

Step 10: 4-(842-cyclopropyl-5-ethoxy-4′-fluoro-[1,1′-biphenyl]-4-yl)methyl)-2-oxo-1,3,8-triazaspiro[4.5]decan-3-yl)benzoic acid (Intermediate C): A solution of 9 (1.3 g, 2.1 mmol, 1 eq) in HCl/dioxane (4 M, 16 mL, 30 eq) was stirred at 25° C. for 1 hour. The mixture was concentrated to give Intermediate C (1.04 g, 81% yield, 96% purity, HCl salt) as a white solid. LCMS: (ES⁺) m/z (M+H)⁺=544.3. ¹H NMR (CD₃OD, 400 MHz) δ 7.99 (dd, J1=8.4 Hz, J₂=6.0 Hz, 2H), 7.68 (dd, J₁=18.8, J₂=8.8 Hz, 2H), 7.45 (dd, J₁=8.4, J₂=5.6 Hz, 2H), 7.23-7.14 (m, 3H), 6.92 (s, 1H), 4.39 (d, J=7.8 Hz, 2H), 4.18 (q, J=6.8 Hz, 2H), 4.02 (s, 1H), 3.85 (s, 1H), 3.59 (br d, J=13.2 Hz, 2H), 3.43-3.32 (m, 2H), 2.22-2.09 (m, 4H), 1.84-1.73 (m, 1H), 1.47 (t, J=6.8 Hz, 3H), 0.86-0.79 (m, 2H), 0.68 (q, J=5.2 Hz, 2H).

Step 11: 4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzamide (Compound 65): A solution of Intermediate C (1.2 g, 2.1 mmol, 1 eq, HCl), HATU (958 mg, 2.5 mmol, 1.2 eq) and DIEA (814 mg, 6.3 mmol, 1.1 mL, 3 eq) in DMF (15 mL) was stirred at 25° C. for 0.5 hour. Then 2-(2-aminoethoxy)ethanol (442 mg, 4.2 mmol, 421 μL, 2 eq) was added. The mixture was stirred at 25° C. for 16 hours. The mixture was purified by reversed-phase HPLC (column: Phenomenex luna C18 150×40 mm×15 μm; mobile phase: [A: water (0.225% FA, v/v), B:ACN]; B%: 12%-42% gradient over 9 min) to give Compound 65 (981 mg, 67% yield, 97% purity, formic acid salt) as a white solid. LCMS: (ES⁺) m/z (M+H)⁺=631.2. ¹H NMR (400 MHz, CD₃OD) δ 7.83 (d, J=8.8 Hz, 2H), 7.66 (d, J=8.8 Hz, 2H), 7.45 (dd, J₁=8.8, J₂=5.2 Hz, 2H), 7.18 (t, J=8.8 Hz, 2H), 7.11 (s, 1H), 6.91 (s, 1H), 4.28 (br s, 2H), 4.16 (q, J=6.8 Hz, 2H), 3.90 (s, 2H), 3.70-3.63 (m, 4H), 3.60-3.55 (m, 4H), 3.36 (br s, 4H), 2.09 (br s, 4H), 1.84-1.73 (m, 1H), 1.46 (t, J=6.8 Hz, 3H), 0.85-0.77 (m, 2H), 0.64 (q, J=5.2 Hz, 2H).

The following compounds were prepared according to the procedures described above using the appropriate intermediates.

| Cpd | Characterization Data |
|---|---|
| 60 | (ES⁺) m/z (M + H)⁺ = 708.5 |
| 61 | (ES⁺) m/z (M + H)⁺ = 632.2 |
| 62 | (ES⁺) m/z (M + H)⁺ = 644.5 |
| 63 | (ES⁺) m/z (M + H)⁺ = 666.1 |
| 64 | (ES⁺) m/z (M + H)⁺ = 665.4 |
| 66 | (ES⁺) m/z (M + H)⁺ = 665.1 |
| 67 | (ES⁺) m/z (M + H)⁺ = 619.2 |
| 68 | (ES⁺) m/z (M + H)⁺ = 653.2 |
| 69 | (ES⁺) m/z (M + H)⁺ = 587.6 |
| 70 | (ES⁺) m/z (M + H)⁺ = 675.6 |
| 71 | (ES⁺) m/z (M + H)⁺ = 651.4 |
| 72 | (ES⁺) m/z (M + H)⁺ = 649.2. |
| 73 | (ES⁺) m/z (M + H)⁺ = 661.2 |
| 74 | (ES⁺) m/z (M + H)⁺ = 632.2 |
| 75 | (ES⁺) m/z (M + H)⁺ = 551.6 |
| 76 | (ES⁺) m/z (M + H)⁺ = 595.5 |
| 77 | (ES⁺) m/z (M + H)⁺ = 607.5 |
| 78 | (ES⁺) m/z (M + H)⁺ = 629.4 |
| 79 | (ES⁺) m/z (M + H)⁺ = 628.5 |

### Example 5: 4-(8-((2-cyclopropyl-5-ethoxy-4′-fluoro-[1,1′-biphenyl]-4-yl)methyl)-2-oxo-1-oxa-3,8-diazaspiro [4.5] decan-3-yl)-N-(3-guanidinopropyl)benzenesulfonamide (Compound 80)

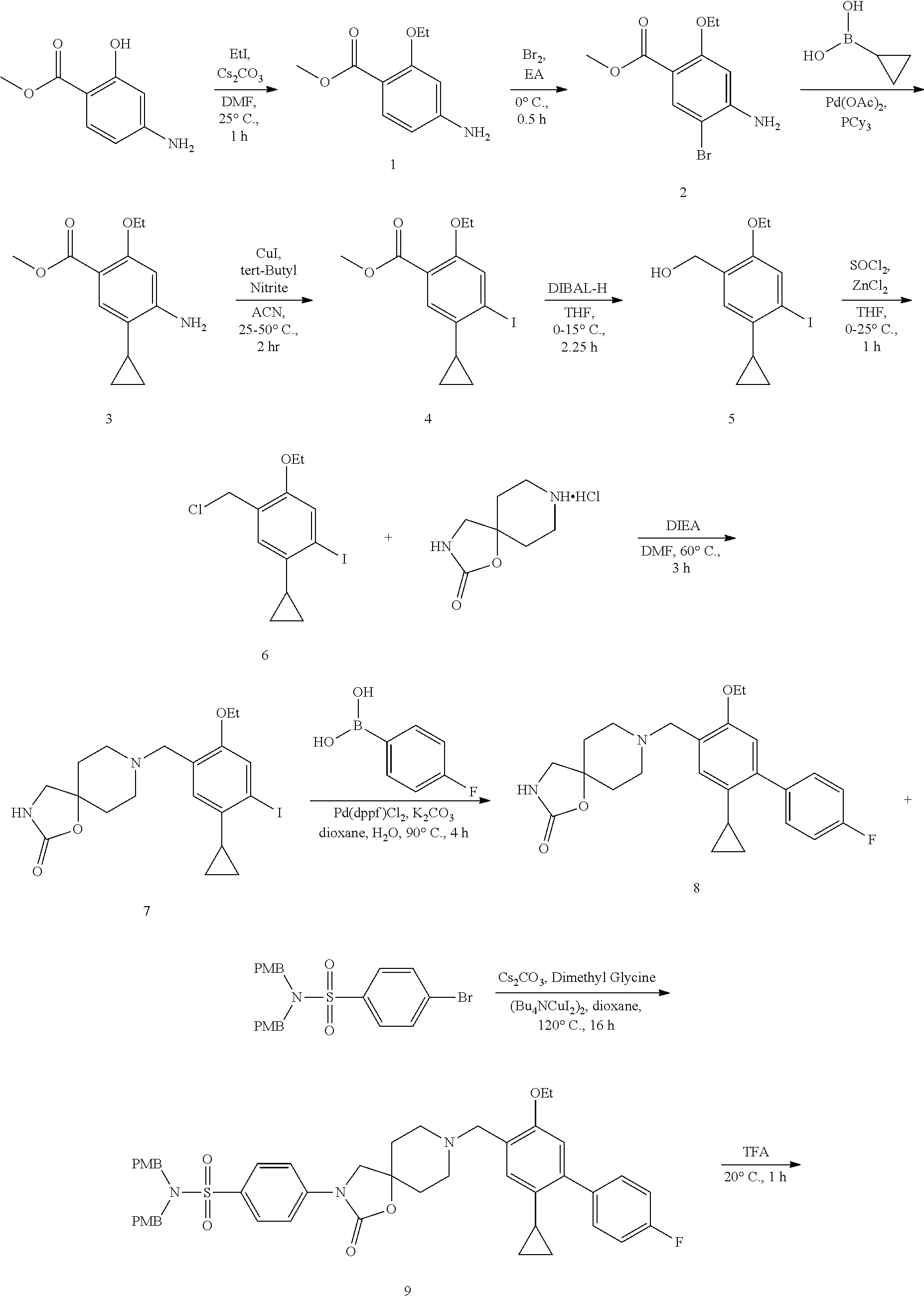

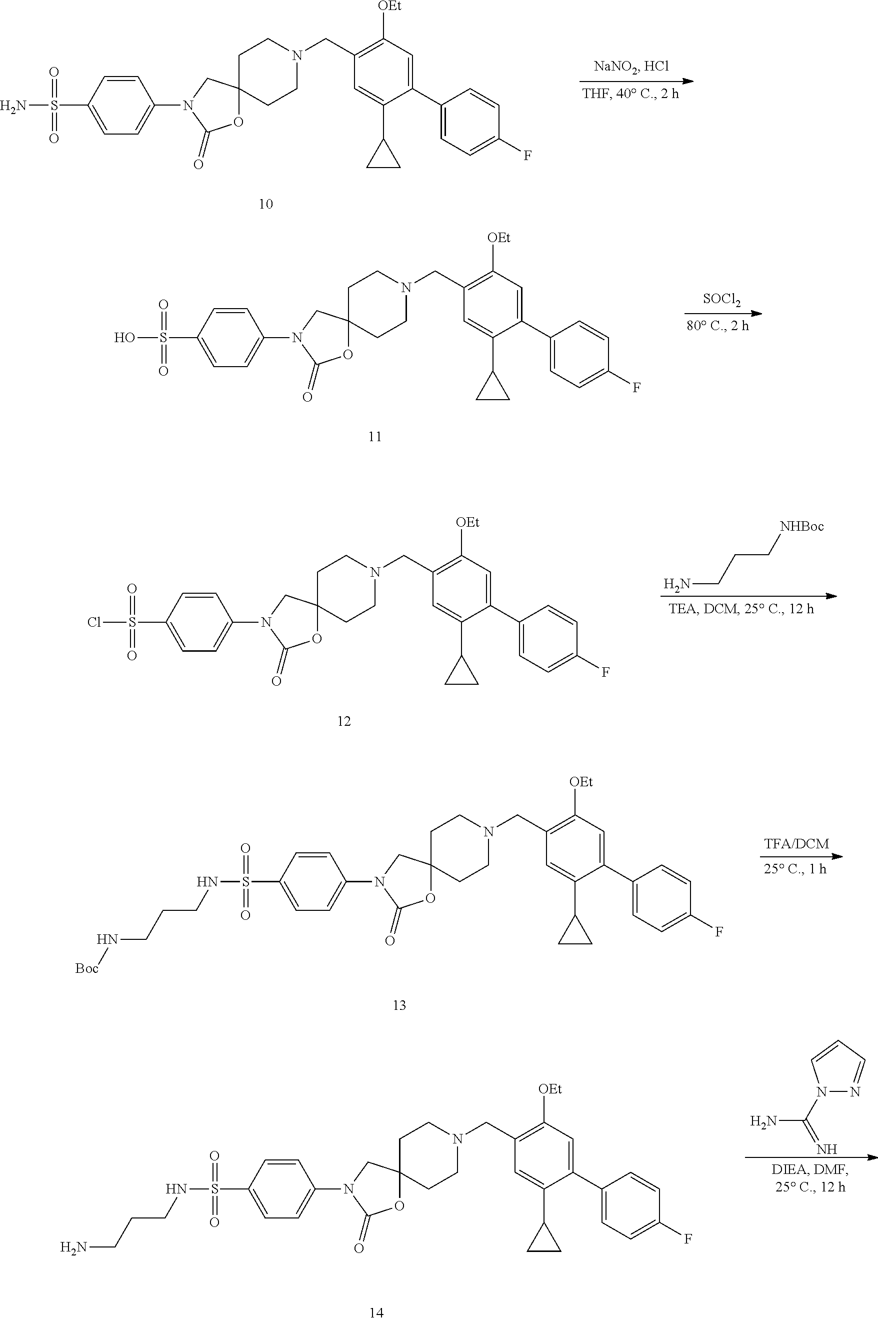

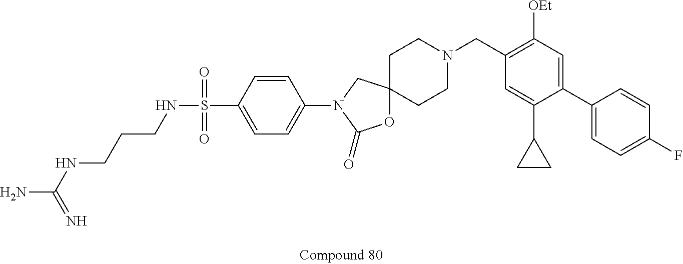 [SMILES: CCOc1cc(-c2ccc(F)cc2)c(C2CC2)cc1CN1CCC2(CC1)CN(c1ccc(S(=O)(=O)NCCCNC(=N)N)cc1)C(=O)O2]

Step 1: methyl 4-amino-2-ethoxybenzoate (1): To a solution of methyl 4-amino-2-hydroxybenzoate (50 g, 299 mmol, 1 eq) and EtI (47 g, 299 mmol, 24 mL, 1 eq) in DMF (300 mL) was added Cs₂CO₃ (117 g, 359 mmol, 1.2 eq), and the mixture was stirred at 25° C. for 2 hours. The mixture was poured into water (400 mL) and then extracted with ethyl acetate (300 mL×3), and the combine organic layers were washed with saturated brine 600 mL (200 mL×2), dried over Na₂SO₄, filtrated and concentrated. The residue was purified by column chromatography (SiO₂, petroleum ether:ethyl acetate, 5:1 to 1:1) to give 1 (26 g, 45% yield) as a yellow solid. LCMS: (ES+) m/z (M−31)⁺=196.1.

Step 2: methyl 4-amino-5-bromo-2-ethoxybenzoate (2): To a solution of 1 (26 g, 133 mmol, 1 eq) in DMF (200 mL) was added NBS (25 g, 140 mmol, 1.05 eq), then the mixture was stirred at 70° C. for 3 hours. The mixture was poured into ice water, and the solid that separated out was isolated by filtration. The filter cake was dried under reduced pressure and then purified by column chramotagraphy (SiO₂, petroleum ether:ethyl acetate, 5:1 to 1:1) to give 2 (25 g, 68% yield) as a brown solid. ¹H NMR (400 MHz, CDCl₃) δ 7.84 (s, 1 H), 6.44 (s, 1 H), 4.06-4.01 (m, 2 H), 3.78 (s, 3 H), 1.42-1.39 (m, J=6.8 Hz, 3 H).

Step 3: methyl 4-amino-5-cyclopropyl-2-ethoxybenzoate (3): To a solution of 2 (18 g, 67 mmol, 1 eq), cyclopropylboronic acid (17 g,202 mmol, 3 eq), tricyclohexylphosphine (3.8 g, 13 mmol, 4.4 mL, 0.2 eq) and K₃PO₄ (43 g, 202 mmol, 3 eq) in toluene (180 mL) and H₂O (18 mL) was added Pd(OAc)₂ (1.5 g, 6.7 mmol, 0.1 eq). Then the mixture was stirred at 110° C. for 16 hours. The reaction mixture was diluted with H₂O (100 mL) and extracted with EA (80 mL×2). The combined organic layers were washed with saturated brine (80 mL×2), dried over Na₂SO₄, filtered and concentrated under reduced pressure to give a residue. The residue was purified by column chromatography (SiO₂, petroleum ether/ethyl acetate, 50/1 to 5/1) to give 3 (16 g, 95% yield) as a yellow solid. LCMS: (ES⁺) m/z (M+H)⁺=235.9.

Step 4: methyl 5-cyclopropyl-2-ethoxy-4-iodobenzoate (4): To a solution of 3 (8.0 g, 34 mmol, 1 eq) in ACN (350 mL) was added CuI (9.7 g, 51 mmol, 1.5 eq) and tert-butyl nitrite (7.0 g, 68 mmol, 8.1 mL, 2 eq) dropwise at 25° C., and the mixture was stirred at 25° C. for 1 hour, then heated to 50° C. for 1 hour. The mixture was poured into 150 mL of H₂O and extracted with EA (100 mL×3). The combined organic layer was washed with water (80 mL×2) and brine (80 mL×2), dried over Na₂SO₄, filtered and concentrated in vacuo. The residue was purified by flash silica gel chromatography (ISCO®; 80 g SepaFlash® Silica Flash Column, eluent of 0 to 6% ethyl acetate/petroleum ether gradient) to give 4 (5.6 g, 45% yield) as a yellow solid. LCMS: (ES⁺) m/z (M+H)⁺=346.9.

Step 5: (5-cyclopropyl-2-ethoxy-4-iodophenyl)methanol (5): To a solution of 4 (5.6 g, 16 mmol, 1 eq) in THF (60 mL) was added DIBAL-H (1 M, 49 mL, 3 eq) dropwise at 0° C. over 15 min. After addition, the resulting mixture was stirred at 25° C. for 2 hours. The reaction mixture was quenched by addition of H₂O at 0° C., then the pH was adjusted to 4 with 6M aqueous HCl. The mixture was diluted with water (30 mL) and extracted with EtOAc (60 mL×3). The combined organic layers were washed with saturated brine (40 mL×2), dried over anhydrous Na₂SO₄, filtered and concentrated under reduced pressure to give 5 (4.3 g, crude) as a yellow solid.

Step 6: 1-(chloromethyl)-5-cyclopropyl-2-ethoxy-4-iodobenzene (6): To a solution of 5 (4.3 g, 14 mmol, 1 eq) in THF (40 mL) was added SOCl₂ (2.4 g, 20 mmol, 1.5 mL, 1.5 eq) and ZnCl₂ (184 mg, 1.4 mmol, 0.1 eq) at 0° C. The mixture was stirred at 0-25° C. for 1 hour. The solution mixture was quenched with slow addition of saturated aqueous NaHCO₃ (10 mL) under stirring and extracted with EA (40 mL×3). The combined organic layer was washed with water (20 mL×2) and brine (20 mL×2), dried over Na₂SO₄ and concentrated in vacuo to give 6 (4.6 g, crude) as a yellow solid.

Step 7: 8-(5-cyclopropyl-2-ethoxy-4-iodobenzyl)-1-oxa-3,8-diazaspiro[4.5]decan-2-one (7): To a mixture of 1-oxa-3,8-diazaspiro[4.5]decan-2-one hydrochloride (150 mg, 779 μmol, 1 eq, HCl) and 6 (262 mg, 779 μmol, 1 eq) in DMF (3 mL) was added DIEA (503 mg, 3.9 mmol, 678 μL, 5 eq). The resulting reaction mixture was stirred at 60° C. for 3 hours. The reaction mixture was poured into water (10 mL) and extracted with EtOAc (20 mL). The organic layer was separated, washed with brine (10 mL), and concentrated to give 7 (350 mg, crude) as a yellow oil that was used in the next step without purification. LCMS: (ES⁺) m/z (M+H)⁺=457.1.

Step 8: 8-((2-cyclopropyl-5-ethoxy-4′-fluoro-[1,1′-biphenyl]-4-yl)methyl)-1-oxa-3,8-diazaspiro[4.5]decan-2-one (8): To a mixture of 7 (300 mg, 657 μmol, 1 eq) and (4-fluorophenyl)boronic acid (276 mg, 2.0 mmol, 3 eq) in dioxane (5 mL) and H₂O (0.5 mL) was added Pd(dppf)Cl₂ (48 mg, 66 μmol, 0.1 eq) and K₂CO₃ (273 mg, 2.0 mmol, 3 eq). The resulting reaction mixture was stirred at 90° C. for 4 hours under N₂. The reaction mixture was concentrated, dissolved in EtOAc (10 mL), and washed with water (10 mL) and brine (10 mL). The organic layer was concentrated to give a residue that was purified by prep-TLC (SiO₂, EtOAc:MeOH, 10:1, Rf=0.3) to afford 8 (300 mg, crude) as a white solid. LCMS: (ES⁺) m/z (M+H)⁺=425.2. ¹H NMR (400 MHz, CDCl₃) δ 7.41 (dd, J=5.6, 8.4 Hz, 2H), 7.17-7.03 (m, 3H), 6.93 (s, 1H), 6.70 (s, 1H), 4.93 (s, 1H), 4.02 (q, J=6.8 Hz, 2H), 3.63 (s, 2H), 3.35 (s, 2H), 2.65 (br s, 4H), 2.02 (br d, J=13.2 Hz, 2H), 1.93-1.72 (m, 3H), 1.40 (t, J=7.2 Hz, 3H), 0.83-0.73 (m, 2H), 0.59 (q, J=5.2 Hz, 2H).

Step 9: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N,N-bis(4-methoxybenzyl)benzenesulfonamide (9): To a solution of 8 (50 mg, 118 μmol, 1 eq) and 4-bromo-N,N-bis(4-methoxybenzyl)benzenesulfonamide (56 mg, 118 μmol, 1 eq) in dioxane (1 mL) was added Cs₂CO₃ (77 mg, 236 μmol, 2 eq), iodocopper;tetrabutylammonium;diiodide (26 mg, 24 μmol, 0.2 eq) and 2-(dimethylamino)acetic acid (4.9 mg, 47 0.4 eq). The resulting reaction mixture was stirred at 120° C. for 16 hours. The residue was dissolved in EtOAc (20 mL) and washed with water (10 mL) and brine (10 mL). The organic layer was concentrated to give a crude product that was purified by silica gel column chromatography (EtOAc:petroleum ether, 4:1) to afford 9 (280 mg, 96.64% yield) as a yellow oil. LCMS: (ES⁺) m/z (M+H)⁺=820.4. ¹H-NMR (400 MHz, CDCl₃): δ 7.75 (d, J=8.8 Hz, 2H), 7.61 (d, J=9.2 Hz, 2H), 7.38-7.31 (m, 2H), 7.04 (t, J=8.8 Hz, 2H), 6.93 (d, J=8.8 Hz, 4H), 6.87 (s, 1H), 6.70 (d, J=8.8 Hz, 4H), 6.64 (s, 1H), 4.16 (s, 4H), 3.96 (q, J=7.2 Hz, 2H), 3.76-3.68 (m, 8H), 3.58 (s, 2H), 2.63 (br s, 4H), 2.28 (s, 1H), 2.30-2.26 (m, 1H), 2.05-1.98 (m, 2H), 1.88 (br d, J=6.8 Hz, 2H), 1.76-1.66 (m, 1H), 1.33 (t, J=7.2 Hz, 4H), 0.92-0.83 (m, 1H), 0.75-0.67 (m, 2H), 0.56-0.49 (m, 2H).

Step 10: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzenesulfonamide (10): A mixture of 9 (230 mg, 281 μmol, 1 eq) in TFA (5 mL) was stirred at 20° C. for 1 hour. The reaction mixture was concentrated. The residue was triturated in saturated aqueous NaHCO₃ (3 mL) for 10 min, then filtered. The filter cake was washed with H₂O (10 mL) and petroleum ether (10 mL) and dried to give 10 (180 mg, crude) as a gray solid. LCMS: (ES⁺) m/z (M+H)⁺=580.2

Step 11: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzenesulfonic acid (11): To a solution of 10 (60 mg, 104 μmol, 1 eq) in concentrated aqueous HCl (1 mL) and THF (0.5 mL) was added NaNO₂ (14 mg, 207 μmol, 2 eq). The resulting reaction mixture was stirred at 40° C. for 2 hours. The reaction mixture was concentrated. The crude product was purified by prep-HPLC (column: Phenomenex Luna C18 150×30 mm×5 μm; mobile phase: [A: water (0.04% HCl v/v), B: ACN]; B%: 35%-65%, over 10 min) to afford 11 (20.9 mg, 32% yield , 96.73% purity, HCl salt) as a white solid. LCMS: (ES⁺) m/z (M+H)⁺=581.2. ¹H NMR (400 MHz, DMSO-d₆) δ 9.25 (br s, 1H), 7.67-7.58 (m, 2H), 7.56-7.47 (m, 4H), 7.31 (br t, J=8.8 Hz, 2H), 7.17 (s, 1H), 6.92 (s, 1H), 4.34 (br s, 2H), 4.19-4.07 (m, 2H), 3.96 (s, 2H), 3.25 (br s, 4H), 2.33 (br s, 2H), 2.20-2.02 (m, 2H), 1.77 (br s, 1H), 1.38 (t, J=6.8 Hz, 3H), 0.81 (br d, J=6.8 Hz, 2H), 0.64 (br d, J=4.4 Hz, 2H).

Step 12: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzene-1-sulfonyl chloride (12): A solution of 11 (200 mg, 334 μmol, 1 eq) in SOCl₂ (2 mL) was stirred at 80° C. for 2 hours. The reaction mixture was concentrated under reduced pressure to give 12 (200 mg, crude) as a yellow solid, which was used in the next step without further purification. LCMS: (ES⁺) m/z (M+H)⁺=599.3.

Step 13: tert-butyl (3-(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenylsulfonamido)propyl)carbamate (13): To a solution of 12 (200 mg, 334 μmol, 1 eq) and tert-butyl (3-aminopropyl)carbamate (116 mg, 668 μmol, 117 μL, 2 eq) in DCM (2 mL) was added Et₃N (169 mg, 1.67 mmol, 232 μL, 5 eq). The mixture was stirred at 25° C. for 12 hours. The reaction mixture was concentrated under reduced pressure to give a residue. The residue was purified by prep-TLC (SiO₂, Dichloromethane: Methanol=10:1) to give 13 (150 mg, 60% yield, 99% purity) as a white solid. LCMS: (ES⁺) m/z (M+H)⁺=737.6.

Step 14: N-(3-aminopropyl)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzenesulfonamide (14): To a solution of 13 (150 mg, 202 μmol, 99% purity, 1 eq) in DCM (1.65 mL) was added TFA (2.54 g, 22 mmol, 1.65 mL, 111 eq). The mixture was stirred at 25° C. for 1 hour. The reaction mixture was concentrated under reduced pressure to give 14 (120 mg, 84% yield, 90% purity) as a white solid. LCMS: (ES⁺) m/z (M+H)⁺=637.3.

Step 15: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(3-guanidinopropyl)benzenesulfonamide (Compound 80): To a solution of 3 (120 mg, 144 μmol, 90% purity, 1 eq, TFA) and 1H-pyrazole-1-carboximidamide (42 mg, 288 μmol, 2 eq, HCl) in DMF (2 mL) was added DIEA (93 mg, 719 μmol, 125 μL, 5 eq). The mixture was stirred at 25° C. for 12 hours. The reaction mixture was concentrated. The residue was purified by prep-HPLC (column: Phenomenex luna C18 150×25 mm×10 μm; mobile phase: [A: water (0.225% FA), B: ACN]; B%: 13%-43%, 10 min) to give Compound 80 (58.55 mg, 57% yield, 95% purity) as a white solid. LCMS: (ES⁺) m/z (M+H)⁺=679.4. ¹H-NMR (CD₃OD, 400 MHz): δ 7.92-7.85 (m, 2H), 7.85-7.79 (m, 2H), 7.52-7.44 (m, 2H), 7.25-7.15 (m, 3H), 6.95 (s, 1H), 4.43 (brs, 2H), 4.19 (q, J=7.2 Hz 2H), 4.06 (brs, 2H), 3.63-3.51 (m, 2H), 3.48-3.38 (m, 2H), 3.28 (t, J=6.8 Hz, 2H), 2.94 (t, J=6.8 Hz, 2H), 2.37-2.27 (m, 4H), 1.84-1.79 (m, 1H), 1.79-1.73 (m, 2H), 1.49 (t, J=7.2 Hz, 3H), 0.87-0.82 (m, 2H), 0.70-0.65 (m, 2H).

The following compounds were prepared according to the procedures described above using the appropriate intermediates.

| Cpd | Characterization Data |
|---|---|
| 81 | LCMS: (ES+) m/z (M + H) + = 668.6. ¹H NMR (400 MHz, CD₃OD-d₄) δ 7.86 (d, J = 8.8 |
| | Hz, 2H), 7.79 (d, J = 9.2 Hz, 2H), 7.44 (t, J = 8.8 Hz, 2H), 7.16 (t, J = 8.8 Hz, 2H), 7.02 (s, |
| | 1H), 6.82 (s, 1H), 4.08 (q, J = 6.8 Hz, 2H), 3.97 (s, 2H), 3.88 (s, 2H), 3.62-3.59 (m, |
| | 2H), 3.45-3.42 (m, 4H), 3.05 (t, J = 5.6 Hz, 2H), 2.98-2.88 (m, 4H), 2.15-2.04 (m, 4H) |
| | 1.80-1.75 (m, 1H) 1.42 (t, J = 6.8 Hz, 3H) 0.80-0.77 (m, 2H) 0.63-0.60 (m, 2H). |
| 82 | LCMS: (ES+) m/z (M + H)⁺ = 669.2. HPLC: tR = 2.579 min, 99.2% purity. ¹H NMR |
| | (400 MHz, METHANOL-d4) δ ppm 0.47-0.64 (m, 2 H) 0.70-0.87 (m, 2 H) 1.44 (t, |
| | J = 6.8 Hz, 3 H) 1.82-1.96 (m, 1 H) 1.98-2.22 (m, 4 H) 2.78-3.02 (m, 4 H) 3.05 (t, |
| | J = 5.6 Hz, 2 H) 3.44 (dt, J = 7.6, 5.00 Hz, 4 H) 3.56-3.68 (m, 2 H) 3.90 (s, 2 H) 3.97 (s, |
| | 2H) 4.11 (q, J = 6.8 Hz, 2 H) 7.00 (s, 1 H)7.14 (s, 1 H) 7.67-7.75 (m, 2 H) 7.76-7.82 |
| | (m, 2 H) 7.83-7.91 (m, 2 H) 8.54 (d, J = 2.4 Hz, 1 H). |
| 83 | LCMS: (ES⁺) m/z (M + H)⁺ = 712.3. 1H-NMR (400 MHz, CD₃OD) δ 7.92-7.87 (m, 2H), |
| | 7.85-7.79 (m, 2H), 7.49-7.43 (m, 2H), 7.22-7.15 (m, 2H), 7.03 (s, 1H), 6.82 (s, 1H), |
| | 4.09 (q, J = 6.8 Hz, 2H), 3.98 (s, 2H), 3.83 (s, 2H), 3.71-3.66 (m, 2H), 3.62-3.52 (m, |
| | 6H), 3.49 (t, J = 5.6 Hz, 2H), 3.06 (t, J = 5.6 Hz, 2H), 2.96-2.78 (m, 4H), 2.19-2.01 |
| | (m, 4H), 1.84-1.76 (m, 1H), 1.44 (t, J = 6.8 Hz, 3H), 0.85-0.76 (m, 2H), 0.66-0.60 |
| | (m, 2H). |

### Example 6: 8-[[5-cyclopropyl-2-ethoxy-4-(4-fluorophenyl)phenyl]methyl]-3-[4-[3-[[[(2S,3R,4R,5R)-2,3,4,5,6-pentahydroxyhexyl]amino]methyl]azetidin-1-yl]sulfonylphenyl]-1-oxa-3,8-diazaspiro[4.5]decan-2-one (Compound 84)

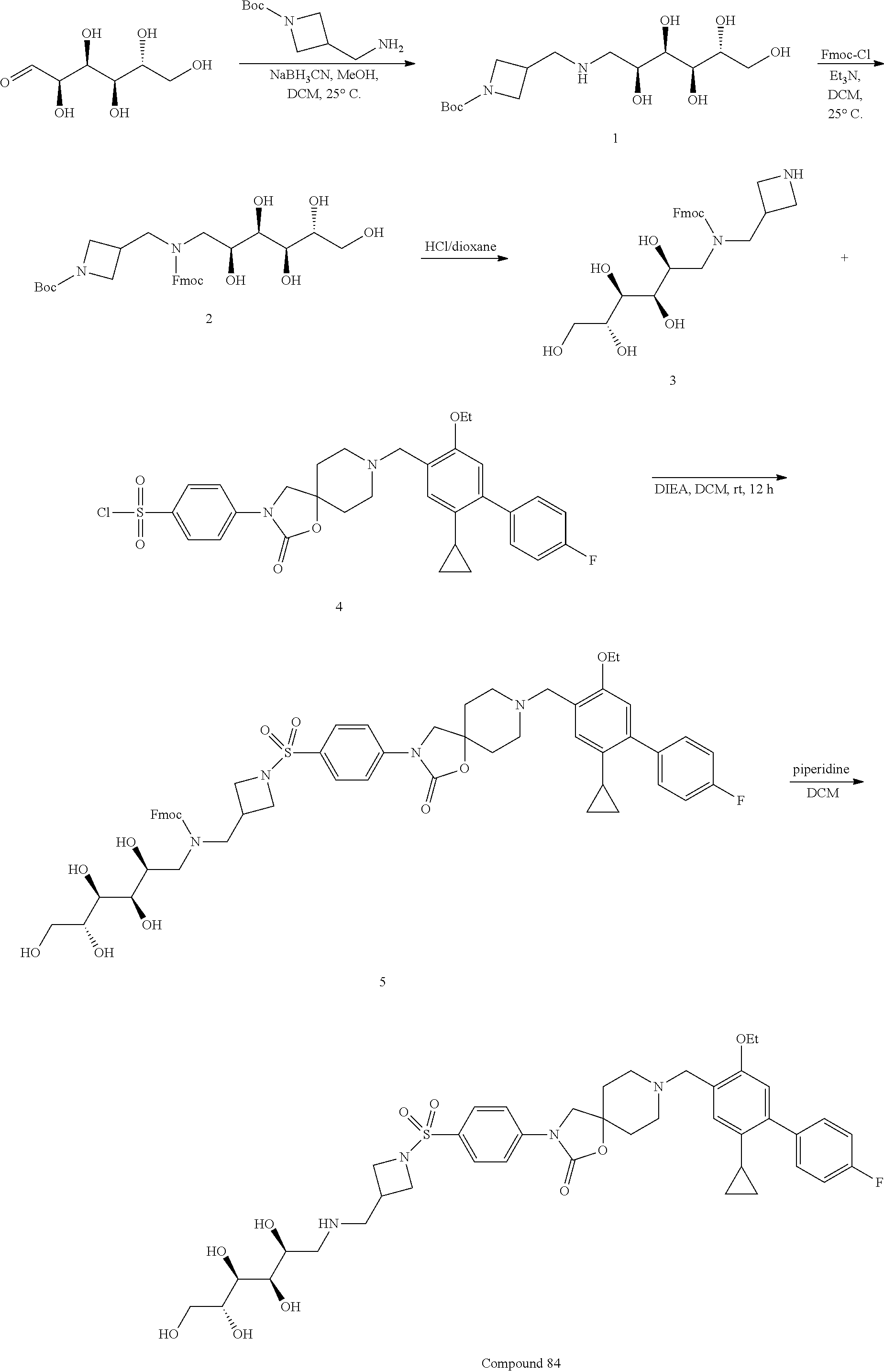

Step 1: tert-butyl 3-[[[(2S,3R,4R,5R)-2,3,4,5,6-pentahydroxyhexyl]amino]methyl]azetidine-1-carboxylate (1): To a solution of tert-butyl 3-(aminomethyl)azetidine-1-carboxylate (5.0 g, 27 mmol, 1 eq) and (2R,3S,4R,5R)-2,3,4,5,6-pentahydroxyhexanal (5.8 g, 32 mmol, 1.2 eq) in MeOH (50 mL) and AcOH (5 mL) was added NaBH₃CN (3.4 g, 54 mmol, 2 eq). The mixture was stirred at 25° C. for 12 hrs. The mixture was concentrated to give 1 (16 g, crude) as a white solid. LCMS: (ES⁺) m/z (M+H)⁺=351.3.

Step 2: tert-butyl 3-[[9H-fluoren-9-ylmethoxycarbonyl-[(2S,3R,4R,5R)-2,3,4,5,6-pentahydroxyhexyl]amino]methyl]azetidine-1-carboxylate (2): To a solution of 1 (4.7 g, 13 mmol, 1 eq) in H₂O (20 mL) and 1,4-dioxane (28 mL) was added NaHCO₃ (2.3 g, 27 mmol, 2 eq) and Fmoc-Cl (4.2 g, 16 mmol, 1.2 eq). The mixture was stirred at 25° C. for 4 hrs. The mixture was concentrated, then diluted with water (150 mL), and extracted with EtOAc (200 mL×3). The combined organic layers were washed with saturated brine (100 mL), dried over anhydrous Na₂SO₄, filtered and concentrated under reduced pressure to give a residue. The residue was triturated with MTBE/Petroleum ether=1:1 (100 mL) for 30 min, then filtered. The solid was collected. The same operation was repeated 3 times. Finally, the solid was dried to give 2 (5.3 g, 50% yield, 72% purity) as a white solid. LCMS: (ES⁺) m/z (M+H)⁺=573.5.

Step 3: 9H-fluoren-9-ylmethyl N-(azetidin-3-ylmethyl)-N-[(2S,3R,4R,5R)-2,3,4,5,6-pentahydroxyhexyl]carbamate (3): A mixture of 2 (0.10 g, 0.17 mmol, 1 eq) and HCl/dioxane (4 M, 5 mL) was stirred at 25° C. for 1 hr. The mixture was concentrated to give 3 (95 mg, HCl salt) as a colorless oil.

Step 4: 8-[[5-cyclopropyl-2-ethoxy-4-(4-fluorophenyl)phenyl]methyl]-3-[4-[3-[[[(2S,3R,4R,5R)-2,3,4,5,6-pentahydroxyhexyl]amino]methyl]azetidin-1-yl]sulfonylphenyl]-1-oxa-3,8-diazaspiro[4.5]decan-2-one (5): To a solution of 3 (85 mg, 0.17 mmol, 2 eq, HCl salt) in DCM (2 mL) was added DIEA (32 mg, 0.25 mmol, 3 eq) and 4-[8-[[5-cyclopropyl-2-ethoxy-4-(4-fluorophenyl)phenyl]methyl]-2-oxo-1-oxa-3,8-diazaspiro[4.5]decan-3-yl]benzenesulfonyl chloride (50 mg, 83 μmol, 1 eq). The mixture was stirred at 25° C. for 12 hrs and then concentrated. The residue was purified by prep-TLC (SiO₂, DCM: MeOH=10:1) to give 5 (27 mg, 30% yield, 95% purity) as a white solid. LCMS: (ES⁺) m/z (M+H)⁺=1035.7.

Step 5: 8-[[5-cyclopropyl-2-ethoxy-4-(4-fluorophenyl)phenyl]methyl]-3-[4-[3-[[[(2S,3R,4R,5R)-2,3,4,5,6-pentahydroxyhexyl]amino]methyl]azetidin-1-yl]sulfonylphenyl]-1-oxa-3,8-diazaspiro[4.5]decan-2-one (Compound 84): To the mixture of 5 (27 mg, 26 μmol, 1 eq) in DCM (1 mL) was added piperidine (0.2 mL). The mixture was stirred at 25° C. for 4 hrs. The mixture was concentrated. The residue was purified by prep-HPLC (column: Welch Xtimate C18 150×25 mm×5 μm; mobile phase: [A: water (0.225% FA), B: ACN]; B%: 12%-42% over 11 min) to give Compound 84 (21 mg, 25 μmol, 93% yield, 95% purity) as a white solid. LCMS: (ES⁺) m/z (M+H)⁺=813.6. ¹H-NMR (400 MHz, CD₃OD) δ=¹⁻H-NMR (400 MHz, CD₃OD) δ=8.51-8.46 (m, 1H), 7.99 (d, J=7.2 Hz, 2H), 7.90-7.87 (m, 2H), 7.46-7.41 (m, 2H), 7.16 (t, J=8.8 Hz, 2H), 7.00 (s, 1H), 6.80 (s, 1H), 4.57 (s, 2H), 4.10-4.03 (m, 2H), 3.98 (s, 2H), 3.93-3.87 (m, 2H), 3.81-3.74 (m, 4H), 3.67-3.60 (m, 3H), 3.58-3.52 (m, 2H), 3.00-2.92 (m, 3H), 2.91-2.82 (m, 2H), 2.81-2.66 (m, 3H), 2.13-1.99 (m, 4H), 1.82-1.75 (m, 1H), 1.42 (t, J=7.2 Hz, 3H), 1.34-1.28 (m, 1H), 0.81-0.74 (m, 2H), 0.63-0.57 (m, 2H).

### Example 7: 4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzenesulfonamide (Compound 85)

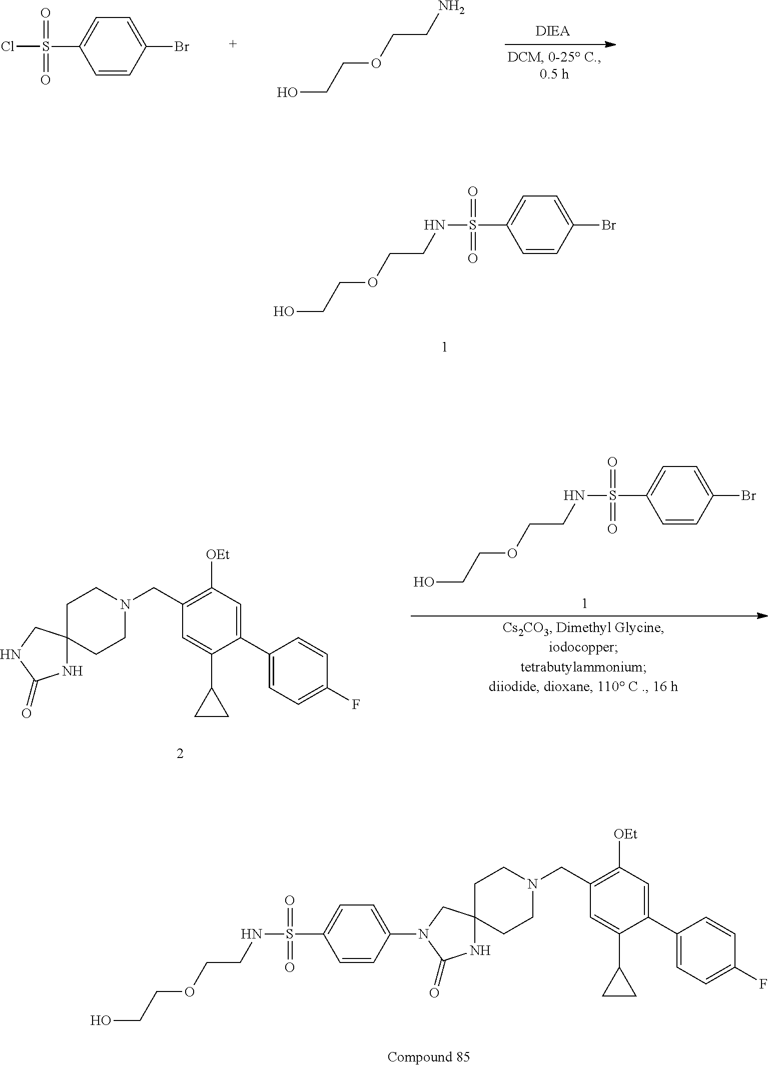 [SMILES: O=S(=O)(Cl)c1ccc(Br)cc1 ; NCCOCCO ; O=S(=O)(NCCOCCO)c1ccc(Br)cc1 ; CCOc1cc(-c2ccc(F)cc2)c(C2CC2)cc1CN1CCC2(CC1)CNC(=O)N2 ; O=S(=O)(NCCOCCO)c1ccc(Br)cc1 ; CCOc1cc(-c2ccc(F)cc2)c(C2CC2)cc1CN1CCC2(CC1)CN(c1ccc(S(=O)(=O)NCCOCCO)cc1)C(=O)N2]

Step 1:4-bromo-N-(2-(2-hydroxyethoxy)ethyl)benzenesulfonamide (1): To a solution of 4-bromobenzenesulfonyl chloride (400 mg, 1.6 mmol, 1 eq) in DCM (4 mL) was added 2-(2-aminoethoxy)ethanol (165 mg, 1.6 mmol, 157 μL, 1 eq) and DIEA (405 mg, 3.13 mmol, 545 μL, 2 eq) at 0° C., then the mixture was stirred at 25° C. for 30 min. The reaction mixture was concentrated under reduced pressure. The residue was purified by reversed-phase HPLC (column: Phenomenex luna C18 250×50 mm×10 μm; mobile phase: [A: water (0.1% FA, v/v), B: ACN]; B%: 40%-45% gradient over 30 min) to give 1 (400 mg, 79% yield) as yellow gum. LCMS: (ES+) m/z (M+H)⁺=324.2.

Step 2: 4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzenesulfonamide (Compound 85): To a solution of 1 (150 mg, 463 μmol, 1.1 eq) and 8-[[5- cyclopropyl-2-ethoxy-4-(4-fluorophenyl)phenyl]methyl]-1,3,8-triazaspiro[4.5]decan-2-one (178 mg, 421 μmol, 1 eq) in toluene (2 mL) was added iodocopper;tetrabutylammonium;diiodide (235 mg, 210 μmol, 0.5 eq), Cs₂CO₃ (274 mg, 841 μmol, 2 eq) and dimethyl glycine (22 mg, 210 μmol, 0.5 eq) in a glove box, and the reaction mixture was stirred at 100° C. for 16 hours. The reaction mixture was diluted with H₂O (20 mL) and NH₃•H₂O (10 mL) and extracted with EA (20 mL×3). The combined organic layers were washed with brine (100 mL), dried over Na₂SO₄, filtered and concentrated under reduced pressure. The residue was purified by prep-HPLC (column: Phenomenex Synergi C18 150×25 mm×10 μm; mobile phase: [A: water (0.225% FA), B: ACN]; B%: 23%-53% over 10min) to give Compound 85 (7 mg, 3% yield). LCMS: (ES+) m/z (M+H)⁺=667.1. ¹H NMR (400 MHz, CDCl₃) δ 0.54-0.68 (m, 2 H), 0.73-0.86 (m, 2 H), 1.35-1.47 (m, 3 H), 1.73-1.82 (m, 1 H), 1.87-2.03 (m, 4 H), 2.61-2.82 (m, 4 H), 3.07-3.23 (m, 2 H), 3.47-3.56 (m, 4 H), 3.65-3.78 (m, 6 H), 3.97-4.16 (m, 2 H), 5.25-5.43 (m, 1 H), 5.47-5.77 (m, 1 H), 6.68-6.76 (m, 1 H), 6.95-7.03 (m, 1 H), 7.07-7.18 (m, 2 H), 7.38-7.46 (m, 2 H), 7.64-7.73 (m, 2 H), 7.78-7.89 (m, 2 H).

### Example 8: 4-[8-[(5-cyclopropyl-2-ethoxy-4-methylsulfonyl-phenyl)methyl]-2-oxo-1,3,8-triazaspiro[4.5]decan-3-yl]-N,N-bis[(4-methoxyphenyl)methyl]benzenesulfonamide (Compound 86)

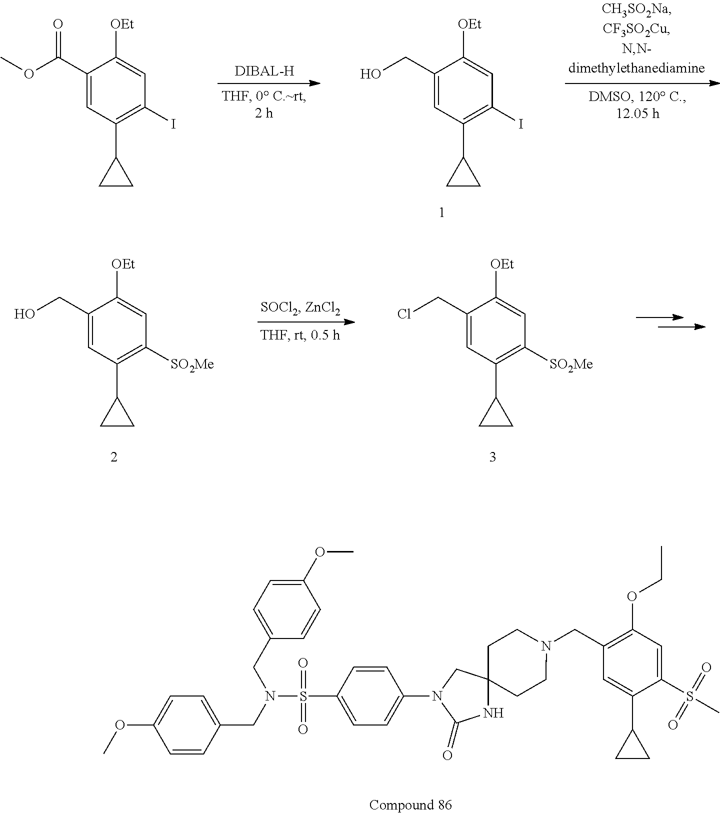

Step 1: (5-cyclopropyl-2-ethoxy-4-iodophenyl)methanol (1): To a solution of methyl 5-cyclopropyl-2-ethoxy-4-iodo-benzoate (1.0 g, 2.9 mmol, 1 eq) in THF (20 mL) was added DIBAL-H (1 M, 4.3 mL, 1.5 eq) dropwise at 0° C. .The mixture was stirred at 0° C. for 2 hours. The reaction mixture was quenched by addition water 20 mL, and then diluted with Ethyl acetate 20 mL, and extracted with Ethyl acetate 20 mL. The combined organic layers were washed with saturated brine (20 mL×2), dried over Na₂SO₄, filtered and concentrated under reduced pressure to give a residue. The residue was purified by prep- HPLC (column: Phenomenex luna C18 250×50 mm×10 μm; mobile phase: A: water(0.225% FA), B: ACN; B%: 33%-63% gradient over 22 min). The solution was lyophilized to give 1 (0.30 g, 0.94 mmol, 33% yield) as a white solid. LCMS: (ES⁺) m/z (M-17)⁺=300.9.

Step 2: (5-cyclopropyl-2-ethoxy-4-(methylsulfonyl)phenyl)methanol (2): To a solution of 1 (0.27 g, 0.85 mmol, 1 eq) and sodium methanesulfinate (0.11 g, 1.1 mmol, 1.32 eq) in DMSO (2.7 mL) was added CF₃SO₂Cu (21 mg, 42 μmol, 0.05 eq). The mixture was stirred at 25° C. for 5 minutes, and then N,N′-dimethylethane-1,2-diamine (82 mg, 0.93 mmol, 0.10 mL, 1.1 eq) was added. The mixture was strried at 110° C. for 12 hours. The residue was diluted with water (20 mL) and extracted with ethyl acetate (20 mL×2). The combined organic layers were washed with saturated brine (20 mL×2), dried over Na₂SO₄, filtered and concentrated under reduced pressure. The residue was purified by column chromatography (SiO₂, petroleum ether/ethyl acetate, 5:1 to 3:1). The spot which Rf=0.2 was collected, and resultant solution was concentrated to give 2 (0.12 g, 52% yield) as a white solid. LCMS: (ES⁺) m/z (M+H)⁺=271.2.

Step 3: 1-(chloromethyl)-5-cyclopropyl-2-ethoxy-4-(methylsulfonyl)benzene (3): To a solution of 2 (0.12 g, 0.44 mmol, 1 eq) in THF (1 mL) was added SOCl₂ (79 mg, 0.67 mmol, 48 μL, 1.5 eq) and ZnCl₂ (6.1 mg, 44 μmol, 0.1 eq). The mixture was stirred at 25° C. for 0.5 hour. The reaction mixture was concentrated under reduced pressure. The residue was diluted with water (20 mL) and extracted with ethyl acetate (20 mL×2), The combined organic layers were washed with saturated brine (20 mL×2), dried over Na₂SO₄, filtered and concentrated under reduced pressure to give 3 (0.13 g, crude) as a white solid.

Following the procedure described above, from 3 and other starting material and intermediates, 4-[8-[(5-cyclopropyl-2-ethoxy-4-methylsulfonyl-phenyl)methyl]-2-oxo-1,3,8-triazaspiro[4.5]decan-3-yl]-N,N-bis[(4-methoxyphenyl)methyl]benzenesulfonamide (Compound 86) was obtained. LCMS: (ES⁺) m/z (M+H)⁺=803.3. ¹H NMR (400 MHz, CDCl₃) δ 7.82 (d, J=8.8 Hz, 2H), 7.71 (br d, J=8.8 Hz, 2H), 7.57 (s, 1H), 7.09 (br s, 1H), 7.02 (br d, J=8.4 Hz, 4H), 6.78 (d, J=8.4 Hz, 4H), 5.06 (br s, 1H), 4.24 (s, 4H), 4.13 (q, J=6.8 Hz, 2H), 3.80 (s, 6H), 3.75 (s, 2H), 3.62 (br s, 2H), 3.25 (s, 3H), 2.80 (br s, 1H), 2.42-2.72 (m, 4H), 1.90 (br s, 4H), 1.46 (br t, J=6.8 Hz, 3H), 1.14 (br d, J=8.0 Hz, 2H), 0.85 (br s, 2H).

### Example 9: 4-(8-((2-cyclopropyl-5-ethoxy-4′-fluoro-[1,1′-biphenyl]-4-yl)methyl)-2,2-dioxido-2-thia-1,3,8-triazaspiro [4.5] decan-3-yl)-N-(2-(2-hydroxyethoxy)ethyl)benzamide (Compound 87)

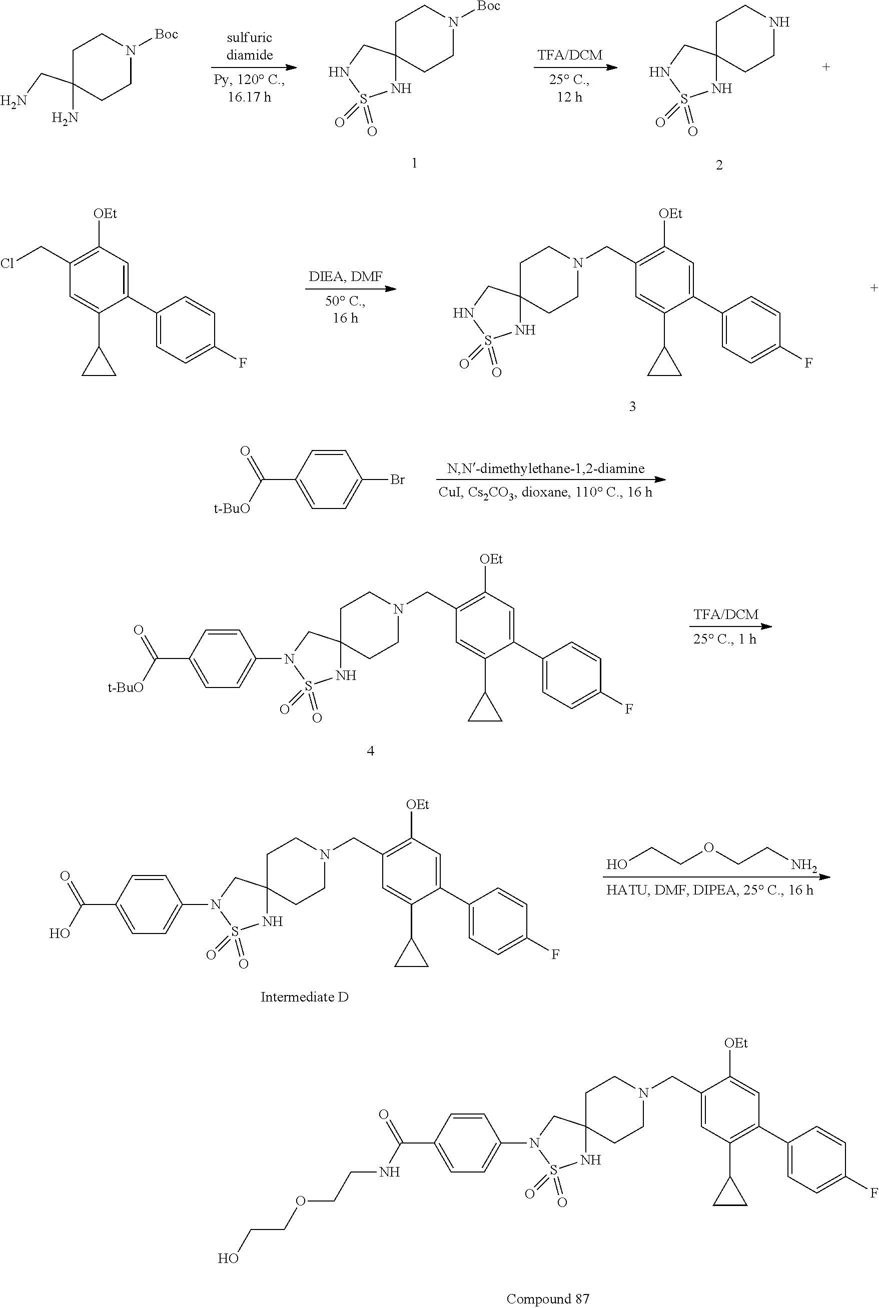

Step 1: tert-butyl 2-thia-1,3,8-triazaspiro[4.5]decane-8-carboxylate 2,2-dioxide (1): To a solution of sulfamide (0.42 g, 4.4 mmol, 0.26 mL, 2 eq) in Py (10 mL) was stirred at 120° C. for 10 min, then tert-butyl 4-amino-4-(aminomethyl)piperidine-1-carboxylate (0.5 g, 2.2 mmol, 1 eq) was added to the solution, and the mixture was stirred at 120° C. for 16 hours. The mixture was concentrated in vacuo, and then the mixture was poured into 20 mL of H₂O and extracted with EA (30 mL×3). The combined organic layer was washed with water (20 mL×2) and brine (20 mL×2), dried over Na₂SO₄ and concentrated in vacuo to give 1 (0.6 g, crude) was obtained as a yellow solid.

Step 2: 2-thia-1,3,8-triazaspiro[4.5]decane 2,2-dioxide (2): To a solution of 1 (0.6 g, 2.1 mmol, 1 eq) in DCM (10 mL) was added TFA (3 mL, 19 eq), and the mixture was stirred at 25° C. for 12 hours. The mixture was concentrated in vacuo to give 2 (0.9 g, crude, TFA) was obtained as a yellow oil.

Step 3: 8-((2-cyclopropyl-5-ethoxy-4′-fluoro-[1,1′-biphenyl]-4-yl)methyl)-2-thia-1,3,8-triazaspiro[4.5]decane 2,2-dioxide (3): To a solution of 2 (0.6 g, 2.0 mmol, 1 eq, TFA) and 4-(chloromethyl)-2-cyclopropyl-5-ethoxy-4′-fluoro-1,1′-biphenyl (0.3 g, 1.0 mmol, 0.5 eq) in DMF (12 mL) was added DIEA (1.0 g, 7.9 mmol, 1.4 mL, 4 eq), and the mixture was stirred at 50° C. for 16 hours. The mixture was poured into 40 mL of H₂O and extracted with EA (40 mL×3). The combined organic layer was washed with water (30 mL×2) and brine (30 mL×2), dried over Na₂SO₄ and concentrated in vacuo. The residue was purified by column chromatography (SiO₂, dichloromethane:methanol, 10:1) to give 3 (0.5 g, 48% yield) was obtained as a yellow oil. LCMS: (ES⁺) m/z (M+H)⁺=460.0.

Step 4: tert-butyl 4-(84(2-cyclopropyl-5-ethoxy-4′-fluoro-[1,1′-biphenyl]-4-yl)methyl)-2,2-dioxido-2-thia-1,3,8-triazaspiro[4.5]decan-3-yl)benzoate (4): To a solution of 3 (0.5 g, 0.9 mmol, 1 eq) and tert-butyl 4-bromobenzoate (0.25 g, 0.98 mmol, 1 eq) in dioxane (10 mL) was added CuI (0.19 g, 0.98 mmol, 1 eq), Cs₂CO₃ (1.3 g, 3.9 mmol, 4 eq), N,N′-dimethylethane-1,2-diamine (86 mg, 0.98 mmol, 0.11 mL, 1 eq), and the mixture was stirred at 110° C. for 12 hours. The mixture was poured 40 ml of H₂O and 10 mL of NH₃.H₂O, and then extracted with EA (50 mL×3). The combined organic layer was washed with water (50mL×2) and brine (50 mL×2), dried over Na₂SO₄ and concentrated in vacuo to give 4 (0.6 g, crude) was obtained as a yellow solid. LCMS: (ES⁺) m/z (M+H)⁺=636.1.

Step 5: 4-(842-cyclopropyl-5-ethoxy-4′-fluoro-[1,1′-biphenyl]-4-yl)methyl)-2,2-dioxido-2-thia-1,3,8-triazaspiro[4.5]decan-3-yl)benzoic acid (Intermediate D): To a solution of 4 (0.2 g, 0.31 mmol, 1 eq) in DCM (4 mL) was added TFA (1.5 g, 13 mmol, 1 mL, 43 eq), and the mixture was stirred at 25° C. for 1 hour. The mixture was concentrated in vacuo. The residue was purified by prep-HPLC:column: Phenomenex Synergi C18 150 mm×25 mm×10 μm; mobile phase: A: water (0.225%FA), B: ACN; B%: 21%-51% gradient over 10min and lyophilization to give Intermediate D (80 mg, 41% yield, 99% purity, FA) as a white solid. LCMS: (ES⁺) m/z (M+H)⁺=580.1. ¹H NMR (400 MHz, DMSO-d₆) δ 8.07 (s, 1H), 7.94 (d, J=8.8 Hz, 2H), 7.50-7.47 (m, 2H), 7.28-7.23 (m, 4H), 6.96 (s, 1H), 6.76 (s, 1H), 4.03 (q, J=6.8 Hz, 2H), 3.82 (s, 2H), 3.53 (s, 2H), 2.59 (s, 4H), 1.95 (s, 2H), 1.84 (s, 2H), 1.78-1.73 (m, 1H), 1.32 (t, J=6.8 Hz, 3H), 0.78-0.74 (m, 2H), 0.56-0.50 (m, 2H).

Step 6: 4-(8-((2-cyclopropyl-5-ethoxy-4′-fluoro-[1,1′-biphenyl]-4-yl)methyl)-2,2-dioxido-2-thia-1,3,8-triazaspiro[4.5]decan-3-yl)-N-(2-(2-hydroxyethoxy)ethyl)benzamide (Compound 87): To a solution of Intermediate D (56 mg, 90 μmol, 1 eq, FA) in DMF (1.5 mL) was added HATU (41 mg, 0.11 mmol, 1.2 eq) and DIEA (35 mg, 0.27 mmol, 3 eq) at 0° C., the mixture was stirred at 25° C. for 0.5 hour. Then 2-(2-aminoethoxy)ethanol (38 mg,0.36 mmol, 4 eq) was added, and the mixture was stirred at 25° C. for 15.5 hours. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give a residue. The residue was purified by prep-HPLC (column: Shim-pack C18 150×25 mm×10 μm; mobile phase: A: water (0.225% FA, v/v), B: ACN; B%: 22%-52% gradient over 10 min) to give Compound 87 (56 mg, 87% yield, FA) as a white solid. LCMS: (ES⁺) m/z (M+H)⁺=667.2. ¹H NMR (400 MHz, CD₃OD) δ 7.86 (dd, J₁=7.2 Hz, J₂=2.0 Hz, 2H), 7.42-7.46 (m, 2H), 7.30 (d, J=8.8 Hz, 2H), 7.15-7.20 (m, 2H), 7.06 (s, 1H), 6.86 (s, 1H), 4.12 (q, J=6.8 Hz, 2H), 4.08 (s, 2H), 3.87 (s, 2H), 3.64-3.70 (m, 4H), 3.57-3.59 (m, 4H), 3.19 (d, J=11.2 Hz, 2H), 3.05 (t, J=11.2 Hz, 2H), 2.26 (d, J=14.0 Hz, 2H), 2.03-2.10 (m, 2H), 1.75-1.81 (m, 1H), 1.44 (t, J=6.8 Hz, 3H), 0.78-0.81 (m, 2H), 0.62-0.63 (m, 2H).

### Example 10: (1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexanecarboxamide (Compound 89) & sodium 3-((1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxamido)propane-1-sulfonate (Compound 88)

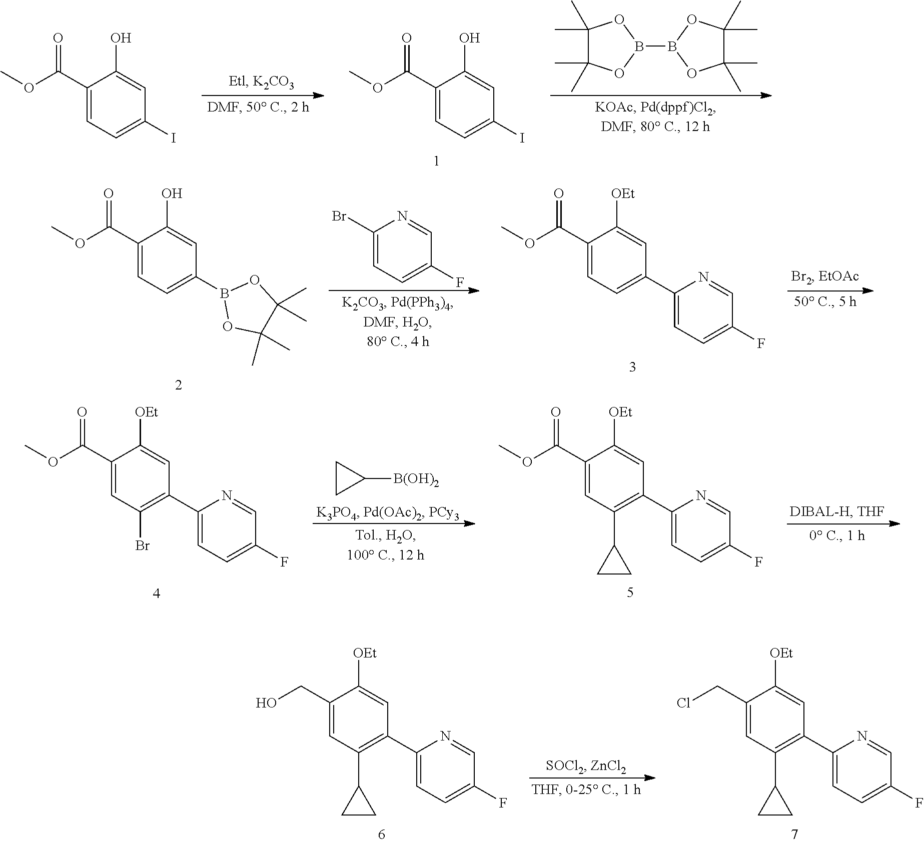

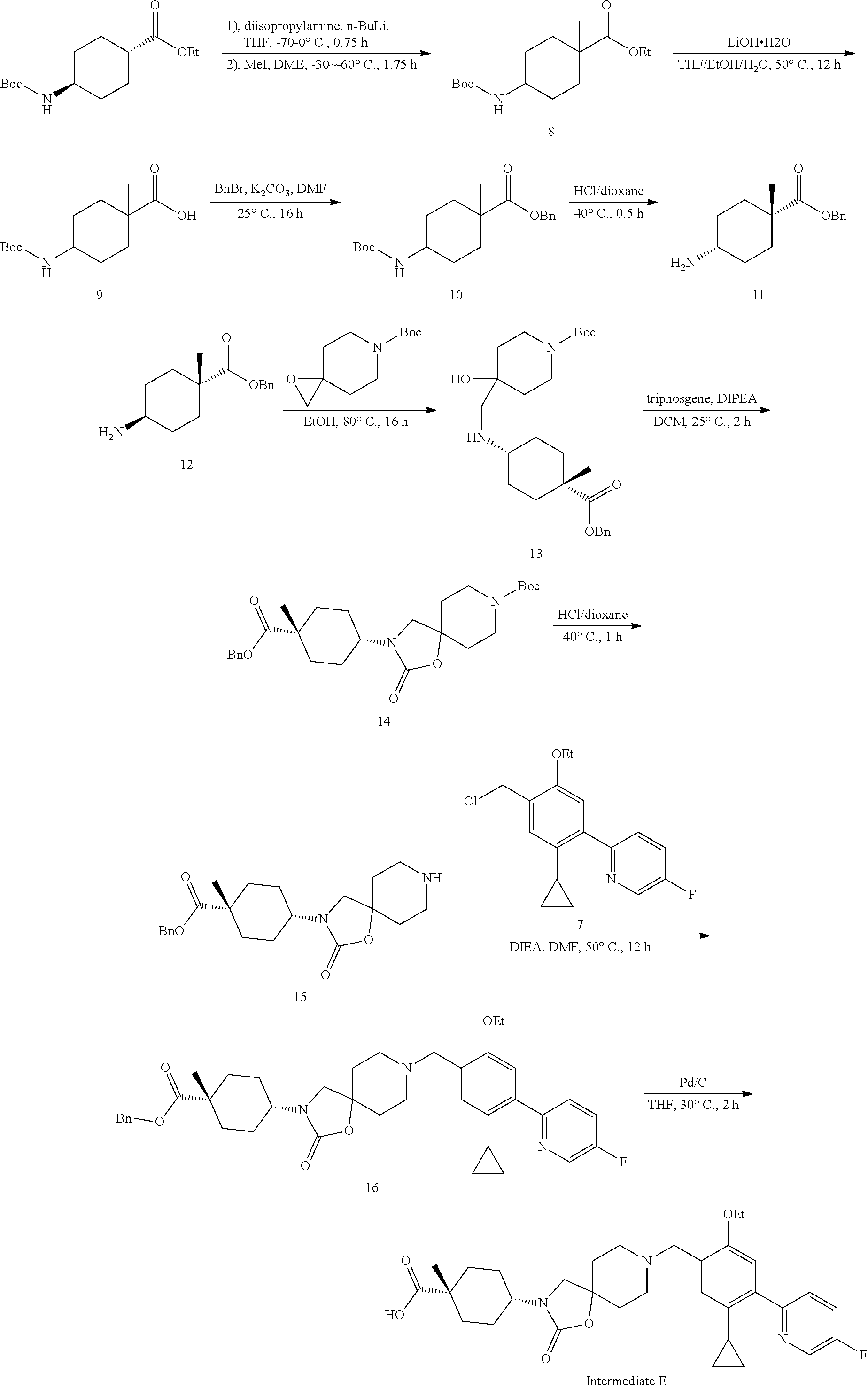

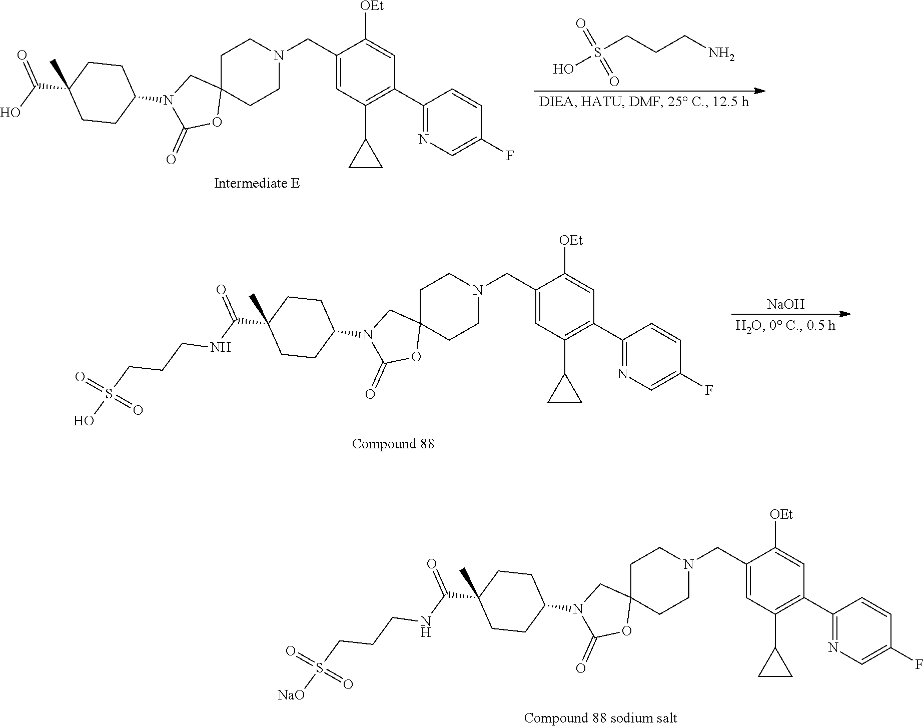

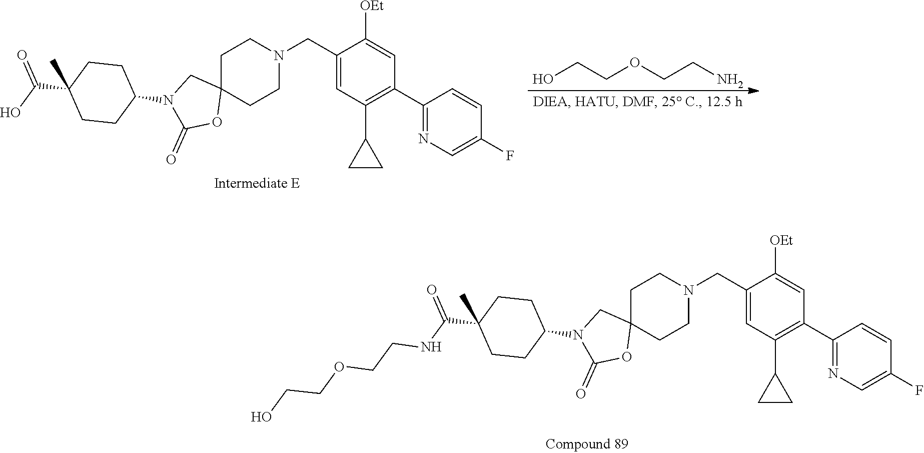 [SMILES: CCOc1cc(-c2ccc(F)cn2)c(C2CC2)cc1CN1CCC2(CC1)CN([C@H]1CC[C@@](C)(C(=O)O)CC1)C(=O)O2 ; NCCOCCO ; CCOc1cc(-c2ccc(F)cn2)c(C2CC2)cc1CN1CCC2(CC1)CN([C@H]1CC[C@@](C)(C(=O)NCCOCCO)CC1)C(=O)O2]

Step 1: methyl 2-ethoxy-4-iodobenzoate (1): To a solution of methyl 2-hydroxy-4-iodo-benzoate (5.0 g, 18 mmol, 1 eq) in DMF (50 mL) was added EtI (3.4 g, 22 mmol, 1.2 eq) and K₂CO₃ (2.5 g, 18 mmol, 1 eq), and the mixture was stirred at 50° C. for 2 hours. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give 1 (5.7 g, crude) as a yellow oil. LCMS: (ES⁺) m/z (M+H)⁺=306.7.

Step 2: methyl 2-ethoxy-4-(4,4,5,5-tetramethyl-1,3,2-dioxaborolan-2-yl)benzoate (2): To a solution of 1 (1.0 g, 3.3 mmol, 1 eq), 4,4,5,5-tetramethyl-2-(4,4,5,5-tetramethyl-1,3,2-dioxaborolan-2-yl)-1,3,2-dioxaborolane (1.2 g, 4.9 mmol, 1.5 eq), and KOAc (0.96 g, 9.8 mmol, 3 eq) in DMF (20 mL) was added Pd(dppf)Cl₂ (0.36g, 0.49 mmol, 0.15 eq) under N₂, and the mixture was stirred at 80° C. for 12 hours. On completion, 2 (1 g, crude) in 20 mL DMF was obtained and used for next step. LCMS: (ES⁺) m/z (M+H)⁺=306.9.

Step 3: methyl 2-ethoxy-4-(5-fluoropyridin-2-yl)benzoate (3): To a solution of 2 (1 g, 3.3 mmol, 1 eq), 2-bromo-5-fluoro-pyridine (1.2 g, 6.5 mmol, 2 eq) and K₂CO₃ (0.9 g, 6.5 mmol, 2 eq) in DMF (10 mL) and H₂O (4 mL) was added Pd(PPh₃)₄ (0.38 g, 0.33 mmol, 0.1 eq) under N₂, and the mixture was stirred at 80° C. for 4 hours. The reaction mixture was diluted with H₂O (30 mL) and extracted with ethyl acetate (30 mL×3). The combined organic layers were washed with brine (30 mL×3), dried over Na₂SO₄, filtered and concentrated under reduced pressure. The residue was purified by column chromatography (SiO₂, Petroleum ether: Ethyl acetate=10:1) to give 3 (0.27g, 22% yield) as a yellow oil. LCMS: (ES⁺) m/z (M+H)⁺=276.1.

Step 4: methyl 5-bromo-2-ethoxy-4-(5-fluoropyridin-2-yl)benzoate (4): To a solution of 3 (0.9 g, 3.3 mmol, 1 eq) in EtOAc (10 mL) was added Br₂ (0.63 g, 3.9 mmol, 1.2 eq), and the mixture was stirred at 50° C. for 5 hours. The reaction mixture was diluted with H₂O (30 mL) and extracted with ethyl acetate (30 mL×3). The combined organic layers were washed with brine (30 mL×3), dried over Na₂SO₄, filtered and concentrated under reduced pressure. The residue was purified by prep-HPLC (column: Phenomenex luna C18 150 mm×40 mm×15 μm; mobile phase: A: water (0.225%FA,v/v), B: ACN; B%: 41%-71% gradient over 9 min) to give 4 (0.8 g, 69% yield, 99.3% purity) as a white solid. LCMS: (ES⁺) m/z (M+H)⁺=355.9. ¹H NMR (400MHz, CDCl₃) δ 8.57 (s, 1H), 8.08 (s, 1H), 7.68 (d, J=8.4 Hz, 1H), 7.50 (t, J=8.4 Hz, 1H), 7.16 (s, 1H), 4.15 (q, J=6.8 Hz, 2H), 3.91 (s, 3H), 1.46 (t, J=6.8 Hz, 3H).

Step 5: methyl 5-cyclopropyl-2-ethoxy-4-(5-fluoropyridin-2-yl)benzoate (5): To a solution of 4 (0.75 g, 2.1 mmol, 1 eq), cyclopropylboronic acid (0.55 g, 6.4 mmol, 3 eq), K₃PO₄ (1.4 g, 6.4 mmol, 3 eq), and tricyclohexylphosphane (59 mg, 0.21 mmol, 0.1 eq) in toluene (8 mL) and H₂O (0.8 mL) was added Pd(OAc)₂ (24 mg, 0.11 mmol, 0.05 eq) under N₂, and the mixture was stirred at 100° C. for 12 hours. The reaction mixture was diluted with H₂O (30 mL) and extracted with ethyl acetate (30 mL×3). The combined organic layers were washed with brine (30 mL×3), dried over Na₂SO₄, filtered and concentrated under reduced pressure. The residue was purified by column chromatography (SiO₂, petroleum ether:ethyl acetate, 10:1) to give 12 (0.36 g, 48% yield) as a yellow oil. LCMS: (ES⁺) m/z (M+H)⁺=316.1.

Step 6: (5-cyclopropyl-2-ethoxy-4-(5-fluoropyridin-2-yl)phenyl)methanol (6): To a solution of 5 (0.36 g, 1.0 mmol, 1 eq) in THF (5 mL) was added DIBAL-H (1 M, 3.05 mL, 3 eq) at 0° C., and the mixture was stirred at 0° C. for 1 hour. The residue was poured into water (50 mL) and stirred for 10 min. Then the mixture was adjust to pH 4 with aqueous HCl (2 M) and extracted with ethyl acetate (30 mL×3). The combined organic layers were washed with brine (30 mL×3), dried over Na₂SO₄, filtered, and concentrated under reduced pressure to give 6 (0.4 g, crude) as a yellow solid.

Step 7: 2-(4-(chloromethyl)-2-cyclopropyl-5-ethoxyphenyl)-5-fluoropyridine (7): To a solution of 6 (0.14 g, 0.49 mmol, 1 eq) and ZnCl₂ (6.6 mg, 49 μmol, 0.1 eq) in THF (3 mL) was added SOCl₂ (87 mg, 0.73 mmol, 1.5 eq) at 0° C., and the mixture was stirred at 25° C. for 1 hour. The reaction mixture was diluted with H₂O (30 mL) and extracted with ethyl acetate (30 mL×3). The combined organic layers were washed with brine (30 mL×3), dried over Na₂SO₄, filtered and concentrated under reduced pressure to give 14 (0.14 g, crude) as a yellow oil. LCMS: (ES⁺) m/z (M+H)⁺=306.0. ¹H NMR (400 MHz, CDCl₃) δ 8.57 (d, J=2.8 Hz, 1H), 7.57-7.60 (m, 1H), 7.45-7.49 (m, 1H), 7.06 (s, 1H), 6.96 (s, 1H), 4.67 (s, 2H), 4.11 (q, J=6.8 Hz, 2H), 1.89-1.92 (m, 1H), 1.44 (t, J=6.8 Hz, 3H), 0.78-0.80 (m, 2H), 0.58-0.60 (m, 2H).

Step 8: ethyl 4-((tert-butoxycarbonyl)amino)-1-methylcyclohexanecarboxylate (8): To a solution of diisopropylamine (7.5 g, 74 mmol, 10 mL, 2.5 eq) dissolved in THF (65 mL) and cooled to 0° C. under N₂. n-BuLi (2.5 M, 30 mL, 2.5 eq) was added slowly. The mixture was stirred at 0° C. for 15 min and then cooled to -70° C. Ethyl 4-(tert-butoxycarbonylamino)cyclohexanecarboxylate (8 g, 30 mmol, 1 eq) dissolved in THF (20 mL) was added. The mixture was warmed to −30° C. and stirred at this temperature for 30 min. Then DME (40 mL) was added to the solution, and the mixture was stirred at −60° C. for another 45 min. MeI (4.60 g, 32.43 mmol, 1.1 eq) in THF (5 mL) was then added, and the solution was stirred for 1 hour at −60° C. The mixture was quenched with saturated aqueous NH₄Cl, and the pH was adjusted to 6 with 1 N aqueous HCl. The mixture was extracted with EtOAc (30 mL×2). The organic phase was washed with water (20 mL) and brine (15 mL×2), dried over Na₂SO₄ and concentrated in vacuo. The residue was purified by column chromatography (SiO₂, petroleum ether/ethyl acetate, 10/1) to give 8 (6.8 g, 81% yield) as a yellow oil. LCMS: (ES⁺) m/z (M-Boc)⁺=186.3. (Notice: TLC showed two new spots, the ratio was 2:1).

Step 9: 4-((tert-butoxycarbonyl)amino)-1-methylcyclohexanecarboxylic acid (9): To a solution of 8 (6.8 g, 24 mmol, 1 eq) in THF (20 mL), EtOH (20 mL) and H₂O (20 mL) was added LiOH.H₂O (4.0 g, 95 mmol, 4 eq). The mixture was stirred at 50° C. for 12 hours. The mixture was concentrated in vacuo. The residue was dissolved with water (50 mL) and extracted with EtOAc (40 mL×2). The aqueous layer was adjusted to pH 4 with 1 M aqueous HCl and then extracted with (EtOAc:THF, 10:1) (40 mL×2). The combined organic layer was washed with brine (40 mL×2), dried over Na₂SO₄ and concentrated in vacuo to give 9 (6.1 g, 99% yield) as a colorless oil. LCMS: (ES⁺) m/z (M-Boc)⁺=158.3.

Step 10: benzyl 4-((tert-butoxycarbonyl)amino)-1-methylcyclohexanecarboxylate (10): To a solution of 9 (6.1 g, 23.7 mmol, 1 eq) in DMF (60 mL) was added K₂CO₃ (6.6 g, 47.4 mmol, 2 eq) and BnBr (6.1 g, 35.6 mmol, 1.5 eq). The mixture was stirred at 25° C. for 16 hours. The mixture was diluted with water 100 mL and extracted with EtOAc (30 mL×3). The combined organic layer was washed with water (20 mL) and brine (20 mL×2), dried over Na₂SO₄ and concentrated in vacuo. The residue was purified by column chromatography (SiO₂, petroleum ether/ethyl acetate, 10/1) to give 10 (5.36 g, 65% yield) as a white solid. LCMS: (ES⁺) m/z (M−Boc)⁺=248.2.

Step 11: (1s,4s)-benzyl 4-amino-1-methylcyclohexanecarboxylate (11) & (1r,4r)-benzyl 4-amino-1-methylcyclohexanecarboxylate (12): A solution of 10 (430 mg, 1.2 mmol) in HCl/dioxane (4 M, 4 mL) was stirred at 40° C. for 0.5 hour. The mixture was concentrated in vacuo. The residue was dissolved in saturated aqueous Na₂CO₃ (20 mL) and extracted with 10:1 EtOAc:IPA (20 mL×2). The combined organic layer was washed with water (15 mL) and brine (15 mL×2), dried over Na₂SO₄, and concentrated in vacuo. The residue was purified by prep-HPLC (column: Phenomenex Synergi C18 150×25 mm×10 μm; mobile phase: A: water (0.225% FA, v/v), B: ACN B%: 3%-33% gradient over 10 min) to give 11 (150 mg, 49% yield, FA salt) and 12 (100 mg, 33% yield, FA salt) both as a white solid. The ¹H NMR of 12: ¹H NMR (CD₃OD, 400 MHz) δ 8.50 (s, 1H), 7.37-7.32 (m, 5H), 5.13 (s, 2H), 3.16-3.09 (m, 1H), 1.90-1.83 (m, 4H), 1.77-1.65 (m, 2H), 1.64-1.58 (m, 2H). (Note: The trans configuration of 12 was established by 2D NMR.)

Step 12: tert-butyl 4-((((1s,4s)-4-((benzyloxy)carbonyl)-4-methylcyclohexyl)amino)methyl)-4-hydroxypiperidine-1-carboxylate (13): A solution of 11 (1.8 g, 7.3 mmol, 1 eq) and tert-butyl 1-oxa-6-azaspiro[2.5]octane-6-carboxylate (1.9 g, 8.7 mmol, 1.2 eq) in EtOH (40 mL) was heated to 80° C. The mixture was stirred at 80° C. for 16 hours. The residue was concentrated in vacuo. The crude product was purified by reverse phase 1VIPLC (column: Phenomenex luna C18 250×50 mm×10 μm; mobile phase: A: water (0.1% TFA, v/v), B: ACN; B%: 5%-34% gradient over 20 min). The eluent was concentrated in vacuo. The residue was diluted with 50 mL of water and the pH adjusted to 10 with saturated aqueous Na₂CO₃. Then mixture was extracted with (DCM: MeOH=10:1) (20 mL×3). The combined organic layer was washed with brine (15 mL×2), dried over Na₂SO₄ and concentrated in vacuo to give 13 (2.3 g, 75% yield) as a colorless oil.

Step 13: tert-butyl 3-((1s,4s)-4-((benzyloxy)carbonyl)-4-methylcyclohexyl)-2-oxo-1-oxa-3,8-diazaspiro[4.5]decane-8-carboxylate (14): To a solution of 13 (4.3 g, 9.34 mmol, 1 eq) and triphosgene (2.8 g, 9.34 mmol, 1 eq) in DCM (150 mL) was added DIPEA (6.0 g, 46.68 mmol, 5 eq). The mixture was stirred at 25° C. for 2 hours. The reaction mixture was quenched with saturated aqueous NaHCO₃ (20 mL) and H₂O (20 mL). The organic phase was separated, washed with aqueous brine (20 mL×2), dried over Na₂SO₄, filtered and concentrated under reduced pressure to give a residue. The residue was purified by column chromatography (SiO₂, petroleum ether/ethyl acetate, 2:1) to give 14 (4 g, 86% yield) as a white solid.

Step 14: (1s,4s)-benzyl 1-methyl-4-(2-oxo-1-oxa-3,8-diazaspiro[4.5]decan-3-yl) cyclohexanecarboxylate (15): A solution of 14 (1.5 g, 3.1 mmol, 1 eq) in HCl/dioxane (4 M, 31 mL) was stirred at 40° C. for 1 hour. The residue was concentrated in vacuo to give 15 (1.3 g, 99% yield, HCl) as a white solid.

Step 15: (1s,4s)-benzyl 4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxylate (16): To a solution of 15 (0.23 g, 0.54 mmol, 1 eq, HCl) and 7 (0.17 g, 0.54 mmol, 1 eq) in DMF (5 mL) was added DIEA (0.21 g, 1.6 mmol, 0.28 mL, 3 eq), and the mixture was stirred at 50° C. for 12 hours. The residue was poured into water (30 mL) and extracted with EA (30 mL×3). The combined organic layer was washed with water (20 mL×3) and brine (20 mL×3), dried over Na₂SO₄ and concentrated in vacuo. The residue was purified by column chromatography (SiO₂, petroleum ether:ethyl acetate, 1:1) to give 16 (0.35 g, 97% yield) as a yellow oil. LCMS: (ES⁺) m/z (M+H)⁺=656.2.

Step 16: (1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxylic acid (Intermediate E): To a solution of 16 (0.35 g, 0.5 mmol, 1 eq) in THF (15 mL) was added 5% Pd/C (0.23 g, 0.11 mmol, 0.2 eq) under N2. The suspension was degassed under vacuum and purged with H₂ several times. The mixture was stirred under H₂ (15 psi) at 30° C. for 2 hours. The mixture was filtered and concentrated in vacuo. The residue was purified by prep-HPLC (column: UniSil 3-100 C18 Ultra (150×25 mm×3 μm); mobile phase: A: water (0.225% FA, v/v), B: ACN; B%: 20%-50% gradient over 10 min) to give Intermediate E (175 mg, 54% yield) as a white solid. LCMS: (ES⁺) m/z (M+H)⁺=566.2. ¹H NMR (400 MHz, DMSO-d₆) δ=8.65 (d, J=2.8 Hz, 1H), 7.82-7.77 (m, 1H), 7.73-7.69 (m, 1H), 6.99 (s, 1H), 6.94 (s, 1H), 4.02 (q, J=6.8 Hz, 2H), 3.50 (s, 2H), 3.19 (s, 2H), 2.50-2.39 (m, 4H), 2.09 (d, J=12 Hz, 2H), 2.01-1.94 (m, 1H), 1.74 (t, J=4.8 Hz, 4H), 1.56 (d, J=10.8 Hz, 2H), 1.42 (q, J=10.4 Hz, 2H), 1.31 (t, J=6.8 Hz, 3H), 1.27-1.21 (m, 2H), 1.1 (s, 3H), 0.77-0.72(m, 2H), 0.48-0.44 (m, 2H).

Step 17: 3-((1s,4s)-4-(8-(5-cyclopropyl-2-ethoxy-4-(5-fluoropyridin-2-yl)benzyl) oxo-1-oxa-3,8-diazaspiro[4.5]decan-3-yl)-1-methylcyclohexanecarboxamido)propane-1-sulfonic acid (Compound 88): A solution of Intermediate E (70 mg, 0.1 mmol, 1 eq) and HATU (56 mg, 0.2 mmol, 1.2 eq) in DMF (1.5 mL) was stirred at 25° C. for 0.5 hour. Then 3-aminopropane-1-sulfonic acid (69 mg, 0.5 mmol, 4 eq) and DIEA (48 mg, 0.4 mmol, 65 μL, 3 eq) was added to the solution and stirred at 25° C. for 12 hours. The reaction mixture was poured into 30 mL of H₂O and extracted with ethyl acetate (30 mL×3). The combined organic layers were washed with brine (20 mL×2), dried over Na₂SO₄, then concentrated in vacuo. The residue was purified by prep-HPLC (column: Waters Xbridge C18 150×50 mm×10 μm; mobile phase: A: water (10 mM NH₄HCO₃), B: ACN; B%: 18%-48% gradient over 10 min) to give Compound 88 (13 mg, 15% yield) as a white solid. LCMS: (ES⁺) m/z (M+H)⁺=687.2.

Step 18: sodium 3-((1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxamido)propane-1-sulfonate (Compound 88 sodium salt): To a solution of 17 (13 mg, 19 μmol, 1 eq) in H₂O (5 mL) was added NaOH (0.1 M, 0.2 mL, 1 eq) at 0° C., and the mixture was stirred at 0° C. for 0.5 hour. The mixture was lyophilized to give Compound 88 sodium salt (13 mg, 99% yield) as a white solid. LCMS: (ES⁺) m/z (M+H)⁺=687.1. ¹H NMR (400 MHz, CD₃OD) δ 8.52 (d, J=2 Hz, 1H), 7.72-7.69 (m, 2H), 7.1(s, 1H), 6.95(s, 1H), 4.07(q, J=7.2 Hz, 2H), 3.73 (s, 2H), 3.67-3.62 (m, 1H), 3.36-3.32 (m, 4H), 2.85-2.78 (m, 4H), 2.67 (s, 2H), 2.28 (d, J=13.2 Hz, 2H), 2.02-1.98 (m, 2H), 1.90-1.89 (m, 4H), 1.88 (s, 1H), 1.60 (d, J=6.4 Hz, 2H), 1.55-1.52 (m, 2H), 1.41 (t, J=7.2 Hz, 3H), 1.33-1.32 (m, 2H), 1.13 (s, 3H), 0.78-0.75 (m, 2H), 0.56-0.55 (m, 2H).

Step 19: (1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexanecarboxamide (Compound 89): To a solution of Intermediate E (70 mg, 0.1 mmol, 1 eq) and HATU (56 mg, 0.2 mmol, 1.2 eq) in DMF (1.5 mL) was stirred at 25° C. for 0.5 hour. Then 2-(2-aminoethoxy)ethanol (52 mg, 0.5 mmol, 50 μL, 4 eq) and DIEA (48 mg, 0.4 mmol, 65 μL, 3 eq) was added to the solution, and the mixture was stirred at 25° C. for 12 hours. The reaction mixture was poured into 30 mL of H₂O and extracted with EA (30 mL×3). The combined organic layers were washed with brine (20 mL×2), dried over Na₂SO₄, then concentrated in vacuo. The residue was purified by prep-HPLC (column: UniSil 3-100 C18 Ultra (150×25 mm×3 μm); mobile phase: A: water (0.225% FA),B: ACN; B%: 20%-50% gradient over 10 min) to give Compound 89 (36 mg, 41% yield) as a white solid. LCMS: (ES⁺) m/z (M+H)⁺=653.2. ¹H NMR (400 MHz, CD₃OD) δ 8.55 (d, J=2.4 Hz, 1H), 7.72-7.69 (m, 2H), 7.1(s, 1H), 6.97(s, 1H), 4.07 (q, J=6.8 Hz, 2H), 3.76 (s, 2H), 3.67-3.64 (m, 2H), 3.56-3.55 (m, 1H), 3.55-3.54 (m, 4H), 3.31(t, J=1.6 Hz, 2H), 3.31-3.3 (m, 2H), 2.78-2.68 (m, 4H), 2.27(d, J=12.8 Hz, 2H), 1.92-1.86(m, 5H), 1.64-1.59(m, 2H), 1.57-1.53(m, 2H), 1.43-1.40 (m, 3H), 1.39-1.33 (m, 2H), 1.13 (s, 3H), 0.77 (q, J=1.6 Hz, 2H), 0.55 (q, J=1.6 Hz, 2H).

### Example 11: (1r, 4r)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexanecarboxamide (Compound 90) & 3-((1r,4r)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxamido)propane-1-sulfonic acid (Compound 91)

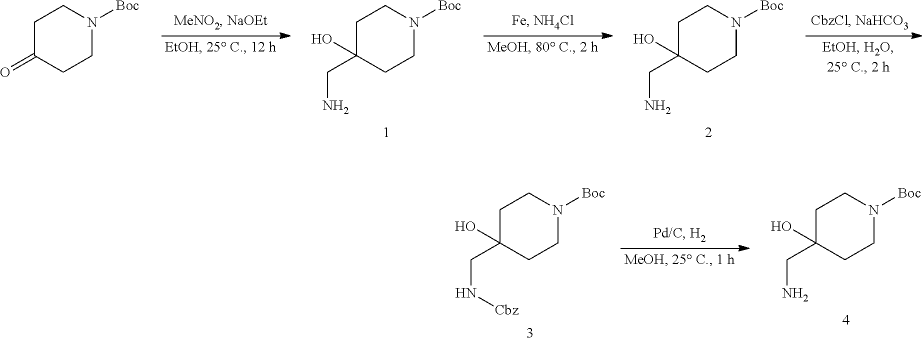 [SMILES: CC(C)(C)OC(=O)N1CCC(=O)CC1 ; CC(C)(C)OC(=O)N1CCC(O)(CN)CC1 ; CC(C)(C)OC(=O)N1CCC(O)(CN)CC1 ; CC(C)(C)OC(=O)N1CCC(O)(CNC(=O)OCc2ccccc2)CC1 ; CC(C)(C)OC(=O)N1CCC(O)(CN)CC1]

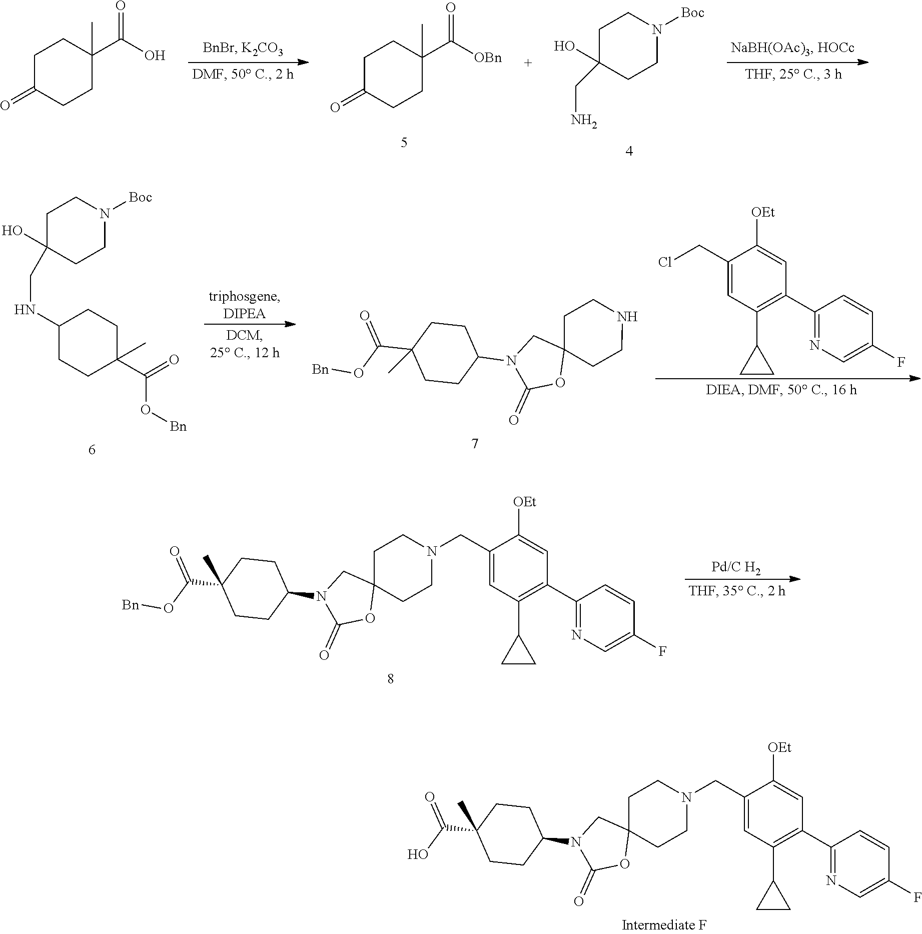

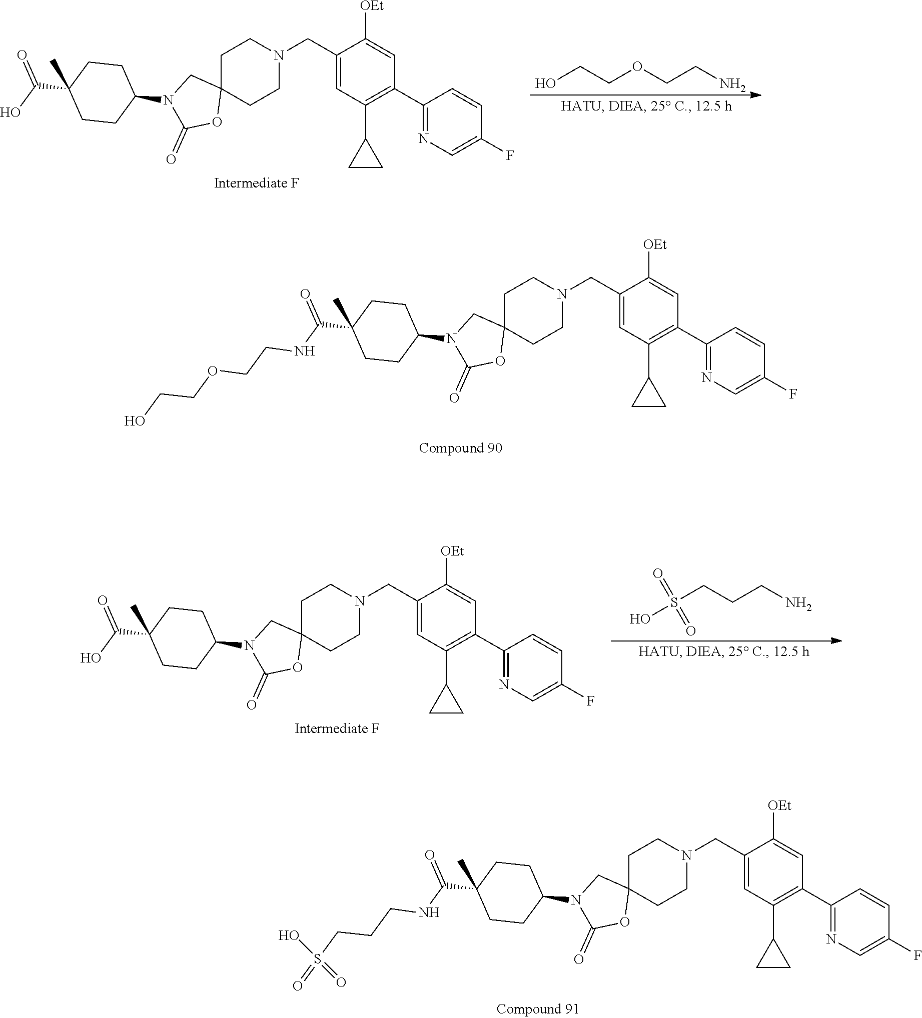 [SMILES: CCOc1cc(-c2ccc(F)cn2)c(C2CC2)cc1CN1CCC2(CC1)CN([C@H]1CC[C@](C)(C(=O)O)CC1)C(=O)O2 ; NCCOCCO ; CCOc1cc(-c2ccc(F)cn2)c(C2CC2)cc1CN1CCC2(CC1)CN([C@H]1CC[C@](C)(C(=O)NCCOCCO)CC1)C(=O)O2 ; CCOc1cc(-c2ccc(F)cn2)c(C2CC2)cc1CN1CCC2(CC1)CN([C@H]1CC[C@](C)(C(=O)O)CC1)C(=O)O2 ; NCCCS(=O)(=O)O ; CCOc1cc(-c2ccc(F)cn2)c(C2CC2)cc1CN1CCC2(CC1)CN([C@H]1CC[C@](C)(C(=O)NCCCS(=O)(=O)O)CC1)C(=O)O2]

Step 1: tert-butyl 4-hydroxy-4-(nitromethyl)piperidine-1-carboxylate (1): To a solution of tert-butyl 4-oxopiperidine-1-carboxylate (10 g, 50 mmol, 1 eq) in EtOH (10 mL) and CH₃NO₂ (4.5 g, 74 mmol, 4 mL, 1.5 eq) was added NaOEt (170 mg, 2.5 mmol, 0.05 eq). Then the mixture was stirred at 25° C. for 12 hours. On completion, the reaction mixture was concentrated under reduced pressure to give a residue. The crude product was purified by re-crystallization from PE, and the filter cake was collected to give 1 (12 g, 47 mmol, 93% yield) as a white solid.

Step 2: tert-butyl 4-(aminomethyl)-4-hydroxy-piperidine-1-carboxylate (2): To a solution of 1 (5 g, 19 mmol, 1 eq) in EtOH (50 mL) and H₂O (25 mL) was added NH₄Cl (4.1 g, 76 mmol, 4.0 eq) and Fe (4.3 g, 76 mmol, 4.0 eq), and the mixture was stirred at 80° C. for 2 hours. The reaction mixture was filtered and concentrated under reduced pressure to give 2 (4.4 g, crude) as a colorless oil.

Step 3: tert-butyl 4-(benzyloxycarbonylaminomethyl)-4-hydroxy-piperidine-1-carboxylate (3): To a solution of 2 (4.4 g, 19 mmol, 1 eq) and NaHCO₃ (4.8 g, 58 mmol, 2.2 mL, 3 eq) in EtOH (100 mL) and H₂O (50 mL) was added CbzCl (3.3 g, 19 mmol, 2.7 mL, 1 eq). Then the mixture was stirred at 25° C. for 2 hours. The reaction mixture was diluted with H₂O (60 mL) and extracted with EA (40 mL×2). The combined organic layers were washed with saturated brine (25 mL×2), dried over Na₂SO₄, filtered and concentrated under reduced pressure to give a residue. The residue was purified by column chromatography (SiO₂, petroleum ether/ethyl acetate, 10/1 to 1/1) to give 3 (4.2 g, 60% yield) as a colorless oil. LCMS: (ES+) m/z (M+H)⁺=265.1.

Step 4: tert-butyl 4-(aminomethyl)-4-hydroxy-piperidine-1-carboxylate (4): To a solution of 3 (4.1 g, 11 mmol, 1 eq) in Me0H (40 mL) was added 5% Pd/C (482 mg, 225 μmol, 0.02 eq) under N₂ atmosphere. The suspension was degassed and purged with H₂ 3 times. The mixture was stirred under H₂ (15 psi) at 25° C. for 1 hours. The reaction mixture was filtered and concentrated under reduced pressure to give 4 (2.3 g, 89% yield) as a yellow solid.

Step 5: benzyl 1-methyl-4-oxo-cyclohexanecarboxylate (5): To a solution of 1-methyl-4-oxo-cyclohexanecarboxylic acid (2 g, 13 mmol, 1 eq) in DMF (15 mL) was added BnBr (3.3 g, 19 mmol, 2.3 mL, 1.5 eq) and K₂CO₃ (3.5 g, 26 mmol, 2 eq). Then the mixture was stirred at 50° C. for 2 hours. The reaction mixture was diluted with H₂O (60 mL) and extracted with EA (40 mL×2). The combined organic layers were washed with saturated brine (25 mL×2), dried over Na₂SO₄, filtered and concentrated under reduced pressure to give a residue. The residue was purified by column chromatography (SiO₂, petroleum ether/ethyl acetate, 50/1 to 20/1) to give 5 (1.9 g, 60% yield) as a yellow oil.

Step 6: tert-butyl 4-[[(4-benzyloxycarbonyl-4-methyl-cyclohexyl)amino]methyl]-4-hydroxy-piperidine-1-carboxylate (6): A solution of 4 (1.7 g, 6.9 mmol, 1 eq), 5 (1.8 g, 7.6 mmol, 1.1 eq) and HOAc (41 mg, 690 μmol, 39 μL, 0.1 eq) in THF (40 mL) was stirred at 25° C. for 0.5 hour. Then to the mixture was added NaBH(OAc)₃ (4.4 g, 21 mmol, 3 eq), and the mixture was stirred for another 2 hours. The reaction mixture was diluted with H₂O (60 mL) and extracted with EA (40 mL×2). The combined organic layers were washed with saturated brine 50 mL (25 mL×2), dried over Na₂SO₄, filtered and concentrated under reduced pressure to give a residue. The residue was purified by column chromatography (SiO₂, petroleum ether/ethyl acetate, 10/1 to 5/1) to give 6 (2.6 g, 82% yield) as a yellow oil. LCMS: (ES+) m/z (M+H)⁺=461.1.

Step 7:benzyl 1-methyl-4-(2-oxo-1-oxa-3,8-diazaspiro[4.5]decan-3-y1)cyclohexanecarboxylate (7): To a solution of 6 (2.6 g, 5.6 mmol, 1 eq) in DCM (30 mL) was added DIPEA (2.9 g, 23 mmol, 3.9 mL, 4 eq) and bis(trichloromethyl) carbonate (3.6 g, 11 mmol, 2 eq), and the mixture was stirred at 25° C. for 12 hours. The reaction mixture was concentrated under reduced pressure to give a residue. The residue was purified by prep-HPLC (column: Phenomenex luna C18 150×40 mm×15 μm; mobile phase: A: water (0.225%FA,v/v), B: ACN; B%: 8%-38% gradient over 9 min) to give 7 (800 mg, 37% yield) as a white solid. LCMS: (ES+) m/z (M+H)⁺=387.1.

Step 8: (1r,4r)-benzyl 4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxylate (8): To a solution of 7 (0.23 g, 0.60 mmol, 1 eq) and 2-(4-(chloromethyl)-2-cyclopropyl-5-ethoxyphenyl)-5-fluoropyridine (0.16 g, 0.54 mmol, 0.9 eq) in DMF (4 mL) was added DIEA (0.23 g, 1.8 mmol, 3 eq), and the mixture was stirred at 50° C. for 16 hours. The reaction mixture was diluted with H₂O (30 mL) and extracted with ethyl acetate (30 mL×3). The combined organic layers were washed with brine (30 mL×3), dried over Na₂SO₄, filtered and concentrated under reduced pressure. The residue was purified by column chromatography (SiO₂, petroleum ether:ethyl acetate, 1:1) to give 8 (0.05 g, 21% yield) as a white solid. LCMS: (ES⁺) m/z (M+H)⁺=656.2. (The trans configuration of 8 was established by comparing with the corresponding intermediates obtained in the synthesis of compounds 88 and 89).

Step 9: (1r,40-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxylic acid (Intermediate F): To a solution of 8 (0.1 g, 0.15 mmol, 1 eq) in MeOH (10 mL) was added 5% Pd/C (65 mg, 31 μmol, 0.2 eq) under N₂. The suspension was degassed under vacuum and purged with H₂ several times. The mixture was stirred under H₂ (15 psi) at 40° C. for 1 hour. The mixture was filtered and concentrated in vacuo. The residue was purified by prep-HPLC (column: Phenomenex Synergi C18 150×25 mm×10 μm; mobile phase: A: water (0.225%FA, v/v), B: ACN; B%: 13%-43% gradient over 10 min) to give Intermediate F (20 mg, 31% yield, 99.9% purity, FA salt) as a white solid. LCMS: (ES+) m/z (M+H) =566.3. ¹H NMR (400 MHz, CD₃OD) δ 8.54 (d, J=2.4 Hz, 1H), 7.75-7.69 (m, 2H), 7.14 (s, 1H), 7.01 (s, 1H), 4.11 (q, J=6.8 Hz, 2H), 3.96 (s, 2H), 3.60-3.53 (m, 1H), 3.44 (s, 2H), 3.01 (s, 2H), 2.95-2.89 (m, 2H), 2.06-1.90 (m, 4H), 1.79-1.74 (m, 1H), 1.79-1.74 (m, 1H), 1.72-1.66 (m, 8H), 1.43 (t, J=6.8 Hz, 3H), 1.25 (s, 3H), 0.81-0.77 (m, 2H), 0.59-0.56 (m, 2H).

Step 10: (1r,4r)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexanecarboxamide (Compound 90): A solution of Intermediate F (30 mg, 53 μmol, 1 eq) and HATU (24 mg, 64 μmol, 1.2 eq) in DMF (1 mL) was stirred at 25° C. for 0.5 hour. Then 2-(2-aminoethoxy)ethanol (22 mg, 0.21 mmol, 4 eq) and DIEA (21 mg, 0.16 mmol, 3 eq) was added to the solution, and the mixture was stirred at 25° C. for another 12 hours. The mixture was poured 20 mL of H₂O and then extracted with EA (30 mL×3). The combined organic layer was washed with water (20 mL×2) and brine (20 mL×2), dried over Na₂SO₄ and concentrated in vacuo. The residue was purified by prep-HPLC (column: Phenomenex Synergi C18 150×25 mm×10 μm; mobile phase: A: water (0.225% FA, v/v), B: ACN; B%: 14%-44% gradient over 10 min) to give Compound 90 (22 mg, 58% yield, 99% purity, FA) as a white solid. LCMS: (ES+) m/z (M+H)⁺=653.3. ¹H NMR (400 MHz, CD₃OD) δ 8.55 (d, J=2.8 Hz, 1H), 7.74-7.71 (m, 2H), 7.58 (t, J=5.6 Hz, 2H), 7.17(s, 1H), 7.04 (s, 1H), 4.13 (q, J=7.2 Hz, 2H), 4.09 (s, 2H), 3.67-3.65 (m, 2H), 3.55-3.52 (m, 5H), 3.46(s, 2H), 3.41-3.36 (m, 2H), 3.14 (s, 2H), 3.05(s, 2H), 2.11-2.01 (m, 4H), 1.92-1.88 (m, 1H), 1.77-1.66 (m, 8H), 1.44(t, J=7.2 Hz, 3H), 1.24 (s, 3H), 0.82-0.77 (m, 2H), 0.60-0.57 (m, 2H).

Step 11: 3-((1r,4r)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexanecarboxamido)propane-1-sulfonic acid (Compound 91): A solution of Intermediate F (35 mg, 62 μmol, 1 eq) and HATU (28 mg, 74 μmol, 1.2 eq) in DMF (1 mL) was stirred at 25° C. for 0.5 hour. Then 3-aminopropane-1-sulfonic acid (34 mg, 0.25 mmol, 4 eq) and DIEA (24 mg, 0.19 mmol, 3 eq) was added to the solution, and the mixture was stirred at 25° C. for another 12 hour. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give a residue. The residue was purified by prep-HPLC (column: Waters Xbridge 150×25 mm×5 μm; mobile phase: A: water (0.05% ammonia hydroxide v/v), B: ACN; B%: 8%-35% gradient over 10 min) to give Compound 91 (27 mg, 62% yield, 97.4% purity) as a white solid. LCMS: (ES+) m/z (M+H)⁼687.4. ¹H NMR (400 MHz, DMSO-d₆) δ 9.39(s, 1H), 8.69 (d, J=2.8 Hz, 1H), 7.87-7.82(m, 1H), 7.77-7.74(m, 1H), 7.66-7.63(m, 1H), 7.24(s, 1H), 7.08 (s, 1H), 4.33(d, J=4.4 Hz, 2H), 4.12 (q, J=7.2 Hz, 2H), 3.40 (s, 1H), 3.36(s, 2H), 3.16-3.07(m, 4H), 2.42-2.39(m, 2H), 2.13-1.97(m, 4H), 1.69-1.62(m, 8H), 1.77-1.66 (m, 8H), 1.37(t, J=7.2 Hz, 3H), 1.28-1.23 (m, 4H), 1.12(d, J=10.4 Hz, 3H), 0.80-0.77 (m, 2H), 0.59-0.58 (m, 2H).

### Example 12: 3-(8-((2-cyclopropyl-5-ethoxy-4′-fluoro-[1,1′-biphenyl]-4-yl)methyl)-2-oxo-1-oxa-3,8-diazaspiro [4.5] decan-3-yl)-N-(2-(2-hydroxyethoxy)ethyl)bicyclo [1.1.1] pentane-1-carboxamide (Compound 92)

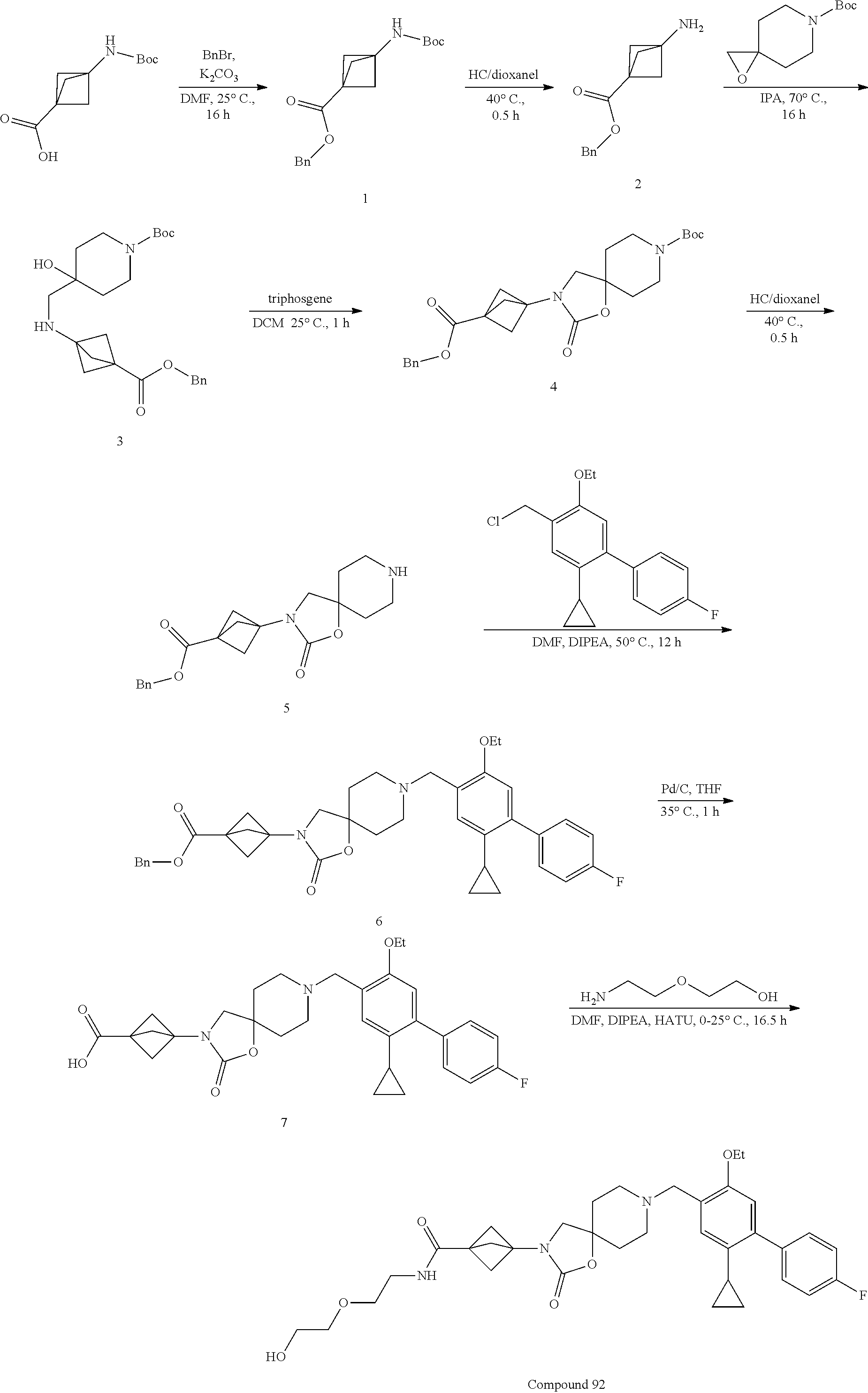

Step 1: benzyl 3-((tert-butoxycarbonyl)amino)bicyclo[1.1.1]pentane-1-carboxylate (1): To a solution of 3-(tert-butoxycarbonylamino)bicyclo[1.1.1]pentane-1-carboxylic acid (0.4 g, 1.8 mmol, 1 eq) in DMF (5 mL) was added BnBr (0.45 g, 2.64 mmol, 1.5 eq) and K₂CO₃ (0.48 g, 3.5 mmol, 2 eq). The mixture was stirred at 25° C. for 16 hours. The mixture was diluted with water (20 mL) and extracted with EtOAc (20 mL×3). The combined organic layer was washed with water (20 mL) and brine (20 mL×2), dried over Na₂SO₄ and concentrated in vacuo. The residue was slurried with petroleum ether/ethyl acetate (10:1, 20 mL) and filtered to give 1 (0.48 g, 86% yield) as a white solid. ¹H NMR (400 MHz, CDCl₃) δ 7.37-7.33 (m, 5 H), 5.13 (s, 2 H), 4.97-4.92 (m, 1 H), 2.31 (s, 6 H), 1.45 (s, 9 H).

Step 2: benzyl 3-aminobicyclo[1.1.1]pentane-1-carboxylate (2): A solution of 1 (0.48 g, 1.5 mmol, 1 eq) in HCl/dioxane (4 M, 5.2 mL) was stirred at 40° C. for 0.5 hour. The mixture was concentrated in vacuo. The solid was triturated with 20 mL petroleum ether/ethyl acetate (10:1) and filtered to give the HCl salt (a pure white solid). The white solid was dissolved in water (20 mL×3) and adjusted to pH 11 with saturated aqueous Na₂CO₃. The mixture was extracted with DCM:MeOH (10:1, 20 mL). The combined organic layer was washed with water (20 mL) and brine (20 mL×3), dried over Na₂SO₄, filtered and concentrated in vacuo to give 2 (320 mg, 94% yield) as a colorless oil.

Step 3: tert-butyl 4-(((3-((benzyloxy)carbonyl)bicyclo[1.1.1]pentan-1-yl)amino)methyl)-4-hydroxypiperidine-1-carboxylate (3): A solution of 2 (320 mg, 1.47 mmol, 1 eq) and tert-butyl 1-oxa-6-azaspiro[2.5]octane-6-carboxylate (345 mg, 1.6 mmol, 1.1 eq) in IPA (5 mL) was heated to 70° C. The mixture was stirred at 70° C. for 16 hours. The mixture was concentrated in vacuo. The residue was purified by prep-HPLC (column: Phenomenex luna C18 150×40 mm×15 μm; mobile phase: [A: water (0.225% FA, v/v), B: ACN]; B%: 3%-43% gradient over 9 min). The eluent was concentrated in vacuo. The residue was diluted with 20 mL of water and adjusted to pH 10 with saturated aqueous Na₂CO₃, then extracted with 10:1 DCM:MeOH (20 mL×3). The combined organic layer was washed with brine (15 mL×2), dried over Na₂SO₄, filtered and concentrated in vacuo to give 3 (200 mg, 28% yield) as a colorless oil.

Step 4: tert-butyl 3-(3-((benzyloxy)carbonyl)bicyclo[1.1.1]pentan-1-yl)-2-oxo-1-oxa-3,8-diazaspiro[4.5]decane-8-carboxylate (4): To a solution of 3 (200 mg, 0.46 mmol, 1 eq) and bis(trichloromethyl) carbonate (138 mg, 0.46 mmol, 1 eq) in DCM (15 mL) was added DIPEA (300 mg, 2.3 mmol, 5 eq). The mixture was stirred at 25° C. for 1 hour. The mixture was quenched with water (40 mL) and extracted with ethyl acetate (30 mL×3). The organic layer was washed with saturated brine (30 mL), dried over Na₂SO₄, filtered and concentrated in vacuo. The residue was purified by column chromatography (SiO₂, petroleum ether/ethyl acetate, 10:1) to give 4 (170 mg, 80% yield) as a white solid.

Step 5: benzyl 3-(2-oxo-1-oxa-3,8-diazaspiro[4.5]decan-3-yl)bicyclo[1.1.1]pentane-1-carboxylate (5): A solution of 4 (170 mg, 0.37 mmol, 1 eq) in HCl/dioxane (4 M, 4 mL) was stirred at 40° C. for 0.5 hour. The mixture was concentrated in vacuo to give 5 (145 mg, 99% yield, HCl salt) as a white solid.

Step 6: benzyl 3-(8-((2-cyclopropyl-5-ethoxy-4′-fluoro-[1, P-biphenyl]-4-yl)methyl)-2-oxo-1-oxa -3,8-diazaspiro[4.5]decan-3-yl)bicyclo[1.1.1]pentane-1-carboxylate (6): To a solution of 5 (145 mg, 0.37 mmol, 1 eq, HCl salt) and 1-(chloromethyl)-5-cyclopropyl-2-ethoxy-4-(4-fluorophenyl)benzene(113 mg, 0.37 mmol, 1 eq) in DMF (2 mL) was added DIPEA (191 mg, 1.5 mmol, 4 eq). The mixture was stirred at 50° C. for 12 hours. The mixture was diluted with water (40 mL) and extracted with ethyl acetate (30 mL×3). The organic layer was washed with saturated brine (30 mL), dried over Na₂SO₄, filtered and concentrated in vacuo. The residue was purified by prep-TLC (SiO₂, petroleum ether/ethyl acetate, 1:2) to give 6 (205 mg, 89% yield) as a white solid.

Step 7: 3-(8-((2-cyclopropyl-5-ethoxy-4′-fluoro-[1,1′-biphenyl]-4-yl)methyl)-2-oxo-1-oxa-3,8-diazaspiro[4.5]decan-3-yl)bicyclo[1.1.1]pentane-1-carboxylic acid (7): To a solution of 6 (205 mg, 0.33 mmol, 1 eq) in THF (10 mL) was added 10% Pd/C (70 mg, 66 μmol, 0.2 eq) under N₂. The suspension was degassed under vacuum and purged with H₂ several times. The mixture was stirred under H₂ (15 psi) at 35° C. for 1 hour. The mixture was filtered, and the filtrate was concentrated in vacuo. The residue was purified by prep-HPLC (column: UniSil 3-100 C18 Ultra (150×25 mm×3 μm); mobile phase: [A: water (0.225% FA, v/v), B: ACN]; B%: 22%-52% gradient over 10 min) to give 7 (98 mg, 55% yield, 99% purity, FA salt) as a white solid. ¹H-NMR (CD₃OD, 400 MHz): δ 7.46-7.42 (m, 2H), 7.20-7.15 (m, 2H), 7.09 (s, 1H), 6.88 (s, 1H), 4.22 (s, 2H), 4.12 (q, J=6.8 Hz, 2H), 3.46 (s, 2H), 3.3-3.31 (m, 2H), 3.23-3.16 (m, 2H), 2.29 (s, 6H), 2.16-2.04 (m, 4H), 1.80-1.76 (m, 1H), 1.44 (t, J=6.8 Hz, 3H), 0.82-0.79 (m, 2H), 0.63-0.62 (m, 2H).

Step 8: 3-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(2-(2-hydroxyethoxy)ethyl)bicyclo[1.1.1]pentane-1-carboxamide (Compound 92): To a solution of 7 (30 mg, 52 μmol, 1 eq, FA salt) in DMF (1 mL) was added DIPEA (13 mg, 0.1 mmol, 2 eq) and HATU (26 mg, 67 μmol, 1.3 eq). The mixture was stirred at 0° C. for 0.5 hour. Then 2-(2-aminoethoxy)ethanol (22 mg, 0.21 mmol, 4 eq) was added to the solution. The mixture was stirred at 25° C. for 16 hours. The mixture was concentrated in vacuo. The residue was purified by prep-HPLC (column: UniSil 3-100 C18 Ultra 150×25 mm×3 μm; mobile phase: [A: water (0.225% FA, v/v), B: ACN]; B%: 24%-54% gradient over 10 min) to give Compound 92 (22 mg, 62% yield, 98% purity, FA salt) as a brown solid. LCMS: (ES+) m/z (M+H)⁺=622.4. ¹H-NMR (400 MHz, CD₃OD) δ 7.45-7.42 (m, 2H), 7.18-7.14 (m, 2H), 7.02 (s, 1H), 6.81 (s, 1H), 4.07 (q, J=6.8 Hz, 2H), 3.90 (s, 2H), 3.67-3.65 (m, 2H), 3.55-3.52 (m, 4H), 3.43 (s, 2H), 3.40-3.37 (m, 2H), 3.00-2.87 (m, 4H), 2.32 (s, 6H), 2.05-1.95 (m, 4H), 1.79-1.75 (m, 1H), 1.41 (t, J=6.8 Hz, 3H), 0.79-0.76 (m, 2H), 0.62-0.59 (m, 2H).

### Example 13: methyl 2-cyclopropyl-5-ethoxy-4-[[4-[[4-[[(2S,3R,4R,5R)-2,3,4,5,6-pentahydroxyhexyl]carbamoyl]phenyl]carbamoyl]piperazin-1-yl]methyl]benzoate (Compound 93)

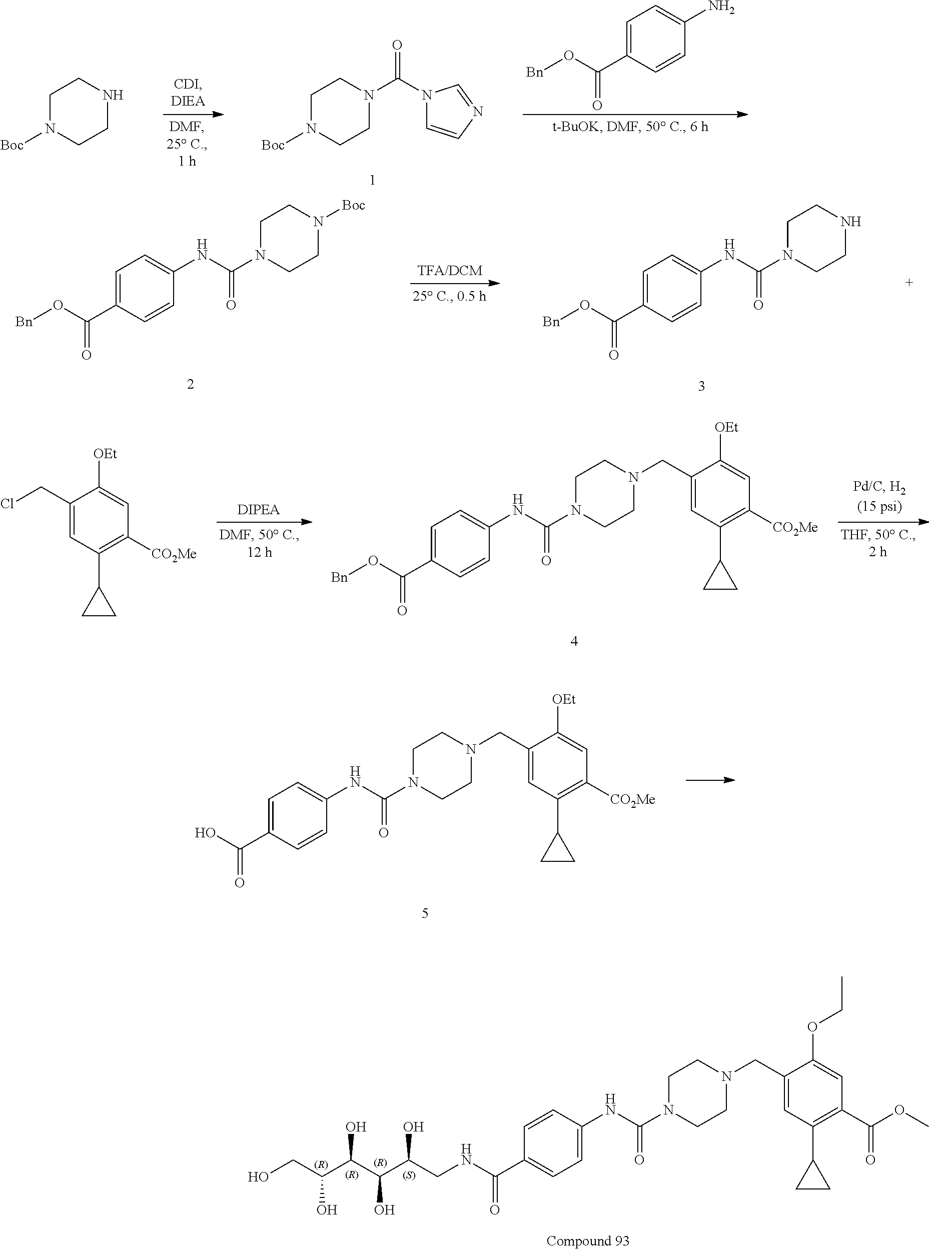

Step 1: tent-butyl 4-(1H-imidazole-1-carbonyl)piperazine-1-carboxylate (1): To a solution of tent-butyl piperazine-1-carboxylate (0.30 g, 1.6 mmol) in DMF (5 mL) was added CDI (0.26 g, 1.6 mmol) and DIPEA (0.21 g, 1.6 mmol, 0.28 mL), and the mixture was stirred at 25° C. for 1 hour to give 1 (0.45 g, crude) in 5 ml DMF was used to next step.

Step 2: tent-butyl 4-((4-((benzyloxy)carbonyl)phenyl)carbamoyl)piperazine-1-carboxylate (2): To a solution of 1 (0.45 g, 1.6 mmol) in DMF (5 mL) was added benzyl 4-aminobenzoate (0.37 g, 1.6 mmol) and t-BuOK (1 M, 3.2 mL). Then the mixture was stirred at 50° C. for 6 hours. The mixture was poured into 30 mL of H₂O and extracted with ethyl acetate (40 mL×3). The combined organic layer was washed with water (30 mL×2) and saturated brine (30 mL×2), dried over Na₂SO₄, and concentrated in vacuo. The residue was purified by column chromatography (SiO₂, petroleum ether:ethyl acetate, 30:1 to 10:1) to give 2 (0.1 g, 10% yield) as a yellow oil. LCMS: (ES⁺) m/z (M−55)⁺=383.9.

Step 3: benzyl 4-(piperazine-1-carboxamido)benzoate (3): To a soluiton of 2 (0.14 g, 0.31 mmol) in DCM (2 mL) was added TFA (0.74 g, 6.5 mmol, 0.48 mL), and the mixture was stirred at 20° C. for 0.5 hour. The mixture was adjust to pH 7 with saturated aqueous NaHCO₃, and the mixture was poured into 30 mL of H₂O, and extracted with ethyl acetate (40 mL×3) . The combined organic layer was washed with water (30 mL×2) and saturated brine (30 mL×2), dried over Na₂SO₄, and concentrated in vacuo to give 3 (0.15 g, crude) as a yellow oil. LCMS: (ES⁺) m/z (M+H)⁺=339.9.

Step 4: methyl 4-((4-((4-((benzyloxy)carbonyl)phenyl)carbamoyl)piperazin-1-yl)methyl)-2-cyclopropyl-5-ethoxybenzoate (4): To a solution of 3 (0.1g, 0.29 mmol) and methyl 4-(chloromethyl)-2-cyclopropyl-5-ethoxybenzoate (53 mg, 0.20 mmol) in DMF (2 mL) was added DIEA (76 mg, 0.59 mmol, 0.1 mL), and the mixture was stirred at 50° C. for 12 hours. The mixture was poured into 20 mL of H₂O and extracted with ethyl acetate (30 mL×3). The combined organic layer was washed with water (20 mL×2) and saturated brine (20 mL×2), dried over Na₂SO₄, and concentrated in vacuo to give 5 (0.18 g, crude) as a yellow oil. LCMS: (ES⁺) m/z (M+H)⁺=572.2.

Step 5: 4-(4-(5-cyclopropyl-2-ethoxy-4-(methoxycarbonyl)benzyl)piperazine-1-carboxamido)benzoic acid (5): To a solution of 4 (0.18 g, 0.31 mmol) in THF (10 mL) was added 5% Pd/C (0.13 g, 63 μmol) under N₂. The suspension was degassed under vacuum and purged with H₂ several times. The mixture was stirred under H₂ (15 psi) at 50° C. for 2 hours. The mixture was filtered, and the filtrate was concentrated in vacuo. The residue was purified by prep-HPLC (column: Phenomenex Synergi C18 150×25 mm×10 μm; mobile phase: A: water (0.225% FA, v/v), B: ACN; B%: 11% - 41% gradient over 10 min) to give 5 (30 mg, 19% yield, 97% purity) as a white solid. LCMS: (ES⁺) m/z (M+H)⁺=482.3. ¹H NMR (400 MHz, CD₃OD) δ 7.91 (d, J=8.8 Hz, 2H), 7.47 (d, J=8.8 Hz, 2H), 7.33 (s, 1H), 7.12 (s, 1H), 4.09 (dd, J₁=4.0 Hz, J₂=7.2 Hz, 2H), 3.90 (s, 4H), 3.74 (s, 2H), 3.60 (s, 4H), 2.67 (s, 4H), 2.49 (s, 1H), 1.42 (t, J=6.8 Hz, 3H), 0.94 (d, J=8.4 Hz, 2H), 0.64-0.60 (m, 2H).

Following the procedures described above, from 5 and appropriate starting materials, methyl 2-cyclopropyl-5-ethoxy-4-[[4-[[4-[[(2S,3R,4R,5R)-2,3,4,5,6-pentahydroxyhexyl]carbamoyl]phenyl]carbamoyl]piperazin-1-yl]methyl]benzoate (Compound 93) was obtained. LCMS : (ES⁺) m/z (M+H)⁺=645.2 ¹H NMR (400 MHz, CD₃OD) δ 7.77 (d, J=8.4 Hz, 2H), 7.48 (d, J=8.8 Hz, 2H), 7.35 (s, 1H), 7.14 (s, 1H), 4.13-4.08 (m, 2H), 3.96-3.95 (m, 1H), 3.91 (s, 3H), 3.84-3.77 (m, 4H), 3.72-3.63 (m, 8H), 3.48-3.43 (m, 1H), 2.78 (s, 4H), 2.53-2.46 (m, 1H), 1.43 (t, J=7.2 Hz, 3H), 0.96-0.92 (m, 2H), 0.65-0.61 (m, 2H).

### Example 14: 4-(((2S,3R,4R,5R)-2,3,4,5,6-pentahydroxyhexyl)carbamoyl)phenyl 44(2-cyclopropyl-5-ethoxy-4′-fluoro-[1,1′-biphenyl]-4-yl)methyl)piperazine-1-carboxylate (Compound 94)

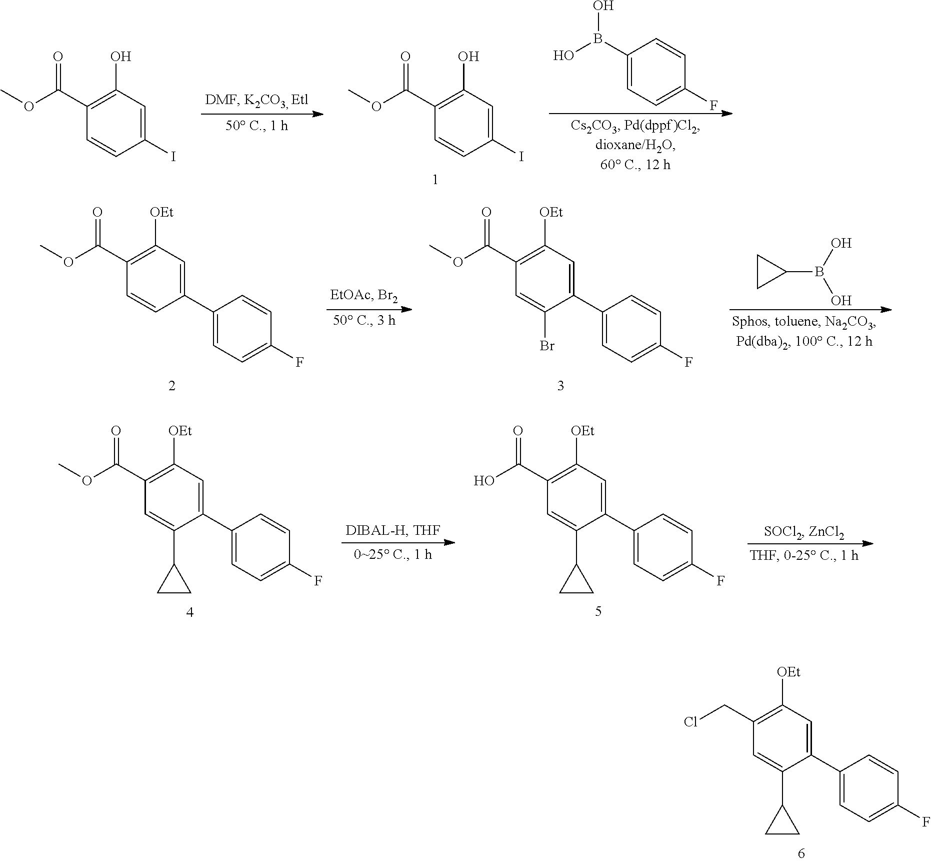

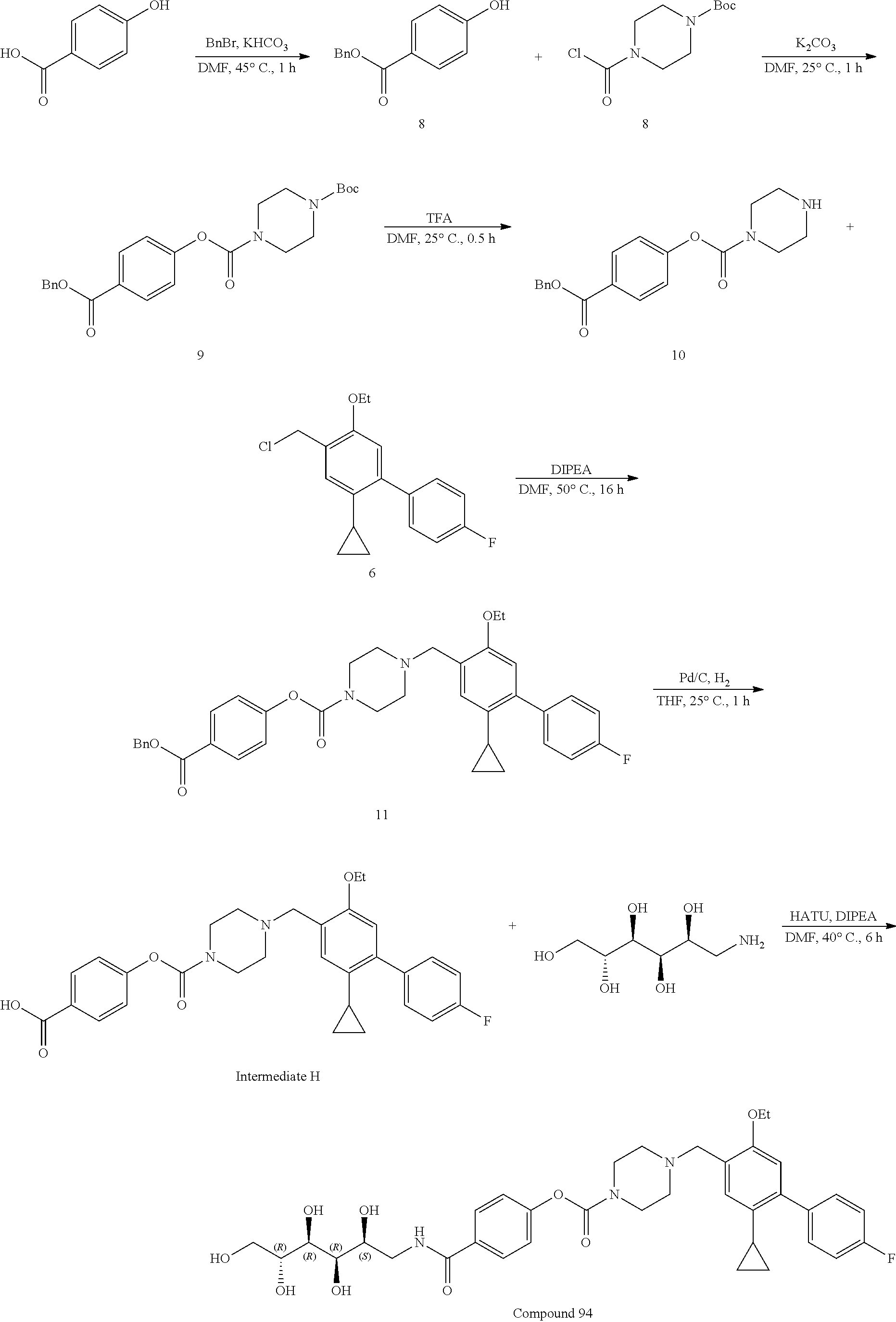

Step 1: methyl 2-ethoxy-4-iodo-benzoate (1): To a solution of methyl 2-hydroxy-4-iodo-benzoate (13 g, 47 mmol, 1 eq) in DMF (130 mL) was added K₂CO₃ (13 g, 94 mmol, 2 eq) and EtI (14.6 g, 94 mmol, 7.5 mL, 2 eq). The mixture was stirred at 50° C. for 1 hour. The reaction mixture was poured into H₂O (50 mL) and extracted with EA (50 mL×3). The combined organic layer was washed with water (50 mL×2) and brine (50 mL×2), dried over Na₂SO₄ and concentrated in vacuo to give 1 (14.2 g, crude) as a yellow oil.

Step 2: methyl 2-ethoxy-4-(4-fluorophenyl)benzoate (2): To a solution of 1 (7.5 g, 25 mmol, 1 eq), (4-fluorophenyl)boronic acid (3.8 g, 27 mmol, 1.1 eq), Cs₂CO₃ (16 g, 49 mmol, 2 eq) and Pd(dppf)Cl₂ (896 mg, 1.2 mmol, 0.05 eq) was added H₂O (20 mL) and dioxane (60 mL). Then the mixture was stirred at 60° C. for 12 hours. The reaction mixture was diluted with H₂O (100 mL) and extracted with EA (90 mL×2). The combined organic layers were washed with saturated brine (50 mL×2), dried over Na₂SO₄, filtered, and concentrated under reduced pressure to give a residue. The residue was purified by column chromatography (SiO₂, petroleum ether/ethyl acetate, 100:1 to 10:1) to give 2 (6.7 g, 99% yield) as a yellow solid.

Step 3: methyl 5-bromo-2-ethoxy-4-(4-fluorophenyl)benzoate (3): To a solution of 2 (7.2 g, 26 mmol, 1 eq) in EtOAc (72 mL) was added Br₂ (5.0 g, 32 mmol, 1.6 mL, 1.2 eq). Then the mixture was stirred at 50° C. for 3 hours. The reaction mixture was diluted with H₂O (120 mL) and extracted with EA (75 mL×2). The combined organic layers were washed with saturated brine (50 mL×2), dried over Na₂SO₄, filtered and concentrated under reduced pressure to give a residue. The residue was purified by column chromatography (SiO₂, petroleum ether/ethyl acetate, 50:1 to 5:1) to give 3 (5.5 g, 59% yield) as a white solid. ¹H NMR (400 MHz, CD₃OD) δ 8.07 (s, 1 H), 7.35-7.42 (m, 2 H), 7.10-7.17 (m, 2 H), 6.90 (s, 1 H), 4.11 (q, J=7.2 Hz, 2 H), 3.91 (s, 3 H), 1.47 (t, J=6.8 Hz, 3 H).

Step 4: methyl 5-cyclopropyl-2-ethoxy-4-(4-fluorophenyl)benzoate (4): To a solution of 3 (5.5 g, 16 mmol, 1 eq), cyclopropylboronic acid (3.3 g, 39 mmol, 2.5 eq) and Na₂CO₃ (4.1 g, 39 mmol, 2.5 eq) in toluene (16 mL) was added SPhos (959 mg, 2.3 mmol, 0.15 eq) and Pd(dba)₂ (269 mg, 467 μmol, 0.03 eq) under N₂. Then the mixture was stirred at 100° C. for 12 hours. The reaction mixture was diluted with H₂O (120 mL) and extracted with EA (75 mL×2). The combined organic layers were washed with saturated brine (50 mL×2), dried over Na₂SO₄, filtered and concentrated under reduced pressure and purified by column chromatography (SiO₂, Petroleum ether/Ethyl acetate=50/1 to 5/1) to give 4 (4.8 g, 98% yield) as a yellow solid.

Step 5: [5-cyclopropyl-2-ethoxy-4-(4-fluorophenyl)phenyl]methanol (5): To a solution of 4 (4.8 g, 15 mmol, 1 eq) in THF (50 mL) was added DIBAL-H (1 M, 46 mL, 3 eq) at 0° C. Then the mixture was stirred at 25° C. for 1 hour. The reaction mixture was quenched by addition H₂O (70 mL) at 0° C. and 1N aqueous HCl (60 mL) and extracted with EA (50 mL×2). The combined organic layers were washed with saturated brine (40 mL×2), dried over Na₂SO₄, filtered, and concentrated under reduced pressure to give 5 (5 g, crude) as a white solid.

Step 6: 1-(chloromethyl)-5-cyclopropyl-2-ethoxy-4-(4-fluorophenyl)benzene (6): To a solution of 5 (5 g, 17 mmol, 1 eq) in THF (50 mL) was added SOCl₂ (3.1 g, 26 mmol, 1.9 mL, 1.5 eq) and ZnCl₂ (238 mg, 1.8 mmol, 82 μL, 0.1 eq) at 0° C. Then the mixture was stirred at 25° C. for 1 hour. The reaction mixture was diluted with H₂O (100 mL) and extracted with EA (90 mL×2). The combined organic layers were washed with saturated brine (50 mL×2), dried over Na₂SO₄, filtered, and concentrated under reduced pressure to give 6 (4.8 g, 90% yield) as a yellow oil.

Step 7: tert-butyl 4-(chlorocarbonyl)piperazine-1-carboxylate (7): A mixture of tert-butyl piperazine-1-carboxylate (2.1 g, 11 mmol, 1 eq) and pyridine (1.3 g, 17 mmol, 1.4 mL, 1.5 eq) in DCM (20 mL) was stirred at 0° C. for 10 minutes under nitrogen atmosphere. Then bis(trichloromethyl)carbonate (4.3 g, 14 mmol, 1.3 eq) in DCM (20 mL) was added dropwise at 0° C. under nitrogen atmosphere. The mixture was stirred at 0° C. for 1 hour. The cooling bath was then removed, and the reaction stirred at 25° C. for a further 1 hour. The mixture was diluted with 1M aqueous HCl (20 mL) to pH 1-2 and extracted with DCM 600 mL (200 mL×3). The combined organic extracts were washed with brine (200 mL) and dried over anhydrous Na₂SO₄, and the drying agent was removed by filtration. The filtrate was concentrated to dryness under reduced pressure to provide 7 (2.4 g, 86% yield) as a yellow solid.

Step 8: benzyl 4-hydroxybenzoate (8): To a solution of 4-hydroxybenzoic acid (2 g, 14 mmol, 1 eq) and KHCO₃ (2.9 g, 29 mmol, 2 eq) in DMF (20 mL) was added bromomethylbenzene (3.7 g, 22 mmol, 2.6 mL, 1.5 eq). The mixture was stirred at 45° C. for 1 hour. The mixture was diluted with water (100 mL) and extracted with EA (100 mL×3). The combined organic layers were washed with brine (100 mL×6), dried over Na₂SO₄, filtered and concentrated under reduced pressure to give a residue. The residue was purified by column chromatography (SiO₂, petroleum ether/ethyl acetate, 20:1 to 4:1) to give 8 (2.8 g, 85% yield) as a white solid. ¹H NMR (400 MHz, CDCl₃) δ 8.06-7.96 (m, 2H), 7.48-7.32 (m, 5H), 6.97-6.77 (m, 2H), 6.26 (s, 1H), 5.36 (s, 2H).

Step 9: 1-(4-((benzyloxy)carbonyl)phenyl) 4-tert-butyl piperazine-1,4-dicarboxylate (9): To a solution of 7 (1.0 g, 4.4 mmol, 1 eq) and 8 (1.2 g, 4.82 mmol, 1.1 eq) in DMF (10 mL) was added K₂CO₃ (0.91 g, 6.6 mmol, 1.5 eq). The mixture was stirred at 25° C. for 1 hour under N₂. The mixture was diluted with water (100 mL) and extracted with EA (100 mL×3). The combined organic layers were washed with brine (100 mL×6), dried over Na₂SO₄, filtered, and concentrated under reduced pressure to give 9 (1.8 g, 93% yield) as a yellow solid. ¹H NMR (400 MHz, CDCl₃) δ 8.13-8.07 (m, 2H), 7.48-7.32 (m, 5H), 7.23-7.16 (m, 2H), 5.37 (s, 2H), 3.66-3.64 (m, 2H), 3.59-3.47 (m, 6H), 1.50 (s, 9H).

Step 10: 4-((benzyloxy)carbonyl)phenyl piperazine-1-carboxylate (10): To a solution of 9 (800 mg, 1.8 mmol, 1 eq) in DCM (4 mL) was added TFA (1.7 g, 3.6 mmol, 1.1 mL, 2 eq). The mixture was stirred at 25° C. for 0.5 hour. The mixture was basified to pH 9-10 with NaHCO₃ solution, and then diluted with water (50 mL) and extracted with DCM (30 mL×3). The combined organic layers were washed with saturated sodium chloride solution (20 mL×3), dried over Na₂SO₄, filtered, and concentrated under reduced pressure to give 10 (0.6 g, 97% yield) as yellow solid. ¹H NMR (400 MHz, CDCl₃) δ 8.13-8.07 (m, 2H), 7.48-7.31 (m, 5H), 7.23-7.16 (m, 2H), 5.36 (s, 2H), 3.70-3.51 (m, 4H), 2.97-2.89 (m, 4H).

Step 11: 4-((benzyloxy)carbonyl)phenyl 4-((2-cyclopropyl-5-ethoxy-4′-fluoro-[1,1′-biphenyl]-4-yl)methyl)piperazine-1-carboxylate (11): To a solution of 6 (148 mg, 0.48 mmol) and 10 (150 mg, 0.44 mmol) in DMF (2 mL) was added DIPEA (114 mg, 0.88 mmol). The mixture was stirred at 50° C. for 16 hours. The mixture was diluted with water (20 mL), extracted with EA (30 mL×3), The combined organic layer was washed with water (20 mL) and brine (20 mL), dried over Na₂SO₄, and concentrated in vacuo to give 11 (260 mg, 97% yield) as a white solid. LCMS: (ES+) m/z (M+H)⁺=609.3.

Step 12: 4-((4-((2-cyclopropyl-5-ethoxy-4′-fluoro-[1,1′-biphenyl]-4-yl)methyl)piperazine-1-carbonyl)oxy)benzoic acid (Intermediate H): To a solution of 11 (0.26 g, 0.43 mmol) in THF (8 mL) was added 5% Pd/C (0.18 g, 85 μmol) under N₂. The suspension was degassed under vacuum and purged with H2 several times. The mixture was stirred under H₂ (15 psi) at 25° C. for 1 hour. The reaction mixture was filtered, and the filtrate was concentrated.

The residue was purified by prep-HPLC (column: Phenomenex Synergi C18 150×25 mm×10 μm; mobile phase: A: [water (0.225%FA, v/v), B: ACN]; B%: 20%-50% gradient over 10 min) to give Intermediate H (138 mg, 62% yield, 99% purity, FA salt) as a white solid. LCMS: (ES+) m/z (M+H)⁺=519.2. ¹H NMR (400 MHz , CD₃OD) δ 8.06-8.02 (m, 2H), 7.45-7.42 (m, 2H), 7.22-7.20 (m, 2H), 7.20-7.13 (m, 2H), 7.03 (s, 1H), 6.80 (s, 1H), 4.07 (q, J=6.8 Hz, 2H), 3.81 (s, 2H), 3.78-3.72 (m, 2H), 3.68-3.58 (m, 2H), 2.80-7.70 (br, 4H), 1.80-1.74 (m, 1H), 1.42 (t, J=6.8 Hz, 3H), 0.79-0.76 (m, 2H), 0.61-0.59 (m, 2H).

Step 13: 4-(((2S,3R,4R,5R)-2,3,4,5,6-pentahydroxyhexyl)carbamoyl)phenyl 4-((2-cyclopropyl-5-ethoxy-4′-fluoro-[1,1′-biphenyl]-4-yl)methyl)piperazine-1-carboxylate (Compound 94): To a solution of Intermediate H (250 mg, 0.48 mmol, 1 eq) and (2R,3R,4R,5S)-6-aminohexane-1,2,3,4,5-pentol (0.13 g, 0.72 mmol, 1.5 eq) in DMF (5 mL) was added HATU (0.22 g, 0.58 mmol, 1.2 eq) and DIPEA (0.19 g, 1.5 mmol, 0.25 mL, 3 eq). The mixture was stirred at 40° C. for 6 hours. The mixture was purified by prep-HPLC (column: Phenomenex Synergi Max-RP 150×50 mm×10 μm; mobile phase: [A: water (0.225%FA, v/v), B: ACN]; B%: 17%-47% gradient over 11 min) to give Compound 94 (111.77 mg, 33% yield, 98% purity) as a white solid. LCMS: (ES⁺) m/z (M+H)⁺=682.5. ¹H NMR (400 MHz , CD₃OD) δ 7.89-7.87 (m, 2H), 7.46-7.42 (m, 2H), 7.24-7.22 (m, 2H), 7.18-7.14 (m, 2H), 7.04 (s, 1H), 6.82 (s, 1H), 4.11-4.06 (m, 2H), 3.99-3.95 (m, 1H), 3.87 (s, 2H), 3.82-3.77 (m, 4H), 3.73-3.61 (m, 6H), 3.50-3.44 (m, 1H), 2.84 (s, 4H), 1.82-1.75 (m, 1H), 1.43 (t, J=7.2 Hz, 3H), 0.80-0.78 (m, 2H), 0.63-0.60 (m, 2H).

### Example 15: [4-[[4-(2-hydroxyethylamino)-4-oxo-butyl]carbamoyl]phenyl]4-[(4-cyano-5-cyclopropyl-2-ethoxy-phenyl)methyl]piperazine-1-carboxylate (Compound 95)

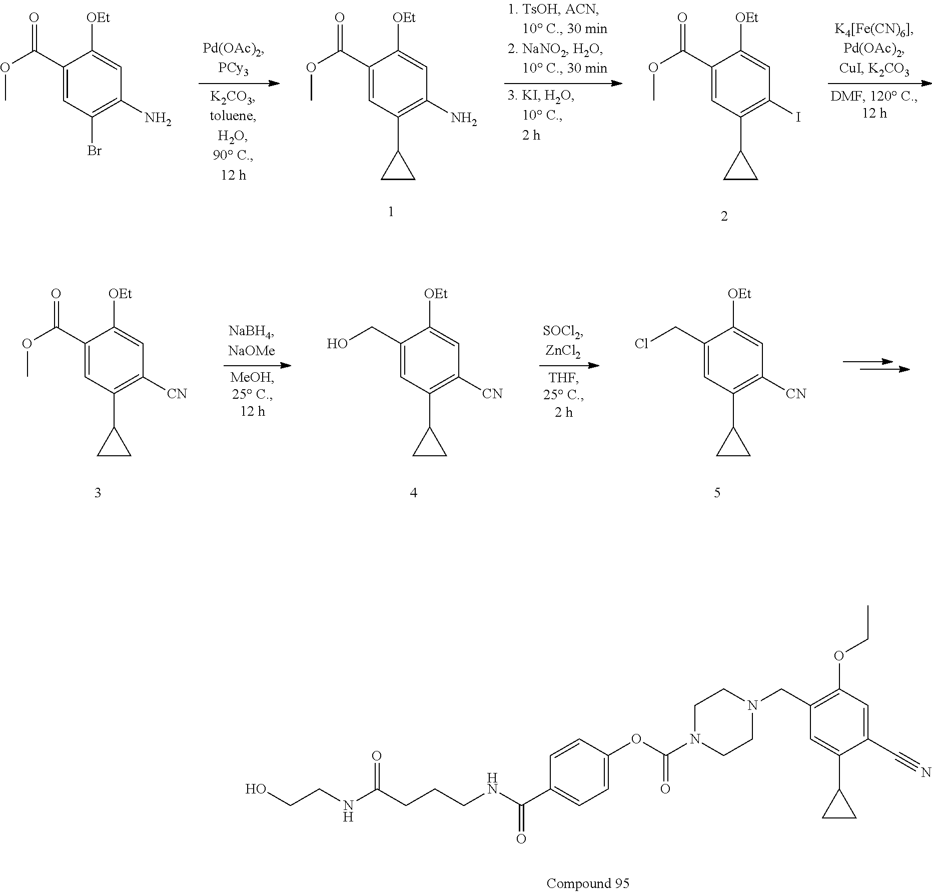

Step 1: methyl 4-amino-5-cyclopropyl-2-ethoxybenzoate (1): To a solution of cyclopropylboronic acid (4.5 g, 53 mmol, 3 eq) and methyl 4-amino-5-bromo-2-ethoxybenzoate (4.8 g, 18 mmol, 1 eq) in toluene (100 mL) H₂O (20 mL) was added Pd(OAc)₂ (0.39 g, 1.8 mmol, 0.1 eq), PCy₃ (0.50 g, 1.8 mmol, 0.57 mL, 0.1 eq), and K₃PO₄ (11 g, 52 mmol, 3 eq). The mixture was stirred at 90° C. for 12 hours. The residue was diluted with water 100 mL and extracted with ethyl acetate (100 mL×2), the combined organic layers were washed with saturated brine (50 mL×2), dried over Na₂SO₄, filtered and concentrated under reduced pressure. The residue was purified by column chromatography (SiO₂, Petroleum ether/Ethyl acetate=4:1 to 3:1). The spot with Rf=0.1 was collected to give 1 (3.0 g, 12 mmol, 69% yield) as a white solid. LCMS: (ES+) m/z (M+1)⁺=236.1.

Step 2: methyl 5-cyclopropyl-2-ethoxy-4-iodobenzoate (2): TsOH (7.7 g, 45 mmol, 3.5 eq) was added to a solution of 1 (3 g, 13 mmol, 1 eq) in ACN (90 mL) at 10° C., and the resultant mixture was stirred for 30 minutes at the same temperature. An aqueous solution (1 mL) of NaNO₂ (5.6 g, 82 mmol, 1 mL, 6.4 eq) was added to the reaction mixture, and the resultant was stirred for 30 minutes at 10° C. An aqueous solution (1 mL) of KI (1.9 g, 12 mmol, 1 mL, 0.93 eq) was added dropwise thereto, and the resultant mixture was stirred for 2 hours at 25° C. The mixture was poured into saturated aqueous NaHCO₃ (20 mL) and concentrated under reduced pressure to give a residue. The residue was purified by prep-TLC (SiO₂, petroleum ether/ethyl acetate, 10:1). The spot with Rf=0.6 was collected to give 2 (0.52 g, 12% yield) as a yellow oil. ¹H NMR (400 MHz, CDCl₃) δ 7.44 (s, 1 H), 7.34 (s, 1 H), 4.08 (q, J=6.8 Hz, 2 H), 3.87 (s, 3 H), 1.95-1.88 (m, 1H), 1.44 (t, J=7.2 Hz, 3 H) , 1.02-0.97(m, 2 H), 0.65-0.61 (m, 2 H).

Step 3: methyl 4-cyano-5-cyclopropyl-2-ethoxybenzoate (3): To a solution of 2 (0.48 g, 1.4 mmol, 1 eq) in DMF (5 mL) was added K₂CO₃ (0.29 g, 2.1 mmol, 1.5 eq), Pd (OAc)₂ (0.30 mg, 1.4 μmol, 0.001 eq) CuI (13 mg, 69 μmol, 0.05 eq), and K4[Fe(CN)₆] (0.31 g, 0.83 mmol, 0.6 eq). The mixture was stirred at 120° C. for 12 hours. The reaction mixture was filtered. The residue was diluted with water (20 mL) and extracted with ethyl acetate (20 mL×2). The combined organic layers were washed with saturated brine (10 mL×2), dried over Na₂SO₄, filtered and concentrated under reduced pressure. The residue was purified by column chromatography (SiO₂, petroleum ether/ethyl acetate, 10/1). The spot with Rf=0.56 was collected to give 3 (0.2 g, 59% yield) as a colorless oil. LCMS: (ES+) m/z (M+H)⁺=246.2.

Step 4: 2-cyclopropyl-5-ethoxy-4-(hydroxymethyl)benzonitrile (4): To a solution of 3 (0.20 g, 0.82 mmol, 1 eq) in MeOH (2 mL) was added NaBH₄ (62 mg, 1.6 mmol, 2 eq) and NaOMe (0.44 mg, 8.2 μmol, 0.01 eq). The mixture was stirred at 25° C. for 12 hours . The residue was diluted with saturated aqueous NH₄Cl (5 mL) and extracted with ethyl acetate (20 mL×2). The combined organic layers were washed with saturated brine (20 mL×2), dried over Na₂SO₄, filtered, and concentrated under reduced pressure to give 4 (0.15 g, crude) as a white solid. ¹H NMR (400 MHz, CDCl₃) δ 7.01 (s, 1 H), 6.94 (s, 1 H), 4.69 (d, J=2.8 Hz, 2 H), 4.07 (q, J=6.8 Hz, 2 H), 2.24-2.19(m, 2 H), 1.45 (t, J=6.8 Hz, 3 H), 1.11-1.07 (m, 2 H), 0.78-0.74 (m, 2 H).

Step 5: 4-(chloromethyl)-2-cyclopropyl-5-ethoxybenzonitrile (5): To a solution of 4 (0.14 g, 0.64 mmol, 1 eq) in THF (2 mL) was added SOCl₂ (0.11 g, 0.97 mmol, 70 pL, 1.5 eq) and ZnCl₂ (8.78 mg, 0.64 mmol, 0.1 eq). The mixture was stirred at 25° C. for 0.5 hour. The residue was diluted with water (15 mL) and extracted with ethyl acetate (15 mL×2). The combined organic layers were washed with saturated brine (10 mL×2), dried over Na₂SO₄, filtered, and concentrated under reduced pressure to give 5 (0.2 g, crude) as a white solid.

Following the procedure described above, from 5 and other starting materials and intermediates, [4-[[4-(2-hydroxyethylamino)-4-oxo-butyl]carbamoyl]phenyl] 4-[(4-cyano-5-cyclopropyl-2-ethoxy-phenyl)methyl]piperazine-1-carboxylate (Compound 95) was obtained. LCMS: (ES⁺) m/z (M+H)⁺=578.2. ¹H NMR (400 MHz, CDCl₃) δ 7.81 (d, J=8.4 Hz, 2 H), 7.18 (d, J=8.4 Hz, 2 H), 7.03 (d, J=6.4 Hz, 2 H), 6.90 (t, J=5.6 Hz, 1 H), 6.54 (s, 1 H), 4.03 (q, J=7.2 Hz, 2 H), 3.70 (s, 1 H), 3.65 (t, J=5.2 Hz, 2 H), 3.6 (s, 3 H), 3.52 (q, J=6.4 Hz, 2 H), 3.39 (q, J=5.2 Hz, 2 H), 2.54 (t, J=4.8 Hz, 4 H), 2.33-2.30 (m, 2 H), 2.23-2.18 (m, 2 H), 2.01-1.93 (m, 3 H), 1.44 (t, J=6.8 Hz, 3 H), 1.14-1.06 (m, 2 H), 0.78-0.70 (m, 2 H).

The following compounds were prepared according to the procedures described above using the appropriate intermediates.

| Cpd | Characterization Data |
|---|---|
| 96 | (ES⁺) m/z (M + H)⁺ = 646.2. ¹H NMR (400 MHz, CD₃OD) δ 8.22 (s, 1H), 7.87 (d, J = 8.8 |
| | Hz, 2H), 7.34 (s, 1H), 7.21 (d, J = 8.4 Hz, 2H), 7.14 (s, 1H), 4.09 (q, J = 7.2 Hz, 2H), |
| | 4.00-3.93 (m, 1H), 3.90 (s, 3H), 3.83-3.79 (m, 2H), 3.78-3.76 (m, 4H), 3.73-3.67 |
| | (m, 3H), 3.66-3.60 (m, 3H), 3.51-3.43 (m, 1H), 2.76-2.67 (m, 4H), 2.56-2.44 (m, |
| | 1H), 1.43 (t, J = 6.8 Hz, 3H), 0.98-0.89 (m, 2H), 0.66-0.58 (m, 2H). |
| 97 | (ES⁺) m/z (M + H)⁺ = 644.3. ¹H NMR (400 MHz, CD₃OD) δ 7.81 (d, J = 8.4 Hz, 2H), |
| | 7.34 (d, J = 8.4 Hz, 2H), 7.30 (s, 1H), 7.06 (s, 1H), 4.04 (q, J = 7.2 Hz, 2H), 3.95-3.90 |
| | (m, 1H), 3.90 (s, 3H), 3.84 (s, 2H), 3.83-3.76 (m, 2H), 3.72-3.68 (m, 2H), 3.65-3.60 |
| | (m, 5H), 3.60-3.56 (m, 2H), 3.47 (dd, Ji = 13.6, J2 = 7.2 Hz, 1H), 3.35 (s, 1H), 2.55- |
| | 2.40 (m, 5H), 1.38 (t, J = 6.8 Hz, 3H), 0.96-0.86 (m, 2H), 0.62-0.56 (m, 2H). |
| 98 | (ES⁺) m/z (M + H)⁺ = 646.4. ¹H NMR (400 MHz, CD₃OD) δ 8.18 (br, 1H), 7.71 (d, J = |
| | 8.0 Hz, 1H), 7.60 (t, J = 1.6 Hz, 1H), 7.48 (t, J = 8.0 Hz, 1H), 7.35 (s, 1H), 7.31-7.29 |
| | (m, 1H), 7.14 (s, 1H), 4.08 (q, J = 6.8 Hz, 2H), 3.91-3.88 (m, 1H), 3.81 (s, 3H), 3.80- |
| | 3.62 (m, 12H), 3.49-3.47 (m, 1H), 2.80-2.70 (m, 4H), 2.53-2.48 (m, 1H), 1.43 (t, J = |
| | 6.8 Hz, 3H), 0.95-0.92 (m, 2H), 0.65-0.62 (m, 2H). |
| 99 | (ES⁺) m/z (M + H)⁺ = 715.4. ¹H NMR (400 MHz, CDCl₃) δ 8.46 (s, 1H), 7.69 (d, J = 8.4 |
| | Hz, 1H), 7.32 (s, 1H), 7.22 (d, J = 8.4 Hz, 2H), 7.11 (s, 1H), 4.56-4.52 (m, 1H), 4.36- |
| | 4.32 (m, 1H), 4.19 (s, 1H), 4.09-4.04 (m, 3H), 3.96 (s, 1H), 3.89 (s, 3H), 3.98-3.95 |
| | (m, 1H), 3.79-3.76 (m, 1H), 3.70-3.64 (m, 7H), 3.56 (s, 2H), 3.35 (s, 2H), 3.22-3.07 |
| | (m, 3H), 2.58-2.46 (m, 5H), 1.42 (t, J = 6.8 Hz, 3H), 0.95-0.90 (m, 2H), 0.63-0.59 |
| | (m, 2H). |

II. Biological Evaluation

### Example A-1: In Vitro Activity Assay

### Inositol Phosphate Accumulation Assay

CHO-K1 cells stably co-expressing human SSTR5 with Gqi5 were developed using Jump-In technology from Thermo-Fisher. Gqi5 is the mouse G alpha q protein, that was modified to interact with Gi-coupled GPCRs as described previously (Coward, P.; Chan, S. D.; Wada, H. G.; Humphries, G. M.; Conklin, B. R. Chimeric G proteins Allow a High-Throughput Signaling Assay of Gi-Coupled Receptors. Anal Biochem. 1999, 270(2),242-248).

Co-expression of Gqi5 with SSTR5 allowed monitoring of SSTR5 activity by following IP1 accumulation. The assay was performed in a 384-well plate format using the IP1 assay kit from Cis-Bio in an antagonist mode, i.e., pre-incubation with antagonist following by receptor activation by agonist at a concentration generating 90% of full activation. Frozen cells expressing human SSTR5 were thawed, washed, and then plated in DMEM supplemented with 10% FBS and non-essential amino acids. 40 μL of 2.5×105 cells/mL were plated on a Poly D-Lysine coated 384-well white plate. The cells were then incubated for 16 hr. at 37° C/5% CO₂. After 16 hour the medium was removed, and 10 μL of stimulation buffer was added to the cells. Test compounds were dissolved in DMSO at at concentrations 2000-fold that of the final assay concentrations. 7.5 nLcompound solutions were transferred to the cell plates using a Labcyte Echo® acoustic liquid handler. The plates were then incubated for 15 minutes at 37° C/5%CO₂. After the first incubation, 5 μL of 30 nM SST28 were added to the cells, and the cells were incubated for 90 minutes at 37° C/5%CO₂. 5 μL of detection buffer (prepared as described in the IP-1 kit) was added to each well, and the plates were incubated at RT for 1 hour.

TR-FRET was measured using a ClarioSTAR plate reader, calculating the ratio between emissions at 665 nm and 620 nm (HTRF ratio). The HTRF ratio for positive (Max) and negative (Min) controls were used to normalize HTRF data and generate values for % inhibition. IC₅₀ and maximal inhibition values were determined using a standard 4-parameter fit.

The table below summarizes the assay data obtained for representative compounds.

| Cpd. | SSTR5 IC₅₀^{a} |
|---|---|
| 1 | ++ |
| 2 | ++ |
| 3 | − |
| 4 | ++ |
| 5 | ++ |
| 6 | ++ |
| 7 | +++ |
| 8 | + |
| 9 | ++ |
| 10 | +++ |
| 11 | +++ |
| 12 | − |
| 13 | − |
| 14 | − |
| 15 | + |
| 16 | − |
| 17 | + |
| 18 | ++ |
| 19 | ++ |
| 20 | +++ |
| 21 | − |
| 22 | + |
| 23 | − |
| 24 | − |
| 25 | ++ |
| 26 | ++ |
| 27 | − |
| 28 | − |
| 29 | ++ |
| 30 | − |
| 31 | − |
| 32 | − |
| 33 | − |
| 34 | + |
| 35 | ++ |
| 36 | ++ |
| 37 | + |
| 38 | ++ |
| 39 | ++ |
| 40 | ++ |
| 41 | − |
| 42 | + |
| 43 | − |
| 44 | ++ |
| 45 | − |
| 46 | − |
| 47 | ++ |
| 48 | ++ |
| 49 | ++ |
| 50 | ++ |
| 51 | ++ |
| 52 | ++ |
| 53 | ++ |
| 54 | +++ |
| 55 | ++ |
| 56 | +++ |
| 57 | +++ |
| 58 | +++ |
| 59 | +++ |
| 60 | +++ |
| 61 | +++ |
| 62 | +++ |
| 63 | +++ |
| 64 | +++ |
| 65 | +++ |
| 66 | +++ |
| 67 | ++ |
| 68 | ++ |
| 69 | +++ |
| 70 | +++ |
| 71 | +++ |
| 72 | +++ |
| 73 | +++ |
| 74 | +++ |
| 75 | ++ |
| 76 | ++ |
| 77 | − |
| 78 | − |
| 79 | ++ |
| 80 | +++ |
| 81 | +++ |
| 82 | +++ |
| 83 | +++ |
| 85 | +++ |
| 86 | +++ |
| 87 | +++ |
| 88 | ++ |
| 89 | ++ |
| 90 | +++ |
| 91 | +++ |
| 92 | +++ |
| 93 | + |
| 94 | +++ |
| 95 | − |
| 96 | ++ |
| 97 | − |
| 98 | ++ |
| 99 | ++ |

^{a}+++≤100 nM<++≤1000 nM<+≤5000 nM<−.

### Example A-2: Oral Bioavailability of the Compounds after Oral Dosing in Rat

Oral bioavailability of the compounds was determined in Sprague Dawley rats (Compound 90) or Wistar Han rats (Compound 81). The table below summarizes the results. Each compound was dosed intravenously (IV) at 1 mg/kg and orally (PO) 5 mg/kg using the respective vehicles listed below. The compounds display low (<10%) oral bioavailability (F%).

| Cpd | F % | IV vehicle | PO vehicle |
|---|---|---|---|
| 81 | 7.0% | 5% DMSO + 30% | 5% DMSO + 30% |
| | | PEG400 + 65% water | PEG400 + 65% water |
| 90 | 2.6% | 5% DMSO + 30% | 5% DMSO + 30% |
| | | PEG400 + 65% water | PEG400 + 65% water |

We claim:

1. A compound of Formula (I): 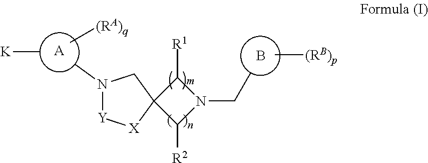
or a pharmaceutically acceptable salt, solvate, stereoisomer, or prodrug thereof, wherein:
X is —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —Z—NR⁶R⁷;
Z is *—(CH₂)ᵣ—C(═O)—, or *—(CH₂)ᵣ—S(═O)₂—, where * represents attachment to Ring A;
R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—;
or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NUR⁸, or C₁₋₈ alkyl which is unsubstituted or substituted by 1-6 R^{C} groups;
r is 0-4;
each s is independently 1-6;
each t is independently 1-6;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;
each R^{C} is independently —OH, —NH₂, —NH(R^{D}), —N(R^{D})₂, —N(R^{D})₃⁺, ═O, ═S, —C(═O)OH, 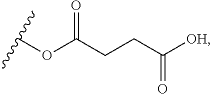 [SMILES: *OC(=O)CCC(=O)O]
G, or G¹;
each G is independently -S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂, 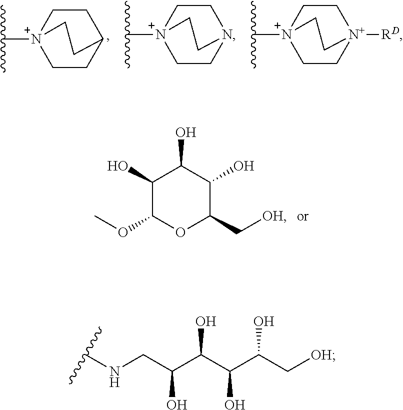
each G¹ is independently a 4- to 6-membered heterocycle which is unsubstituted or substituted with 1, 2, 3, or 4 substituents selected from C₁-C₆ alkyl, —O—(C₁-C₆ alkyl), —OH, ═O and ═S;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁C₆ fluoroalkyl), C₃-C₆ cycloalkyl, 3- to 6-membered heterocycloalkyl, and 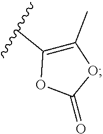 [SMILES: *c1oc(=O)oc1C]
or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)2, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 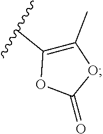 [SMILES: *c1oc(=O)oc1C]
m is 1 or 2;
n is 1 or 2;
p is 0-4; and
q is 0-4.

2. The compound of claim 1, or a pharmaceutically acceptable salt, solvate, stereoisomer, or prodrug thereof, wherein:
Ring B is phenyl or 6-membered heteroaryl;
each R¹ and R² is independently hydrogen or C₁₋₆ alkyl;
m is 2; and
n is 2.

3. The compound of claim 1, or a pharmaceutically acceptable salt, solvate, stereoisomer, or prodrug thereof, wherein the compound has the structure of Formula (Ia-1), or a pharmaceutically acceptable salt, solvate, stereoisomer, or prodrug thereof:

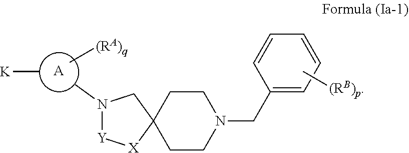

4. The compound of any one of claims 1-3, or a pharmaceutically acceptable salt, solvate, stereoisomer, or prodrug thereof, wherein:
X is —O—, and Y is —C(═O)—;
or X is —NR³—, and Y is —C(═O)—;
or X is —C(R⁴)₂—; and Y is —C(═O)—;
or X is —O—, and Y is —S(═O)₂—;
or X is —NR³—, and Y is —S(═O)₂—;
or X is —C(R⁴)₂—; and Y is —S(═O)₂—.

5. The compound of any one of claims 1-4, or a pharmaceutically acceptable salt, solvate, stereoisomer, or prodrug thereof, wherein:
X is —O—, and Y is —C(═O)—;
or X is —NR³—, and Y is —C(═O)—;
or X is —C(R⁴)₂—; and Y is —C(═O)—;
or X is —NR³—, and Y is —S(═O)₂—.

6. The compound of claim 1, or a pharmaceutically acceptable salt, solvate, stereoisomer, or prodrug thereof, wherein the compound has the structure of Formula (Ib), Formula (Ic), Formula (Id), or Formula (Ie), or a pharmaceutically acceptable salt, solvate, stereoisomer, or prodrug thereof:

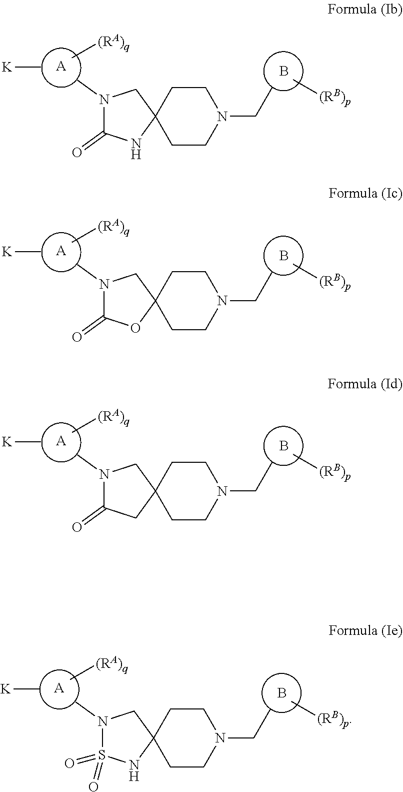

7. The compound of any one of claims 1-6, or a pharmaceutically acceptable salt, solvate, stereoisomer, or prodrug thereof, wherein:
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)_{2, —NR}⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
p is 1-4.

8. The compound of any one of claims 1-7, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —0—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl.

9. The compound of any one of claims 1-8, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, and C₁-C₆ fluoroalkyl.

10. The compound of claim 1, or a pharmaceutically acceptable salt, solvate, stereoisomer, or prodrug thereof, wherein the compound has the structure of Formula (If), or a pharmaceutically acceptable salt, solvate, stereoisomer, or prodrug thereof:

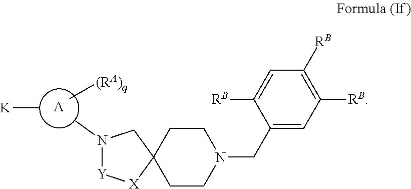

11. The compound of claim 1, or a pharmaceutically acceptable salt, solvate, stereoisomer, or prodrug thereof, wherein the compound has the structure of Formula (Ig), or a pharmaceutically acceptable salt, solvate, stereoisomer, or prodrug thereof:

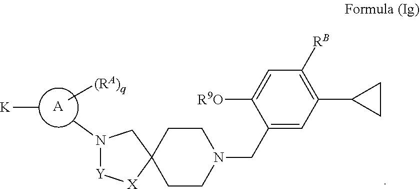

12. The compound of claim 11, or a pharmaceutically acceptable salt, solvate, stereoisomer, or prodrug thereof, wherein:
R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

13. The compound of any one of claims 1-12, or a pharmaceutically acceptable salt, solvate, stereoisomer, or prodrug thereof, wherein:
Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl;
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

14. The compound of any one of claims 1-12, or a pharmaceutically acceptable salt, solvate, stereoisomer, or prodrug thereof, wherein:
Ring A is phenyl, monocyclic C₃-C₆ cycloalkyl, or bridged cycloalkyl;
each R^{A} is independently halogen, —OH, —O—(C₁-C₆ alkyl), or C₁-C₆ alkyl; and
q is 0-2.

15. The compound of any one of claims 1-12, or a pharmaceutically acceptable salt, solvate, stereoisomer, or prodrug thereof, wherein:
Ring A is phenyl, cyclohexyl, or 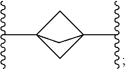 [SMILES: *C12CC(*)(C1)C2]
each R^{A} is independently halogen, —OH, —O—(C₁-C₆ alkyl), or C₁-C₆ alkyl; and
q is 0-2.

16. The compound of any one of claims 1-15, or a pharmaceutically acceptable salt, solvate, stereoisomer, or prodrug thereof, wherein:
Ring A is phenyl; and
q is 0.

17. The compound of any one of claims 1-12, or a pharmaceutically acceptable salt, solvate, stereoisomer, or prodrug thereof, wherein:
X is —O—, and Y is —C(═O)—.

18. The compound of claim 17, or a pharmaceutically acceptable salt, solvate, stereoisomer, or prodrug thereof, wherein:
Ring A is phenyl or heteroaryl.

19. The compound of claim 18, or a pharmaceutically acceptable salt, solvate, stereoisomer, or prodrug thereof, wherein:
Ring A is phenyl.

20. The compound of claim 17, or a pharmaceutically acceptable salt, solvate, stereoisomer, or prodrug thereof, wherein:
Ring A is monocyclic cycloalkyl, spirocyclic cycloalkyl, bridged cycloalkyl, monocyclic heterocycloalkyl, spirocyclic heterocycloalkyl, or bridged heterocycloalkyl.

21. The compound of claim 20, or a pharmaceutically acceptable salt, solvate, stereoisomer, or prodrug thereof, wherein:
Ring A is monocyclic C₃-C₆ cycloalkyl, or bridged cycloalkyl.

22. The compound of claim 21, or a pharmaceutically acceptable salt, solvate, stereoisomer, or prodrug thereof, wherein:
Ring A is cyclohexyl or

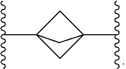 [SMILES: *C12CC(*)(C1)C2]

23. The compound of any one of claims 17-22, or a pharmaceutically acceptable salt, solvate, stereoisomer, or prodrug thereof, wherein:
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

24. The compound of any claim 23, or a pharmaceutically acceptable salt, solvate, stereoisomer, or prodrug thereof, wherein:
each R^{A} is independently halogen, —OH, —O—(C₁-C₆ alkyl), or C₁-C₆ alkyl.

25. The compound of claim 24, or a pharmaceutically acceptable salt, solvate, stereoisomer, or prodrug thereof, wherein:
each R^{A} is independently C₁-C₆ alkyl.

26. The compound of any one of claims 17-22, or a pharmaceutically acceptable salt, solvate, stereoisomer, or prodrug thereof, wherein:
q is 0.

27. The compound of any one of claims 1-16, or a pharmaceutically acceptable salt, solvate, stereoisomer, or prodrug thereof, wherein:
X is —NR³—, and Y is —C(═O)—;
or X is —C(R⁴)₂—; and Y is —C(═O)—;
or X is —O—, and Y is —S(═O)₂—;
or X is —NR³—, and Y is —S(═O)₂—;
or X is —C(R⁴)₂—; and Y is —S(═O)₂—.

28. The compound of claim 1, or a pharmaceutically acceptable salt, solvate, stereoisomer, or prodrug thereof, wherein the compound has the structure of Formula (Ih-1), or a pharmaceutically acceptable salt, solvate, stereoisomer, or prodrug thereof:

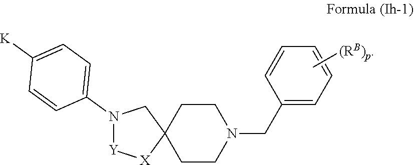

29. The compound of claim 1, or a pharmaceutically acceptable salt, solvate, stereoisomer, or prodrug thereof, wherein the compound has the structure of Formula (Ii), Formula (Ij), Formula (Ik), or Formula (Il), or a pharmaceutically acceptable salt, solvate, stereoisomer, or prodrug thereof:

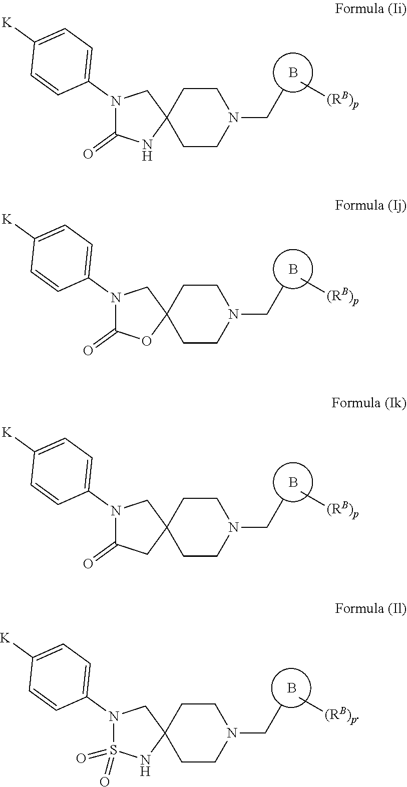

30. The compound of any one of claims 1-29, or a pharmaceutically acceptable salt, solvate, stereoisomer, or prodrug thereof, wherein:
Z is *—(CH₂)ᵣ—C(═O)—, or *—(CH₂)ᵣ—S(═O)₂—, where * represents attachment to Ring A; and
r is 0 or 1.

31. The compound of any one of claims 1-30, or a pharmaceutically acceptable salt, solvate, stereoisomer, or prodrug thereof, wherein:
Z is —C(═O)— or —S(═O)₂—.

32. The compound of any one of claims 1-31, or a pharmaceutically acceptable salt, solvate, stereoisomer, or prodrug thereof, wherein:
Z is —C(═O)—.

33. The compound of any one of claims 1-32, or a pharmaceutically acceptable salt, solvate, stereoisomer, or prodrug thereof, wherein:
R⁶ is hydrogen or C₁₋₆ alkyl which is unsubstituted or substituted by 1-6 R^{C} groups; and
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and
each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—.

34. The compound of any one of claims 1-33, or a pharmaceutically acceptable salt, solvate, stereoisomer, or prodrug thereof, wherein:
R⁶ is hydrogen or C₁₋₆ alkyl which is unsubstituted or substituted by 1-6 —OH groups;
R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups;
each V is independently —CH₂O—, —CH₂NR^{D}—, —CH₂N⁺(R^{D})₂—, —NH—C(═O)—NH—, or —C(═O)NH—;
s is 1-4;
t is 1-6;
R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂, 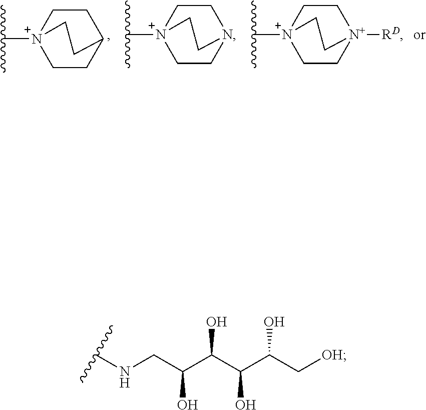
G¹ is 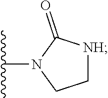 [SMILES: *N1CCNC1=O]
and
each R^{D} is independently C₁₋₆ alkyl.

35. The compound of any one of claims 1-34, or a pharmaceutically acceptable salt, solvate, stereoisomer, or prodrug thereof, wherein:
R⁶ is hydrogen;
R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl is substituted by 1-6 R^{C} groups;
each V is independently —CH₂O—, —CH₂NR^{D}—, —CH₂N⁺(R^{D})₂—, —NH—C(═O)—NH—, or —C(═O)NH—;
s is 1-4;
t is 1-3;
R⁸ is hydrogen or C₁₋₈ alkyl, wherein the alkyl is substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), 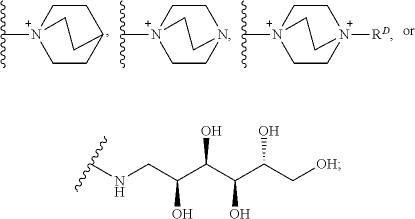
G¹ is 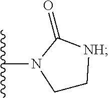 [SMILES: *N1CCNC1=O]
and
each R^{D} is independently C₁₋₆ alkyl.

36. The compound of any one of claims 1-35, or a pharmaceutically acceptable salt, solvate, stereoisomer, or prodrug thereof, wherein:
R⁶ is hydrogen;
R⁷ is C₁₋₈ alkyl which is substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), or 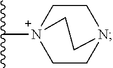 [SMILES: *[N+]12CCN(CC1)CC2]
G¹ is 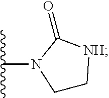 [SMILES: *N1CCNC1=O]
and
each R^{D} is independently C₁₋₆ alkyl.

37. The compound of any one of claims 1-32, or a pharmaceutically acceptable salt, solvate, stereoisomer, or prodrug thereof, wherein:
R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂, 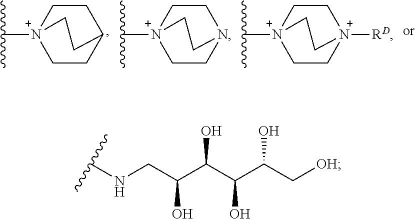
G¹ is 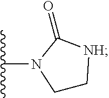 [SMILES: *N1CCNC1=O]
and
each R^{D} is independently C₁₋₆ alkyl.

38. The compound of claim 37, or a pharmaceutically acceptable salt, solvate, stereoisomer, or prodrug thereof, wherein:
R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen or C₁₋₈ alkyl which is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), 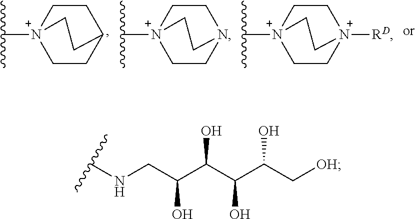
G¹ is 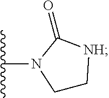 [SMILES: *N1CCNC1=O]
and
each R^{D} is independently C₁₋₆ alkyl.

39. The compound of claim 37 or 38, or a pharmaceutically acceptable salt, solvate, stereoisomer, or prodrug thereof, wherein:
R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen or C₁₋₈ alkyl which is unsubstituted or substituted by 1-6 R^{C} groups;
each R^{C} is independently —OH, —NH₂, —N(R^{D})₂, —N(R^{D})₃⁺, —C(═O)OH, or G;
each G is independently —S(═O)₂OH, —S(═O)₂NH₂, —P(═O)(OH)₂, or —P(═O)(OH)(R^{D}); and
each R^{D} is independently C₁₋₆ alkyl.

40. The compound of claim 1, or a pharmaceutically acceptable salt, solvate, stereoisomer, or prodrug thereof, wherein the compound has the structure of Formula (B), or a pharmaceutically acceptable salt, solvate, stereoisomer, or prodrug thereof:

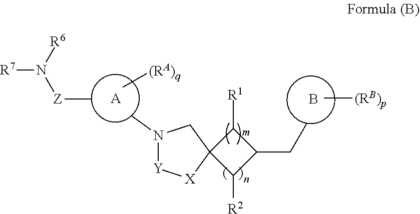

41. The compound of claim 40, or a pharmaceutically acceptable salt, solvate, stereoisomer, or prodrug thereof, wherein the compound has the structure of Formula (B4), or a pharmaceutically acceptable salt, solvate, stereoisomer, or prodrug thereof:

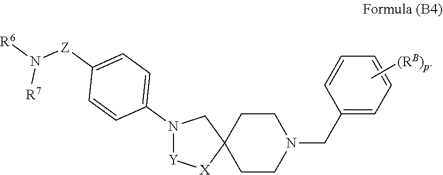

42. The compound of claim 1, wherein the compound is:
4-(8-((2,6-diethoxy-4′-fluoro-[1,1′-biphenyl]-4-yl)methyl)-2-oxo-1-oxa-3,8-diazaspiro[4.5]decan-3-yl)-N-(4-((2-hydroxyethyl)amino)-4-oxobutyl)benzamide;
4-(((2R,3R,4R,5S)-6-(4-(8-((2,6-diethoxy-4′-fluoro-[1,1′-biphenyl]-4-yl)methyl)-2-oxo-1-oxa-3,8-diazaspiro[4.5]decan-3-yl)benzamido)-2,3,4,5-tetrahydroxyhexyl)oxy)-4-oxobutanoic acid;
4-(8-((2-ethoxy-4′-fluoro-6-(hydroxymethyl)-[1,1′-biphenyl]-4-yl)methyl)-2-oxo-1-oxa-3,8-diazaspiro[4.5]decan-3-yl)-N-((2S,3R,4R,5R)-2,3,4,5,6-pentahydroxyhexyl)benzamide;
4-(8-((2,6-diethoxy-4′-fluoro-[1,1′-biphenyl]-4-yl)methyl)-2-oxo-1-oxa-3,8-diazaspiro[4.5]decan-3-yl)-N-((2S,3R,4R,5R)-2,3,4,5,6-pentahydroxyhexyl)benzamide;
4-(8-((2,6-diethoxy-4′-fluoro-[1,1′-biphenyl]-4-yl)methyl)-2-oxo-1-oxa-3,8-diazaspiro[4.5]decan-3-yl)-N-(4-((1,3-dihydroxy-2-(hydroxymethyl)propan-2-yl)amino)-4-oxobutyl)benzamide;
3-(4-(8-((2,6-diethoxy-4′-fluoro-[1,1′-biphenyl]-4-yl)methyl)-2-oxo-1-oxa-3,8-diazaspiro[4.5]decan-3-yl)benzamido)-N,N,N-trimethylpropan-1-aminium;
4-(8-((2,6-diethoxy-4′-fluoro-[1,1′-biphenyl]-4-yl)methyl)-2-oxo-1-oxa-3,8-diazaspiro[4.5]decan-3-yl)-N-(2-(3-(1,3-dihydroxy-2-(hydroxymethyl)propan yl)ureido)ethyl)benzamide;
3-(1-(4-(8-((2,6-diethoxy-4′-fluoro-[1,1′-biphenyl]-4-yl)methyl)-2-oxo-1-oxa-3,8-diazaspiro[4.5]decan-3-yl)benzoyl)azetidine-3-carboxamido)-N,N,N-trimethylpropan-1-aminium;
(4-(8-((2,6-diethoxy-4′-fluoro-[1,1′-biphenyl]-4-yl)methyl)-2-oxo-1-oxa-3,8-diazaspiro[4.5]decan-3-yl)benzoyl)-L-aspartic acid;
4-(4-(8-((2,6-diethoxy-4′-fluoro-[1,1′-biphenyl]-4-yl)methyl)-2-oxo-1-oxa-3,8-diazaspiro[4.5]decan-3-yl)benzamido)butane-1-sulfonic acid;
3-(4-(8-((2,6-diethoxy-4′-fluoro-[1,1′-biphenyl]-4-yl)methyl)-2-oxo-1-oxa-3,8-diazaspiro[4.5]decan-3-yl)benzamido)propane-1-sulfonic acid;
2-(4-(8-((2,6-diethoxy-4′-fluoro-[1,1′-biphenyl]-4-yl)methyl)-2-oxo-1-oxa-3,8-diazaspiro[4.5]decan-3-yl)benzamido)ethane-1-sulfonic acid;
methyl 2-cyclopropyl-5-ethoxy-4-((2-(4-((2-(3-(2-hydroxyethyl)ureido)ethyl)carbamoyl)phenyl)-3-oxo-2,8-diazaspiro[4.5]decan-8-yl)methyl)benzoate;
methyl 2-cyclopropyl-4-((2-(4-(2-((2-(3-(1,3-dihydroxy-2-(hydroxymethyl)propan-2-yl)ureido)ethyl)amino)-2-oxoethyl)phenyl)-3-oxo-2,8-diazaspiro[4.5]decan-8-yl)methyl)-5-ethoxybenzoate;
methyl 2-cyclopropyl-5-ethoxy-4-((2-(4-((4-((2-hydroxyethyl)amino)-4-oxobutyl)carbamoyl)phenyl)-3-oxo-2,8-diazaspiro[4.5]decan-8-yl)methyl)benzoate;
methyl 2-cyclopropyl-4-((2-(4-((2-(3-(1,3-dihydroxypropan-2-yl)ureido)ethyl)carbamoyl)phenyl)-3-oxo-2,8-diazaspiro[4.5]decan-8-yl)methyl)-5-ethoxybenzoate;
methyl 2-cyclopropyl-4-((2-(4-(44(14(2-(dimethylamino)ethyl)amino)-2-methyl-1-oxopropan-2-yl)amino)-4-oxobutyl)phenyl)-3-oxo-2,8-diazaspiro[4.5]decan-8-yl)methyl)-5-ethoxybenzoate;
methyl 2-cyclopropyl-4-((2-(4-((2-(3-(1,3-dihydroxy-2-(hydroxymethyl)propan-2-yl)ureido)ethyl)carbamoyl)phenyl)-3-oxo-2,8-diazaspiro[4.5]decan-8-yl)methyl)-5-ethoxybenzoate;
methyl 2-cyclopropyl-4-((2-(4-((1,3-dihydroxypropan-2-yl)carbamoyl)phenyl) oxo-2,8-diazaspiro[4.5]decan-8-yl)methyl)-5-ethoxybenzoate;
methyl 2-cyclopropyl-5-ethoxy-4-((2-(4-((2-hydroxyethyl)carbamoyl)phenyl)-3-oxo-2,8-diazaspiro[4.5]decan-8-yl)methyl)benzoate;
methyl 2-cyclopropyl-4-((2-(4-((1,3-dihydroxy-2-(hydroxymethyl)propan yl)carbamoyl)phenyl)-3-oxo-2,8-diazaspiro[4.5]decan-8-yl)methyl)-5-ethoxybenzoate;
2-(4-(8-(5-cyclopropyl-2-ethoxy-4-(methoxycarbonyl)benzyl)-3-oxo-2,8-diazaspiro[4.5]decan-2-yl)benzamido)-N,N,N-trimethylethan-1-aminium;
4-(4-(8-(5-cyclopropyl-2-ethoxy-4-(methoxycarbonyl)benzyl)-3-oxo-2,8-diazaspiro[4.5]decan-2-yl)benzamido)butanoic acid;
(5-methyl-2-oxo-1,3-dioxo1-4-yl)methyl 2-cyclopropyl-5-ethoxy-4-((3-oxo-2-(4-(((2S,3R,4R,5R)-2,3,4,5,6-pentahydroxyhexyl)carbamoyl)phenyl)-2,8-diazaspiro[4.5]decan-8-yl)methyl)benzoate;
3-(4-(8-(5-cyclopropyl-2-ethoxy-4-(methoxycarbonyl)benzyl)-3-oxo-2,8-diazaspiro[4.5]decan-2-yl)benzamido)propanoic acid;
methyl 2-cyclopropyl-5-ethoxy-4-((2-(4-((2-(2-hydroxyethoxy)ethyl)carbamoyl)phenyl)-3-oxo-2,8-diazaspiro[4.5]decan-8-yl)methyl)benzoate;
methyl 2-cyclopropyl-5-ethoxy-4-((2-(4-((3-hydroxypropyl)carbamoyl)phenyl)-3-oxo-2,8-diazaspiro[4.5]decan-8-yl)methyl)benzoate;
3-(4-(8-(5-cyclopropyl-2-ethoxy-4-(methoxycarbonyl)benzyl)-3-oxo-2,8-diazaspiro[4.5]decan-2-yl)benzamido)-N,N,N-trimethylpropan-1-aminium;
2-(4-(8-(5-cyclopropyl-2-ethoxy-4-(methoxycarbonyl)benzyl)-3-oxo-2,8-diazaspiro[4.5]decan-2-yl)benzamido)ethane-1-sulfonic acid;
3-(4-(8-(5-cyclopropyl-2-ethoxy-4-(methoxycarbonyl)benzyl)-3-oxo-2,8-diazaspiro[4.5]decan-2-yl)benzamido)propane-1-sulfonic acid;
4-(4-(8-(5-cyclopropyl-2-ethoxy-4-(methoxycarbonyl)benzyl)-3-oxo-2,8-diazaspiro[4.5]decan-2-yl)benzamido)butane-1-sulfonic acid;
(3-(4-(8-(5-cyclopropyl-2-ethoxy-4-(methoxycarbonyl)benzyl)-3-oxo-2,8-diazaspiro[4.5]decan-2-yl)benzamido)propyl)phosphonic acid;
(3-(4-(8-(5-cyclopropyl-2-ethoxy-4-(methoxycarbonyl)benzyl)-3-oxo-2,8-diazaspiro[4.5]decan-2-yl)benzamido)propyl)(methyl)phosphinic acid;
methyl 4-((2-(4-(bis(2-hydroxyethyl)carbamoyl)phenyl)-3-oxo-2,8-diazaspiro[4.5]decan-8-yl)methyl)-2-cyclopropyl-5-ethoxybenzoate;
methyl (S)-2-cyclopropyl-4-((2-(4-((2,3-dihydroxypropyl)carbamoyl)phenyl)-3-oxo-2,8-diazaspiro[4.5]decan-8-yl)methyl)-5-ethoxybenzoate;
methyl (R)-2-cyclopropyl-4-((2-(4-((2,3-dihydroxypropyl)carbamoyl)phenyl)-3-oxo-2,8-diazaspiro[4.5]decan-8-yl)methyl)-5-ethoxybenzoate;
methyl 2-cyclopropyl-5-ethoxy-4-((3-oxo-2-(4-(((2S,3R,4R,5R)-2,3,4,5,6-pentahydroxyhexyl)carbamoyl)phenyl)-2,8-diazaspiro[4.5]decan-8-yl)methyl)benzoate;
methyl 2-cyclopropyl-5-ethoxy-4-((3-oxo-2-(4-((2-ureidoethyl)carbamoyl)phenyl)-2,8-diazaspiro[4.5]decan-8-yl)methyl)benzoate;
methyl 2-cyclopropyl-5-ethoxy-4-((3-oxo-2-(4-((3-ureidopropyl)carbamoyl)phenyl)-2,8-diazaspiro[4.5]decan-8-yl)methyl)benzoate;
methyl 2-cyclopropyl-5-ethoxy-4-((3-oxo-2-(4-((2-(2-oxoimidazolidin-1-yl)ethyl)carbamoyl)phenyl)-2,8-diazaspiro[4.5]decan-8-yl)methyl)benzoate;
4-(4-(8-(5-cyclopropyl-2-ethoxy-4-(methoxycarbonyl)benzyl)-3-oxo-2,8-diazaspiro[4.5]decan-2-yl)-N-methylbenzamido)butanoic acid;
methyl 4-((2-(4-(3,3-bis(hydroxymethyl)azetidine-1-carbonyl)phenyl)-3-oxo-2,8-diazaspiro[4.5]decan-8-yl)methyl)-2-cyclopropyl-5-ethoxybenzoate;
methyl 2-cyclopropyl-4-((2-(4-((3S,4S)-3,4-dihydroxypyrrolidine-1-carbonyl)phenyl)-3-oxo-2,8-diazaspiro[4.5]decan-8-yl)methyl)-5-ethoxybenzoate;
methyl 2-cyclopropyl-5-ethoxy-4-((3-oxo-2-(4-((3-sulfamoylpropyl)carbamoyl)phenyl)-2,8-diazaspiro[4.5]decan-8-yl)methyl)benzoate;
1-(2-(2-(4-(8-(5-cyclopropyl-2-ethoxy-4-(methoxycarbonyl)benzyl)-3-oxo-2,8-diazaspiro[4.5]decan-2-yl)benzamido)ethoxy)ethyl)-1,4-diazabicyclo[2.2.2]octan-1-ium;
methyl 4-((2-(4-((3-aminopropyl)carbamoyl)phenyl)-3-oxo-2,8-diazaspiro[4.5]decan-8-yl)methyl)-2-cyclopropyl-5-ethoxybenzoate;
(4-(4-(8-(5-cyclopropyl-2-ethoxy-4-(methoxycarbonyl)benzyl)-3-oxo-2,8-diazaspiro[4.5]decan-2-yl)benzamido)butyl)phosphonic acid;
methyl 2-cyclopropyl-4-((2-(4-((3,4-dihydroxybutyl)carbamoyl)phenyl)-3-oxo-2,8-diazaspiro[4.5]decan-8-yl)methyl)-5-ethoxybenzoate;
methyl 2-cyclopropyl-5-ethoxy-4-((2-(4-((2-(2-(2-hydroxyethoxy)ethoxy)ethyl)carbamoyl)phenyl)-3-oxo-2,8-diazaspiro[4.5]decan-8-yl)methyl)benzoate;
methyl 2-cyclopropyl-5-ethoxy-4-((3-oxo-2-(4-((2-sulfamoylethyl)carbamoyl)phenyl)-2,8-diazaspiro[4.5]decan-8-yl)methyl)benzoate;
methyl 2-cyclopropyl-5-ethoxy-4-((3-oxo-2-(4-((4-sulfamoylbutyl)carbamoyl)phenyl)-2,8-diazaspiro[4.5]decan-8-yl)methyl)benzoate;
methyl 2-cyclopropyl-5-ethoxy-4-((3-oxo-2-(4-(4-sulfamoylpiperidine-1-carbonyl)phenyl)-2,8-diazaspiro[4.5]decan-8-yl)methyl)benzoate;
methyl 2-cyclopropyl-5-ethoxy-4-((3-oxo-2-(4-(4-(sulfamoylmethyl)piperidine-1-carbonyl)phenyl)-2,8-diazaspiro[4.5]decan-8-yl)methyl)benzoate;
methyl 2-cyclopropyl-5-ethoxy-4-((2-oxo-3-(4-(((2S,3R,4R,5R)-2,3,4,5,6-pentahydroxyhexyl)carbamoyl)phenyl)-1-oxa-3,8-diazaspiro[4.5]decan yl)methyl)benzoate;
methyl 2-cyclopropyl-5-ethoxy-4-((2-(4-((3-guanidinopropyl)carbamoyl)phenyl)-3-oxo-2,8-diazaspiro[4.5]decan-8-yl)methyl)benzoate;
methyl 2-cyclopropyl-5-ethoxy-4-((3-(4-((2-(2-hydroxyethoxy)ethyl)carbamoyl)phenyl)-2-oxo-1-oxa-3,8-diazaspiro[4.5]decan-8-yl)methyl)benzoate;
methyl 2-cyclopropyl-5-ethoxy-4-((2-oxo-3-(4-((3-ureidopropyl)carbamoyl)phenyl)-1-oxa-3,8-diazaspiro[4.5]decan-8-yl)methyl)benzoate;
3-(4-(8-(5-cyclopropyl-2-ethoxy-4-(methoxycarbonyl)benzyl)-2-oxo-1-oxa-3,8-diazaspiro[4.5]decan-3-yl)benzamido)propane-1-sulfonic acid;
methyl 2-cyclopropyl-5-ethoxy-4-((2-oxo-3-(4-((3-sulfamoylpropyl)carbamoyl)phenyl)-1-oxa-3,8-diazaspiro[4.5]decan-8-yl)methyl)benzoate;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-((2S,3R,4R,5R)-2,3,4,5,6-pentahydroxyhexyl)benzamide;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(2-(2-hydroxyethoxy)ethyl)benzamide;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(3-ureidopropyl)benzamide;
3-(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzamido)propane-1-sulfonic acid;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(3-sulfamoylpropyl)benzamide;
4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzamide;
3-(4-(8-((2-cyclopropyl-5-ethoxy-4′-fluoro-[1,1′-biphenyl]-4-yl)methyl)-2-oxo-1,3,8-triazaspiro[4.5]decan-3-yl)benzamido)propane-1-sulfonic acid;
4-(8-(5-cyclopropyl-2-ethoxy-4-(3-methyl-1,2,4-oxadiazol-5-yl)benzyl)-2-oxo-1,3,8-triazaspiro[4.5]decan-3-yl)-N-(2-(2-hydroxyethoxy)ethyl)benzamide;
3-(4-(8-(5-cyclopropyl-2-ethoxy-4-(3-methyl-1,2,4-oxadiazol-5-yl)benzyl)-2-oxo-1,3,8-triazaspiro[4.5]decan-3-yl)benzamido)propane-1-sulfonic acid;
4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-hydroxyethyl)benzamide;
4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-(2-hydroxyethoxy)ethoxy)ethyl)benzamide;
2-(4-(8-((2-cyclopropyl-5-ethoxy-4′-fluoro-[1,1′-biphenyl]-4-yl)methyl)-2-oxo-1,3,8-triazaspiro[4.5]decan-3-yl)benzamido)ethane-1-sulfonic acid;
4-(8-((2-cyclopropyl-5-ethoxy-2′,4′-difluoro-[1,1′-biphenyl]-4-yl)methyl)-2-oxo-1,3,8-triazaspiro[4.5]decan-3-yl)-N-(2-(2-hydroxyethoxy)ethyl)benzamide;
4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-((1,3-dihydroxypropan-2-yl)oxy)ethyl)benzamide;
4-(8-(5-cyclopropyl-2-ethoxy-4-(5-fluoropyridin-2-yl)benzyl)-2-oxo-1,3,8-triazaspiro[4.5]decan-3-yl)-N-(2-(2-hydroxyethoxy)ethyl)benzamide;
methyl 2-cyclopropyl-5-ethoxy-4-((3-(4-((2-hydroxyethyl)carbamoyl)phenyl)-2-oxo-1,3,8-triazaspiro[4.5]decan-8-yl)methyl)benzoate;
methyl 2-cyclopropyl-5-ethoxy-4-((3-(4-((2-(2-hydroxyethoxy)ethyl)carbamoyl)phenyl)-2-oxo-1,3,8-triazaspiro[4.5]decan-8-yl)methyl)benzoate;
methyl 2-cyclopropyl-5-ethoxy-4-((2-oxo-3-(4-((3-ureidopropyl)carbamoyl)phenyl)-1,3,8-triazaspiro[4.5]decan-8-yl)methyl)benzoate;
3-(4-(8-(5-cyclopropyl-2-ethoxy-4-(methoxycarbonyl)benzyl)-2-oxo-1,3,8-triazaspiro[4.5]decan-3-yl)benzamido)propane-1-sulfonic acid;
methyl 2-cyclopropyl-5-ethoxy-4-((2-oxo-3-(4-((3-sulfamoylpropyl)carbamoyl)phenyl)-1,3,8-triazaspiro[4.5]decan-8-yl)methyl)benzoate;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(3-guanidinopropyl)benzenesulfonamide;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(2-(2-hydroxyethoxy)ethyl)benzenesulfonamide;
4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)benzenesulfonamide;
4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(2-(2-(2-hydroxyethoxy)ethoxy)ethyl)benzenesulfonamide;
8-[[5-cyclopropyl-2-ethoxy-4-(4-fluorophenyl)phenyl]methyl]-3-[4-[3-[[[(2S,3R,4R,5R)-2,3,4,5,6-pentahydroxyhexyl]amino]methyl]azetidin-1-yl]sulfonylphenyl]-1-oxa-3,8-diazaspiro[4.5]decan-2-one;
4-(8-((2-cyclopropyl-5-ethoxy-4′-fluoro-[1,1′-biphenyl]-4-yl)methyl)-2-oxo-1,3,8-triazaspiro[4.5]decan-3-yl)-N-(2-(2-hydroxyethoxy)ethyl)benzenesulfonamide;
4-(8-(5-cyclopropyl-2-ethoxy-4-(methylsulfonyl)benzyl)-2-oxo-1,3,8-triazaspiro[4.5]decan-3-yl)-N,N-bis(4-methoxybenzyl)benzenesulfonamide;
4-(8-((2-cyclopropyl-5-ethoxy-4′-fluoro-[1,1′-biphenyl]-4-yl)methyl)-2,2-dioxido-2-thia-1,3,8-triazaspiro[4.5]decan-3-yl)-N-(2-(2-hydroxyethoxy)ethyl)benzamide;
(1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexane-1-carboxamide;
3-((1s,4s)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexane-1-carboxamido)propane-1-sulfonic acid;
(1r,4r)-4-(8-(5-cyclopropyl-2-ethoxy-4-(5-fluoropyridin-2-yl)benzyl)-2-oxo-1-oxa-3,8-diazaspiro[4.5]decan-3-yl)-N-(2-(2-hydroxyethoxy)ethyl)-1-methylcyclohexane-1-carboxamide;
3-((1r,4r)-4-(8-(5-cyclopropyl-2-ethoxy-4-(5-fluoropyridin-2-yl)benzyl)-2-oxo-1-oxa-3,8-diazaspiro[4.5]decan-3-yl)-1-methylcyclohexane-1-carboxamido)propane-1-sulfonic acid;
3-(8-((2-cyclopropyl-5-ethoxy-4′-fluoro-[1,1′-biphenyl]-4-yl)methyl)-2-oxo-1-oxa-3,8-diazaspiro[4.5]decan-3-yl)-N-(2-(2-hydroxyethoxy)ethyl)bicyclo[1.1.1]pentane-1-carboxamide;
or a pharmaceutically acceptable salt, solvate, stereoisomer, or prodrug thereof.

43. A compound of Formula (II): 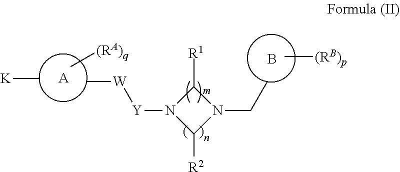
or a pharmaceutically acceptable salt, solvate, stereoisomer, or prodrug thereof, wherein:
W is a bond, —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —Z—NR⁶R⁷;
Z is *—(CH₂)ᵣ—C(═O)—, or *—(CH₂)ᵣ—S(═O)₂—, where * represents attachment to Ring A;
R⁶ is hydrogen, C₁₋₆ alkyl, C₁₋₆ fluoroalkyl, C₃₋₆ cycloalkyl, or benzyl wherein the alkyl, fluoroalkyl, cycloalkyl, or benzyl is unsubstituted or substituted by 1-6 R^{C} groups;
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups;
each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—;
or R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
r is 0-4;
each s is independently 1-6;
each t is independently 1-6;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
R⁸ is hydrogen, C₁₋₈ alkyl, C₂₋₈ alkenyl, C₂₋₈ alkynyl, C₃₋₈ cycloalkyl, C₅₋₈ cycloalkenyl, or 3- to 8-membered heterocycloalkyl, wherein the alkyl, alkenyl, alkynyl, cycloalkyl, cycloalkenyl, or heterocycloalkyl is unsubstituted or substituted by 1-6 R^{C} groups;
each R^{C} is independently —OH, —NH₂, —NH(R^{D}), —N(R^{D})₂, —N(R^{D})₃⁺, —C(═O)OH, 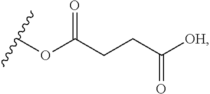 [SMILES: *OC(=O)CCC(=O)O]
G, or G¹;
each G is independently —S(═O)₂OH, —S(═O)OH, —S(═O)₂NH₂, —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂, 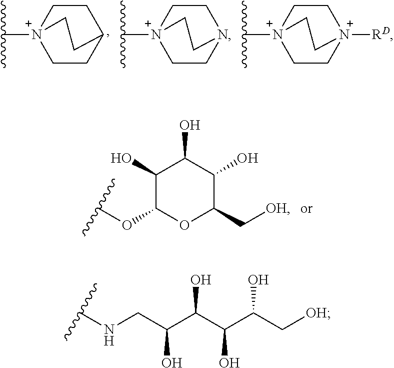
each G¹ is independently a 4- to 6-membered heterocycle which is unsubstituted or substituted with 1, 2, 3, or 4 substituents selected from C₁-C₆ alkyl, —O—(C₁-C₆ alkyl), —OH, ═O and ═S;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 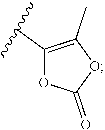 [SMILES: *c1oc(=O)oc1C]
or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 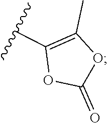 [SMILES: *c1oc(=O)oc1C]
m is 1 or 2;
n is 1 or 2;
p is 1-4; and
q is 0-4.

44. The compound of claim 43, or a pharmaceutically acceptable salt, solvate, stereoisomer, or prodrug thereof, wherein:
Ring B is phenyl or 6-membered heteroaryl;
each R^{l} and R² is independently hydrogen or C₁₋₆ alkyl;
m is 2; and
n is 2.

45. The compound of claim 43, or a pharmaceutically acceptable salt, solvate, stereoisomer, or prodrug thereof, wherein the compound has the structure of Formula (IIa-1), or a pharmaceutically acceptable salt, solvate, stereoisomer, or prodrug thereof:

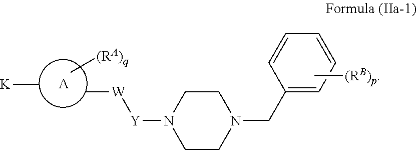

46. The compound of any one of claims 43-45, or a pharmaceutically acceptable salt, solvate, stereoisomer, or prodrug thereof, wherein:
W is a bond, and Y is —C(═O)—;
or W is —O—, and Y is —C(═O)—;
or W is —NR³—, and Y is —C(═O)—;
or W is —C(R⁴)₂—; and Y is —C(═O)—;
or W is a bond, and Y is —S(═O)₂—;
or W is —O—, and Y is —S(═O)₂—;
or W is —NR³—, and Y is —S(═O)₂—;
or W is —C(R⁴)₂—; and Y is —S(═O)₂—.

47. The compound of any one of claims 43-46, or a pharmaceutically acceptable salt, solvate, stereoisomer, or prodrug thereof, wherein:
W is —O—, and Y is —C(═O)—;
or W is —NR³—, and Y is —C(═O)—;
or W is —C(R⁴)₂—; and Y is —C(═O)—;
or W is a bond, and Y is —C(═O)—.

48. The compound of claim 43, or a pharmaceutically acceptable salt, solvate, stereoisomer, or prodrug thereof, wherein the compound has the structure of Formula (IIb), Formula (IIc), Formula (IId), or Formula (IIe), or a pharmaceutically acceptable salt, solvate, stereoisomer, or prodrug thereof:

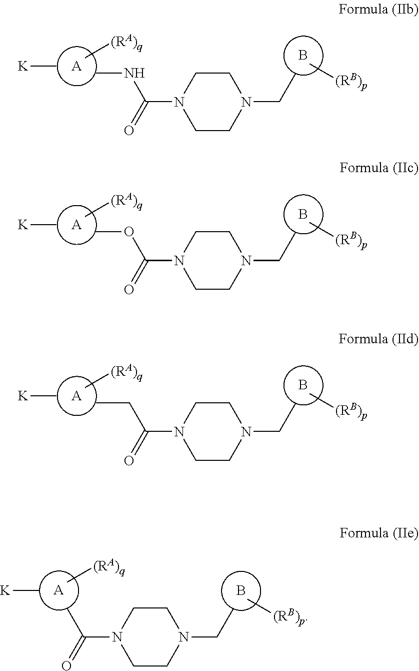

49. The compound of any one of claims 43-48, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl.

50. The compound of any one of claims 43-49, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

51. The compound of claim 50, or a pharmaceutically acceptable salt, solvate, stereoisomer, or prodrug thereof, wherein the compound has the structure of Formula (IIf), or a pharmaceutically acceptable salt, solvate, stereoisomer, or prodrug thereof:

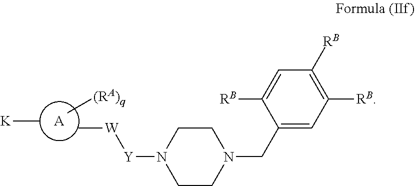

52. The compound of claim 50, or a pharmaceutically acceptable salt, solvate, stereoisomer, or prodrug thereof, wherein the compound has the structure of Formula (IIg), or a pharmaceutically acceptable salt, solvate, stereoisomer, or prodrug thereof:

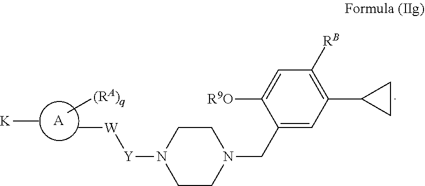

53. The compound of claim 52, or a pharmaceutically acceptable salt, solvate, stereoisomer, or prodrug thereof, wherein:
R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

54. The compound of any one of claims 43-53, or a pharmaceutically acceptable salt, solvate, stereoisomer, or prodrug thereof, wherein:
Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl;
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

55. The compound of any one of claims 43-53, or a pharmaceutically acceptable salt, solvate, stereoisomer, or prodrug thereof, wherein:
Ring A is phenyl, monocyclic C₃-C₆ cycloalkyl, or bridged cycloalkyl;
each R^{A} is independently halogen, —OH, —O—(C₁-C₆ alkyl), or C₁-C₆ alkyl; and
q is 0-2.

56. The compound of any one of claims 43-53, or a pharmaceutically acceptable salt, solvate, stereoisomer, or prodrug thereof, wherein:
Ring A is phenyl, cyclohexyl, or 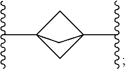 [SMILES: *C12CC(*)(C1)C2]
each R^{A} is independently halogen, —OH, —O—(C₁-C₆ alkyl), or C₁-C₆ alkyl; and
q is 0-2.

57. The compound of any one of claims 43-53, or a pharmaceutically acceptable salt, solvate, stereoisomer, or prodrug thereof, wherein:
Ring A is phenyl; and
q is 0.

58. The compound of claim 43, or a pharmaceutically acceptable salt, solvate, stereoisomer, or prodrug thereof, wherein the compound has the structure of Formula (IIh), or a pharmaceutically acceptable salt, solvate, stereoisomer, or prodrug thereof:

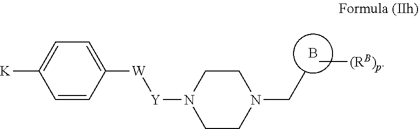

59. The compound of any one of claims 43-58, or a pharmaceutically acceptable salt, solvate, stereoisomer, or prodrug thereof, wherein:
Z is *—(CH₂)ᵣ—C(═O)—, or *—(CH₂)ᵣ—S(═O)₂—, where * represents attachment to Ring A; and
r is 0 or 1.

60. The compound of any one of claims 43-59, or a pharmaceutically acceptable salt, solvate, stereoisomer, or prodrug thereof, wherein:
Z is —C(═O)— or —S(═O)₂—.

61. The compound of any one of claims 43-60, or a pharmaceutically acceptable salt, solvate, stereoisomer, or prodrug thereof, wherein:
Z is —C(═O)—.

62. The compound of any one of claims 43-61, or a pharmaceutically acceptable salt, solvate, stereoisomer, or prodrug thereof, wherein:
R⁶ is hydrogen or C₁₋₆ alkyl which is unsubstituted or substituted by 1-6 R^{C} groups; and
R⁷ is C₁₋₈ alkyl, C₂₋₈ alkenyl, C₂₋₈ alkynyl, benzyl, C₃₋₈ cycloalkyl, C₅₋₈ cycloalkenyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein each alkyl, alkenyl, alkynyl, benzyl, cycloalkyl, cycloalkenyl, and heterocycloalkyl is substituted by 1-6 R^{C} groups; and
each V is independently —CH₂O—, —CH₂NR^{D}—, —CH₂N⁺(R^{D})₂—, —NH—C(═O)—NH—, —C(═O)NH—, —CH₂S(═O)₂—, or —CH₂S(═O)—.

63. The compound of any one of claims 43-62, or a pharmaceutically acceptable salt, solvate, stereoisomer, or prodrug thereof, wherein:
R⁶ is hydrogen or C₁₋₆ alkyl which is unsubstituted or substituted by 1-6 —OH groups;
R⁷ is C₁₋₈ alkyl, C₃₋₈ cycloalkyl, 3- to 8-membered heterocycloalkyl, —[(CH₂)ₛ—V]ₜ—R⁸, —[(CHR^{D})ₛ—V]ₜ—R⁸, or —[(C(R^{D})₂)ₛ—V]ₜ—R⁸; wherein the alkyl, cycloalkyl, or 3- to 8-membered heterocycloalkyl is substituted by 1-6 R^{C} groups;
each V is independently —CH₂O—, —CH₂NR^{D}—, —CH₂N⁺(R^{D})₂—, —NH—C(═O)—NH—, or —C(═O)NH—;
s is 1-4;
t is 1-6;
R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂, 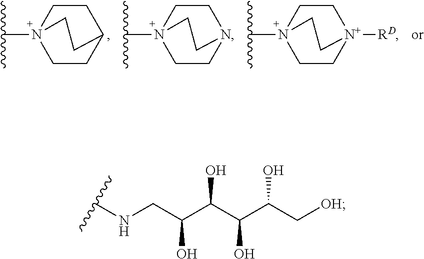
G¹ is 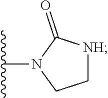 [SMILES: *N1CCNC1=O]
and
each R^{D} is independently C₁₋₆ alkyl.

64. The compound of any one of claims 43-63, or a pharmaceutically acceptable salt, solvate, stereoisomer, or prodrug thereof, wherein:
R⁶ is hydrogen;
R⁷ is C₁₋₈ alkyl, —[(CH₂)ₛ—V]ₜ—R⁸, —[(CHR^{D})ₛ—V]ₜ—R⁸, or —[(C(R^{D})₂)ₛ—V]ₜ—R⁸;
wherein the alkyl is substituted by 1-6 R^{C} groups;
each V is independently —CH₂O—, —CH₂NR^{D}—, —CH₂N⁺(R^{D})₂—, —NH—C(═O)—NH—, or —C(═O)NH—;
s is 1-4;
t is 1-3;
R⁸ is hydrogen or C₁₋₈ alkyl, wherein the alkyl is substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), 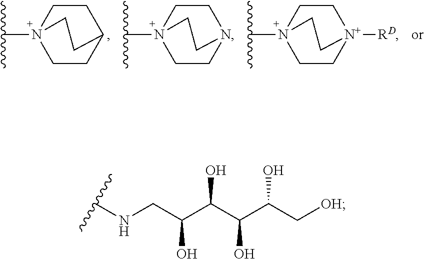
G¹ is 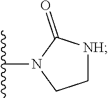 [SMILES: *N1CCNC1=O]
and
each R^{D} is independently C₁₋₆ alkyl.

65. The compound of any one of claims 43-64, or a pharmaceutically acceptable salt, solvate, stereoisomer, or prodrug thereof, wherein:
R⁶ is hydrogen;
R⁷ is C₁₋₈ alkyl which is substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), or 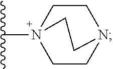
G¹ is 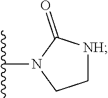 [SMILES: *N1CCNC1=O]
and
each R^{D} is independently C₁₋₆ alkyl.

66. The compound of any one of claims 43-61, or a pharmaceutically acceptable salt, solvate, stereoisomer, or prodrug thereof, wherein:
R⁶ and R⁷ are taken together with the nitrogen to which they are attached to form a 3- to 8-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen, C₁₋₈ alkyl, C₃₋₈ cycloalkyl, or 3- to 8-membered heterocycloalkyl, wherein the alkyl, cycloalkyl, or 3- to 8-membered heterocycloalkyl is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)OH, —S(═O)₂NH₂, —P(═O)(OH)₂, —P(═O)(OH)(R^{D}), —P(═O)(OH)(OR^{D}), —N(R^{D})CONHS(═O)₂(R^{D}), —C(═O)NHS(═O)₂(R^{D}), —NHC(═O)NH₂, —NHC(═NH)NH₂, 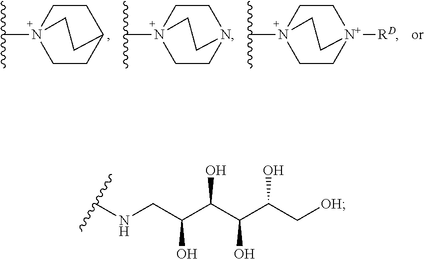
G¹ is 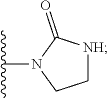 [SMILES: *N1CCNC1=O]
and
each R^{D} is independently C₁₋₆ alkyl.

67. The compound of claim 66, or a pharmaceutically acceptable salt, solvate, stereoisomer, or prodrug thereof, wherein:
R⁶ and R⁷ are taken together with the nitrogen to which they are attached to form a 4- to 6-membered heterocycloalkyl, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen or C₁₋₈ alkyl which is unsubstituted or substituted by 1-6 R^{C} groups;
each R^{C} is independently —OH, —NH₂, —N(R^{D})₂, —N(R^{D})₃⁺, —C(═O)OH, G, or G¹;
each G is independently —S(═O)₂OH, —S(═O)₂NH₂, —P(═O)(OH)₂, —P(═O)(OH)(R^{D}), 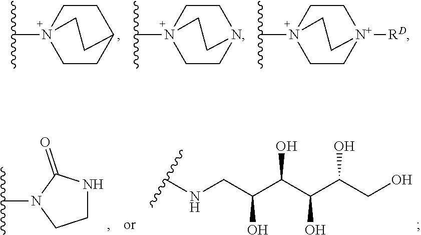
G¹ is 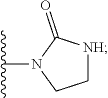 [SMILES: *N1CCNC1=O]
and
each R^{D} is independently C₁₋₆ alkyl.

68. The compound of claim 66 or 67, or a pharmaceutically acceptable salt, solvate, stereoisomer, or prodrug thereof, wherein:
R⁶ and R⁷ are taken together with the nitrogen to which they are attached to form an azetidine or a piperidine, which is substituted by 1-6 groups selected from R^{C}, —C(═O)NHR⁸, —CH₂NHR⁸, or C₁₋₈ alkyl which is unsubstituted or substituted by 1-6 R^{C} groups;
R⁸ is hydrogen or C₁₋₈ alkyl which is unsubstituted or substituted by 1-6 R^{C} groups;
each R^{C} is independently —OH, —NH₂, —N(R^{D})₂, —N(R^{D})₃⁺, —C(═O)OH, or G;
each G is independently —S(═O)₂OH, —S(═O)₂NH₂, —P(═O)(OH)₂, or —P(═O)(OH)(R^{D}); and
each R^{D} is independently C₁₋₆ alkyl.

69. The compound of claim 43, or a pharmaceutically acceptable salt, solvate, stereoisomer, or prodrug thereof, wherein the compound has the structure of Formula (D), or a pharmaceutically acceptable salt, solvate, stereoisomer, or prodrug thereof:

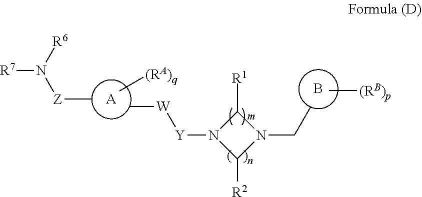

70. The compound of claim 43, or a pharmaceutically acceptable salt, solvate, stereoisomer, or prodrug thereof, wherein the compound has the structure of Formula (D4), or a pharmaceutically acceptable salt, solvate, stereoisomer, or prodrug thereof:

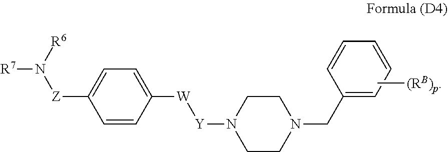

71. The compound of claim 43, wherein the compound is:
methyl 2-cyclopropyl-5-ethoxy-4-((4-((4-(((2S,3R,4R,5R)-2,3,4,5,6-pentahydroxyhexyl)carbamoyl)phenyl)carbamoyl)piperazin-1-yl)methyl)benzoate;
4-(((2R,3S,4S,5S)-2,3,4,5,6-pentahydroxyhexyl)carbamoyl)phenyl 4-((2-cyclopropyl-5-ethoxy-4′-fluoro-[1,1′-biphenyl]-4-yl)methyl)piperazine-1-carboxylate;
4-((4-((2-hydroxyethyl)amino)-4-oxobutyl)carbamoyl)phenyl 4-(4-cyano cyclopropyl-2-ethoxybenzyl)piperazine-1-carboxylate;
4-(((2S,3R,4R,5R)-2,3,4,5,6-pentahydroxyhexyl)carbamoyl)phenyl 4-(5-cyclopropyl-2-ethoxy-4-(methoxycarbonyl)benzyl)piperazine-1-carboxylate;
methyl 2-cyclopropyl-5-ethoxy-4-((4-(2-(4-(((2S,3R,4R,5R)-2,3,4,5,6-pentahydroxyhexyl)carbamoyl)phenyl)acetyl)piperazin-1-yl)methyl)benzoate;
3-(((2S,3R,4R,5R)-2,3,4,5,6-pentahydroxyhexyl)carbamoyl)phenyl 4-(5-cyclopropyl-2-ethoxy-4-(methoxycarbonyl)benzyl)piperazine-1-carboxylate;
4-(3-((((2S,3R,4R,5R)-2,3,4,5,6-pentahydroxyhexyl)amino)methyl)azetidine-1-carbonyl)phenyl 4-(5-cyclopropyl-2-ethoxy-4-(methoxycarbonyl)benzyl)piperazine-1-carboxylate;
or a pharmaceutically acceptable salt, solvate, stereoisomer, or prodrug thereof.

72. A pharmaceutical composition comprising a compound of any one of claims 1-71, or a pharmaceutically acceptable salt, solvate, stereoisomer, or prodrug thereof, and at least one pharmaceutically acceptable excipient.

73. A method of treating a condition or disorder involving the gut-brain axis in a subject in need thereof, the method comprising administering to the subject a therapeutically effective amount of a compound of any one of claims 1-71, or a pharmaceutically acceptable salt, solvate, stereoisomer, or prodrug thereof.

74. The method of claim 73, wherein the condition or disorder is associated with SSTR5 activity.

75. The method of claim 73 or 74, wherein the condition or disorder is a metabolic disorder.

76. The method of claim 75, wherein the condition or disorder is type 2 diabetes, hyperglycemia, metabolic syndrome, obesity, hypercholesterolemia, nonalcoholic steatohepatitis, or hypertension.

77. The method of claim 73 or 74, wherein the condition or disorder is a nutritional disorder.

78. The method of claim 77, wherein the condition or disorder is short bowel syndrome, intestinal failure, or intestinal insufficiency.

79. A method of augmenting weight loss or preventing weigth gain or weight regain, the method comprising administering to the subject a therapeutically effective amount of a compound of any one of claims 1-71, or a pharmaceutically acceptable salt, solvate, stereoisomer, or prodrug thereof.

80. The method of claim 79, wherein the subject has had bariatric surgery.

81. A method of treating gastrointestinal injury resulting from toxic insults such as radiation or chemotherapy in a subject in need thereof, the method comprising administering to the subject a therapeutically effective amount of a compound of any one of claims 1-71, or a pharmaceutically acceptable salt, solvate, stereoisomer, or prodrug thereof.

82. The method of any one of claims 73-81, wherein the compound is gut-restricted.

83. The method of claim 82, wherein the compound has low systemic exposure.

84. The method of any one of claims 73-83, further comprising administering one or more additional therapeutic agents to the subject.

85. The method of claim 84, wherein the one or more additional therapeutic agents are selected from a TGR5 agonist, a GPR40 agonist, a GPR119 agonist, a CCK1 agonist, a PDE4 inhibitor, a DPP-4 inhibitor, a GLP-1 receptor agonist, metformin, or a combination thereof.

86. The method of claim 85, wherein the TGR5 agonist, GPR40 agonist, GPR119 agonist, or CCK1 agonist is gut-restricted.
